(19) United States
(12) Patent Application Publication
(10) Pub. No.: US 2010/0199380 A1
(43) Pub. Date: Aug. 5, 2010

(54) PLANTS HAVING ENHANCED YIELD-RELATED TRAITS AND A METHOD FOR MAKING THE SAME

(75) Inventors: Valerie Frankard, Waterloo (BE); Ana Isabel Sanz Molinero, Gentbrugge (BE); Christophe Reuzeau, Tocan Saint Apre (FR)

Correspondence Address: CONNOLLY BOVE LODGE & HUTZ, LLP, P O BOX 2207, WILMINGTON, DE, 19899, US

(73) Assignee: BASF Plant Science GmbH, Ludwigshafen (DE)

(21) Appl. No.: 12/666,317

(22) PCT Filed: Jun. 30, 2008
(86) PCT No.: PCT/EP2008/058370

§ 371 Date: Dec. 23, 2009

Related U.S. Application Data
(60) Provisional application No. 61/057,921, filed on Jun. 2, 2008.

(30) Foreign Application Priority Data
Jun. 29, 2007 (EP) 07111425.0
Jul. 17, 2007 (EP) 07112614.8
Jul. 17, 2007 (EP) 07112616.3
Jul. 18, 2007 (EP) 07112724.5
Jul. 18, 2007 (EP) 07112725.2
Jun. 2, 2008 (EP) 08157433.7

(51) Int. Cl. A01H 1/00; A01H 5/00; C12N 15/74; C12N 5/10

(52) U.S. Cl. 800/278; 800/298; 435/320.1; 435/419

(57) ABSTRACT

The present invention relates generally to the field of molecular biology and concerns a method for enhancing various economically important yield-related traits in plants. More specifically, the present invention concerns a method for enhancing yield-related traits in plants by modulating expression in a plant of a nucleic acid encoding a yield increasing polypeptide. The present invention also concerns plants having modulated expression of a nucleic acid encoding a yield increasing polypeptide, which plants have enhanced yield-related traits relative to control plants.

The present invention relates generally to the field of molecular biology and concerns a method for enhancing various plant yield-related traits by modulating expression in a plant of a nucleic acid encoding a yield increasing polypeptide selected from the group consisting of:
bHLH6-like (basic Helix-Loop-Helix 6-like) protein,
a GRP (Growth Regulating Protein), wherein said GRP is selected from the group consisting of:
   an RrmJ/FtsJ ribosomal RNA methyltransferase polypeptide (RrmJ/FtsJ polypeptide)
   a basic-helix-loop-helix 4 (bHLH4) polypeptide
   an isopentenyl transferase (IPT) polypeptide
an STO (Salt Tolerance) protein
a UGE (UDP-Glucose 4-Epimerase or UDP-Gal 4-Epimerase) polypeptide.

The present invention also concerns plants having modulated expression of a nucleic acid encoding a yield increasing polypeptide, which plants have enhanced yield-related relative to corresponding wild type plants or other control plants. The invention also provides constructs useful in the methods of the invention.

The ever-increasing world population and the dwindling supply of arable land available for agriculture fuels research towards increasing the efficiency of agriculture. Conventional means for crop and horticultural improvements utilise selective breeding techniques to identify plants having desirable characteristics. However, such selective breeding techniques have several drawbacks, namely that these techniques are typically labour intensive and result in plants that often contain heterogeneous genetic components that may not always result in the desirable trait being passed on from parent plants. Advances in molecular biology have allowed mankind to modify the germplasm of animals and plants. Genetic engineering of plants entails the isolation and manipulation of genetic material (typically in the form of DNA or RNA) and the subsequent introduction of that genetic material into a plant. Such technology has the capacity to deliver crops or plants having various improved economic, agronomic or horticultural traits.

A trait of particular economic interest is increased yield. Yield is normally defined as the measurable produce of economic value from a crop. This may be defined in terms of quantity and/or quality. Yield is directly dependent on several factors, for example, the number and size of the organs, plant architecture (for example, the number of branches), seed production, leaf senescence and more. Root development, nutrient uptake, stress tolerance and early vigour may also be important factors in determining yield. Optimizing the above-mentioned factors may therefore contribute to increasing crop yield.

Seed yield is a particularly important trait, since the seeds of many plants are important for human and animal nutrition. Crops such as corn, rice, wheat, canola and soybean account for over half the total human caloric intake, whether through direct consumption of the seeds themselves or through consumption of meat products raised on processed seeds. They are also a source of sugars, oils and many kinds of metabolites used in industrial processes. Seeds contain an embryo (the source of new shoots and roots) and an endosperm (the source of nutrients for embryo growth during germination and during early growth of seedlings). The development of a seed involves many genes, and requires the transfer of metabolites from the roots, leaves and stems into the growing seed. The endosperm, in particular, assimilates the metabolic precursors of carbohydrates, oils and proteins and synthesizes them into storage macromolecules to fill out the grain.

A further interesting trait is the flowering time of a plant. The life span of a plant can be divided in phases such as germination, vegetative growth, reproductive growth and senescence. The flowering time is the time elapsed between sowing and start of reproductive growth. It is a crucial moment in the life of a plant that determines the transition from vegetative to reproductive growth, which in some plants coincides with the start of senescence. In many plants, this is the point in time at which the shoot apical meristem stops making leaves and starts making flowers which has a great impact on morphogenesis affecting, for example, the number of organs formed and the overall size and shape of the plant. The flowering time also impacts other yield-related traits in plants. Typically an early flowering variety shows less branching or tillering and therefore is less bushy. Such traits may be advantageous to the farmer to, for example, simplify crop management. On the other hand, delayed flowering may result in plants with more vegetative organs, for example more leaves which is a desirable trait in many crops, particularly in crops where the vegetative organs are harvested, such as lettuce. The relative duration of vegetative and reproductive phase of a plant directly affects its seed yield. In some plants, control of flowering time is a mechanism used to avoid negative impact of stresses such as drought. Flowering time may also affect quality traits of crops, for example herbage quality in forage crops, where delay in flowering may result in higher digestibility. The flowering time affects the length of the cultivation season. Modification of flowering time of a crop may result in the possibility to extend the geographic area of cultivation and therefore increase the cultivated acreage. It may also result in plants being more amenable to agriculture in a given environment, for example early flowering may allow late planting in areas where crop establishment may be negatively affected by low temperatures or may allow early harvest to avoid biotic and abiotic pressure at the end of the season, resulting therefore in an increase in the yield of the crop. Therefore, the ability to control flowering time is an important factor with many industrial applications in the field of agriculture.

Another important trait for many crops is early vigour. Improving early vigour is an important objective of modern rice breeding programs in both temperate and tropical rice cultivars. Long roots are important for proper soil anchorage in water-seeded rice. Where rice is sown directly into flooded fields, and where plants must emerge rapidly through water, longer shoots are associated with vigour. Where drill-seeding is practiced, longer mesocotyls and coleoptiles are important for good seedling emergence. The ability to engineer early vigour into plants would be of great importance in agriculture. For example, poor early vigour has been a limitation to the introduction of maize (Zea mays L.) hybrids based on Corn Belt germplasm in the European Atlantic.

A further important trait is that of improved abiotic stress tolerance. Abiotic stress is a primary cause of crop loss worldwide, reducing average yields for most major crop plants by more than 50% (Wang et al., Planta (2003) 218: 1-14). Abiotic stresses may be caused by drought, salinity, extremes of temperature, chemical toxicity, from 59348: excess or lack of nutrients (macroelements and/or microelemtens), radiation and oxidative stress. The ability to improve plant tolerance to abiotic stress would be of great economic advantage to farmers worldwide and would allow for the cultivation of crops during adverse conditions and in territories where cultivation of crops may not otherwise be possible.

Crop yield may therefore be increased by optimising one of the above-mentioned factors.

Depending on the end use, the modification of certain yield traits may be favoured over others. For example for applications such as forage or wood production, or bio-fuel resource, an increase in the vegetative parts of a plant may be desirable, and for applications such as flour, starch or oil production, an increase in seed parameters may be particularly desirable. Even amongst the seed parameters, some may be favoured over others, depending on the application. Various mechanisms may contribute to increasing seed yield, whether that is in the form of increased seed size or increased seed number.

One approach to increasing yield (seed yield and/or biomass) in plants may be through modification of the inherent growth mechanisms of a plant, such as the cell cycle or various signalling pathways involved in plant growth or in defense mechanisms.

It has now been found that various yield-related traits may be enhanced in plants by modulating expression in a plant of a nucleic acid encoding a

yield increasing polypeptide selected from the group consisting of:
bHLH6-like (basic Helix-Loop-Helix 6-like) protein,
a GRP (Growth Regulating Protein), wherein said GRP is selected from the group consisting of:
   an RrmJ/FtsJ ribosomal RNA methyltransferase polypeptide (RrmJ/FtsJ polypeptide)
   a basic-helix-loop-helix 4 (bHLH4) polypeptide
   an isopentenyl transferase (IPT) polypeptide
an STO (Salt Tolerance) protein
a UGE (UDP-Glucose 4-Epimerase or UDP-Gal 4-Epimerase) polypeptide.

### BACKGROUND

bHLH6-Like (Basic Helix-Loop-Helix 6-Like) Protein

Transcription factors are usually defined as proteins that show sequence-specific DNA binding and that are capable of activating and/or repressing transcription. The basic Helix-Loop-Helix transcription factor family is one of the largest families of transcription factors that have been characterised in Arabidopsis thaliana (Toledo-Ortiz et al., Plant Cell 15, 1749-1770, 2003; Bailey et al., Plant Cell 15, 2497-2501, 2003) and in rice (Li et al Plant Physiol. 141, 1167-1184, 2006). The distinguishing characteristic of the bHLH transcription factor family is the presence of a bipartite domain consisting of approximately 60 amino acids. This bipartite domain is comprised of a DNA-binding basic region, which binds to a consensus hexanucleotide E-box and two α-helices separated by a variable loop region, located C-terminally of the basic domain. The two α-helices promote dimerisation, allowing the formation of homo- and heterodimers between different family members. While the bHLH domain is evolutionarily conserved, there is little sequence similarity between clades beyond the domain. Li et al. (2006) classify the rice and Arabidopsis bHLH transcription factors into 22 subfamilies, based on the sequence of the bHLH domains.

AtbHLH6 (AtMYC2) is a 68 kDa MYC-related transcriptional activator with a bHLH type DNA-binding domain (Abe et al, Plant Cell 9, 1859-1868, 1997). It is induced by dehydration stress and upon treatment with abscisic acid (ABA). AtMYC2 recognises a MYC recognition site in the promoter of the rd22 gene, an ABA responsive protein without an ABRE-element in the promoter. Plants overexpressing AtMYC2 have a higher sensitivity to ABA, with enhanced ABA-induced gene expression, whereas an insertion mutant showed the opposite (reduced ABA sensitivity and decreased ABA-induced gene expression); furthermore AtMYC2 overexpressing plants were more resistant to osmotic stress (Abe et al., Plant Cell 15, 63-78, 2003). Plants overexpressing AtMYC2 reportedly did not show morphological changes compared to wild type plants and also the cell shape in leaves was not affected. Furthermore, AtMYC2 was shown to be involved in pathogen and wound responses; in tryptophan and tryptophan-derived secondary metabolism and in tolerance to methyl viologen (Paraquat) (Dombrecht et al., Plant Cell 19, 2225-2245, 2007).

### STO (Salt Tolerance) Protein

STO protein, Salt Tolerance Protein, was first identified from Arabidopsis thaliana in a screen for genes suppressing defective salt response in a calcienurin mutant in yeast (Lippuner et al. 1996). Methods for conferring salt tolerance in a plant by expressing the Arabidopsis STO have been previously disclosed (U.S. Pat. No. 5,859,337; Nagaoka and Takano 2003, J. Exp. Bot. 54, 391-396).

Structurally STO is characterized by the presence of a well-known domain called the B-box domain in which the structure CX2CX16CX2C is conserved. The B-box domain is a promiscuous domain present in proteins of archeobacterial, prokaryotic and eukaryotic origin. It is found essentially in transcription factors, ribonucleoproteins and protooncoproteins. The B-box domain in STO is structurally similar to that found in the Arabidopsis thaliana protein CONSTANS, a transcription factor involved in repression of flowering time. However STO and CONSTANs belong to different protein families, with CONSTANS being characterized by the presence of a plant-specific conserved domain, the so called the CCT box in addition to the B-box, while in the STO family of proteins only the B-box is present (Griffiths et al. 2003, Plant Phys, 131, 1855-1867; Lagercrants et al. 2000 Mol. Biol. Evol. 17, 1499-1507).

The biological function of STO in Arabidopsis has been further investigated using gain and loss of function mutants (Indorf, et al. 2007, Plant J. 51(4):563-74.). Data reported from those studies suggested a role for STO in light signalling where it was mentioned that STO could act as a negative regulator in phytochrome and blue light signalling. These studies were directed to understanding, the role of STO in the salt stress and light response.

### UGE (UDP-Glucose 4-Epimerase or UDP-Gal 4-Epimerase) Polypeptide

The biosynthesis of carbohydrates in plants requires specific glycosyl-transferases that act on activated sugars, typically uridine (UDP), adenosine or guanosine diphosphate hexoses and pentoses. Nucleotide sugars are modified at their glycosyl moities by nucleotide sugar interconversion enzymes to generate different sugars that are intermediates in the uptake of the free sugars released from the breakdown of nutritional or storage carbohydrates and other sources. One of the best-characterized nucleotide sugar interconversion enzymes is UGE (EC.5.1.3.2 according to the IUBMB—International Union of Biochemistry and Molecular Biology—Enzyme Nomenclature).

UGE, UDP-Glucose 4-Epimerase or UDP-Gal 4-Epimerase (EC.5.1.3.2) catalyses the interconversion of UDP-glucose and UDP-galactose. Varying concentrations of the cofactor NAD+/NADH (Nicotinamide Adenine Dinucleotide-oxidized/reduced-), can result in differential stimulation of the reaction. X-ray crystallography and other studies suggest that UGEs are dimeric, although active monomers and higher polymers have also been reported under certain experimental conditions (Thoden et al. 2005 J. Biol. Chem 280, 21900-21907).

UGE is essential for de novo biosynthesis of UDP-Gal, a precursor for the biosynthesis of numerous different carbohydrates, glycolipids and glycosides. UGE is also required for the catabolic uptake of galactose into the central metabolism, and therefore UGE deficiency exarcerbates galactose toxicity in plants (Dormann and Benning, 1998; Plant J. 13, 641-652) and in yeast and it also leads to different forms of human galactosemia.

UGE enzymes are encoded in the genome of microorganisms as well as of higher organisms. In the model plant Arabidopsis thaliana five different paralogous genes enconding UGE isoforms have been identified. The rice and poplar genomes encode at least seven predicted UGE isoforms.

The enzymatic properties and genetic role of UGE isoforms has been addressed in Arabidopsis thaliana in biochemical studies as well as by characterization of gain and loss of function mutants. All five Arabidopsis UGEs are enzymatically active in similar ranges of temperature (30-40 C) and PH (PH7-PH9). Homo and heterodimers can be formed between UGE isoforms. The five isoforms are functional in vivo and can complement the loss of function in the yeast GAL10 mutant lacking UGE function. The latter reflects the functional conservation of UGE enzymatic activities between UGEs of microorganisms and higher plants (Barber et al. 2006; JBC 281, 17276-17285). In Arabidopsis the 5 paralogous genes encoding UGEs are expressed preferentially in the roots. Mutations or loss of UGE function typically resulted in general growth defects of vegetative organs and flowers were smaller and abnormally shaped. Surprisingly the floral number and position was not altered, though sterility was reported (Rosti et al. 2007; The Plant Cell 19, 1565-1579). In accordance with UGE activity the cell wall galactose content in these mutants was altered. U.S. Pat. No. 6,992,236 discloses plant-derived UGE genes and teaches methods to express them in plants to allow modification of carbohydrate metabolism in plants.

### SUMMARY

Surprisingly, it has now been found that modulating expression of a nucleic acid encoding a yield increasing polypeptide selected from the group consisting of:
bHLH6-like (basic Helix-Loop-Helix 6-like) protein,
a GRP (Growth Regulating Protein), wherein said GRP is selected from the group consisting of:
   an RrmJ/FtsJ ribosomal RNA methyltransferase polypeptide (RrmJ/FtsJ polypeptide)
   a basic-helix-loop-helix 4 (bHLH4) polypeptide
   an isopentenyl transferase (IPT) polypeptide
an STO (Salt Tolerance) protein
a UGE (UDP-Glucose 4-Epimerase or UDP-Gal 4-Epimerase) polypeptide gives plants having enhanced yield-related traits relative to control plants.

According one embodiment, there is provided a method for enhancing yield related traits of a plant relative to control plants, comprising modulating expression of a nucleic acid encoding a bHLH6-like polypeptide in a plant. The enhanced yield related traits comprised increased increased yield and increased emergence vigour.

According one embodiment, there is provided a method for enhancing yield-related traits of a plant relative to control plants, comprising modulating expression of a nucleic acid sequence encoding a GRP polypeptide in a plant, wherein said GRP polypeptide is an RrmJ/FtsJ ribosomal RNA methyltransferase polypeptide (RrmJ/FtsJ polypeptide). The enhanced yield-related traits comprise increased seed yield, increased number of filled seeds, increased seed fill rate, and increased harvest index.

According to one embodiment, there is provided a method for enhancing yield-related traits of a plant relative to control plants, comprising modulating expression (preferably, increasing) of a nucleic acid sequence encoding a GRP polypeptide in a plant, wherein said GRP polypeptide is a basic-helix-loop-helix 4 (bHLH4) polypeptide. The enhanced yield-related traits are preferably enhanced seed yield-related traits, comprising one or more of: increased early vigour, increased greenness index, increased total seed yield per plant, increased number of filled seeds, increased seed fill rate, increased, and increased harvest index.

According to one embodiment, there is provided a method for enhancing yield-related traits of a plant relative to control plants, comprising modulating expression (preferably, increasing) of a nucleic acid sequence encoding a GRP polypeptide in a plant, wherein said GRP polypeptide is an isopentenyl transferase (IPT) polypeptide. The enhanced yield-related traits are one or more of: increased total seed yield per plant, increased number of filled seeds, increased total number of seeds, and increased harvest index.

According one embodiment, there is provided a method for enhancing yield-related traits plants, particularly increasing early vigour and altering flowering time, particularly shortening flowering time relative to control plants, comprising modulating expression of a nucleic acid encoding an STO polypeptide in a plant.

According to one embodiment, there is provided a method for enhancing yield related traits of a plant relative to control plants, in particular increased yield, comprising modulating expression of a nucleic acid encoding a UGE polypeptide in a plant.

### DEFINITIONS

The terms improving and/or enhancing and/or improved and/or enhanced plant yield-related traits are used herein encompass improving and/or enhancing and/or improved and/or enhanced plant growth characteristics.

### Polypeptide(s)/Protein(s)

The terms “polypeptide” and “protein” are used interchangeably herein and refer to amino acids in a polymeric form of any length, linked together by peptide bonds.

### Polynucleotide(S)/Nucleic Acid(S)/Nucleic Acid Sequence(S)/Nucleotide Sequence(S)

The terms “polynucleotide(s)”, “nucleic acid sequence(s)”, “nucleotide sequence(s)”, “nucleic acid(s)”, “nucleic acid molecule” are used interchangeably herein and refer to nucleotides, either ribonucleotides or deoxyribonucleotides or a combination of both, in a polymeric unbranched form of any length.

### Control Plant(s)

The choice of suitable control plants is a routine part of an experimental setup and may include corresponding wild type plants or corresponding plants without the gene of interest. The control plant is typically of the same plant species or even of the same variety as the plant to be assessed. The control plant may also be a nullizygote of the plant to be assessed.

Nullizygotes are individuals missing the transgene by segregation. A “control plant” as used herein refers not only to whole plants, but also to plant parts, including seeds and seed parts.

### Homoloque(s)

“Homologues” of a protein encompass peptides, oligopeptides, polypeptides, proteins and enzymes having amino acid substitutions, deletions and/or insertions relative to the unmodified protein in question and having similar biological and functional activity as the unmodified protein from which they are derived.

A deletion refers to removal of one or more amino acids from a protein.

An insertion refers to one or more amino acid residues being introduced into a predetermined site in a protein. Insertions may comprise N-terminal and/or C-terminal fusions as well as intra-sequence insertions of single or multiple amino acids. Generally, insertions within the amino acid sequence will be smaller than N- or C-terminal fusions, of the order of about 1 to 10 residues. Examples of N- or C-terminal fusion proteins or peptides include the binding domain or activation domain of a transcriptional activator as used in the yeast two-hybrid system, phage coat proteins, (histidine)-6-tag, glutathione S-transferase-tag, protein A, maltose-binding protein, dihydrofolate reductase, Tag•100 epitope, c-myc epitope, FLAG®-epitope, lacZ, CMP (calmodulin-binding peptide), HA epitope, protein C epitope and VSV epitope.

A substitution refers to replacement of amino acids of the protein with other amino acids having similar properties (such as similar hydrophobicity, hydrophilicity, antigenicity, propensity to form or break α-helical structures or β-sheet structures). Amino acid substitutions are typically of single residues, but may be clustered depending upon functional constraints placed upon the polypeptide; insertions will usually be of the order of about 1 to 10 amino acid residues. The amino acid substitutions are preferably conservative amino acid substitutions. Conservative substitution tables are well known in the art (see for example Creighton (1984) Proteins. W.H. Freeman and Company (Eds) and Table 1 below).

TABLE 1
Examples of conserved amino acid substitutions

| | Conservative |
|---|---|
| Residue | Substitutions |
| Ala | Ser |
| Arg | Lys |
| Asn | Gln; His |
| Asp | Glu |
| Gln | Asn |
| Cys | Ser |
| Glu | Asp |
| Gly | Pro |
| His | Asn; Gln |
| Ile | Leu, Val |
| Leu | Ile; Val |
| Lys | Arg; Gln |
| Met | Leu; Ile |
| Phe | Met; Leu; Tyr |
| Ser | Thr; Gly |
| Thr | Ser; Val |
| Trp | Tyr |
| Tyr | Trp; Phe |
| Val | Ile; Leu |

Amino acid substitutions, deletions and/or insertions may readily be made using peptide synthetic techniques well known in the art, such as solid phase peptide synthesis and the like, or by recombinant DNA manipulation. Methods for the manipulation of DNA sequences to produce substitution, insertion or deletion variants of a protein are well known in the art. For example, techniques for making substitution mutations at predetermined sites in DNA are well known to those skilled in the art and include M13 mutagenesis, T7-Gen in vitro mutagenesis (USB, Cleveland, Ohio), QuickChange Site Directed mutagenesis (Stratagene, San Diego, Calif.), PCR-mediated site-directed mutagenesis or other site-directed mutagenesis protocols.

### Derivatives

“Derivatives” include peptides, oligopeptides, polypeptides which may, compared to the amino acid sequence of the naturally-occurring form of the protein, such as the protein of interest, comprise substitutions of amino acids with non-naturally occurring amino acid residues, or additions of non-naturally occurring amino acid residues. “Derivatives” of a protein also encompass peptides, oligopeptides, polypeptides which comprise naturally occurring altered (glycosylated, acylated, prenylated, phosphorylated, myristoylated, sulphated etc.) or non-naturally altered amino acid residues compared to the amino acid sequence of a naturally-occurring form of the polypeptide. A derivative may also comprise one or more non-amino acid substituents or additions compared to the amino acid sequence from which it is derived, for example a reporter molecule or other ligand, covalently or non-covalently bound to the amino acid sequence, such as a reporter molecule which is bound to facilitate its detection, and non-naturally occurring amino acid residues relative to the amino acid sequence of a naturally-occurring protein. Furthermore, “derivatives” also include fusions of the naturally-occurring form of the protein with tagging peptides such as FLAG, HIS6 or thioredoxin (for a review of tagging peptides, see Terpe, Appl. Microbiol. Biotechnol. 60, 523-533, 2003).

### Ortholoque(s)/Paraloque(s)

Orthologues and paralogues encompass evolutionary concepts used to describe the ancestral relationships of genes. Paralogues are genes within the same species that have originated through duplication of an ancestral gene; orthologues are genes from different organisms that have originated through speciation, and are also derived from a common ancestral gene.

### Domain

The term “domain” refers to a set of amino acids conserved at specific positions along an alignment of sequences of evolutionarily related proteins. While amino acids at other positions can vary between homologues, amino acids that are highly conserved at specific positions indicate amino acids that are likely essential in the structure, stability or function of a protein. Identified by their high degree of conservation in aligned sequences of a family of protein homologues, they can be used as identifiers to determine if any polypeptide in question belongs to a previously identified polypeptide family.

### Motif/Consensus Sequence/Signature

The term “motif” or “consensus sequence” or “signature” refers to a short conserved region in the sequence of evolutionarily related proteins. Motifs are frequently highly conserved parts of domains, but may also include only part of the domain, or be located outside of conserved domain (if all of the amino acids of the motif fall outside of a defined domain).

### Hybridisation

The term “hybridisation” as defined herein is a process wherein substantially homologous complementary nucleotide sequences anneal to each other. The hybridisation process can occur entirely in solution, i.e. both complementary nucleic acids are in solution. The hybridisation process can also occur with one of the complementary nucleic acids immobilised to a matrix such as magnetic beads, Sepharose beads or any other resin. The hybridisation process can furthermore occur with one of the complementary nucleic acids immobilised to a solid support such as a nitro-cellulose or nylon membrane or immobilised by e.g. photolithography to, for example, a siliceous glass support (the latter known as nucleic acid arrays or microarrays or as nucleic acid chips). In order to allow hybridisation to occur, the nucleic acid molecules are generally thermally or chemically denatured to melt a double strand into two single strands and/or to remove hairpins or other secondary structures from single stranded nucleic acids.

The term “stringency” refers to the conditions under which a hybridisation takes place. The stringency of hybridisation is influenced by conditions such as temperature, salt concentration, ionic strength and hybridisation buffer composition. Generally, low stringency conditions are selected to be about 30° C. lower than the thermal melting point (Tₘ) for the specific sequence at a defined ionic strength and pH. Medium stringency conditions are when the temperature is 20° C. below Tₘ, and high stringency conditions are when the temperature is 10° C. below Tₘ. High stringency hybridisation conditions are typically used for isolating hybridising sequences that have high sequence similarity to the target nucleic acid sequence. However, nucleic acids may deviate in sequence and still encode a substantially identical polypeptide, due to the degeneracy of the genetic code. Therefore medium stringency hybridisation conditions may sometimes be needed to identify such nucleic acid molecules.

The Tm is the temperature under defined ionic strength and pH, at which 50% of the target sequence hybridises to a perfectly matched probe. The Tₘ is dependent upon the solution conditions and the base composition and length of the probe. For example, longer sequences hybridise specifically at higher temperatures. The maximum rate of hybridisation is obtained from about 16° C. up to 32° C. below Tₘ. The presence of monovalent cations in the hybridisation solution reduce the electrostatic repulsion between the two nucleic acid strands thereby promoting hybrid formation; this effect is visible for sodium concentrations of up to 0.4M (for higher concentrations, this effect may be ignored). Formamide reduces the melting temperature of DNA-DNA and DNA-RNA duplexes with 0.6 to 0.7° C. for each percent formamide, and addition of 50% formamide allows hybridisation to be performed at 30 to 45° C., though the rate of hybridisation will be lowered. Base pair mismatches reduce the hybridisation rate and the thermal stability of the duplexes. On average and for large probes, the Tm decreases about 1° C. per % base mismatch. The Tm may be calculated using the following equations, depending on the types of hybrids:
1) DNA-DNA hybrids (Meinkoth and Wahl, Anal. Biochem., 138: 267-284, 1984):

Tₘ=81.5° C.+16.6×log₁₀[Na⁺]^{a}+0.41×%[G/C^{b}]−500×[L^{c}]⁻¹−0.61×% formamide

2) DNA-RNA or RNA-RNA hybrids:

Tm=79.8+18.5(log₁₀[Na⁺]^{a})+0.58 (% G/C^{b})+11.8(% G/C^{b})²−820/L^{c}

3) oligo-DNA or oligo-RNA^{d} hybrids:

For <20 nucleotides: Tₘ=2(lₙ)

For 20-35 nucleotides: Tₘ=22+1.46(lₙ)

^{a} or for other monovalent cation, but only accurate in the 0.01-0.4 M range.
^{b} only accurate for % GC in the 30% to 75% range.
^{c} L=length of duplex in base pairs.
^{d} oligo, oligonucleotide; lₙ,=effective length of primer=2×(no. of G/C)+(no. of A/T).

Non-specific binding may be controlled using any one of a number of known techniques such as, for example, blocking the membrane with protein containing solutions, additions of heterologous RNA, DNA, and SDS to the hybridisation buffer, and treatment with Rnase. For non-homologous probes, a series of hybridizations may be performed by varying one of (i) progressively lowering the annealing temperature (for example from 68° C. to 42° C.) or (ii) progressively lowering the formamide concentration (for example from 50% to 0%). The skilled artisan is aware of various parameters which may be altered during hybridisation and which will either maintain or change the stringency conditions.

Besides the hybridisation conditions, specificity of hybridisation typically also depends on the function of post-hybridisation washes. To remove background resulting from non-specific hybridisation, samples are washed with dilute salt solutions. Critical factors of such washes include the ionic strength and temperature of the final wash solution: the lower the salt concentration and the higher the wash temperature, the higher the stringency of the wash. Wash conditions are typically performed at or below hybridisation stringency. A positive hybridisation gives a signal that is at least twice of that of the background. Generally, suitable stringent conditions for nucleic acid hybridisation assays or gene amplification detection procedures are as set forth above. More or less stringent conditions may also be selected. The skilled artisan is aware of various parameters which may be altered during washing and which will either maintain or change the stringency conditions.

For example, typical high stringency hybridisation conditions for DNA hybrids longer than 50 nucleotides encompass hybridisation at 65° C. in 1×SSC or at 42° C. in 1×SSC and 50% formamide, followed by washing at 65° C. in 0.3×SSC. Examples of medium stringency hybridisation conditions for DNA hybrids longer than 50 nucleotides encompass hybridisation at 50° C. in 4×SSC or at 40° C. in 6×SSC and 50% formamide, followed by washing at 50° C. in 2×SSC. The length of the hybrid is the anticipated length for the hybridising nucleic acid. When nucleic acids of known sequence are hybridised, the hybrid length may be determined by aligning the sequences and identifying the conserved regions described herein. 1×SSC is 0.15M NaCl and 15 mM sodium citrate; the hybridisation solution and wash solutions may additionally include 5×Denhardt's reagent, 0.5-1.0% SDS, 100 μg/ml denatured, fragmented salmon sperm DNA, 0.5% sodium pyrophosphate.

For the purposes of defining the level of stringency, reference can be made to Sambrook et al. (2001) Molecular Cloning: a laboratory manual, 3^{rd} Edition, Cold Spring Harbor Laboratory Press, CSH, New York or to Current Protocols in Molecular Biology, John Wiley & Sons, N.Y. (1989 and yearly updates).

### Splice Variant

The term “splice variant” as used herein encompasses variants of a nucleic acid sequence in which selected introns and/or exons have been excised, replaced, displaced or added, or in which introns have been shortened or lengthened. Such variants will be ones in which the biological activity of the protein is substantially retained; this may be achieved by selectively retaining functional segments of the protein. Such splice variants may be found in nature or may be manmade. Methods for predicting and isolating such splice variants are well known in the art (see for example Foissac and Schiex (2005) BMC Bioinformatics 6: 25).

### Allelic Variant

Alleles or allelic variants are alternative forms of a given gene, located at the same chromosomal position. Allelic variants encompass Single Nucleotide Polymorphisms (SNPs), as well as Small Insertion/Deletion Polymorphisms (INDELs). The size of INDELs is usually less than 100 bp. SNPs and INDELs form the largest set of sequence variants in naturally occurring polymorphic strains of most organisms.

### Gene Shuffling/Directed Evolution

Gene shuffling or directed evolution consists of iterations of DNA shuffling followed by appropriate screening and/or selection to generate variants of nucleic acids or portions thereof encoding proteins having a modified biological activity (Castle et al., (2004) Science 304(5674): 1151-4; U.S. Pat. Nos. 5,811,238 and 6,395,547).

### Regulatory Element/Control Sequence/Promoter

The terms “regulatory element”, “control sequence” and “promoter” are all used interchangeably herein and are to be taken in a broad context to refer to regulatory nucleic acid sequences capable of effecting expression of the sequences to which they are ligated. The term “promoter” typically refers to a nucleic acid control sequence located upstream from the transcriptional start of a gene and which is involved in recognising and binding of RNA polymerase and other proteins, thereby directing transcription of an operably linked nucleic acid. Encompassed by the aforementioned terms are transcriptional regulatory sequences derived from a classical eukaryotic genomic gene (including the TATA box which is required for accurate transcription initiation, with or without a CCAAT box sequence) and additional regulatory elements (i.e. upstream activating sequences, enhancers and silencers) which alter gene expression in response to developmental and/or external stimuli, or in a tissue-specific manner. Also included within the term is a transcriptional regulatory sequence of a classical prokaryotic gene, in which case it may include a −35 box sequence and/or −10 box transcriptional regulatory sequences. The term “regulatory element” also encompasses a synthetic fusion molecule or derivative that confers, activates or enhances expression of a nucleic acid molecule in a cell, tissue or organ.

A “plant promoter” comprises regulatory elements, which mediate the expression of a coding sequence segment in plant cells. Accordingly, a plant promoter need not be of plant origin, but may originate from viruses or micro-organisms, for example from viruses which attack plant cells. The “plant promoter” can also originate from a plant cell, e.g. from the plant which is transformed with the nucleic acid sequence to be expressed in the inventive process and described herein. This also applies to other “plant” regulatory signals, such as “plant” terminators. The promoters upstream of the nucleotide sequences useful in the methods of the present invention can be modified by one or more nucleotide substitution(s), insertion(s) and/or deletion(s) without interfering with the functionality or activity of either the promoters, the open reading frame (ORF) or the 3′-regulatory region such as terminators or other 3′ regulatory regions which are located away from the ORF. It is furthermore possible that the activity of the promoters is increased by modification of their sequence, or that they are replaced completely by more active promoters, even promoters from heterologous organisms. For expression in plants, the nucleic acid molecule must, as described above, be linked operably to or comprise a suitable promoter which expresses the gene at the right point in time and with the required spatial expression pattern.

For the identification of functionally equivalent promoters, the promoter strength and/or expression pattern of a candidate promoter may be analysed for example by operably linking the promoter to a reporter gene and assaying the expression level and pattern of the reporter gene in various tissues of the plant. Suitable well-known reporter genes include for example beta-glucuronidase or beta-galactosidase. The promoter activity is assayed by measuring the enzymatic activity of the beta-glucuronidase or beta-galactosidase. The promoter strength and/or expression pattern may then be compared to that of a reference promoter (such as the one used in the methods of the present invention). Alternatively, promoter strength may be assayed by quantifying mRNA levels or by comparing mRNA levels of the nucleic acid used in the methods of the present invention, with mRNA levels of housekeeping genes such as 18S rRNA, using methods known in the art, such as Northern blotting with densitometric analysis of autoradiograms, quantitative real-time PCR or RT-PCR (Heid et al., 1996 Genome Methods 6: 986-994). Generally by “weak promoter” is intended a promoter that drives expression of a coding sequence at a low level. By “low level” is intended at levels of about 1/10,000 transcripts to about 1/100,000 transcripts, to about 1/500,000 transcripts per cell. Conversely, a “strong promoter” drives expression of a coding sequence at high level, or at about 1/10 transcripts to about 1/100 transcripts to about 1/1000 transcripts per cell. An example of a strong promoter is the CaMV 35S promoter.

### Operably Linked

The term “operably linked” as used herein refers to a functional linkage between the promoter sequence and the gene of interest, such that the promoter sequence is able to initiate transcription of the gene of interest.

### Constitutive Promoter

A “constitutive promoter” refers to a promoter that is transcriptionally active during most, but not necessarily all, phases of growth and development and under most environmental conditions, in at least one cell, tissue or organ. Table 2a below gives examples of constitutive promoters.

TABLE 2a
Examples of constitutive promoters

| Gene Source | Reference |
|---|---|
| Actin | McElroy et al, Plant Cell, 2: 163-171, 1990 |
| HMGP | WO 2004/070039 |
| CAMV 35S | Odell et al, Nature, 313: 810-812, 1985 |
| CaMV 19S | Nilsson et al., Physiol. Plant. 100: 456-462, 1997 |
| GOS2 | de Pater et al, Plant J Nov; 2(6): 837-44, 1992, WO |
| | 2004/065596 |
| Ubiquitin | Christensen et al, Plant Mol. Biol. 18: 675-689, 1992 |
| Rice cyclophilin | Buchholz et al, Plant Mol Biol. 25(5): 837-43, 1994 |
| Maize H3 histone | Lepetit et al, Mol. Gen. Genet. 231: 276-285, 1992 |
| Alfalfa H3 | Wu et al. Plant Mol. Biol. 11: 641-649, 1988 |
| histone | |
| Actin 2 | An et al, Plant J. 10(1); 107-121, 1996 |
| 34S FMV | Sanger et al., Plant. Mol. Biol., 14, 1990: 433-443 |
| Rubisco | U.S. Pat. No. 4,962,028 |
| small subunit | |
| OCS | Leisner (1988) Proc Natl Acad Sci USA 85(5): 2553 |
| SAD1 | Jain et al., Crop Science, 39 (6), 1999: 1696 |
| SAD2 | Jain et al., Crop Science, 39 (6), 1999: 1696 |
| nos | Shaw et al. (1984) Nucleic Acids Res. 12(20): |
| | 7831-7846 |
| V-ATPase | WO 01/14572 |
| Super promoter | WO 95/14098 |
| G-box proteins | WO 94/12015 |

### Ubiquitous Promoter

A ubiquitous promoter is active in substantially all tissues or cells of an organism.

### Developmentally-Regulated Promoter

A developmentally-regulated promoter is active during certain developmental stages or in parts of the plant that undergo developmental changes.

### Inducible Promoter

An inducible promoter has induced or increased transcription initiation in response to a chemical (for a review see Gatz 1997, Annu. Rev. Plant Physiol. Plant Mol. Biol., 48:89-108), environmental or physical stimulus, or may be “stress-inducible”, i.e. activated when a plant is exposed to various stress conditions, or a “pathogen-inducible” i.e. activated when a plant is exposed to exposure to various pathogens.

### Organ-Specific/Tissue-Specific Promoter

An organ-specific or tissue-specific promoter is one that is capable of preferentially initiating transcription in certain organs or tissues, such as the leaves, roots, seed tissue etc. For example, a “root-specific promoter” is a promoter that is transcriptionally active predominantly in plant roots, substantially to the exclusion of any other parts of a plant, whilst still allowing for any leaky expression in these other plant parts. Promoters able to initiate transcription in certain cells only are referred to herein as “cell-specific”.

A seed-specific promoter is transcriptionally active predominantly in seed tissue, but not necessarily exclusively in seed tissue (in cases of leaky expression). The seed-specific promoter may be active during seed development and/or during germination. from 59392: Examples of seed-specific promoters are given in Qing Qu and Takaiwa (Plant Biotechnol. J. 2, 113-125, 2004).

### From 59348:

For example, the seed-specific promoter may drive expression in one or more seed tissues such as endosperm, aleurone, embryo. Examples of seed-specific promoters are shown in Table 2b below. Further examples of seed-specific promoters are given in Qing Qu and Takaiwa (Plant Biotechnol. J. 2, 113-125, 2004), which disclosure is incorporated by reference herein as if fully set forth.

TABLE 2b
Examples of seed-specific promoters

| Gene source | Reference |
|---|---|
| seed-specific genes | Simon et al., Plant Mol. Biol. 5: 191, 1985; |
| | Scofield et al., J. Biol. Chem. 262: 12202, 1987.; |
| | Baszczynski et al., Plant Mol. Biol. 14: 633, 1990. |
| Brazil Nut albumin | Pearson et al., Plant Mol. Biol. 18: 235-245, 1992. |
| Legumin | Ellis et al., Plant Mol. Biol. 10: 203-214, 1988. |
| glutelin (rice) | Takaiwa et al., Mol. Gen. Genet. 208: 15-22, 1986; |
| | Takaiwa et al., FEBS Letts. 221: 43-47, 1987. |
| Zein | Matzke et al Plant Mol Biol, 14(3): 323-32 1990 |
| NapA | Stalberg et al, Planta 199: 515-519, 1996. |
| Wheat LMW and HMW glutenin-1 | Mol Gen Genet 216: 81-90, 1989; NAR 17: 461-2, 1989 |
| Wheat SPA | Albani et al, Plant Cell, 9: 171-184, 1997 |
| Wheat α, β, γ-gliadins | EMBO J. 3: 1409-15, 1984 |
| Barley ltr1 promoter | Diaz et al. (1995) Mol Gen Genet 248(5): 592-8 |
| Barley B1, C, D, hordein | Theor Appl Gen 98: 1253-62, 1999; Plant J 4: 343-55, 1993; |
| | Mol Gen Genet 250: 750-60, 1996 |
| Barley DOF | Mena et al, The Plant Journal, 116(1): 53-62, 1998 |
| blz2 | EP99106056.7 |
| Synthetic promoter | Vicente-Carbajosa et al., Plant J. 13: 629-640, 1998. |
| rice prolamin NRP33 | Wu et al, Plant Cell Physiology 39(8) 885-889, 1998 |
| rice a-globulin Glb-1 | Wu et al, Plant Cell Physiology 39(8) 885-889, 1998 |
| rice OSH1 | Sato et al, Proc. Natl. Acad. Sci. USA, 93: 8117-8122, |
| | 1996 |
| rice α-globulin REB/OHP-1 | Nakase et al. Plant Mol. Biol. 33: 513-522, 1997 |
| rice ADP-glucose pyrophosphorylase | Trans Res 6: 157-68, 1997 |
| Maize ESR gene family | Plant J 12: 235-46, 1997 |
| Sorghum α-kafirin | DeRose et al. Plant Mol. Biol 32: 1029-35, 1996 |
| KNOX | Postma-Haarsma et al, Plant Mol. Biol. 39: 257-71, 1999 |
| rice oleosin | Wu et al, J. Biochem. 123: 386, 1998 |
| sunflower oleosin | Cummins et al., Plant Mol. Biol. 19: 873-876, 1992 |
| PRO0117, putative rice 40S | WO 2004/070039 |
| ribosomal protein | |
| PRO0136, rice alanine | Unpublished |
| aminotransferase | |
| PRO0147, trypsin inhibitor ITR1 | Unpublished |
| (barley) | |
| PRO0151, rice WSI18 | WO 2004/070039 |
| PRO0175, rice RAB21 | WO 2004/070039 |
| PRO005 | WO 2004/070039 |
| PRO0095 | WO 2004/070039 |
| α-amylase (Amy32b) | Lanahan et al, Plant Cell 4: 203-211, 1992; Skriver et al, |
| | Proc Natl Acad Sci USA 88: 7266-7270, 1991 |
| Cathepsin β-like gene | Cejudo et al, Plant Mol Biol 20: 849-856, 1992 |
| Barley Ltp2 | Kalla et al., Plant J. 6: 849-60, 1994 |
| Chi26 | Leah et al., Plant J. 4: 579-89, 1994 |
| Maize B-Peru | Selinger et al., Genetics 149; 1125-38, 1998 |

A green tissue-specific promoter as defined herein is a promoter that is transcriptionally active predominantly in green tissue, substantially to the exclusion of any other parts of a plant, whilst still allowing for any leaky expression in these other plant parts. From 59392: Examples of green tissue-specific promoters are given in Nomura et al., 2000 Plant Mol Biol. 44(1):99-106; WO 2004/070039.

Another example of a tissue-specific promoter is a meristem-specific promoter, which is transcriptionally active predominantly in meristematic tissue, substantially to the exclusion of any other parts of a plant, whilst still allowing for any leaky expression in these other plant parts.

### Terminator

The term “terminator” encompasses a control sequence which is a DNA sequence at the end of a transcriptional unit which signals 3′ processing and polyadenylation of a primary transcript and termination of transcription. The terminator can be derived from the natural gene, from a variety of other plant genes, or from T-DNA. The terminator to be added may be derived from, for example, the nopaline synthase or octopine synthase genes, or alternatively from another plant gene, or less preferably from any other eukaryotic gene.

### Modulation

The term “modulation” means in relation to expression or gene expression, a process in which the expression level is changed by said gene expression in comparison to the control plant, the expression level may be increased or decreased. The original, unmodulated expression may be of any kind of expression of a structural RNA (rRNA, tRNA) or mRNA with subsequent translation. The term “modulating the activity” shall mean any change of the expression of the inventive nucleic acid sequences or encoded proteins, which leads to increased yield and/or increased growth of the plants.

### Expression

The term “expression” or “gene expression” means the transcription of a specific gene or specific genes or specific genetic construct. The term “expression” or “gene expression” in particular means the transcription of a gene or genes or genetic construct into structural RNA (rRNA, tRNA) or mRNA with or without subsequent translation of the latter into a protein. The process includes transcription of DNA and processing of the resulting mRNA product.

### Increased Expression/Overexpression

The term “increased expression” or “overexpression” as used herein means any form of expression that is additional to the original wild-type expression level.

Methods for increasing expression of genes or gene products are well documented in the art and include, for example, overexpression driven by appropriate promoters, the use of transcription enhancers or translation enhancers. Isolated nucleic acids which serve as promoter or enhancer elements may be introduced in an appropriate position (typically upstream) of a non-heterologous form of a polynucleotide so as to upregulate expression of a nucleic acid encoding the polypeptide of interest. For example, endogenous promoters may be altered in vivo by mutation, deletion, and/or substitution (see, Kmiec, U.S. Pat. No. 5,565,350; Zarling et al., WO9322443), or isolated promoters may be introduced into a plant cell in the proper orientation and distance from a gene of the present invention so as to control the expression of the gene.

If polypeptide expression is desired, it is generally desirable to include a polyadenylation region at the 3′-end of a polynucleotide coding region. The polyadenylation region can be derived from the natural gene, from a variety of other plant genes, or from T-DNA. The 3′ end sequence to be added may be derived from, for example, the nopaline synthase or octopine synthase genes, or alternatively from another plant gene, or less preferably from any other eukaryotic gene.

An intron sequence may also be added to the 5′ untranslated region (UTR) or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg (1988) Mol. Cell biol. 8: 4395-4405; Callis et al. (1987) Genes Dev 1:1183-1200). Such intron enhancement of gene expression is typically greatest when placed near the 5′ end of the transcription unit. Use of the maize introns Adh1-S intron 1, 2, and 6, the Bronze-1 intron are known in the art. For general information see: The Maize Handbook, Chapter 116, Freeling and Walbot, Eds., Springer, N.Y. (1994).

### Endogenous Gene

Reference herein to an “endogenous” gene not only refers to the gene in question as found in a plant in its natural form (i.e., without there being any human intervention), but also refers to that same gene (or a substantially homologous nucleic acid/gene) in an isolated form subsequently (re)introduced into a plant (a transgene). For example, a transgenic plant containing such a transgene may encounter a substantial reduction of the transgene expression and/or substantial reduction of expression of the endogenous gene. The isolated gene may be isolated from an organism or may be manmade, for example by chemical synthesis.

### Decreased Expression

Reference herein to “decreased expression” or “reduction or substantial elimination” of expression is taken to mean a decrease in endogenous gene expression and/or polypeptide levels and/or polypeptide activity relative to control plants. The reduction or substantial elimination is in increasing order of preference at least 10%, 20%, 30%, 40% or 50%, 60%, 70%, 80%, 85%, 90%, or 95%, 96%, 97%, 98%, 99% or more reduced compared to that of control plants.

For optimal performance, the gene silencing techniques used for reducing expression in a plant of an endogenous gene requires the use of nucleic acid sequences from monocotyledonous plants for transformation of monocotyledonous plants, and from dicotyledonous plants for transformation of dicotyledonous plants. Preferably, a nucleic acid sequence from any given plant species is introduced into that same species. For example, a nucleic acid sequence from rice is transformed into a rice plant. However, it is not an absolute requirement that the nucleic acid sequence to be introduced originates from the same plant species as the plant in which it will be introduced. It is sufficient that there is substantial homology between the endogenous target gene and the nucleic acid to be introduced.

### From 59391:

For the reduction or substantial elimination of expression an endogenous gene in a plant, a sufficient length of substantially contiguous nucleotides of a nucleic acid sequence is required. In order to perform gene silencing, this may be as little as 20, 19, 18, 17, 16, 15, 14, 13, 12, 11, 10 or fewer nucleotides, alternatively this may be as much as the entire gene (including the 5′ and/or 3′ UTR, either in part or in whole). The stretch of substantially contiguous nucleotides may be derived from the nucleic acid encoding the protein of interest (target gene), or from any nucleic acid capable of encoding an orthologue, paralogue or homologue of the protein of interest. Preferably, the stretch of substantially contiguous nucleotides is capable of forming hydrogen bonds with the target gene (either sense or antisense strand), more preferably, the stretch of substantially contiguous nucleotides has, in increasing order of preference, 50%, 60%, 70%, 80%, 85%, 90%, 95%, 96%, 97%, 98%, 99%, 100% sequence identity to the target gene (either sense or antisense strand). A nucleic acid sequence encoding a (functional) polypeptide is not a requirement for the various methods discussed herein for the reduction or substantial elimination of expression of an endogenous gene.

This reduction or substantial elimination of expression may be achieved using routine tools and techniques. A preferred method for the reduction or substantial elimination of endogenous gene expression is by introducing and expressing in a plant a genetic construct into which the nucleic acid (in this case a stretch of substantially contiguous nucleotides derived from the gene of interest, or from any nucleic acid capable of encoding an orthologue, paralogue or homologue of any one of the protein of interest) is cloned as an inverted repeat (in part or completely), separated by a spacer (non-coding DNA).

In such a preferred method, expression of the endogenous gene is reduced or substantially eliminated through RNA-mediated silencing using an inverted repeat of a nucleic acid or a part thereof (in this case a stretch of substantially contiguous nucleotides derived from the gene of interest, or from any nucleic acid capable of encoding an orthologue, paralogue or homologue of the protein of interest), preferably capable of forming a hairpin structure. The inverted repeat is cloned in an expression vector comprising control sequences. A non-coding DNA nucleic acid sequence (a spacer, for example a matrix attachment region fragment (MAR), an intron, a polylinker, etc.) is located between the two inverted nucleic acids forming the inverted repeat. After transcription of the inverted repeat, a chimeric RNA with a self-complementary structure is formed (partial or complete). This double-stranded RNA structure is referred to as the hairpin RNA (hpRNA). The hpRNA is processed by the plant into siRNAs that are incorporated into an RNA-induced silencing complex (RISC). The RISC further cleaves the mRNA transcripts, thereby substantially reducing the number of mRNA transcripts to be translated into polypeptides. For further general details see for example, Grierson et al. (1998) WO 98/53083; Waterhouse et al. (1999) WO 99/53050).

Performance of the methods of the invention does not rely on introducing and expressing in a plant a genetic construct into which the nucleic acid is cloned as an inverted repeat, but any one or more of several well-known “gene silencing” methods may be used to achieve the same effects.

One such method for the reduction of endogenous gene expression is RNA-mediated silencing of gene expression (downregulation). Silencing in this case is triggered in a plant by a double stranded RNA sequence (dsRNA) that is substantially similar to the target endogenous gene. This dsRNA is further processed by the plant into about 20 to about 26 nucleotides called short interfering RNAs (siRNAs). The siRNAs are incorporated into an RNA-induced silencing complex (RISC) that cleaves the mRNA transcript of the endogenous target gene, thereby substantially reducing the number of mRNA transcripts to be translated into a polypeptide. Preferably, the double stranded RNA sequence corresponds to a target gene.

Another example of an RNA silencing method involves the introduction of nucleic acid sequences or parts thereof (in this case a stretch of substantially contiguous nucleotides derived from the gene of interest, or from any nucleic acid capable of encoding an orthologue, paralogue or homologue of the protein of interest) in a sense orientation into a plant. “Sense orientation” refers to a DNA sequence that is homologous to an mRNA transcript thereof. Introduced into a plant would therefore be at least one copy of the nucleic acid sequence. The additional nucleic acid sequence will reduce expression of the endogenous gene, giving rise to a phenomenon known as co-suppression. The reduction of gene expression will be more pronounced if several additional copies of a nucleic acid sequence are introduced into the plant, as there is a positive correlation between high transcript levels and the triggering of co-suppression.

Another example of an RNA silencing method involves the use of antisense nucleic acid sequences. An “antisense” nucleic acid sequence comprises a nucleotide sequence that is complementary to a “sense” nucleic acid sequence encoding a protein, i.e. complementary to the coding strand of a double-stranded cDNA molecule or complementary to an mRNA transcript sequence. The antisense nucleic acid sequence is preferably complementary to the endogenous gene to be silenced. The complementarity may be located in the “coding region” and/or in the “non-coding region” of a gene. The term “coding region” refers to a region of the nucleotide sequence comprising codons that are translated into amino acid residues. The term “non-coding region” refers to 5′ and 3′ sequences that flank the coding region that are transcribed but not translated into amino acids (also referred to as 5′ and 3′ untranslated regions).

Antisense nucleic acid sequences can be designed according to the rules of Watson and Crick base pairing. The antisense nucleic acid sequence may be complementary to the entire nucleic acid sequence (in this case a stretch of substantially contiguous nucleotides derived from the gene of interest, or from any nucleic acid capable of encoding an orthologue, paralogue or homologue of the protein of interest), but may also be an oligonucleotide that is antisense to only a part of the nucleic acid sequence (including the mRNA 5′ and 3′ UTR). For example, the antisense oligonucleotide sequence may be complementary to the region surrounding the translation start site of an mRNA transcript encoding a polypeptide. The length of a suitable antisense oligonucleotide sequence is known in the art and may start from about 50, 45, 40, 35, 30, 25, 20, 15 or 10 nucleotides in length or less. An antisense nucleic acid sequence according to the invention may be constructed using chemical synthesis and enzymatic ligation reactions using methods known in the art. For example, an antisense nucleic acid sequence (e.g., an antisense oligonucleotide sequence) may be chemically synthesized using naturally occurring nucleotides or variously modified nucleotides designed to increase the biological stability of the molecules or to increase the physical stability of the duplex formed between the antisense and sense nucleic acid sequences, e.g., phosphorothioate derivatives and acridine substituted nucleotides may be used. Examples of modified nucleotides that may be used to generate the antisense nucleic acid sequences are well known in the art. Known nucleotide modifications include methylation, cyclization and ‘caps’ and substitution of one or more of the naturally occurring nucleotides with an analogue such as inosine. Other modifications of nucleotides are well known in the art.

The antisense nucleic acid sequence can be produced biologically using an expression vector into which a nucleic acid sequence has been subcloned in an antisense orientation (i.e., RNA transcribed from the inserted nucleic acid will be of an antisense orientation to a target nucleic acid of interest). Preferably, production of antisense nucleic acid sequences in plants occurs by means of a stably integrated nucleic acid construct comprising a promoter, an operably linked antisense oligonucleotide, and a terminator.

The nucleic acid molecules used for silencing in the methods of the invention (whether introduced into a plant or generated in situ) hybridize with or bind to mRNA transcripts and/or genomic DNA encoding a polypeptide to thereby inhibit expression of the protein, e.g., by inhibiting transcription and/or translation. The hybridization can be by conventional nucleotide complementarity to form a stable duplex, or, for example, in the case of an antisense nucleic acid sequence which binds to DNA duplexes, through specific interactions in the major groove of the double helix. Antisense nucleic acid sequences may be introduced into a plant by transformation or direct injection at a specific tissue site. Alternatively, antisense nucleic acid sequences can be modified to target selected cells and then administered systemically. For example, for systemic administration, antisense nucleic acid sequences can be modified such that they specifically bind to receptors or antigens expressed on a selected cell surface, e.g., by linking the antisense nucleic acid sequence to peptides or antibodies which bind to cell surface receptors or antigens. The antisense nucleic acid sequences can also be delivered to cells using the vectors described herein.

According to a further aspect, the antisense nucleic acid sequence is an a-anomeric nucleic acid sequence. An a-anomeric nucleic acid sequence forms specific double-stranded hybrids with complementary RNA in which, contrary to the usual b-units, the strands run parallel to each other (Gaultier et al. (1987) Nucl Ac Res 15: 6625-6641). The antisense nucleic acid sequence may also comprise a 2′-o-methylribonucleotide (Inoue et al. (1987) Nucl Ac Res 15, 6131-6148) or a chimeric RNA-DNA analogue (Inoue et al. (1987) FEBS Lett. 215, 327-330).

The reduction or substantial elimination of endogenous gene expression may also be performed using ribozymes. Ribozymes are catalytic RNA molecules with ribonuclease activity that are capable of cleaving a single-stranded nucleic acid sequence, such as an mRNA, to which they have a complementary region. Thus, ribozymes (e.g., hammerhead ribozymes (described in Haselhoff and Gerlach (1988) Nature 334, 585-591) can be used to catalytically cleave mRNA transcripts encoding a polypeptide, thereby substantially reducing the number of mRNA transcripts to be translated into a polypeptide. A ribozyme having specificity for a nucleic acid sequence can be designed (see for example: Cech et al. U.S. Pat. No. 4,987,071; and Cech et al. U.S. Pat. No. 5,116,742). Alternatively, mRNA transcripts corresponding to a nucleic acid sequence can be used to select a catalytic RNA having a specific ribonuclease activity from a pool of RNA molecules (Bartel and Szostak (1993) Science 261, 1411-1418). The use of ribozymes for gene silencing in plants is known in the art (e.g., Atkins et al. (1994) WO 94/00012; Lenne et al. (1995) WO 95/03404; Lutziger et al. (2000) WO 00/00619; Prinsen et al. (1997) WO 97/13865 and Scott et al. (1997) WO 97/38116).

Gene silencing may also be achieved by insertion mutagenesis (for example, T-DNA insertion or transposon insertion) or by strategies as described by, among others, Angell and Baulcombe ((1999) Plant J 20(3): 357-62), (Amplicon VIGS WO 98/36083), or Baulcombe (WO 99/15682).

Gene silencing may also occur if there is a mutation on an endogenous gene and/or a mutation on an isolated gene/nucleic acid subsequently introduced into a plant. The reduction or substantial elimination may be caused by a non-functional polypeptide. For example, a polypeptide may bind to various interacting proteins; one or more mutation(s) and/or truncation(s) may therefore provide for a polypeptide that is still able to bind interacting proteins (such as receptor proteins) but that cannot exhibit its normal function (such as signalling ligand).

A further approach to gene silencing is by targeting nucleic acid sequences complementary to the regulatory region of the gene (e.g., the promoter and/or enhancers) to form triple helical structures that prevent transcription of the gene in target cells. See Helene, C., Anticancer Drug Res. 6, 569-84, 1991; Helene et al., Ann. N.Y. Acad. Sci. 660, 27-36 1992; and Maher, L. J. Bioassays 14, 807-15, 1992.

Other methods, such as the use of antibodies directed to an endogenous polypeptide for inhibiting its function in planta, or interference in the signalling pathway in which a polypeptide is involved, will be well known to the skilled man. In particular, it can be envisaged that manmade molecules may be useful for inhibiting the biological function of a target polypeptide, or for interfering with the signalling pathway in which the target polypeptide is involved.

Alternatively, a screening program may be set up to identify in a plant population natural variants of a gene, which variants encode polypeptides with reduced activity. Such natural variants may also be used for example, to perform homologous recombination.

Artificial and/or natural microRNAs (miRNAs) may be used to knock out gene expression and/or mRNA translation. Endogenous miRNAs are single stranded small RNAs of typically 19-24 nucleotides long. They function primarily to regulate gene expression and/or mRNA translation. Most plant microRNAs (miRNAs) have perfect or near-perfect complementarity with their target sequences. However, there are natural targets with up to five mismatches. They are processed from longer non-coding RNAs with characteristic fold-back structures by double-strand specific RNases of the Dicer family. Upon processing, they are incorporated in the RNA-induced silencing complex (RISC) by binding to its main component, an Argonaute protein. MiRNAs serve as the specificity components of RISC, since they base-pair to target nucleic acids, mostly mRNAs, in the cytoplasm. Subsequent regulatory events include target mRNA cleavage and destruction and/or translational inhibition. Effects of miRNA overexpression are thus often reflected in decreased mRNA levels of target genes.

Artificial microRNAs (amiRNAs), which are typically 21 nucleotides in length, can be genetically engineered specifically to negatively regulate gene expression of single or multiple genes of interest. Determinants of plant microRNA target selection are well known in the art. Empirical parameters for target recognition have been defined and can be used to aid in the design of specific amiRNAs, (Schwab et al., Dev. Cell 8, 517-527, 2005). Convenient tools for design and generation of amiRNAs and their precursors are also available to the public (Schwab et al., Plant Cell 18, 1121-1133, 2006).

For optimal performance, the gene silencing techniques used for reducing expression in a plant of an endogenous gene requires the use of nucleic acid sequences from monocotyledonous plants for transformation of monocotyledonous plants, and from dicotyledonous plants for transformation of dicotyledonous plants. Preferably, a nucleic acid sequence from any given plant species is introduced into that same species. For example, a nucleic acid sequence from rice is transformed into a rice plant. However, it is not an absolute requirement that the nucleic acid sequence to be introduced originates from the same plant species as the plant in which it will be introduced. It is sufficient that there is substantial homology between the endogenous target gene and the nucleic acid to be introduced.

Described above are examples of various methods for the reduction or substantial elimination of expression in a plant of an endogenous gene. A person skilled in the art would readily be able to adapt the aforementioned methods for silencing so as to achieve reduction of expression of an endogenous gene in a whole plant or in parts thereof through the use of an appropriate promoter, for example.

A person skilled in the art is familiar with the various techniques for decreasing expression of a gene.

Selectable Marker (Gene)/Reporter Gene

“Selectable marker”, “selectable marker gene” or “reporter gene” includes any gene that confers a phenotype on a cell in which it is expressed to facilitate the identification and/or selection of cells that are transfected or transformed with a nucleic acid construct of the invention. These marker genes enable the identification of a successful transfer of the nucleic acid molecules via a series of different principles. Suitable markers may be selected from markers that confer antibiotic or herbicide resistance, that introduce a new metabolic trait or that allow visual selection. Examples of selectable marker genes include genes conferring resistance to antibiotics (such as nptII that phosphorylates neomycin and kanamycin, or hpt, phosphorylating hygromycin, or genes conferring resistance to, for example, bleomycin, streptomycin, tetracyclin, chloramphenicol, ampicillin, gentamycin, geneticin (G418), spectinomycin or blasticidin), to herbicides (for example bar which provides resistance to Basta®; aroA or gox providing resistance against glyphosate, or the genes conferring resistance to, for example, imidazolinone, phosphinothricin or sulfonylurea), or genes that provide a metabolic trait (such as manA that allows plants to use mannose as sole carbon source or xylose isomerase for the utilisation of xylose, or antinutritive markers such as the resistance to 2-deoxyglucose). Expression of visual marker genes results in the formation of colour (for example β-glucuronidase, GUS or β-galactosidase with its coloured substrates, for example X-Gal), luminescence (such as the luciferin/luceferase system) or fluorescence (Green Fluorescent Protein, GFP, and derivatives thereof). This list represents only a small number of possible markers. The skilled worker is familiar with such markers. Different markers are preferred, depending on the organism and the selection method.

It is known that upon stable or transient integration of nucleic acids into plant cells, only a minority of the cells takes up the foreign DNA and, if desired, integrates it into its genome, depending on the expression vector used and the transfection technique used. To identify and select these integrants, a gene coding for a selectable marker (such as the ones described above) is usually introduced into the host cells together with the gene of interest. These markers can for example be used in mutants in which these genes are not functional by, for example, deletion by conventional methods. Furthermore, nucleic acid molecules encoding a selectable marker can be introduced into a host cell on the same vector that comprises the sequence encoding the polypeptides of the invention or used in the methods of the invention, or else in a separate vector. Cells which have been stably transfected with the introduced nucleic acid can be identified for example by selection (for example, cells which have integrated the selectable marker survive whereas the other cells die).

Since the marker genes, particularly genes for resistance to antibiotics and herbicides, are no longer required or are undesired in the transgenic host cell once the nucleic acids have been introduced successfully, the process according to the invention for introducing the nucleic acids advantageously employs techniques which enable the removal or excision of these marker genes. One such a method is what is known as co-transformation. The co-transformation method employs two vectors simultaneously for the transformation, one vector bearing the nucleic acid according to the invention and a second bearing the marker gene(s). A large proportion of transformants receives or, in the case of plants, comprises (up to 40% or more of the transformants), both vectors. In case of transformation with Agrobacteria, the transformants usually receive only a part of the vector, i.e. the sequence flanked by the T-DNA, which usually represents the expression cassette. The marker genes can subsequently be removed from the transformed plant by performing crosses. In another method, marker genes integrated into a transposon are used for the transformation together with desired nucleic acid (known as the Ac/Ds technology). The transformants can be crossed with a transposase source or the transformants are transformed with a nucleic acid construct conferring expression of a transposase, transiently or stable. In some cases (approx. 10%), the transposon jumps out of the genome of the host cell once transformation has taken place successfully and is lost. In a further number of cases, the transposon jumps to a different location. In these cases the marker gene must be eliminated by performing crosses. In microbiology, techniques were developed which make possible, or facilitate, the detection of such events. A further advantageous method relies on what is known as recombination systems; whose advantage is that elimination by crossing can be dispensed with. The best-known system of this type is what is known as the Cre/lox system. Cre1 is a recombinase that removes the sequences located between the loxP sequences. If the marker gene is integrated between the loxP sequences, it is removed once transformation has taken place successfully, by expression of the recombinase. Further recombination systems are the HIN/HIX, FLP/FRT and REP/STB system (Tribble et al., J. Biol. Chem., 275, 2000: 22255-22267; Velmurugan et al., J. Cell Biol., 149, 2000: 553-566). A site-specific integration into the plant genome of the nucleic acid sequences according to the invention is possible. Naturally, these methods can also be applied to microorganisms such as yeast, fungi or bacteria.

### Transgenic/Transgene/Recombinant

For the purposes of the invention, “transgenic”, “transgene” or “recombinant” means with regard to, for example, a nucleic acid sequence, an expression cassette, gene construct or a vector comprising the nucleic acid sequence or an organism transformed with the nucleic acid sequences, expression cassettes or vectors according to the invention, all those constructions brought about by recombinant methods in which either
(a) the nucleic acid sequences encoding proteins useful in the methods of the invention, or
(b) genetic control sequence(s) which is operably linked with the nucleic acid sequence according to the invention, for example a promoter, or
(c) a) and b)
   are not located in their natural genetic environment or have been modified by recombinant methods, it being possible for the modification to take the form of, for example, a substitution, addition, deletion, inversion or insertion of one or more nucleotide residues. The natural genetic environment is understood as meaning the natural genomic or chromosomal locus in the original plant or the presence in a genomic library. In the case of a genomic library, the natural genetic environment of the nucleic acid sequence is preferably retained, at least in part. The environment flanks the nucleic acid sequence at least on one side and has a sequence length of at least 50 bp, preferably at least 500 bp, especially preferably at least 1000 bp, most preferably at least 5000 bp. A naturally occurring expression cassette—for example the naturally occurring combination of the natural promoter of the nucleic acid sequences with the corresponding nucleic acid sequence encoding a polypeptide useful in the methods of the present invention, as defined above—becomes a transgenic expression cassette when this expression cassette is modified by non-natural, synthetic (“artificial”) methods such as, for example, mutagenic treatment. Suitable methods are described, for example, in U.S. Pat. No. 5,565,350 or WO 00/15815.

A transgenic plant for the purposes of the invention is thus understood as meaning, as above, that the nucleic acids used in the method of the invention are not at their natural locus in the genome of said plant, it being possible for the nucleic acids to be expressed homologously or heterologously. However, as mentioned, transgenic also means that, while the nucleic acids according to the invention or used in the inventive method are at their natural position in the genome of a plant, the sequence has been modified with regard to the natural sequence, and/or that the regulatory sequences of the natural sequences have been modified. Transgenic is preferably understood as meaning the expression of the nucleic acids according to the invention at an unnatural locus in the genome, i.e. homologous or, preferably, heterologous expression of the nucleic acids takes place. Preferred transgenic plants are mentioned herein.

### Transformation

The term “introduction” or “transformation” as referred to herein encompasses the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated there from. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid.

Alternatively, it may be integrated into the host genome. The resulting transformed plant cell may then be used to regenerate a transformed plant in a manner known to persons skilled in the art.

The transfer of foreign genes into the genome of a plant is called transformation. Transformation of plant species is now a fairly routine technique. Advantageously, any of several transformation methods may be used to introduce the gene of interest into a suitable ancestor cell. The methods described for the transformation and regeneration of plants from plant tissues or plant cells may be utilized for transient or for stable transformation. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts (Krens, F. A. et al., (1982) Nature 296, 72-74; Negrutiu I et al. (1987) Plant Mol Biol 8: 363-373); electroporation of protoplasts (Shillito R. D. et al. (1985) Bio/Technol 3, 1099-1102); microinjection into plant material (Crossway A et al., (1986) Mol. Gen Genet 202: 179-185); DNA or RNA-coated particle bombardment (Klein TM et al., (1987) Nature 327: 70) infection with (non-integrative) viruses and the like. Transgenic plants, including transgenic crop plants, are preferably produced via Agrobacterium-mediated transformation. An advantageous transformation method is the transformation in planta. To this end, it is possible, for example, to allow the agrobacteria to act on plant seeds or to inoculate the plant meristem with agrobacteria. It has proved particularly expedient in accordance with the invention to allow a suspension of transformed agrobacteria to act on the intact plant or at least on the flower primordia. The plant is subsequently grown on until the seeds of the treated plant are obtained (Clough and Bent, Plant J. (1998) 16, 735-743). Methods for Agrobacterium-mediated transformation of rice include well known methods for rice transformation, such as those described in any of the following: European patent application EP 1198985 A1, Aldemita and Hodges (Planta 199: 612-617, 1996); Chan et al. (Plant Mol Biol 22 (3): 491-506, 1993), Hiei et al. (Plant J 6 (2): 271-282, 1994), which disclosures are incorporated by reference herein as if fully set forth. In the case of corn transformation, the preferred method is as described in either Ishida et al. (Nat. Biotechnol 14(6): 745-50, 1996) or Frame et al. (Plant Physiol 129(1): 13-22, 2002), which disclosures are incorporated by reference herein as if fully set forth. Said methods are further described by way of example in B. Jenes et al., Techniques for Gene Transfer, in: Transgenic Plants, Vol. 1, Engineering and Utilization, eds. S. D. Kung and R. Wu, Academic Press (1993) 128-143 and in Potrykus Annu. Rev. Plant Physiol. Plant Molec. Biol. 42 (1991) 205-225). The nucleic acids or the construct to be expressed is preferably cloned into a vector, which is suitable for transforming Agrobacterium tumefaciens, for example pBin19 (Bevan et al., Nucl. Acids Res. 12 (1984) 8711). Agrobacteria transformed by such a vector can then be used in known manner for the transformation of plants, such as plants used as a model, like Arabidopsis (Arabidopsis thaliana is within the scope of the present invention not considered as a crop plant), or crop plants such as, by way of example, tobacco plants, for example by immersing bruised leaves or chopped leaves in an agrobacterial solution and then culturing them in suitable media. The transformation of plants by means of Agrobacterium tumefaciens is described, for example, by Hofgen and Willmitzer in Nucl. Acid Res. (1988) 16, 9877 or is known inter alia from F. F. White, Vectors for Gene Transfer in Higher Plants; in Transgenic Plants, Vol. 1, Engineering and Utilization, eds. S. D. Kung and R. Wu, Academic Press, 1993, pp. 15-38.

In addition to the transformation of somatic cells, which then have to be regenerated into intact plants, it is also possible to transform the cells of plant meristems and in particular those cells which develop into gametes. In this case, the transformed gametes follow the natural plant development, giving rise to transgenic plants. Thus, for example, seeds of Arabidopsis are treated with agrobacteria and seeds are obtained from the developing plants of which a certain proportion is transformed and thus transgenic [Feldman, K A and Marks M D (1987). Mol Gen Genet 208:274-289; Feldmann K (1992). In: C Koncz, N-H Chua and J Shell, eds, Methods in Arabidopsis Research. Word Scientific, Singapore, pp. 274-289]. Alternative methods are based on the repeated removal of the inflorescences and incubation of the excision site in the center of the rosette with transformed agrobacteria, whereby transformed seeds can likewise be obtained at a later point in time (Chang (1994). Plant J. 5: 551-558; Katavic (1994). Mol Gen Genet, 245: 363-370). However, an especially effective method is the vacuum infiltration method with its modifications such as the “floral dip” method. In the case of vacuum infiltration of Arabidopsis, intact plants under reduced pressure are treated with an agrobacterial suspension [Bechthold, N (1993). C R Acad Sci Paris Life Sci, 316: 1194-1199], while in the case of the “floral dip” method the developing floral tissue is incubated briefly with a surfactant-treated agrobacterial suspension [Clough, S J and Bent A F (1998) The Plant J. 16, 735-743]. A certain proportion of transgenic seeds are harvested in both cases, and these seeds can be distinguished from non-transgenic seeds by growing under the above-described selective conditions. In addition the stable transformation of plastids is of advantages because plastids are inherited maternally is most crops reducing or eliminating the risk of transgene flow through pollen. The transformation of the chloroplast genome is generally achieved by a process which has been schematically displayed in Klaus et al., 2004 [Nature Biotechnology 22 (2), 225-229]. Briefly the sequences to be transformed are cloned together with a selectable marker gene between flanking sequences homologous to the chloroplast genome. These homologous flanking sequences direct site specific integration into the plastome.

Plastidal transformation has been described for many different plant species and an overview is given in Bock (2001) Transgenic plastids in basic research and plant biotechnology. J Mol Biol. 2001 Sep. 21; 312 (3):425-38 or Maliga, P (2003) Progress towards commercialization of plastid transformation technology. Trends Biotechnol. 21, 20-28. Further biotechnological progress has recently been reported in form of marker free plastid transformants, which can be produced by a transient co-integrated maker gene (Klaus et al., 2004, Nature Biotechnology 22(2), 225-229).

### T-DNA Activation Tagging

T-DNA activation tagging (Hayashi et al. Science (1992) 1350-1353), involves insertion of T-DNA, usually containing a promoter (may also be a translation enhancer or an intron), in the genomic region of the gene of interest or 10 kb up- or downstream of the coding region of a gene in a configuration such that the promoter directs expression of the targeted gene. Typically, regulation of expression of the targeted gene by its natural promoter is disrupted and the gene falls under the control of the newly introduced promoter. The promoter is typically embedded in a T-DNA. This T-DNA is randomly inserted into the plant genome, for example, through Agrobacterium infection and leads to modified expression of genes near the inserted T-DNA. The resulting transgenic plants show dominant phenotypes due to modified expression of genes close to the introduced promoter.

### TILLING

The term “TILLING” is an abbreviation of “Targeted Induced Local Lesions In Genomes” and refers to a mutagenesis technology useful to generate and/or identify nucleic acids encoding proteins with modified expression and/or activity. TILLING also allows selection of plants carrying such mutant variants. These mutant variants may exhibit modified expression, either in strength or in location or in timing (if the mutations affect the promoter for example). These mutant variants may exhibit higher activity than that exhibited by the gene in its natural form. TILLING combines high-density mutagenesis with high-throughput screening methods. The steps typically followed in TILLING are: (a) EMS mutagenesis (Redei G P and Koncz C (1992) In Methods in Arabidopsis Research, Koncz C, Chua N H, Schell J, eds. Singapore, World Scientific Publishing Co, pp. 16-82; Feldmann et al., (1994) In Meyerowitz E M, Somerville C R, eds, Arabidopsis. Cold Spring Harbor Laboratory Press, Cold Spring Harbor, N.Y., pp 137-172; Lightner J and Caspar T (1998) In J Martinez-Zapater, J Salinas, eds, Methods on Molecular Biology, Vol. 82. Humana Press, Totowa, N.J., pp 91-104); (b) DNA preparation and pooling of individuals; (c) PCR amplification of a region of interest; (d) denaturation and annealing to allow formation of heteroduplexes; (e) DHPLC, where the presence of a heteroduplex in a pool is detected as an extra peak in the chromatogram; (f) identification of the mutant individual; and (g) sequencing of the mutant PCR product. Methods for TILLING are well known in the art (McCallum et al., (2000) Nat Biotechnol 18: 455-457; reviewed by Stemple (2004) Nat Rev Genet 5(2): 145-50).

### Homologous Recombination

Homologous recombination allows introduction in a genome of a selected nucleic acid at a defined selected position. Homologous recombination is a standard technology used routinely in biological sciences for lower organisms such as yeast or the moss Physcomitrella. Methods for performing homologous recombination in plants have been described not only for model plants (Offring a et al. (1990) EMBO J 9(10): 3077-84) but also for crop plants, for example rice

(Terada et al. (2002) Nat Biotech 20(10): 1030-4; Iida and Terada (2004) Curr Opin Biotech 15(2): 132-8), and approaches exist that are generally applicable regardless of the target organism (Miller et al, Nature Biotechnol. 25, 778-785, 2007).

### Yield

The term “yield” in general means a measurable produce of economic value, typically related to a specified crop, to an area, and to a period of time. Individual plant parts directly contribute to yield based on their number, size and/or weight, or the actual yield is the yield per square meter for a crop and year, which is determined by dividing total production (includes both harvested and appraised production) by planted square meters. The term “yield” of a plant may relate to vegetative biomass (root and/or shoot biomass), to reproductive organs, and/or to propagules (such as seeds) of that plant.

### Early Vigour

“Early vigour” refers to active healthy well-balanced growth especially during early stages of plant growth, and may result from increased plant fitness due to, for example, the plants being better adapted to their environment (i.e. optimizing the use of energy resources and partitioning between shoot and root). Plants having early vigour also show increased seedling survival and a better establishment of the crop, which often results in highly uniform fields (with the crop growing in uniform manner, i.e. with the majority of plants reaching the various stages of development at substantially the same time), and often better and higher yield. Therefore, early vigour may be determined by measuring various factors, such as thousand kernel weight, percentage germination, percentage emergence, seedling growth, seedling height, root length, root and shoot biomass and many more.

### Increase/Improve/Enhance

The terms “increase”, “improve” or “enhance” are interchangeable and shall mean in the sense of the application at least a 3%, 4%, 5%, 6%, 7%, 8%, 9% or 10%, preferably at least 15% or 20%, more preferably 25%, 30%, 35% or 40% more yield and/or growth in comparison to control plants as defined herein.

### Seed Yield

Increased seed yield may manifest itself as one or more of the following: a) an increase in seed biomass (total seed weight) which may be on an individual seed basis and/or per plant and/or per square meter; b) increased number of flowers per plant; c) increased number of (filled) seeds; d) increased seed filling rate (which is expressed as the ratio between the number of filled seeds divided by the total number of seeds); e) increased harvest index, which is expressed as a ratio of the yield of harvestable parts, such as seeds, divided by the total biomass; and f) increased thousand kernel weight (TKW), which is extrapolated from the number of filled seeds counted and their total weight. An increased TKW may result from an increased seed size and/or seed weight, and may also result from an increase in embryo and/or endosperm size.

An increase in seed yield may also be manifested as an increase in seed size and/or seed volume. Furthermore, an increase in seed yield may also manifest itself as an increase in seed area and/or seed length and/or seed width and/or seed perimeter. Increased yield may also result in modified architecture, or may occur because of modified architecture.

### Greenness Index

The “greenness index” as used herein is calculated from digital images of plants. For each pixel belonging to the plant object on the image, the ratio of the green value versus the red value (in the RGB model for encoding color) is calculated. The greenness index is expressed as the percentage of pixels for which the green-to-red ratio exceeds a given threshold. Under normal growth conditions, under salt stress growth conditions, and under reduced nutrient availability growth conditions, the greenness index of plants is measured in the last imaging before flowering. In contrast, under drought stress growth conditions, the greenness index of plants is measured in the first imaging after drought.

### Plant

The term “plant” as used herein encompasses whole plants, ancestors and progeny of the plants and plant parts, including seeds, shoots, stems, leaves, roots (including tubers), flowers, and tissues and organs, wherein each of the aforementioned comprise the gene/nucleic acid of interest. The term “plant” also encompasses plant cells, suspension cultures, callus tissue, embryos, meristematic regions, gametophytes, sporophytes, pollen and microspores, again wherein each of the aforementioned comprises the gene/nucleic acid of interest.

Plants that are particularly useful in the methods of the invention include all plants which belong to the superfamily Viridiplantae, in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp., amongst others.

### DETAILED DESCRIPTION OF THE INVENTION

Surprisingly, it has now been found that modulating expression in a plant of a nucleic acid encoding a bHLH6-like polypeptide gives plants having enhanced yield-related traits relative to control plants. According to a first embodiment, the present invention provides a method for enhancing yield-related traits in plants relative to control plants, comprising modulating expression in a plant of a nucleic acid encoding a bHLH6-like polypeptide.

A preferred method for modulating (preferably, increasing) expression of a nucleic acid encoding a bHLH6-like polypeptide is by introducing and expressing in a plant a nucleic acid encoding a bHLH6-like polypeptide.

In one embodiment any reference hereinafter to a “protein useful in the methods of the invention” is taken to mean a bHLH6-like polypeptide as defined herein. In the same embodiment any reference hereinafter to a “nucleic acid useful in the methods of the invention” is taken to mean a nucleic acid capable of encoding such a bHLH6-like polypeptide. In one embodiment the nucleic acid to be introduced into a plant (and therefore useful in performing the methods of the invention) is any nucleic acid encoding the type of protein which will now be described, hereafter also named “bHLH6-like nucleic acid” or “bHLH6-like gene”.

A “bHLH6-like polypeptide” as defined herein refers to any polypeptide comprising a basic domain followed by a HLH domain (HMMPFam PF00010, ProfileScan PS50888, SMART SM00353) thereby forming a basic helix-loop-helix domain (Interpro IPR001092). Preferably, the bHLH6-like polypeptide comprises at least one, preferably two, more preferably three or more of the following motifs:

Motif 1 (SEQ ID NO: 3): L (G/E/T) WX (D/E) (G/S) X
(Y/F) (K/N) G (E/D)

wherein X in position 4 can be any amino acid, preferably a turnlike amino acid, more preferably one of G, R, K, T, and S; and wherein X in position 7 can be any amino acid, preferably a hydrophobic or polar amino acid, more preferably one of Y, F, N, and H.

Motif 2 (SEQ ID NO: 4): G (V/I) (V/I/L) E
(V/L/I) (G/A) (S/V/T/A) (T/L/S) (E/D/S)
Motif 3 (SEQ ID NO: 5): (D/E) K (A/V/I) S (L/I/V)
L (G/D/E/A)
Motif 4 (SEQ ID NO: 6): (T/N/S) LQ (Q/H) RLQ
Motif 5 (SEQ ID NO: 7): (K/F) (I/F/V) (I/L/V/M/S)
G (W/L/R/N/E) (E/D) AM (I/V) (G/R) (V/I) (Q/N/E/Y)
Motif 6 (SEQ ID NO: 8): (H/Y) (A/S) (S/N) (V/C/M/
L/T) (S/Q) (V/C/S) (V/MF) (K/N/S) (D/C/E) (L/Q/F/
M) (M/R/L) (I/F/L) (Q/D/H) (Q/D/V)

Alternatively, the homologue of a bHLH6-like protein has in increasing order of preference at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the amino acid represented by SEQ ID NO: 2, provided that the homologous protein comprises the conserved motifs as outlined above. The overall sequence identity is determined using a global alignment algorithm, such as the Needleman Wunsch algorithm in the program GAP (GCG Wisconsin Package, Accelrys), preferably with default parameters. Compared to overall sequence identity, the sequence identity will generally be higher when only conserved domains or motifs are considered. The sequence conservation is much higher in the region of the bHLH domain (see Table B2 in Example 3 and FIG. 2). Therefore the bHLH domain is a good criterion for the defining the group of bHLH6-like proteins. Preferably, the bHLH6-like polypeptide comprises the sequence of Motif 7 (SEQ ID NO: 9): PKKRGRKPAN GREEPLNHVEAERQRREKLNQRFYALRAVVPNVSKMDKASLLGDAIAYINELKSKVVKTE or a sequence that has, in increasing order of preference, at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 9. The HLH domain as determined by SMART spans residue 454 to 503 in SEQ ID NO: 2 and is comprised in Motif 7.

Preferably, the polypeptide sequence which when used in the construction of a phylogenetic tree, such as the one depicted in FIG. 6 in Li et al. (2006), clusters within subgroup N (which comprises the amino acid sequence represented by SEQ ID NO: 2), rather than with any other group.

Preferably, the genomic DNA sequence encoding the bHLH6-like polypeptide does not comprise introns.

The terms “domain”, “signature” and “motif” are defined in the “definitions” section herein. Specialist databases exist for the identification of domains, for example, SMART (Schultz et al. (1998) Proc. Natl. Acad. Sci. USA 95, 5857-5864; Letunic et al. (2002) Nucleic Acids Res 30, 242-244), InterPro (Mulder et al., (2003) Nucl. Acids. Res. 31, 315-318), Prosite (Bucher and Bairoch (1994), A generalized profile syntax for biomolecular sequences motifs and its function in automatic sequence interpretation. (In) ISMB-94; Proceedings 2nd International Conference on Intelligent Systems for Molecular Biology. Altman R., Brutlag D., Karp P., Lathrop R., Searls D., Eds., pp 53-61, AAAI Press, Menlo Park; Hula et al., Nucl. Acids. Res. 32:D134-D137, (2004) ), or Pfam (Bateman et al., Nucleic Acids Research 30(1): 276-280 (2002) ). A set of tools for in silico analysis of protein sequences is available on the ExPASy proteomics server (Swiss Institute of Bioinformatics (Gasteiger et al., ExPASy: the proteomics server for in-depth protein knowledge and analysis, Nucleic Acids Res. 31:3784-3788 (2003) ). Domains or motifs may also be identified using routine techniques, such as by sequence alignment.

Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the global (i.e. spanning the complete sequences) alignment of two sequences that maximizes the number of matches and minimizes the number of gaps. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information (NCBI). Homologues may readily be identified using, for example, the ClustalW multiple sequence alignment algorithm (version 1.83), with the default pairwise alignment parameters, and a scoring method in percentage. Global percentages of similarity and identity may also be determined using one of the methods available in the MatGAT software package (Campanella et al., BMC Bioinformatics. 2003 Jul. 10; 4:29. MatGAT: an application that generates similarity/identity matrices using protein or DNA sequences.). Minor manual editing may be performed to optimise alignment between conserved motifs, as would be apparent to a person skilled in the art. Furthermore, instead of using full-length sequences for the identification of homologues, specific domains may also be used. The sequence identity values may be determined over the entire nucleic acid or amino acid sequence or over selected domains or conserved motif(s), using the programs mentioned above using the default parameters. For local alignments, the Smith-Waterman algorithm is particularly useful (Smith T F, Waterman M S (1981) J. Mol. Biol 147(1); 195-7).

Furthermore, bHLH6-like polypeptides (at least in their native form) typically have DNA binding activity. Tools and techniques for measuring DNA binding activity are well known in the art. In addition, as shown in the present invention, a bHLH6-like protein, such as SEQ ID NO: 2, when overexpressed in rice, gives plants having enhanced yield-related traits, in particular emergence vigour and/or increased number of flowers per panicle. Other bioassays are provided in Dombrecht et al. (Plant Cell 19, 2225-2245, 2007). Further details are provided in Examples section.

The present invention is illustrated by transforming plants with the nucleic acid sequence represented by SEQ ID NO: 1, encoding the polypeptide sequence of SEQ ID NO: 2. However, performance of the invention is not restricted to these sequences; the methods of the invention may advantageously be performed using any bHLH6-like-encoding nucleic acid or bHLH6-like polypeptide as defined herein.

Examples of nucleic acids encoding bHLH6-like polypeptides are given in Table A1 of Example 1 herein. Such nucleic acids are useful in performing the methods of the invention. The amino acid sequences given in Table A1 of Example 1 are example sequences of orthologues and paralogues of the bHLH6-like polypeptide represented by SEQ ID NO: 2, the terms “orthologues” and “paralogues” being as defined herein. Further orthologues and paralogues may readily be identified by performing a so-called reciprocal blast search. Typically, this involves a first BLAST involving BLASTing a query sequence (for example using any of the sequences listed in Table A1 of Example 1) against any sequence database, such as the publicly available NCBI database. BLASTN or TBLASTX (using standard default values) are generally used when starting from a nucleotide sequence, and BLASTP or TBLASTN (using standard default values) when starting from a protein sequence. The BLAST results may optionally be filtered. The full-length sequences of either the filtered results or non-filtered results are then BLASTed back (second BLAST) against sequences from the organism from which the query sequence is derived (where the query sequence is SEQ ID NO: 1 or SEQ ID NO: 2, the second BLAST would therefore be against Arabidopsis thaliana sequences). The results of the first and second BLASTs are then compared. A paralogue is identified if a high-ranking hit from the first blast is from the same species as from which the query sequence is derived, a BLAST back then ideally results in the query sequence amongst the highest hits; an orthologue is identified if a high-ranking hit in the first BLAST is not from the same species as from which the query sequence is derived, and preferably results upon BLAST back in the query sequence being among the highest hits.

High-ranking hits are those having a low E-value. The lower the E-value, the more significant the score (or in other words the lower the chance that the hit was found by chance). Computation of the E-value is well known in the art. In addition to E-values, comparisons are also scored by percentage identity. Percentage identity refers to the number of identical nucleotides (or amino acids) between the two compared nucleic acid (or polypeptide) sequences over a particular length. In the case of large families, ClustalW may be used, followed by a neighbour joining tree, to help visualize clustering of related genes and to identify orthologues and paralogues.

Nucleic acid variants may also be useful in practising the methods of the invention. Examples of such variants include nucleic acids encoding homologues and derivatives of any one of the amino acid sequences given in Table A1 of Example 1, the terms “homologue” and “derivative” being as defined herein. Also useful in the methods of the invention are nucleic acids encoding homologues and derivatives of orthologues or paralogues of any one of the amino acid sequences given in Table A1 of Example 1. Homologues and derivatives useful in the methods of the present invention have substantially the same biological and functional activity as the unmodified protein from which they are derived.

Further nucleic acid variants useful in practising the methods of the invention include portions of nucleic acids encoding bHLH6-like polypeptides, nucleic acids hybridising to nucleic acids encoding bHLH6-like polypeptides, splice variants of nucleic acids encoding bHLH6-like polypeptides, allelic variants of nucleic acids encoding bHLH6-like polypeptides and variants of nucleic acids encoding bHLH6-like polypeptides obtained by gene shuffling. The terms hybridising sequence, splice variant, allelic variant and gene shuffling are as described herein.

Nucleic acids encoding bHLH6-like polypeptides need not be full-length nucleic acids, since performance of the methods of the invention does not rely on the use of full-length nucleic acid sequences. According to the present invention, there is provided a method for enhancing yield-related traits in plants, comprising introducing and expressing in a plant a portion of any one of the nucleic acid sequences given in Table A1 of Example 1, or a portion of a nucleic acid encoding an orthologue, paralogue or homologue of any of the amino acid sequences given in Table A1 of Example 1.

A portion of a nucleic acid may be prepared, for example, by making one or more deletions to the nucleic acid. The portions may be used in isolated form or they may be fused to other coding (or non-coding) sequences in order to, for example, produce a protein that combines several activities. When fused to other coding sequences, the resultant polypeptide produced upon translation may be bigger than that predicted for the protein portion.

Portions useful in the methods of the invention, encode a bHLH6-like polypeptide as defined herein, and have substantially the same biological activity as the amino acid sequences given in Table A1 of Example 1. Preferably, the portion is a portion of any one of the nucleic acids given in Table A1 of Example 1, or is a portion of a nucleic acid encoding an orthologue or paralogue of any one of the amino acid sequences given in Table A1 of Example 1. Preferably the portion is at least 500, 600, 700, 800, 900, 1000, 1100, 1200; 1300, 1400, 1500, 1600, 1700, 1800, 1900, 2000, 2100, 2200 consecutive nucleotides in length, the consecutive nucleotides being of any one of the nucleic acid sequences given in Table A1 of Example 1, or of a nucleic acid encoding an orthologue or paralogue of any one of the amino acid sequences given in Table A1 of Example 1. Most preferably the portion is a portion of the nucleic acid of SEQ ID NO: 1. Preferably, the portion encodes a fragment of an amino acid sequence which, when used in the construction of a phylogenetic tree, such as the one depicted in FIG. 6 in Li et al. (2006), clusters within subgroup N (which comprises the amino acid sequence represented by SEQ ID NO: 2), rather than with any other group.

Another nucleic acid variant useful in the methods of the invention is a nucleic acid capable of hybridising, under reduced stringency conditions, preferably under stringent conditions, with a nucleic acid encoding a bHLH6-like polypeptide as defined herein, or with a portion as defined herein.

According to the present invention, there is provided a method for enhancing yield-related traits in plants, comprising introducing and expressing in a plant a nucleic acid capable of hybridizing to any one of the nucleic acids given in Table A1 of Example 1, or comprising introducing and expressing in a plant a nucleic acid capable of hybridising to a nucleic acid encoding an orthologue, paralogue or homologue of any of the nucleic acid sequences given in Table A1 of Example 1.

Hybridising sequences useful in the methods of the invention encode a bHLH6-like polypeptide as defined herein, having substantially the same biological activity as the amino acid sequences given in Table A1 of Example 1. Preferably, the hybridising sequence is capable of hybridising to any one of the nucleic acids given in Table A1 of Example 1, or to a portion of any of these sequences, a portion being as defined above, or the hybridising sequence is capable of hybridising to a nucleic acid encoding an orthologue or paralogue of any one of the amino acid sequences given in Table A1 of Example 1. Most preferably, the hybridising sequence is capable of hybridising to a nucleic acid as represented by SEQ ID NO: 1 or to a portion thereof.

Preferably, the hybridising sequence encodes a polypeptide with an amino acid sequence which, when full-length and used in the construction of a phylogenetic tree, such as the one depicted in FIG. 6 in Li et al. (2006), clusters within subgroup N (which comprises the amino acid sequence represented by SEQ ID NO: 2), rather than with any other group.

Another nucleic acid variant useful in the methods of the invention is a splice variant encoding a bHLH6-like polypeptide as defined hereinabove, a splice variant being as defined herein.

According to the present invention, there is provided a method for enhancing yield-related traits in plants, comprising introducing and expressing in a plant a splice variant of any one of the nucleic acid sequences given in Table A1 of Example 1, or a splice variant of a nucleic acid encoding an orthologue, paralogue or homologue of any of the amino acid sequences given in Table A1 of Example 1.

Preferred splice variants are splice variants of a nucleic acid represented by SEQ ID NO: 1, or a splice variant of a nucleic acid encoding an orthologue or paralogue of SEQ ID NO: 2. Preferably, the amino acid sequence encoded by the splice variant, when used in the construction of a phylogenetic tree, such as the one depicted in FIG. 6 in Li et al. (2006), clusters within subgroup N (which comprises the amino acid sequence represented by SEQ ID NO: 2), rather than with any other group.

Another nucleic acid variant useful in performing the methods of the invention is an allelic variant of a nucleic acid encoding a bHLH6-like polypeptide as defined hereinabove, an allelic variant being as defined herein.

According to the present invention, there is provided a method for enhancing yield-related traits in plants, comprising introducing and expressing in a plant an allelic variant of any one of the nucleic acids given in Table A1 of Example 1, or comprising introducing and expressing in a plant an allelic variant of a nucleic acid encoding an orthologue, paralogue or homologue of any of the amino acid sequences given in Table A1 of Example 1.

The polypeptides encoded by allelic variants useful in the methods of the present invention have substantially the same biological activity as the bHLH6-like polypeptide of SEQ ID NO: 2 and any of the amino acids depicted in Table A1 of Example 1. Allelic variants exist in nature, and encompassed within the methods of the present invention is the use of these natural alleles. Preferably, the allelic variant is an allelic variant of SEQ ID NO: 1 or an allelic variant of a nucleic acid encoding an orthologue or paralogue of SEQ ID NO: 2. Preferably, the amino acid sequence encoded by the allelic variant, when used in the construction of a phylogenetic tree, such as the one depicted in FIG. 6 in Li et al. (2006), clusters within subgroup N (which comprises the amino acid sequence represented by SEQ ID NO: 2), rather than with any other group.

Gene shuffling or directed evolution may also be used to generate variants of nucleic acids encoding bHLH6-like polypeptides as defined above; the term “gene shuffling” being as defined herein. An example of optimisation of bHLH proteins is provided by Pattanaik et al. (BBA1759, 308-318, 2006). This approach may be used for selecting optimised bHLH6-like proteins for use in enhancing yield related traits in plants.

According to the present invention, there is provided a method for enhancing yield-related traits in plants, comprising introducing and expressing in a plant a variant of any one of the nucleic acid sequences given in Table A1 of Example 1, or comprising introducing and expressing in a plant a variant of a nucleic acid encoding an orthologue, paralogue or homologue of any of the amino acid sequences given in Table A1 of Example 1, which variant nucleic acid is obtained by gene shuffling.

Preferably, the amino acid sequence encoded by the variant nucleic acid obtained by gene shuffling, when used in the construction of a phylogenetic tree such as the one depicted in FIG. 6 in Li et al. (2006), clusters within subgroup N (which comprises the amino acid sequence represented by SEQ ID NO: 2), rather than with any other group.

Furthermore, nucleic acid variants may also be obtained by site-directed mutagenesis. Several methods are available to achieve site-directed mutagenesis, the most common being PCR based methods (Current Protocols in Molecular Biology. Wiley Eds.).

Nucleic acids encoding bHLH6-like polypeptides may be derived from any natural or artificial source. The nucleic acid may be modified from its native form in composition and/or genomic environment through deliberate human manipulation. Preferably the bHLH6-like polypeptide-encoding nucleic acid is from a plant, further preferably from a dicotyledonous plant, more preferably from the family Brassicaceae, most preferably the nucleic acid is from Arabidopsis thaliana.

Performance of the methods of the invention gives plants having enhanced yield-related traits. In particular performance of the methods of the invention gives plants having increased yield, especially increased seed yield relative to control plants. The terms “yield” and “seed yield” are described in more detail in the “definitions” section herein.

Reference herein to enhanced yield-related traits is taken to mean an increase in biomass (weight) of one or more parts of a plant, which may include aboveground (harvestable) parts and/or (harvestable) parts below ground. In particular, such harvestable parts are seeds, and performance of the methods of the invention results in plants having increased seed yield relative to the seed yield of control plants. It should be noted that the enhanced yield related traits do not encompass increased resistance to osmotic stress, increased oxidative stress resistance or increased resistance to insects.

Taking corn as an example, a yield increase may be manifested as one or more of the following: increase in the number of plants established per square meter, an increase in the number of ears per plant, an increase in the number of rows, number of kernels per row, kernel weight, thousand kernel weight, ear length/diameter, increase in the seed filling rate (which is the number of filled seeds divided by the total number of seeds and multiplied by 100), among others. Taking rice as an example, a yield increase may manifest itself as an increase in one or more of the following: number of plants per square meter, number of panicles per plant, number of spikelets per panicle, number of flowers (florets) per panicle (which is expressed as a ratio of the number of filled seeds over the number of primary panicles), increase in the seed filling rate (which is the number of filled seeds divided by the total number of seeds and multiplied by 100), increase in thousand kernel weight, among others.

The present invention provides a method for increasing yield, especially seed yield of plants, relative to control plants, which method comprises modulating expression in a plant of a nucleic acid encoding a bHLH6-like polypeptide as defined herein.

Since the transgenic plants according to the present invention have increased yield, it is likely that these plants exhibit an increased growth rate (during at least part of their life cycle), relative to the growth rate of control plants at a corresponding stage in their life cycle.

The increased growth rate may be specific to one or more parts of a plant (including seeds), or may be throughout substantially the whole plant. Plants having an increased growth rate may have a shorter life cycle. The life cycle of a plant may be taken to mean the time needed to grow from a dry mature seed up to the stage where the plant has produced dry mature seeds, similar to the starting material. This life cycle may be influenced by factors such as early vigour, growth rate, greenness index, flowering time and speed of seed maturation. The increase in growth rate may take place at one or more stages in the life cycle of a plant or during substantially the whole plant life cycle. Increased growth rate during the early stages in the life cycle of a plant may reflect enhanced vigour. The increase in growth rate may alter the harvest cycle of a plant allowing plants to be sown later and/or harvested sooner than would otherwise be possible (a similar effect may be obtained with earlier flowering time). If the growth rate is sufficiently increased, it may allow for the further sowing of seeds of the same plant species (for example sowing and harvesting of rice plants followed by sowing and harvesting of further rice plants all within one conventional growing period). Similarly, if the growth rate is sufficiently increased, it may allow for the further sowing of seeds of different plants species (for example the sowing and harvesting of corn plants followed by, for example, the sowing and optional harvesting of soybean, potato or any other suitable plant). Harvesting additional times from the same rootstock in the case of some crop plants may also be possible. Altering the harvest cycle of a plant may lead to an increase in annual biomass production per square meter (due to an increase in the number of times (say in a year) that any particular plant may be grown and harvested). An increase in growth rate may also allow for the cultivation of transgenic plants in a wider geographical area than their wild-type counterparts, since the territorial limitations for growing a crop are often determined by adverse environmental conditions either at the time of planting (early season) or at the time of harvesting (late season). Such adverse conditions may be avoided if the harvest cycle is shortened. The growth rate may be determined by deriving various parameters from growth curves, such parameters may be: T-Mid (the time taken for plants to reach 50% of their maximal size) and T-90 (time taken for plants to reach 90% of their maximal size), amongst others.

According to a preferred feature of the present invention, performance of the methods of the invention gives plants having an increased growth rate relative to control plants. Therefore, according to the present invention, there is provided a method for increasing the growth rate of plants, which method comprises modulating expression in a plant of a nucleic acid encoding a bHLH6-like polypeptide as defined herein.

An increase in yield and/or growth rate occurs whether the plant is under non-stress conditions or whether the plant is exposed to various stresses compared to control plants. Plants typically respond to exposure to stress by growing more slowly. In conditions of severe stress, the plant may even stop growing altogether. Mild stress on the other hand is defined herein as being any stress to which a plant is exposed which does not result in the plant ceasing to grow altogether without the capacity to resume growth. Mild stress in the sense of the invention leads to a reduction in the growth of the stressed plants of less than 40%, 35% or 30%, preferably less than 25%, 20% or 15%, more preferably less than 14%, 13%, 12%, 11% or 10% or less in comparison to the control plant under non-stress conditions. Due to advances in agricultural practices (irrigation, fertilization, pesticide treatments) severe stresses are not often encountered in cultivated crop plants. As a consequence, the compromised growth induced by mild stress is often an undesirable feature for agriculture. Mild stresses are the everyday biotic and/or abiotic (environmental) stresses to which a plant is exposed. Abiotic stresses may be due to drought or excess water, anaerobic stress, salt stress, chemical toxicity, oxidative stress and hot, cold or freezing temperatures. The abiotic stress may be an osmotic stress caused by a water stress (particularly due to drought), salt stress, oxidative stress or an ionic stress. Biotic stresses are typically those stresses caused by pathogens, such as bacteria, viruses, fungi, nematodes and insects.

In particular, the methods of the present invention may be performed under non-stress conditions or under conditions of mild drought to give plants having increased yield relative to control plants. As reported in Wang et al. (Planta (2003) 218: 1-14), abiotic stress leads to a series of morphological, physiological, biochemical and molecular changes that adversely affect plant growth and productivity. Drought, salinity, extreme temperatures and oxidative stress are known to be interconnected and may induce growth and cellular damage through similar mechanisms. Rabbani et al. (Plant Physiol (2003) 133: 1755-1767) describes a particularly high degree of “cross talk” between drought stress and high-salinity stress. For example, drought and/or salinisation are manifested primarily as osmotic stress, resulting in the disruption of homeostasis and ion distribution in the cell. Oxidative stress, which frequently accompanies high or low temperature, salinity or drought stress, may cause denaturing of functional and structural proteins. As a consequence, these diverse environmental stresses often activate similar cell signalling pathways and cellular responses, such as the production of stress proteins, up-regulation of anti-oxidants, accumulation of compatible solutes and growth arrest. The term “non-stress” conditions as used herein are those environmental conditions that allow optimal growth of plants. Persons skilled in the art are aware of normal soil conditions and climatic conditions for a given location.

Performance of the methods of the invention gives plants grown under non-stress conditions or under mild drought conditions increased yield relative to control plants grown under comparable conditions. Therefore, according to the present invention, there is provided a method for increasing yield in plants grown under non-stress conditions or under mild drought conditions, which method comprises modulating expression in a plant of a nucleic acid encoding a bHLH6-like polypeptide.

Performance of the methods of the invention gives plants grown under conditions of nutrient deficiency, particularly under conditions of nitrogen deficiency, increased yield relative to control plants grown under comparable conditions. Therefore, according to the present invention, there is provided a method for increasing yield in plants grown under conditions of nutrient deficiency, which method comprises modulating expression in a plant of a nucleic acid encoding a bHLH6-like polypeptide. Nutrient deficiency may result from a lack of nutrients such as nitrogen, phosphates and other phosphorous-containing compounds, potassium, calcium, cadmium, magnesium, manganese, iron and boron, amongst others.

The present invention encompasses plants or parts thereof (including seeds) obtainable by the methods according to the present invention. The plants or parts thereof comprise a nucleic acid transgene encoding a bHLH6-like polypeptide as defined above.

The invention also provides genetic constructs and vectors to facilitate introduction and/or expression in plants of nucleic acids encoding bHLH6-like polypeptides. The gene constructs may be inserted into vectors, which may be commercially available, suitable for transforming into plants and suitable for expression of the gene of interest in the transformed cells. The invention also provides use of a gene construct as defined herein in the methods of the invention.

More specifically, the present invention provides a construct comprising:
(a) a nucleic acid encoding a bHLH6-like polypeptide as defined above;
(b) one or more control sequences capable of driving expression of the nucleic acid sequence of (a); and optionally
(c) a transcription termination sequence.

Preferably, the nucleic acid encoding a bHLH6-like polypeptide is as defined above. The term “control sequence” and “termination sequence” are as defined herein.

Plants are transformed with a vector comprising any of the nucleic acids described above. The skilled artisan is well aware of the genetic elements that must be present on the vector in order to successfully transform, select and propagate host cells containing the sequence of interest. The sequence of interest is operably linked to one or more control sequences (at least to a promoter).

Advantageously, any type of promoter, whether natural or synthetic, may be used to drive expression of the nucleic acid sequence. A constitutive promoter of plant origin is particularly useful in the methods. Preferably the constitutive promoter is also a ubiquitous promoter. See the “Definitions” section herein for definitions of the various promoter types. Preferably, the plant constitutive promoter is a medium strength promoter and is less strong than the CaMV 35S promoter.

It should be clear that the applicability of the present invention is not restricted to the bHLH6-like polypeptide-encoding nucleic acid represented by SEQ ID NO: 1, nor is the applicability of the invention restricted to expression of a bHLH6-like polypeptide-encoding nucleic acid when driven by a constitutive promoter.

The constitutive promoter is preferably a GOS2 promoter, preferably a GOS2 promoter from rice. Further preferably the constitutive promoter is represented by a nucleic acid sequence substantially similar to SEQ ID NO: 12, most preferably the constitutive promoter is as represented by SEQ ID NO: 12. See Table 2a in the “Definitions” section herein for further examples of constitutive promoters.

Optionally, one or more terminator sequences may be used in the construct introduced into a plant. Preferably, the construct comprises an expression cassette essentially similar or identical to SEQ ID NO 56, comprising the GOS2 promoter and the nucleic acid encoding the bHLH6-like polypeptide.

Additional regulatory elements may include transcriptional as well as translational enhancers. Those skilled in the art will be aware of terminator and enhancer sequences that may be suitable for use in performing the invention. An intron sequence may also be added to the 5′ untranslated region (UTR) or in the coding sequence to increase the amount of the mature message that accumulates in the cytosol, as described in the definitions section. Other control sequences (besides promoter, enhancer, silencer, intron sequences, 3′UTR and/or 5′UTR regions) may be protein and/or RNA stabilizing elements. Such sequences would be known or may readily be obtained by a person skilled in the art.

The genetic constructs of the invention may further include an origin of replication sequence that is required for maintenance and/or replication in a specific cell type. One example is when a genetic construct is required to be maintained in a bacterial cell as an episomal genetic element (e.g. plasmid or cosmid molecule). Preferred origins of replication include, but are not limited to, the f1-ori and colE1.

For the detection of the successful transfer of the nucleic acid sequences as used in the methods of the invention and/or selection of transgenic plants comprising these nucleic acids, it is advantageous to use marker genes (or reporter genes). Therefore, the genetic construct may optionally comprise a selectable marker gene. Selectable markers are described in more detail in the “definitions” section herein. The marker genes may be removed or excised from the transgenic cell once they are no longer needed. Techniques for marker removal are known in the art, useful techniques are described above in the definitions section.

The invention also provides a method for the production of transgenic plants having enhanced yield-related traits relative to control plants, comprising introduction and expression in a plant of any nucleic acid encoding a bHLH6-like polypeptide as defined hereinabove.

More specifically, the present invention provides a method for the production of transgenic plants having increased enhanced yield-related traits, particularly increased emergence vigour and/or seed yield, which method comprises:
(i) introducing and expressing in a plant or plant cell a bHLH6-like polypeptide-encoding nucleic acid; and
(ii) cultivating the plant cell under conditions promoting plant growth and development.

The nucleic acid of (i) may be any of the nucleic acids capable of encoding a bHLH6-like polypeptide as defined herein.

The nucleic acid may be introduced directly into a plant cell or into the plant itself (including introduction into a tissue, organ or any other part of a plant). According to a preferred feature of the present invention, the nucleic acid is preferably introduced into a plant by transformation. The term “transformation” is described in more detail in the “definitions” section herein.

The genetically modified plant cells can be regenerated via all methods with which the skilled worker is familiar. Suitable methods can be found in the abovementioned publications by S. D. Kung and R. Wu, Potrykus or Hagen and Willmitzer.

Generally after transformation, plant cells or cell groupings are selected for the presence of one or more markers which are encoded by plant-expressible genes co-transferred with the gene of interest, following which the transformed material is regenerated into a whole plant. To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as the ones described above.

Following DNA transfer and regeneration, putatively transformed plants may also be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

The present invention clearly extends to any plant cell or plant produced by any of the methods described herein, and to all plant parts and propagules thereof. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced by the parent in the methods according to the invention.

The invention also includes host cells containing an isolated nucleic acid encoding a bHLH6-like polypeptide as defined hereinabove. Preferred host cells according to the invention are plant cells. Host plants for the nucleic acids or the vector used in the method according to the invention, the expression cassette or construct or vector are, in principle, advantageously all plants, which are capable of synthesizing the polypeptides used in the inventive method.

The methods of the invention are advantageously applicable to any plant. Plants that are particularly useful in the methods of the invention include all plants which belong to the superfamily Viridiplantae, in particular monocotyledonous and dicotyledonous plants including fodder or forage legumes, ornamental plants, food crops, trees or shrubs. According to a preferred embodiment of the present invention, the plant is a crop plant. Examples of crop plants include soybean, sunflower, canola, alfalfa, rapeseed, linseed, cotton, tomato, potato and tobacco. Further preferably, the plant is a monocotyledonous plant. Examples of monocotyledonous plants include sugarcane. More preferably the plant is a cereal. Examples of cereals include rice, maize, wheat, barley, millet, rye, triticale, sorghum, emmer, spelt, secale, einkorn, teff, milo and oats.

The invention also extends to harvestable parts of a plant such as, but not limited to seeds, leaves, fruits, flowers, stems, roots, rhizomes, tubers and bulbs. The invention furthermore relates to products derived, preferably directly derived, from a harvestable part of such a plant, such as dry pellets or powders, oil, fat and fatty acids, starch or proteins.

According to a preferred feature of the invention, the modulated expression is increased expression. Methods for increasing expression of nucleic acids or genes, or gene products, are well documented in the art and examples are provided in the definitions section.

As mentioned above, a preferred method for modulating expression of a nucleic acid encoding a bHLH6-like polypeptide is by introducing and expressing in a plant a nucleic acid encoding a bHLH6-like polypeptide; however the effects of performing the method, i.e. enhancing yield-related traits may also be achieved using other well known techniques, including but not limited to T-DNA activation tagging, TILLING, homologous recombination. A description of these techniques is provided in the definitions section.

The present invention also encompasses use of nucleic acids encoding bHLH6-like polypeptides as described herein and use of these bHLH6-like polypeptides in enhancing any of the aforementioned yield-related traits in plants.

Nucleic acids encoding bHLH6-like polypeptide described herein, or the bHLH6-like polypeptides themselves, may find use in breeding programmes in which a DNA marker is identified which may be genetically linked to a bHLH6-like polypeptide-encoding gene. The nucleic acids/genes, or the bHLH6-like polypeptides themselves may be used to define a molecular marker. This DNA or protein marker may then be used in breeding programmes to select plants having enhanced yield-related traits as defined hereinabove in the methods of the invention.

Allelic variants of a bHLH6-like polypeptide-encoding nucleic acid/gene may also find use in marker-assisted breeding programmes. Such breeding programmes sometimes require introduction of allelic variation by mutagenic treatment of the plants, using for example EMS mutagenesis; alternatively, the programme may start with a collection of allelic variants of so called “natural” origin caused unintentionally. Identification of allelic variants then takes place, for example, by PCR. This is followed by a step for selection of superior allelic variants of the sequence in question and which give increased yield. Selection is typically carried out by monitoring growth performance of plants containing different allelic variants of the sequence in question. Growth performance may be monitored in a greenhouse or in the field. Further optional steps include crossing plants in which the superior allelic variant was identified with another plant. This could be used, for example, to make a combination of interesting phenotypic features.

Nucleic acids encoding bHLH6-like polypeptides may also be used as probes for genetically and physically mapping the genes that they are a part of, and as markers for traits linked to those genes. Such information may be useful in plant breeding in order to develop lines with desired phenotypes. Such use of bHLH6-like polypeptide-encoding nucleic acids requires only a nucleic acid sequence of at least 15 nucleotides in length. The bHLH6-like polypeptide-encoding nucleic acids may be used as restriction fragment length polymorphism (RFLP) markers. Southern blots (Sambrook J, Fritsch E F and Maniatis T (1989) Molecular Cloning, A Laboratory Manual) of restriction-digested plant genomic DNA may be probed with the bHLH6-like-encoding nucleic acids. The resulting banding patterns may then be subjected to genetic analyses using computer programs such as MapMaker (Lander et al. (1987) Genomics 1: 174-181) in order to construct a genetic map. In addition, the nucleic acids may be used to probe Southern blots containing restriction endonuclease-treated genomic DNAs of a set of individuals representing parent and progeny of a defined genetic cross. Segregation of the DNA polymorphisms is noted and used to calculate the position of the bHLH6-like polypeptide-encoding nucleic acid in the genetic map previously obtained using this population (Botstein et al. (1980) Am. J. Hum. Genet. 32:314-331).

The production and use of plant gene-derived probes for use in genetic mapping is described in Bernatzky and Tanksley (1986) Plant Mol. Biol. Reporter 4: 37-41. Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

The nucleic acid probes may also be used for physical mapping (i.e., placement of sequences on physical maps; see Hoheisel et al. In: Non-mammalian Genomic Analysis: A Practical Guide, Academic press 1996, pp. 319-346, and references cited therein).

In another embodiment, the nucleic acid probes may be used in direct fluorescence in situ hybridisation (FISH) mapping (Trask (1991) Trends Genet. 7:149-154). Although current methods of FISH mapping favour use of large clones (several kb to several hundred kb; see Laan et al. (1995) Genome Res. 5:13-20), improvements in sensitivity may allow performance of FISH mapping using shorter probes.

A variety of nucleic acid amplification-based methods for genetic and physical mapping may be carried out using the nucleic acids. Examples include allele-specific amplification (Kazazian (1989) J. Lab. Clin. Med 11:95-96), polymorphism of PCR-amplified fragments (CAPS; Sheffield et al. (1993) Genomics 16:325-332), allele-specific ligation (Landegren et al. (1988) Science 241:1077-1080), nucleotide extension reactions (Sokolov (1990) Nucleic Acid Res. 18:3671), Radiation Hybrid Mapping (Walter et al. (1997) Nat. Genet. 7:22-28) and Happy Mapping (Dear and Cook (1989) Nucleic Acid Res. 17:6795-6807). For these methods, the sequence of a nucleic acid is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for mapping methods.

The methods according to the present invention result in plants having enhanced yield-related traits, as described hereinbefore. These traits may also be combined with other economically advantageous traits, such as further yield-enhancing traits, tolerance to other abiotic and biotic stresses, traits modifying various architectural features and/or biochemical and/or physiological features.

In one embodiment the invention relates to subject mater summarized as follows:

Item 1: A method for enhancing yield-related traits in plants relative to control plants, comprising modulating expression in a plant of a nucleic acid encoding a bHLH6-like polypeptide, wherein said bHLH6-like polypeptide comprises a HLH domain.
Item 2: A method according to item 1, wherein said bHLH6-like polypeptide comprises one or more of the following motifs:
Motif 1 (SEQ ID NO: 3),
Motif 2 (SEQ ID NO: 4),
Motif 3: (SEQ ID NO: 5)
Motif 7 (SEQ ID NO: 9) or a sequence that has at least 80% sequence identity to SEQ ID NO: 9.
   Item 3: Method according to item 1 or 2, wherein said modulated expression is effected by introducing and expressing in a plant a nucleic acid encoding a bHLH6-like polypeptide.
   Item 4: Method according to any preceding claim, wherein said nucleic acid encoding a bHLH6-like polypeptide encodes any one of the proteins listed in Table A1 or is a portion of such a nucleic acid, or a nucleic acid capable of hybridising with such a nucleic acid.
   Item 5: Method according to any preceding claim, wherein said nucleic acid sequence encodes an orthologue or paralogue of any of the proteins given in Table A1.
   Item 6: Method according to any preceding claim, wherein said enhanced yield-related traits comprise increased yield, preferably increased emergence vigour and/or increased seed yield relative to control plants.
   Item 7: Method according to any one of items 1 to 6, wherein said enhanced yield-related traits are obtained under non-stress conditions.
   Item 8: Method according to any one of items 3 to 7, wherein said nucleic acid is operably linked to a constitutive promoter, preferably to a GOS2 promoter, most preferably to a GOS2 promoter from rice.
   Item 9: Method according to any preceding item, wherein said nucleic acid encoding a bHLH6-like polypeptide is of plant origin, preferably from a dicotyledonous plant, further preferably from the family Brassicaceae, more preferably from the genus Arabidopsis, most preferably from Arabidopsis thaliana.

Item 10: Plant or part thereof, including seeds, obtainable by a method according to any preceding item, wherein said plant or part thereof comprises a recombinant nucleic acid encoding a bHLH6-like polypeptide.

Item 11: Construct comprising:
(a) nucleic acid encoding a bHLH6-like polypeptide as defined in items 1 or 2;
(b) one or more control sequences capable of driving expression of the nucleic acid sequence of (a); and optionally
(c) a transcription termination sequence.
   Item 12: Construct according to item 11, wherein one of said control sequences is a constitutive promoter, preferably a GOS2 promoter, most preferably a GOS2 promoter from rice.
   Item 13: Use of a construct according to item 11 or 12 in a method for making plants having increased yield, particularly increased emergence vigour and/or increased seed yield relative to control plants.
   Item 14: Plant, plant part or plant cell transformed with a construct according to item 10 or 11.
   Item 15: Method for the production of a transgenic plant having increased yield, particularly increased biomass and/or increased seed yield relative to control plants, comprising:
(i) introducing and expressing in a plant a nucleic acid encoding a bHLH6-like polypeptide as defined in item 1 or 2; and
(ii) cultivating the plant cell under conditions promoting plant growth and development.
   Item 16: Transgenic plant having increased yield, particularly increased biomass and/or increased seed yield, relative to control plants, resulting from modulated expression of a nucleic acid encoding a bHLH6-like polypeptide as defined in item 1 or 2, or a transgenic plant cell derived from said transgenic plant.
   Item 17: Transgenic plant according to item 10, 14 or 16, or a transgenic plant cell derived thereof, wherein said plant is a crop plant or a monocot or a cereal, such as rice, maize, wheat, barley, millet, rye, triticale, sorghum emmer, spelt, secale, einkorn, teff, milo and oats.
   Item 18: Harvestable parts of a plant according to item 17, wherein said harvestable parts are preferably shoot biomass and/or seeds.
   Item 19: Products derived from a plant according to item 17 and/or from harvestable parts of a plant according to item 19.
   Item 20: Use of a nucleic acid encoding a bHLH6-like polypeptide in increasing yield, particularly in increasing seed yield and/or shoot biomass in plants, relative to control plants.

In another embodiment of the invention it has now been found that modulating expression in a plant of a nucleic acid sequence encoding a GRP polypeptide, wherein said GRP polypeptide is an RrmJ/FtsJ ribosomal RNA methyltransferase polypeptide, gives plants having enhanced yield-related traits relative to control plants. According to a first embodiment, the present invention provides a method for enhancing yield-related traits in plants relative to control plants, comprising modulating expression in a plant of a nucleic acid sequence encoding a GRP polypeptide, wherein said GRP polypeptide is an RrmJ/FtsJ ribosomal RNA methyltransferase polypeptide (RrmJ/FtsJ polypeptide).

A preferred method for modulating (preferably, increasing) expression of a nucleic acid sequence encoding a GRP polypeptide is by introducing and expressing in a plant a nucleic acid sequence encoding a GRP polypeptide.

In one embodiment any reference hereinafter to a “protein useful in the methods of the invention” is taken to mean a GRP polypeptide as defined herein. In the same embodiment any reference hereinafter to a “nucleic acid useful in the methods of the invention” is taken to mean a nucleic acid sequence capable of encoding such a GRP polypeptide. In one embodiment the nucleic acid sequence to be introduced into a plant (and therefore useful in performing the methods of the invention) is any nucleic acid sequence encoding the type of protein which will now be described, hereafter also named “GRP nucleic acid” or “GRP gene”.

In one embodiment a “GRP polypeptide” as defined herein refers the proteins represented by SEQ ID NO: 58, by SEQ ID NO: 60, and to homologues (orthologues and paralogues) thereof. Table A2 of Example 1 herein presents such orthologues and paralogues.

Preferably, the homologues of SEQ ID NO: 58 or of SEQ ID NO: 60 have a ribosomal RNA methyltransferase RrmJ/FtsJ domain (InterPro entries IPR002877 and IPR015507; Pfam entry PF01728; PANTHER entry PTHR10920).

Alternatively or additionally, a “GRP polypeptide” as defined herein refers to any polypeptide having in increasing order of preference at least 50%, 55%, 60%, 65%, 70%, 75%, 80%, 85%, 90%, 95%, 98%, 99% or more polypeptide sequence identity to the GRP polypeptide as represented by SEQ ID NO: 58, or by SEQ ID NO: 60, or to any of the polypeptide sequences given in Table A2 of Example 1 herein.

In one embodiment the methods of the present invention make use of a partial GRP polypeptide, wherein the GRP polypeptide is an RrmJ/FtsJ polypeptide. GRP polypeptides may be identified by the presence of one or more of several well-known features (see above). Upon identification of a GRP polypeptide, a person skilled in the art could easily derive, using routine techniques, a corresponding partial GRP-encoding nucleic acid sequence useful in performing the methods of the invention.

The term “domain” and “motif” is defined in the “definitions” section herein. Specialist databases exist for the identification of domains, for example, SMART (Schultz et al. (1998) Proc. Natl. Acad. Sci. USA 95, 5857-5864; Letunic et al. (2002) Nucleic Acids Res 30, 242-244), InterPro (Mulder et al., (2003) Nucl. Acids. Res. 31, 315-318), Prosite (Bucher and Bairoch (1994), A generalized profile syntax for biomolecular sequences motifs and its function in automatic sequence interpretation. (In) ISMB-94; Proceedings 2nd International Conference on Intelligent Systems for Molecular Biology. Altman R., Brutlag D., Karp P., Lathrop R., Searls D., Eds., pp 53-61, AAAI Press, Menlo Park; Hulo et al., Nucl. Acids. Res. 32:D134-D137, (2004) ), or Pfam (Bateman et al., Nucleic Acids Research 30(1): 276-280 (2002) ). A set of tools for in silico analysis of protein sequences is available on the ExPASy proteomics server (Swiss Institute of Bioinformatics (Gasteiger et al., ExPASy: the proteomics server for in-depth protein knowledge and analysis, Nucleic Acids Res. 31:3784-3788 (2003) ). Domains or motifs may also be identified using routine techniques, such as by sequence alignment.

Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the global (i.e. spanning the complete sequences) alignment of two sequences that maximizes the number of matches and minimizes the number of gaps. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information (NCBI). Homologues may readily be identified using, for example, the ClustalW multiple sequence alignment algorithm (version 1.83), with the default pairwise alignment parameters, and a scoring method in percentage. Global percentages of similarity and identity may also be determined using one of the methods available in the MatGAT software package (Campanella et al., BMC Bioinformatics. 2003 Jul. 10; 4:29. MatGAT: an application that generates similarity/identity matrices using protein or DNA sequences). Minor manual editing may be performed to optimise alignment between conserved motifs, as would be apparent to a person skilled in the art. Furthermore, instead of using full-length sequences for the identification of homologues, specific domains may also be used. The sequence identity values may be determined over the entire nucleic acid sequence or polypeptide sequence or over selected domains or conserved motif(s), using the programs mentioned above using the default parameters.

Furthermore, GRP polypeptides, as far as SEQ ID NO: 58, SEQ ID NO: 60, and its homologues are concerned, typically have the capability to methylate 23 S ribosomal RNA at position U2552 in the aminoacyl (A)1-site of the ribosome, only when the 23 S rRNA is present in 50 S ribosomal subunits. Tools and techniques for assaying the rRNA methyltransferase activity are well known in the art, for example in Hager et al. (2004) J Bacteriol 186(19): 6634-6642.

The present invention is illustrated by transforming plants with the nucleic acid sequence represented by SEQ ID NO: 57, encoding the polypeptide sequence of SEQ ID NO: 58. However, performance of the invention is not restricted to these sequences; the methods of the invention may advantageously be performed using any GRP-encoding nucleic acid sequence or GRP polypeptide as defined herein.

Examples of nucleic acids encoding GRP polypeptides may be found in databases known in the art. Such nucleic acids are useful in performing the methods of the invention. Orthologues and paralogues, the terms “orthologues” and “paralogues” being as defined herein, may readily be identified by performing a so-called reciprocal blast search. Typically, this involves a first BLAST involving BLASTing a query sequence (for example using SEQ ID NO: 58 or SEQ ID NO: 60) against any sequence database, such as the publicly available NCBI database. BLASTN or TBLASTX (using standard default values) are generally used when starting from a nucleotide sequence, and BLASTP or TBLASTN (using standard default values) when starting from a protein sequence. The BLAST results may optionally be filtered. The full-length sequences of either the filtered results or non-filtered results are then BLASTed back (second BLAST) against sequences from the organism from which the query sequence is derived (where the query sequence is SEQ ID NO: 57, SEQ ID NO: 58, SEQ ID NO: 59, or SEQ ID NO: 60, the second BLAST would therefore be against Nicotiana tabacum sequences). The results of the first and second BLASTs are then compared. A paralogue is identified if a high-ranking hit from the first blast is from the same species as from which the query sequence is derived, a BLAST back then ideally results in the query sequence amongst the highest hits; an orthologue is identified if a high-ranking hit in the first BLAST is not from the same species as from which the query sequence is derived, and preferably results upon BLAST back in the query sequence being among the highest hits.

High-ranking hits are those having a low E-value. The lower the E-value, the more significant the score (or in other words the lower the chance that the hit was found by chance). Computation of the E-value is well known in the art. In addition to E-values, comparisons are also scored by percentage identity. Percentage identity refers to the number of identical nucleotides (or amino acids) between the two compared nucleic acid sequence (or polypeptide) sequences over a particular length. In the case of large families, ClustalW may be used, followed by a neighbour joining tree, to help visualize clustering of related genes and to identify orthologues and paralogues.

Nucleic acid variants encoding homologues and derivatives of SEQ ID NO: 58 or of SEQ ID NO: 60, may also be useful in practising the methods of the invention, the terms “homologue” and “derivative” being as defined herein. Also useful in the methods of the invention are nucleic acids encoding homologues and derivatives of orthologues or paralogues of SEQ ID NO: 58 or of SEQ ID NO: 60. Homologues and derivatives useful in the methods of the present invention have substantially the same biological and functional activity as the unmodified protein from which they are derived.

Further nucleic acid sequence variants useful in practising the methods of the invention include portions of nucleic acids encoding GRP polypeptides, nucleic acids hybridising to nucleic acids encoding GRP polypeptides, splice variants of nucleic acids encoding GRP polypeptides, allelic variants of nucleic acids encoding GRP polypeptides and variants of nucleic acids encoding GRP polypeptides obtained by gene shuffling. The terms hybridising sequence, splice variant, allelic variant and gene shuffling are as described herein.

Nucleic acids encoding GRP polypeptides need not be full-length nucleic acids, since performance of the methods of the invention does not rely on the use of full-length nucleic acid sequences. According to the present invention, there is provided a method for enhancing yield-related traits in plants, comprising introducing and expressing in a plant a portion of SEQ ID NO: 57, or a portion of SEQ ID NO: 59, or a portion of a nucleic acid sequence encoding an orthologue, paralogue or homologue of SEQ ID NO: 60.

A portion of a nucleic acid sequence may be prepared, for example, by making one or more deletions to the nucleic acid. The portions may be used in isolated form or they may be fused to other coding (or non-coding) sequences in order to, for example, produce a protein that combines several activities. When fused to other coding sequences, the resultant polypeptide produced upon translation may be bigger than that predicted for the protein portion.

Portions useful in the methods of the invention, encode a GRP polypeptide as defined herein, and have substantially the same biological activity as the polypeptide sequences given in SEQ ID NO: 58 or of SEQ ID NO: 60. Preferably, the portion is a portion of the nucleic acid given in SEQ ID NO: 57 or SEQ ID NO: 59, or is a portion of a nucleic acid sequence encoding an orthologue or paralogue of the polypeptide sequences given in SEQ ID NO: 58 or of SEQ ID NO: 60. Preferably the portion is at least 400, 450, 500, 550, 600, 650, 700, 750 consecutive nucleotides in length, the consecutive nucleotides being of SEQ ID NO: 57 or of SEQ ID NO: 59, or of a nucleic acid sequence encoding an orthologue or paralogue of SEQ ID NO: 58 or of SEQ ID NO: 60. Most preferably the portion is a portion of the nucleic acid sequence of SEQ ID NO: 57 or of SEQ ID NO: 59.

Another nucleic acid sequence variant useful in the methods of the invention is a nucleic acid sequence capable of hybridising, under reduced stringency conditions, preferably under stringent conditions, with a nucleic acid sequence encoding a GRP polypeptide as defined herein, or with a portion as defined herein.

According to the present invention, there is provided a method for enhancing yield-related traits in plants, comprising introducing and expressing in a plant a nucleic acid sequence capable of hybridizing to SEQ ID NO: 57 or to SEQ ID NO: 59, or comprising introducing and expressing in a plant a nucleic acid sequence capable of hybridising to a nucleic acid sequence encoding an orthologue, paralogue or homologue of SEQ ID NO: 58 or of SEQ ID NO: 60.

Hybridising sequences useful in the methods of the invention encode a GRP polypeptide as defined herein, having substantially the same biological activity as the polypeptide sequences given in SEQ ID NO: 58. Preferably, the hybridising sequence is capable of hybridising to SEQ ID NO: 57 or to SEQ ID NO: 59, or to a portion of any of these sequences, a portion being as defined above, or the hybridising sequence is capable of hybridising to a nucleic acid sequence encoding an orthologue or paralogue of SEQ ID NO: 58 or of SEQ ID NO: 60.

Another nucleic acid sequence variant useful in the methods of the invention is a splice variant encoding a GRP polypeptide as defined hereinabove, a splice variant being as defined herein.

According to the present invention, there is provided a method for enhancing yield-related traits in plants, comprising introducing and expressing in a plant a splice variant of SEQ ID NO: 57 or of SEQ ID NO: 59, or a splice variant of a nucleic acid sequence encoding an orthologue, paralogue or homologue of SEQ ID NO: 58 or of SEQ ID NO: 60.

Another nucleic acid sequence variant useful in performing the methods of the invention is an allelic variant of a nucleic acid sequence encoding a GRP polypeptide as defined hereinabove, an allelic variant being as defined herein.

According to the present invention, there is provided a method for enhancing yield-related traits in plants, comprising introducing and expressing in a plant an allelic variant of SEQ ID NO: 57 or of SEQ ID NO: 59, or comprising introducing and expressing in a plant an allelic variant of a nucleic acid sequence encoding an orthologue, paralogue or homologue of the polypeptide sequences represented by SEQ ID NO: 58 or of SEQ ID NO: 60.

The allelic variants useful in the methods of the present invention have substantially the same biological activity as the GRP polypeptide of SEQ ID NO: 58 or of SEQ ID NO: 60. Allelic variants exist in nature, and encompassed within the methods of the present invention is the use of these natural alleles. Gene shuffling or directed evolution may also be used to generate variants of nucleic acids encoding GRP polypeptides as defined above; the term “gene shuffling” being as defined herein.

According to the present invention, there is provided a method for enhancing yield-related traits in plants, comprising introducing and expressing in a plant a variant of SEQ ID NO: 57 or of SEQ ID NO: 59, or comprising introducing and expressing in a plant a variant of a nucleic acid sequence encoding an orthologue, paralogue or homologue of SEQ ID NO: 58 or of SEQ ID NO: 60, which variant nucleic acid sequence is obtained by gene shuffling.

Furthermore, nucleic acid sequence variants may also be obtained by site-directed mutagenesis. Several methods are available to achieve site-directed mutagenesis, the most common being PCR based methods (Current Protocols in Molecular Biology. Wiley Eds.).

Nucleic acids encoding GRP polypeptides may be derived from any natural or artificial source. The nucleic acid sequence may be modified from its native form in composition and/or genomic environment through deliberate human manipulation. Preferably the GRP polypeptide-encoding nucleic acid sequence is from a plant. In the case of SEQ ID NO: 57 or of SEQ ID NO: 59, the GRP polypeptide encoding nucleic acid sequence is preferably from a dicotyledonous plant, more preferably from the family Solanaceae, most preferably the nucleic acid sequence is from Nicotiana tabacum.

Performance of the methods of the invention gives plants having enhanced yield-related traits. In particular performance of the methods of the invention gives plants having increased early vigour and increased yield, especially increased biomass and increased seed yield relative to control plants. The terms “yield” and “seed yield” are described in more detail in the “definitions” section herein.

Reference herein to enhanced yield-related traits is taken to mean an increase in early vigour and/or in biomass (weight) of one or more parts of a plant, which may include aboveground (harvestable) parts and/or (harvestable) parts below ground. In particular, such harvestable parts are biomass and/or seeds, and performance of the methods of the invention results in plants having increased early vigour, biomass and/or seed yield relative to the early vigour, biomass or seed yield of control plants.

Taking corn as an example, a yield increase may be manifested as one or more of the following: increase in the number of plants established per hectare or acre, an increase in the number of ears per plant, an increase in the number of rows, number of kernels per row, kernel weight, thousand kernel weight, ear length/diameter, increase in the seed filling rate (which is the number of filled seeds divided by the total number of seeds and multiplied by 100), among others. Taking rice as an example, a yield increase may manifest itself as an increase in one or more of the following: number of plants per hectare or acre, number of panicles per plant, number of spikelets per panicle, number of flowers (florets) per panicle (which is expressed as a ratio of the number of filled seeds over the number of primary panicles), increase in the seed filling rate (which is the number of filled seeds divided by the total number of seeds and multiplied by 100), increase in thousand kernel weight, among others.

The present invention provides a method for enhancing yield-related traits, especially biomass and/or seed yield of plants, relative to control plants, which method comprises modulating expression, preferably increasing expression, in a plant of a nucleic acid sequence encoding a GRP polypeptide as defined herein.

Since the transgenic plants according to the present invention have enhanced yield-related traits, it is likely that these plants exhibit an increased growth rate (during at least part of their life cycle), relative to the growth rate of control plants at a corresponding stage in their life cycle.

The increased growth rate may be specific to one or more parts of a plant (including seeds), or may be throughout substantially the whole plant. Plants having an increased growth rate may have a shorter life cycle. The life cycle of a plant may be taken to mean the time needed to grow from a dry mature seed up to the stage where the plant has produced dry mature seeds, similar to the starting material. This life cycle may be influenced by factors such as early vigour, growth rate, greenness index, flowering time and speed of seed maturation. The increase in growth rate may take place at one or more stages in the life cycle of a plant or during substantially the whole plant life cycle. Increased growth rate during the early stages in the life cycle of a plant may reflect enhanced vigour. The increase in growth rate may alter the harvest cycle of a plant allowing plants to be sown later and/or harvested sooner than would otherwise be possible (a similar effect may be obtained with earlier flowering time). If the growth rate is sufficiently increased, it may allow for the further sowing of seeds of the same plant species (for example sowing and harvesting of rice plants followed by sowing and harvesting of further rice plants all within one conventional growing period). Similarly, if the growth rate is sufficiently increased, it may allow for the further sowing of seeds of different plants species (for example the sowing and harvesting of corn plants followed by, for example, the sowing and optional harvesting of soybean, potato or any other suitable plant). Harvesting additional times from the same rootstock in the case of some crop plants may also be possible. Altering the harvest cycle of a plant may lead to an increase in annual biomass production per acre (due to an increase in the number of times (say in a year) that any particular plant may be grown and harvested). An increase in growth rate may also allow for the cultivation of transgenic plants in a wider geographical area than their wild-type counterparts, since the territorial limitations for growing a crop are often determined by adverse environmental conditions either at the time of planting (early season) or at the time of harvesting (late season). Such adverse conditions may be avoided if the harvest cycle is shortened. The growth rate may be determined by deriving various parameters from growth curves, such parameters may be: T-Mid (the time taken for plants to reach 50% of their maximal size) and T-90 (time taken for plants to reach 90% of their maximal size), amongst others.

According to a preferred feature of the present invention, performance of the methods of the invention gives plants having an increased growth rate relative to control plants. Therefore, according to the present invention, there is provided a method for increasing the growth rate of plants, which method comprises modulating expression, preferably increasing expression, in a plant of a nucleic acid sequence encoding a GRP polypeptide as defined herein.

An enhancement of yield-related traits (an increase in yield and/or growth rate) occurs whether the plant is under non-stress conditions or whether the plant is exposed to various stresses compared to control plants. Plants typically respond to exposure to stress by growing more slowly. In conditions of severe stress, the plant may even stop growing altogether. Mild stress on the other hand is defined herein as being any stress to which a plant is exposed which does not result in the plant ceasing to grow altogether without the capacity to resume growth. Mild stress in the sense of the invention leads to a reduction in the growth of the stressed plants of less than 40%, 35% or 30%, preferably less than 25%, 20% or 15%, more preferably less than 14%, 13%, 12%, 11% or 10% or less in comparison to the control plant under non-stress conditions. Due to advances in agricultural practices (irrigation, fertilization, pesticide treatments) severe stresses are not often encountered in cultivated crop plants. As a consequence, the compromised growth induced by mild stress is often an undesirable feature for agriculture. Mild stresses are the everyday biotic and/or abiotic (environmental) stresses to which a plant is exposed. Abiotic stresses may be due to drought or excess water, anaerobic stress, salt stress, chemical toxicity, oxidative stress and hot, cold or freezing temperatures. The abiotic stress may be an osmotic stress caused by a water stress (particularly due to drought), salt stress, oxidative stress or an ionic stress. Biotic stresses are typically those stresses caused by pathogens, such as bacteria, viruses, fungi and insects. The term “non-stress” conditions as used herein are those environmental conditions that allow optimal growth of plants. Persons skilled in the art are aware of normal soil conditions and climatic conditions for a given location.

Performance of the methods of the invention gives plants grown under non-stress conditions or under mild drought conditions enhanced yield-related traits relative to control plants grown under comparable conditions. Therefore, according to the present invention, there is provided a method for enhancing yield-related traits in plants grown under non-stress conditions or under mild drought conditions, which method comprises modulating, preferably increasing, expression in a plant of a nucleic acid sequence encoding a GRP polypeptide.

Performance of the methods according to the present invention results in plants grown under abiotic stress conditions having enhanced yield-related traits, preferably enhanced seed yield-related traits, relative to control plants grown under comparable stress conditions. As reported in Wang et al. (Planta (2003) 218: 1-14), abiotic stress leads to a series of morphological, physiological, biochemical and molecular changes that adversely affect plant growth and productivity. Drought, salinity, extreme temperatures and oxidative stress are known to be interconnected and may induce growth and cellular damage through similar mechanisms. Rabbani et al. (Plant Physiol (2003) 133: 1755-1767) describes a particularly high degree of “cross talk” between drought stress and high-salinity stress. For example, drought and/or salinisation are manifested primarily as osmotic stress, resulting in the disruption of homeostasis and ion distribution in the cell. Oxidative stress, which frequently accompanies high or low temperature, salinity or drought stress, may cause denaturing of functional and structural proteins. As a consequence, these diverse environmental stresses often activate similar cell signalling pathways and cellular responses, such as the production of stress proteins, up-regulation of anti-oxidants, accumulation of compatible solutes and growth arrest. Since diverse environmental stresses activate similar pathways, the exemplification of the present invention with drought stress should not be seen as a limitation to drought stress, but more as a screen to indicate the involvement of GRP polypeptides as defined above, in enhancing yield-related traits, preferably enhancing seed yield-related traits, relative to control plants grown in comparable stress conditions, in abiotic stresses in general.

The term “abiotic stress” as defined herein is taken to mean any one or more of: water stress (due to drought or excess water), anaerobic stress, salt stress, temperature stress (due to hot, cold or freezing temperatures), chemical toxicity stress and oxidative stress. According to one aspect of the invention, the abiotic stress is an osmotic stress, selected from water stress, salt stress, oxidative stress and ionic stress. Preferably, the water stress is drought stress. The term salt stress is not restricted to common salt (NaCl), but may be any stress caused by one or more of: NaCl, KCl, LiCl, MgCl2, CaCl2, amongst others.

Performance of the methods of the invention gives plants having enhanced yield-related traits, preferably enhanced seed yield-related traits, under abiotic stress conditions relative to control plants grown in comparable stress conditions. Therefore, according to the present invention, there is provided a method for enhancing yield-related traits, preferably enhancing seed yield-related traits, in plants grown under abiotic stress conditions, which method comprises modulatingexpression in a plant of a nucleic acid sequence encoding a GRP polypeptide. According to one aspect of the invention, the abiotic stress is an osmotic stress, selected from one or more of the following: water stress, salt stress, oxidative stress and ionic stress.

Performance of the methods of the invention gives plants grown under conditions of nutrient deficiency, particularly under conditions of nitrogen deficiency, enhanced yield-related traits relative to control plants grown under comparable conditions. Therefore, according to the present invention, there is provided a method for enhancing yield-related traits in plants grown under conditions of nutrient deficiency, which method comprises modulating, preferably increasing, expression in a plant of a nucleic acid sequence encoding a GRP polypeptide. Nutrient deficiency may result from a lack of nutrients such as nitrogen, phosphates and other phosphorous-containing compounds, potassium, calcium, cadmium, magnesium, manganese, iron and boron, amongst others.

The present invention encompasses plants, parts thereof (including seeds), or plant cells obtainable by the methods according to the present invention. The plants, parts thereof, or plant cells comprise a nucleic acid transgene encoding a GRP polypeptide as defined above.

The invention also provides genetic constructs and vectors to facilitate introduction and/or expression in plants of nucleic acids encoding GRP polypeptides. The gene constructs may be inserted into vectors, which may be commercially available, suitable for transforming into plants and suitable for expression of the gene of interest in the transformed cells. The invention also provides use of a gene construct as defined herein in the methods of the invention.

More specifically, the present invention provides a construct comprising:
(a) a nucleic acid sequence encoding a GRP polypeptide as defined above;
(b) one or more control sequences capable of driving expression of the nucleic acid sequence of (a); and optionally
(c) a transcription termination sequence.
Preferably, the nucleic acid sequence encoding a GRP polypeptide is as defined above. The term “control sequence” and “termination sequence” are as defined herein.

Plants are transformed with a vector comprising any of the nucleic acids described above. The skilled artisan is well aware of the genetic elements that must be present on the vector in order to successfully transform, select and propagate host cells containing the sequence of interest. The sequence of interest is operably linked to one or more control sequences (at least to a promoter).

Advantageously, any type of promoter, whether natural or synthetic, may be used to drive expression of the nucleic acid sequence. A seed-specific promoter is particularly useful in the methods of the invention. See the “Definitions” section herein for definitions of the various promoter types.

It should be clear that the applicability of the present invention is not restricted to the GRP polypeptide-encoding nucleic acid sequence represented by SEQ ID NO: 57 or of SEQ ID NO: 59, nor is the applicability of the invention restricted to expression of a GRP polypeptide-encoding nucleic acid sequence when driven by a seed-specific promoter.

The seed-specific promoter is preferably an oleosin promoter, preferably a oleosin promoter from rice. Further preferably the oleosin promoter is represented by a nucleic acid sequence substantially similar to SEQ ID NO: 91, most preferably the oleosin promoter is as represented by SEQ ID NO: 91. See Table 2b in the “Definitions” section herein for further examples of seed-specific promoters.

Optionally, one or more terminator sequences may be used in the construct introduced into a plant. Additional regulatory elements may include transcriptional as well as translational enhancers. Those skilled in the art will be aware of terminator and enhancer sequences that may be suitable for use in performing the invention. An intron sequence may also be added to the 5′ untranslated region (UTR) or in the coding sequence to increase the amount of the mature message that accumulates in the cytosol, as described in the definitions section. Other control sequences (besides promoter, enhancer, silencer, intron sequences, 3′UTR and/or 5′UTR regions) may be protein and/or RNA stabilizing elements. Such sequences would be known or may readily be obtained by a person skilled in the art.

The genetic constructs of the invention may further include an origin of replication sequence that is required for maintenance and/or replication in a specific cell type. One example is when a genetic construct is required to be maintained in a bacterial cell as an episomal genetic element (e.g. plasmid or cosmid molecule). Preferred origins of replication include, but are not limited to, the f1-ori and colE1.

For the detection of the successful transfer of the nucleic acid sequences as used in the methods of the invention and/or selection of transgenic plants comprising these nucleic acids, it is advantageous to use marker genes (or reporter genes). Therefore, the genetic construct may optionally comprise a selectable marker gene. Selectable markers are described in more detail in the “definitions” section herein. The marker genes may be removed or excised from the transgenic cell once they are no longer needed. Techniques for marker removal are known in the art, useful techniques are described above in the definitions section.

The invention also provides a method for the production of transgenic plants having enhanced yield-related traits relative to control plants, comprising introduction and expression in a plant of any nucleic acid sequence encoding a GRP polypeptide as defined hereinabove.

More specifically, the present invention provides a method for the production of transgenic plants having enhanced yield-related traits which method comprises:
(i) introducing and expressing in a plant, plant part, or plant cell, a nucleic acid sequence encoding a GRP polypeptide; and
(ii) cultivating the plant, plant part or plant cell under conditions promoting plant growth and development.

The nucleic acid sequence of (i) may be any of the nucleic acids capable of encoding a GRP polypeptide as defined herein.

The nucleic acid sequence may be introduced directly into a plant cell or into the plant itself (including introduction into a tissue, organ or any other part of a plant). According to a preferred feature of the present invention, the nucleic acid sequence is preferably introduced into a plant by transformation. The term “transformation” is described in more detail in the “definitions” section herein.

The genetically modified plant cells can be regenerated via all methods with which the skilled worker is familiar. Suitable methods can be found in the abovementioned publications by S. D. Kung and R. Wu, Potrykus or Hofgen and Willmitzer.

Generally after transformation, plant cells or cell groupings are selected for the presence of one or more markers which are encoded by plant-expressible genes co-transferred with the gene of interest, following which the transformed material is regenerated into a whole plant. To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as the ones described above.

Following DNA transfer and regeneration, putatively transformed plants may also be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

The present invention clearly extends to any plant cell or plant produced by any of the methods described herein, and to all plant parts and propagules thereof. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced by the parent in the methods according to the invention.

The invention also includes host cells containing an isolated nucleic acid sequence encoding a GRP polypeptide as defined hereinabove. Preferred host cells according to the invention are plant cells. Host plants for the nucleic acids or the vector used in the method according to the invention, the expression cassette or construct or vector are, in principle, advantageously all plants, which are capable of synthesizing the polypeptides used in the inventive method.

The methods of the invention are advantageously applicable to any plant. Plants that are particularly useful in the methods of the invention include all plants which belong to the superfamily Viridiplantae, in particular monocotyledonous and dicotyledonous plants including fodder or forage legumes, ornamental plants, food crops, trees or shrubs. According to a preferred embodiment of the present invention, the plant is a crop plant. Examples of crop plants include soybean, sunflower, canola, alfalfa, rapeseed, cotton, tomato, potato and tobacco. Further preferably, the plant is a monocotyledonous plant. Examples of monocotyledonous plants include sugarcane. More preferably the plant is a cereal. Examples of cereals include rice, maize, wheat, barley, millet, rye, triticale, sorghum and oats.

The invention also extends to harvestable parts of a plant such as, but not limited to seeds, leaves, fruits, flowers, stems, roots, rhizomes, tubers and bulbs. The invention furthermore relates to products derived, preferably directly derived, from a harvestable part of such a plant, such as dry pellets or powders, oil, fat and fatty acids, starch or proteins.

According to a preferred feature of the invention, the modulated expression is increased expression. Methods for increasing expression of nucleic acids or genes, or gene products, are well documented in the art and examples are provided in the definitions section.

As mentioned above, a preferred method for modulating (preferably, increasing) expression of a nucleic acid sequence encoding a GRP polypeptide is by introducing and expressing in a plant a nucleic acid sequence encoding a GRP polypeptide; however the effects of performing the method, i.e. enhancing yield-related traits may also be achieved using other well known techniques, including but not limited to T-DNA activation tagging, TILLING, homologous recombination. A description of these techniques is provided in the definitions section.

The present invention also encompasses use of nucleic acids encoding GRP polypeptides as described herein and use of these GRP polypeptides in enhancing any of the aforementioned yield-related traits in plants.

Nucleic acids encoding GRP polypeptide described herein, or the GRP polypeptides themselves, may find use in breeding programmes in which a DNA marker is identified which may be genetically linked to a GRP polypeptide-encoding gene. The nucleic acids/genes, or the GRP polypeptides themselves may be used to define a molecular marker. This DNA or protein marker may then be used in breeding programmes to select plants having enhanced yield-related traits as defined hereinabove in the methods of the invention.

Allelic variants of a GRP polypeptide-encoding nucleic acid/gene may also find use in marker-assisted breeding programmes. Such breeding programmes sometimes require introduction of allelic variation by mutagenic treatment of the plants, using for example EMS mutagenesis; alternatively, the programme may start with a collection of allelic variants of so called “natural” origin caused unintentionally. Identification of allelic variants then takes place, for example, by PCR. This is followed by a step for selection of superior allelic variants of the sequence in question and which give enhanced yield-related traits. Selection is typically carried out by monitoring growth performance of plants containing different allelic variants of the sequence in question. Growth performance may be monitored in a greenhouse or in the field. Further optional steps include crossing plants in which the superior allelic variant was identified with another plant. This could be used, for example, to make a combination of interesting phenotypic features.

Nucleic acids encoding GRP polypeptides may also be used as probes for genetically and physically mapping the genes that they are a part of, and as markers for traits linked to those genes. Such information may be useful in plant breeding in order to develop lines with desired phenotypes. Such use of GRP polypeptide-encoding nucleic acids requires only a nucleic acid sequence of at least 15 nucleotides in length. The GRP polypeptide-encoding nucleic acids may be used as restriction fragment length polymorphism (RFLP) markers. Southern blots (Sambrook J, Fritsch E F and Maniatis T (1989) Molecular Cloning, A Laboratory Manual) of restriction-digested plant genomic DNA may be probed with the GRP-encoding nucleic acids. The resulting banding patterns may then be subjected to genetic analyses using computer programs such as MapMaker (Lander et al. (1987) Genomics 1: 174-181) in order to construct a genetic map. In addition, the nucleic acids may be used to probe Southern blots containing restriction endonuclease-treated genomic DNAs of a set of individuals representing parent and progeny of a defined genetic cross. Segregation of the DNA polymorphisms is noted and used to calculate the position of the GRP polypeptide-encoding nucleic acid sequence in the genetic map previously obtained using this population (Botstein et al. (1980) Am. J. Hum. Genet. 32:314-331).

The production and use of plant gene-derived probes for use in genetic mapping is described in Bernatzky and Tanksley (1986) Plant Mol. Biol. Reporter 4: 37-41. Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

The nucleic acid sequence probes may also be used for physical mapping (i.e., placement of sequences on physical maps; see Hoheisel et al. In: Non-mammalian Genomic Analysis: A Practical Guide, Academic press 1996, pp. 319-346, and references cited therein).

In another embodiment, the nucleic acid sequence probes may be used in direct fluorescence in situ hybridisation (FISH) mapping (Trask (1991) Trends Genet. 7:149-154). Although current methods of FISH mapping favour use of large clones (several kb to several hundred kb; see Laan et al. (1995) Genome Res. 5:13-20), improvements in sensitivity may allow performance of FISH mapping using shorter probes.

A variety of nucleic acid sequence amplification-based methods for genetic and physical mapping may be carried out using the nucleic acids. Examples include allele-specific amplification (Kazazian (1989) J. Lab. Clin. Med 11:95-96), polymorphism of PCR-amplified fragments (CAPS; Sheffield et al. (1993) Genomics 16:325-332), allele-specific ligation (Landegren et al. (1988) Science 241:1077-1080), nucleotide extension reactions (Sokolov (1990) nucleic acid sequence Res. 18:3671), Radiation Hybrid Mapping (Walter et al. (1997) Nat. Genet. 7:22-28) and Happy Mapping (Dear and Cook (1989) nucleic acid sequence Res. 17:6795-6807). For these methods, the sequence of a nucleic acid sequence is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for mapping methods.

The methods according to the present invention result in plants having enhanced yield-related traits, as described hereinbefore. These traits may also be combined with other economically advantageous traits, such as further yield-enhancing traits, tolerance to other abiotic and biotic stresses, traits modifying various architectural features and/or biochemical and/or physiological features.

The term “table A2” used in this specification is to be taken to specify the content of table A2a and table A2b. The term “table A2a” used in this specification is to be taken to specify the content of table A2a. The term “table A2b” used in this specification is to be taken to specify the content of table A2b. In one preferred embodiment, the term “table A2” means table A2b.

In one embodiment the invention relates to subject mater summarized as follows:

Item 21: A method for enhancing yield-related traits in plants relative to control plants, comprising modulating expression in a plant of a nucleic acid sequence encoding a GRP, wherein said GRP is an RrmJ/FtsJ ribosomal RNA methyltransferase polypeptide (RrmJ/FtsJ polypeptide), and optionally selecting for plants having enhance yield-related traits.
Item 22: Method according to item 21, wherein said GRP polypeptide comprises a ribosomal RNA methyltransferase RrmJ/FtsJ domain (InterPro entries IPR002877 and IPR015507; Pfam entry PF01728; PANTHER entry PTHR10920).
Item 23: Method according to item 21 or 22, wherein said GRP polypeptide has in increasing order of preference at least 50%, 55%, 60%, 65%, 70%, 75%, 80%, 85%, 90%, 95%, 98%, 99% or more polypeptide sequence identity to the GRP polypeptide as represented by SEQ ID NO: 58, or by SEQ ID NO: 60, or to any of the polypeptide sequences given in Table A2 of Example 1.
Item 24: Method according to any of the items 21 to 23, wherein said nucleic acid sequence encoding a GRP polypeptide is represented by any one of the nucleic acid sequence SEQ ID NOs given in Table A2 or a portion thereof, or a sequence capable of hybridising with any one of the nucleic acid sequences SEQ ID NOs given in Table A2 of Example 1.
Item 25: Method according to any preceding item wherein said modulated expression is effected by introducing and expressing in a plant a nucleic acid sequence encoding said GRP polypeptide.
Item 26: Method according to any preceding item, wherein said enhanced yield-related traits comprise increased seed yield, increased number of filled seeds, increased seed fill rate, and increased harvest index, relative to control plants.
Item 27: Method according to any preceding item, wherein said modulated expression is increased expression.
Item 28: Method according to any preceding item, wherein said nucleic acid sequence is operably linked to a seed-specific promoter, preferably to an oleosin promoter, most preferably to an oleosin promoter from rice.
Item 29: Method according to any preceding item, wherein said nucleic acid sequence encoding a GRP polypeptide is of plant origin, preferably from a dicotyledonous plant, further preferably from the family Solanaceae, more preferably from Nicotiana tabacum.
Item 30: An isolated nucleic acid sequence comprising a nucleic acid sequence selected from the group consisting of:
a) an isolated nucleic acid molecule encoding the polypeptide as depicted in SEQ ID NO: 97, 99, 101 and/or 103 and/or table A2b;
b) an isolated nucleic acid molecule as depicted in SEQ ID NO: 96, 98, 100 and/or 102 and/or table A2b;
c) an isolated nucleic acid molecule, which, as a result of the degeneracy of the genetic code, can be derived from a polypeptide sequence depicted in table A2 and confers enhanced yield-related traits relative to control plants;
d) an isolated nucleic acid molecule having at least 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with the nucleic acid molecule sequence of a polynucleotide comprising the nucleic acid molecule depicted in table A2 and confers enhanced yield-related traits relative to control plants;
e) an isolated nucleic acid molecule encoding a polypeptide having at least 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of the polypeptide encoded by the nucleic acid molecule of (a) to (c) and confers enhanced yield-related traits relative to control plants;
f) an isolated nucleic acid molecule which hybridizes with a nucleic acid molecule of (a) to (c) under stringent hybridization conditions and confers enhanced yield-related traits relative to control plants;
g) an isolated nucleic acid molecule encoding a polypeptide comprising the consensus sequence or one or more polypeptide motifs as shown FIG. 5 and preferably having the activity represented by a nucleic acid molecule comprising a polynucleotide as depicted in table A2;
h) an isolated nucleic acid molecule encoding a polypeptide having the activity represented by a protein as depicted in table A2 and confering enhanced yield-related traits relative to control plants;
and
i) a nucleic acid molecule which is obtainable by screening a suitable nucleic acid library under stringent hybridization conditions with a probe comprising a complementary sequence of a nucleic acid molecule of (a) or (b) or with a fragment thereof, having at least 15 nt, preferably 20 nt, 30 nt, 50 nt, 100 nt, 200 nt or 500 nt of a nucleic acid molecule complementary to a nucleic acid molecule sequence characterized in (a) to (e) and encoding a polypeptide having the activity represented by a protein comprising a polypeptide as depicted in table A2;
   whereby the nucleic acid molecule according to (a) to (i) is at least in one or more nucleotides different from the sequence depicted in table A2a and preferably which encodes a protein which differs at least in one or more amino acids from the protein sequences depicted in table A2a.
   Item 31: Plant, part thereof (including seeds), or plant cell obtainable by a method according to any preceding item, wherein said plant, part thereof, or plant cell comprises a nucleic acid transgene encoding a GRP polypeptide, preferably according to item 30.
   Tem 32: Construct comprising:
(i) nucleic acid sequence encoding a GRP polypeptide as defined in items 21 to 24 or of a polypeptide encoded by a nucleic acid molecule according to item 30;
(ii) one or more control sequences capable of driving expression of the nucleic acid sequence of (i); and optionally
(iii) a transcription termination sequence.
   Item 33: Construct according to item 32, wherein one of said control sequences is a seed-specific promoter, preferably an oleosin promoter, most preferably a oleosin promoter from rice.
   Item 34: Use of a construct according to any of items 32 or 33 or of a nucleic acid molecule according to item 30 in a method for making plants having enhanced yield-related traits, particularly increased seed yield, increased number of filled seeds, increased seed fill rate, and increased harvest index, relative to control plants.
   Item 35: Plant, plant part or plant cell transformed with a construct according to any of items 32 or 33.
   Item 36: Method for the production of a transgenic plant having enhanced-yield related traits relative to control plants, comprising:
8I9 introducing and expressing in a plant, plant part, or plant cell, a nucleic acid sequence encoding a GRP polypeptide as defined in items 21 to 24 or of a polypeptide encoded by a nucleic acid molecule according to item 30; and
(ii) cultivating the plant, plant part, or plant cell under conditions promoting plant growth and development.
   Item 37: Transgenic plant having enhanced yield-related traits, particularly increased seed yield, increased number of filled seeds, increased seed fill rate, and increased harvest index, relative to control plants, resulting from increased expression of a nucleic acid sequence encoding a GRP polypeptide as defined in items 21 to 24 or of a polypeptide encoded by a nucleic acid molecule according to item 30, or a transgenic plant cell derived from said transgenic plant.
   Item 38: Transgenic plant according to item 31, 35 or 37, or a transgenic plant cell derived thereof, wherein said plant is a crop plant or a monocot or a cereal, such as rice, maize, wheat, barley, millet, rye, triticale, sorghum and oats.
   Item 39: Harvestable parts of a plant comprising a nucleic acid sequence encoding a GRP polypeptide according to item 38, wherein said harvestable parts are preferably seeds.
   Item 40: Products derived from a plant according to item 38 and/or from harvestable parts of a plant according to item 39.
   Item 41: Use of a nucleic acid sequence encoding a GRP polypeptide in enhanced yield-related traits in plants, particularly in increasing particularly increased seed yield, increased number of filled seeds, increased seed fill rate, and increased harvest index, relative to control plants.

According to a further embodiment of the present invention, there is also provided an isolated polypeptide comprising a polypeptide sequence selected from the group consisting of:

(i) an amino acid sequence encoded by any one of the nucleic acids represented by Item 30;
(ii) an amino acid sequence having, in increasing order of preference, at least 50%, 55%, 60%, 65%, 70%, 75%, 80%, 85%, 90%, 95%, 96%, 97%, 98%, 99% or more sequence identity to the amino acid sequence of (i);
(iii) derivatives of any of the amino acid sequences given in (i) or (ii) above.

In an other embodiment of the invention it has now been found that modulating expression (preferably, increasing) in a plant of a nucleic acid sequence encoding a GRP polypeptide, wherein said GRP polypeptide is a basic-helix-loop-helix 4 (bHLH4) polypeptide, gives plants having enhanced yield-related traits, preferably enhanced seed yield-related traits, relative to control plants. According to a first embodiment, the present invention provides a method for enhancing yield-related traits, preferably enhanced seed yield-related traits, in plants relative to control plants, comprising modulating expression (preferably, increasing) in a plant of a nucleic acid sequence encoding a GRP polypeptide, wherein said GRP polypeptide is a basic-helix-loop-helix 4 (bHLH4) polypeptide.

A preferred method for modulating (preferably, increasing) expression of a nucleic acid sequence encoding a GRP polypeptide is by introducing and expressing in a plant a nucleic acid sequence encoding a GRP polypeptide.

Any reference hereinafter to a “protein useful in the methods of the invention” is taken to mean a GRP polypeptide as defined herein. Any reference hereinafter to a “nucleic acid sequence useful in the methods of the invention” is taken to mean a nucleic acid sequence capable of encoding such a GRP polypeptide. The nucleic acid sequence to be introduced into a plant (and therefore useful in performing the methods of the invention) is any nucleic acid sequence encoding the type of protein which will now be described, hereafter also named “GRP nucleic acid sequence” or “GRP gene”.

In one embodiment of the invention a “GRP polypeptide” as defined herein refers the proteins represented by SEQ ID NO: 105, and to orthologues, paralogues, and homologues thereof. Table A3 of Example 1 herein presents such orthologues and paralogues.

Preferably, the orthologues, paralogues, and homologues of SEQ ID NO: 105 have a basic-helix-loop-helix dimerisation region with InterPro entry IPR001092, and helix-loop-helix DNA-binding with InterPro entry IPR011598.

Alternatively or additionally, a “GRP polypeptide” as defined herein refers to any polypeptide having in increasing order of preference at least 50%, 55%, 60%, 65%, 70%, 75%, 80%, 85%, 90%, 95%, 98%, 99% or more amino acid sequence identity to the GRP polypeptide as represented by SEQ ID NO: 105, or to any of the polypeptide sequences given in Table A3 of Example 1 herein.

The term “domain” and “motif” is defined in the “definitions” section herein. Specialist databases exist for the identification of domains, for example, SMART (Schultz et al. (1998) Proc. Natl. Acad. Sci. USA 95, 5857-5864; Letunic et al. (2002) Nucleic Acids Res 30, 242-244), InterPro (Mulder et al., (2003) Nucl. Acids. Res. 31, 315-318), Prosite (Bucher and Bairoch (1994), A generalized profile syntax for biomolecular sequences motifs and its function in automatic sequence interpretation. (In) ISMB-94; Proceedings 2nd International Conference on Intelligent Systems for Molecular Biology. Altman R., Brutlag D., Karp P., Lathrop R., Searls D., Eds., pp 53-61, AAAI Press, Menlo Park; Hulo et al., Nucl. Acids. Res. 32:D134-D137, (2004) ), or Pfam (Bateman et al., Nucleic Acids Research 30(1): 276-280 (2002) ). A set of tools for in silico analysis of protein sequences is available on the ExPASy proteomics server (Swiss Institute of Bioinformatics (Gasteiger et al., ExPASy: the proteomics server for in-depth protein knowledge and analysis, Nucleic Acids Res. 31:3784-3788 (2003) ). Domains or motifs may also be identified using routine techniques, such as by sequence alignment.

Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the global (i.e. spanning the complete sequences) alignment of two sequences that maximizes the number of matches and minimizes the number of gaps. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information (NCBI). Homologues may readily be identified using, for example, the ClustalW multiple sequence alignment algorithm (version 1.83), with the default pairwise alignment parameters, and a scoring method in percentage. Global percentages of similarity and identity may also be determined using one of the methods available in the MatGAT software package (Campanella et al., BMC Bioinformatics. 2003 Jul. 10; 4:29. MatGAT: an application that generates similarity/identity matrices using protein or DNA sequences). Minor manual editing may be performed to optimise alignment between conserved motifs, as would be apparent to a person skilled in the art. Furthermore, instead of using full-length sequences for the identification of homologues, specific domains may also be used. The sequence identity values may be determined over the entire nucleic acid sequence or polypeptide sequence or over selected domains or conserved motif(s), using the programs mentioned above using the default parameters.

Furthermore, GRP polypeptides, as far as SEQ ID NO: 105, and its orthologues, paralogues, and homologues are concerned, typically have the capability to bind DNA (at least in their native form), but not necessarily, have transcriptional regulatory activity and capacity to interact with other proteins. Therefore, GRP polypeptides with reduced transcriptional regulatory activity, without transcriptional regulatory activity, with reduced protein-protein interaction capacity, or with no protein-protein interaction capacity, may equally be useful in the methods of the present invention. DNA-binding activity and protein-protein interactions may readily be determined in vitro or in vivo using techniques well known in the art (for example in Current Protocols in Molecular Biology, Volumes 1 and 2, Ausubel et al. (1994), Current Protocols). To determine the DNA binding activity of GRP polypeptides, several assays are available, such as DNA binding gel-shift assays (or gel retardation assays; Korfhage et al. (1994) Plant C 6: 695-708), in vitro DNA binding assays (Schindler et al. (1993) Plant J 4(1): 137-150), or transcriptional activation of GRP polypeptides in yeast, animal and plant cells. GRP polypeptides typically bind to a consensus sequence called an E-box, CANNTG. The canonical E-box is CACGTG (palindromic), however some bHLH4 transcription factors bind to different sequences, which are often similar to the E-box. Specific DNA binding sequences can be determined using the random oligonucleotide selection technique (Viola & Gonzalez (May 26, 2007) Biochemistry).

The present invention is illustrated by transforming plants with the nucleic acid sequence represented by SEQ ID NO: 104, encoding the polypeptide sequence of SEQ ID NO: 105. However, performance of the invention is not restricted to these sequences; the methods of the invention may advantageously be performed using any GRP-encoding nucleic acid sequence or GRP polypeptide as defined herein.

Examples of nucleic acid sequences encoding GRP polypeptides may be found in databases known in the art. Such nucleic acid sequences are useful in performing the methods of the invention. Orthologues and paralogues, the terms “orthologues” and “paralogues” being as defined herein, may readily be identified by performing a so-called reciprocal blast search. Typically, this involves a first BLAST involving BLASTing a query sequence (for example using SEQ ID NO: 105) against any sequence database, such as the publicly available NCBI database. BLASTN or TBLASTX (using standard default values) are generally used when starting from a nucleotide sequence, and BLASTP or TBLASTN (using standard default values) when starting from a protein sequence. The BLAST results may optionally be filtered. The full-length sequences of either the filtered results or non-filtered results are then BLASTed back (second BLAST) against sequences from the organism from which the query sequence is derived (where the query sequence is SEQ ID NO: 104 or SEQ ID NO: 105, the second BLAST would therefore be against Arabidopsis thaliana sequences). The results of the first and second BLASTs are then compared. A paralogue is identified if a high-ranking hit from the first blast is from the same species as from which the query sequence is derived, a BLAST back then ideally results in the query sequence amongst the highest hits; an orthologue is identified if a high-ranking hit in the first BLAST is not from the same species as from which the query sequence is derived, and preferably results upon BLAST back in the query sequence being among the highest hits.

High-ranking hits are those having a low E-value. The lower the E-value, the more significant the score (or in other words the lower the chance that the hit was found by chance). Computation of the E-value is well known in the art. In addition to E-values, comparisons are also scored by percentage identity. Percentage identity refers to the number of identical nucleotides (or amino acids) between the two compared nucleic acid sequence (or polypeptide) sequences over a particular length. In the case of large families, ClustalW may be used, followed by a neighbour joining tree, to help visualize clustering of related genes and to identify orthologues and paralogues.

Nucleic acid sequence variants encoding homologues and derivatives of SEQ ID NO: 105 may also be useful in practising the methods of the invention, the terms “homologue” and “derivative” being as defined herein. Also useful in the methods of the invention are nucleic acid sequences encoding homologues and derivatives of orthologues or paralogues of SEQ ID NO: 105. Homologues and derivatives useful in the methods of the present invention have substantially the same biological and functional activity as the unmodified protein from which they are derived.

Further nucleic acid sequence variants useful in practising the methods of the invention include portions of nucleic acid sequences encoding GRP polypeptides, nucleic acid sequences hybridising to nucleic acid sequences encoding GRP polypeptides, splice variants of nucleic acid sequences encoding GRP polypeptides, allelic variants of nucleic acid sequences encoding GRP polypeptides and variants of nucleic acid sequences encoding GRP polypeptides obtained by gene shuffling. The terms hybridising sequence, splice variant, allelic variant and gene shuffling are as described herein.

Nucleic acid sequences encoding GRP polypeptides need not be full-length nucleic acid sequences, since performance of the methods of the invention does not rely on the use of full-length nucleic acid sequences. According to the present invention, there is provided a method for enhancing yield-related traits in plants, preferably enhanced seed yield-related traits, comprising introducing and expressing in a plant a portion of SEQ ID NO: 104, or a portion of a nucleic acid sequence encoding an orthologue, paralogue or homologue of SEQ ID NO: 105.

A portion of a nucleic acid sequence may be prepared, for example, by making one or more deletions to the nucleic acid sequence. The portions may be used in isolated form or they may be fused to other coding (or non-coding) sequences in order to, for example, produce a protein that combines several activities. When fused to other coding sequences, the resultant polypeptide produced upon translation may be bigger than that predicted for the protein portion.

Portions useful in the methods of the invention, encode a GRP polypeptide as defined herein, and have substantially the same biological activity as the polypeptide sequence given in SEQ ID NO: 105. Preferably, the portion is a portion of the nucleic acid sequence given in SEQ ID NO: 104, or is a portion of a nucleic acid sequence encoding an orthologue or paralogue of the polypeptide sequence given in SEQ ID NO: 105. Preferably the portion is at least 500, 600, 700, 800, 850, 900, 950, 1000, 1050, 1100, 1150, 1200, 1250, 1300, 1350, 1400, 1450, 1500, 1550, 1600, 1650, 1700, 1750 or more consecutive nucleotides in length, the consecutive nucleotides being of SEQ ID NO: 104, or of a nucleic acid sequence encoding an orthologue or paralogue of SEQ ID NO: 105. Most preferably the portion is a portion of the nucleic acid sequence of SEQ ID NO: 104.

Another nucleic acid sequence variant useful in the methods of the invention is a nucleic acid sequence capable of hybridising, under reduced stringency conditions, preferably under stringent conditions, with a nucleic acid sequence encoding a GRP polypeptide as defined herein, or with a portion as defined herein.

According to the present invention, there is provided a method for enhancing yield-related traits in plants, preferably enhanced seed yield-related traits, comprising introducing and expressing in a plant a nucleic acid sequence capable of hybridizing to SEQ ID NO: 104, or comprising introducing and expressing in a plant a nucleic acid sequence capable of hybridising to a nucleic acid sequence encoding an orthologue, paralogue or homologue of SEQ ID NO: 105.

Hybridising sequences useful in the methods of the invention encode a GRP polypeptide as defined herein, having substantially the same biological activity as the polypeptide sequence given in SEQ ID NO: 105. Preferably, the hybridising sequence is capable of hybridising to SEQ ID NO: 104, or to a portion of this sequence, a portion being as defined above, or the hybridising sequence is capable of hybridising to a nucleic acid sequence encoding an orthologue or paralogue of SEQ ID NO: 105, or to a portion thereof.

Another nucleic acid sequence variant useful in the methods of the invention is a splice variant encoding a GRP polypeptide as defined hereinabove, a splice variant being as defined herein.

According to the present invention, there is provided a method for enhancing yield-related traits in plants, preferably enhancing seed yield-related traits, comprising introducing and expressing in a plant a splice variant of SEQ ID NO: 104, or a splice variant of a nucleic acid sequence encoding an orthologue, paralogue or homologue of SEQ ID NO: 105.

Another nucleic acid sequence variant useful in performing the methods of the invention is an allelic variant of a nucleic acid sequence encoding a GRP polypeptide as defined hereinabove, an allelic variant being as defined herein.

According to the present invention, there is provided a method for enhancing yield-related traits in plants, preferably enhanced seed yield-related traits, comprising introducing and expressing in a plant an allelic variant of SEQ ID NO: 104, or comprising introducing and expressing in a plant an allelic variant of a nucleic acid sequence encoding an orthologue, paralogue or homologue of the polypeptide sequence represented by SEQ ID NO: 105.

The allelic variants useful in the methods of the present invention have substantially the same biological activity as the GRP polypeptide of SEQ ID NO: 105. Allelic variants exist in nature, and encompassed within the methods of the present invention is the use of these natural alleles. Gene shuffling or directed evolution may also be used to generate variants of nucleic acid sequences encoding GRP polypeptides as defined above; the term “gene shuffling” being as defined herein.

According to the present invention, there is provided a method for enhancing yield-related traits in plants, preferably enhanced seed yield-related traits, comprising introducing and expressing in a plant a variant nucleic acid sequence of SEQ ID NO: 104, or comprising introducing and expressing in a plant a variant of a nucleic acid sequence encoding an orthologue, paralogue or homologue of SEQ ID NO: 105, which variant nucleic acid sequence is obtained by gene shuffling.

Furthermore, nucleic acid sequence variants may also be obtained by site-directed mutagenesis. Several methods are available to achieve site-directed mutagenesis, the most common being PCR based methods (Current Protocols in Molecular Biology. Wiley Eds.).

Nucleic acid sequences encoding GRP polypeptides may be derived from any natural or artificial source. The nucleic acid sequence may be modified from its native form in composition and/or genomic environment through deliberate human manipulation. Preferably the GRP polypeptide-encoding nucleic acid sequence is from a plant. In the case of SEQ ID NO: 104, the GRP polypeptide encoding nucleic acid sequence is preferably from a dicotyledonous plant, more preferably from the family Brassicaceae, most preferably the nucleic acid sequence is from Arabidopsis thaliana.

Performance of the methods of the invention gives plants having enhanced yield-related traits, in particular enhanced seed yield-related traits. The terms “yield” and “seed yield” are described in more detail in the “definitions” section herein.

Reference herein to enhanced yield-related traits is taken to mean an increase in early vigour and/or in biomass (weight) of one or more parts of a plant, which may include aboveground (harvestable) parts and/or (harvestable) parts below ground. In particular, such harvestable parts are biomass and/or seeds, and performance of the methods of the invention results in plants having increased early vigour, biomass and/or seed yield relative to the early vigour, biomass or seed yield of control plants.

Taking corn as an example, a yield increase may be manifested as one or more of the following: increase in the number of plants established per hectare or acre, an increase in the number of ears per plant, an increase in the number of rows, number of kernels per row, kernel weight, thousand kernel weight, ear length/diameter, increase in the seed filling rate (which is the number of filled seeds divided by the total number of seeds and multiplied by 100), among others. Taking rice as an example, a yield increase may manifest itself as an increase in one or more of the following: number of plants per hectare or acre, number of panicles per plant, number of spikelets per panicle, number of flowers (florets) per panicle (which is expressed as a ratio of the number of filled seeds over the number of primary panicles), increase in the seed filling rate (which is the number of filled seeds divided by the total number of seeds and multiplied by 100), increase in thousand kernel weight, among others.

The present invention provides a method for enhancing yield-related traits, especially biomass and/or seed yield of plants, relative to control plants, which method comprises modulating expression (preferably, increasing) in a plant of a nucleic acid sequence encoding a GRP polypeptide as defined herein.

Since the transgenic plants according to the present invention have enhanced yield-related traits, preferably enhanced seed yield-related traits, it is likely that these plants exhibit an increased growth rate (during at least part of their life cycle), relative to the growth rate of control plants at a corresponding stage in their life cycle.

The increased growth rate may be specific to one or more parts of a plant (including seeds), or may be throughout substantially the whole plant. Plants having an increased growth rate may have a shorter life cycle. The life cycle of a plant may be taken to mean the time needed to grow from a dry mature seed up to the stage where the plant has produced dry mature seeds, similar to the starting material. This life cycle may be influenced by factors such as early vigour, growth rate, greenness index, flowering time and speed of seed maturation. The increase in growth rate may take place at one or more stages in the life cycle of a plant or during substantially the whole plant life cycle. Increased growth rate during the early stages in the life cycle of a plant may reflect enhanced vigour. The increase in growth rate may alter the harvest cycle of a plant allowing plants to be sown later and/or harvested sooner than would otherwise be possible (a similar effect may be obtained with earlier flowering time). If the growth rate is sufficiently increased, it may allow for the further sowing of seeds of the same plant species (for example sowing and harvesting of rice plants followed by sowing and harvesting of further rice plants all within one conventional growing period). Similarly, if the growth rate is sufficiently increased, it may allow for the further sowing of seeds of different plants species (for example the sowing and harvesting of corn plants followed by, for example, the sowing and optional harvesting of soybean, potato or any other suitable plant). Harvesting additional times from the same rootstock in the case of some crop plants may also be possible. Altering the harvest cycle of a plant may lead to an increase in annual biomass production per acre (due to an increase in the number of times (say in a year) that any particular plant may be grown and harvested). An increase in growth rate may also allow for the cultivation of transgenic plants in a wider geographical area than their wild-type counterparts, since the territorial limitations for growing a crop are often determined by adverse environmental conditions either at the time of planting (early season) or at the time of harvesting (late season). Such adverse conditions may be avoided if the harvest cycle is shortened. The growth rate may be determined by deriving various parameters from growth curves, such parameters may be: T-Mid (the time taken for plants to reach 50% of their maximal size) and T-90 (time taken for plants to reach 90% of their maximal size), amongst others.

According to a preferred feature of the present invention, performance of the methods of the invention gives plants having an increased growth rate relative to control plants. Therefore, according to the present invention, there is provided a method for increasing the growth rate of plants, which method comprises modulating expression (preferably, increasing) in a plant of a nucleic acid sequence encoding a GRP polypeptide as defined herein.

An enhancement of yield-related traits (an increase in seed yield and/or growth rate) occurs whether the plant is under non-stress conditions or whether the plant is exposed to various stresses compared to control plants. Plants typically respond to exposure to stress by growing more slowly. In conditions of severe stress, the plant may even stop growing altogether. Mild stress on the other hand is defined herein as being any stress to which a plant is exposed which does not result in the plant ceasing to grow altogether without the capacity to resume growth. Mild stress in the sense of the invention leads to a reduction in the growth of the stressed plants of less than 40%, 35% or 30%, preferably less than 25%, 20% or 15%, more preferably less than 14%, 13%, 12%, 11% or 10% or less in comparison to the control plant under non-stress conditions. Due to advances in agricultural practices (irrigation, fertilization, pesticide treatments) severe stresses are not often encountered in cultivated crop plants. As a consequence, the compromised growth induced by mild stress is often an undesirable feature for agriculture. Mild stresses are the everyday biotic and/or abiotic (environmental) stresses to which a plant is exposed. Abiotic stresses may be due to drought or excess water, anaerobic stress, salt stress, chemical toxicity, oxidative stress and hot, cold or freezing temperatures. The abiotic stress may be an osmotic stress caused by a water stress (particularly due to drought), salt stress, oxidative stress or an ionic stress. Biotic stresses are typically those stresses caused by pathogens, such as bacteria, viruses, nematodes, fungi and insects. The term “non-stress” conditions as used herein are those environmental conditions that allow optimal growth of plants. Persons skilled in the art are aware of normal soil conditions and climatic conditions for a given location.

Performance of the methods of the invention gives plants grown under non-stress conditions or under mild stress conditions having enhanced yield-related traits, preferably enhanced seed yield-related traits, relative to control plants grown under comparable conditions. Therefore, according to the present invention, there is provided a method for enhancing yield-related traits, preferably enhanced seed yield-related traits, in plants grown under non-stress conditions or under mild stress conditions, which method comprises modulating (preferably, increasing) expression in a plant of a nucleic acid sequence encoding a GRP polypeptide.

Performance of the methods according to the present invention results in plants grown under abiotic stress conditions having enhanced yield-related traits, preferably enhanced seed yield-related traits, relative to control plants grown under comparable stress conditions. As reported in Wang et al. (Planta (2003) 218: 1-14), abiotic stress leads to a series of morphological, physiological, biochemical and molecular changes that adversely affect plant growth and productivity. Drought, salinity, extreme temperatures and oxidative stress are known to be interconnected and may induce growth and cellular damage through similar mechanisms. Rabbani et al. (Plant Physiol (2003) 133: 1755-1767) describes a particularly high degree of “cross talk” between drought stress and high-salinity stress. For example, drought and/or salinisation are manifested primarily as osmotic stress, resulting in the disruption of homeostasis and ion distribution in the cell. Oxidative stress, which frequently accompanies high or low temperature, salinity or drought stress, may cause denaturing of functional and structural proteins. As a consequence, these diverse environmental stresses often activate similar cell signalling pathways and cellular responses, such as the production of stress proteins, up-regulation of anti-oxidants, accumulation of compatible solutes and growth arrest. Since diverse environmental stresses activate similar pathways, plants having enhanced yield-related traits, preferably enhanced seed yield-related traits, under one type of abiotic stress growth conditions, relative to control plants grown under comparable abiotic stress conditions, should not be seen as a limitation to that type of abiotic stress growth conditions, but more as a screen to indicate the involvement of GRP polypeptides as defined above, in enhancing yield-related traits in abiotic stress growth conditions in general.

The term “abiotic stress” as defined herein is taken to mean any one or more of: water stress (due to drought or excess water), anaerobic stress, salt stress, temperature stress (due to hot, cold or freezing temperatures), chemical toxicity stress and oxidative stress. According to one aspect of the invention, the abiotic stress is an osmotic stress, selected from water stress, salt stress, oxidative stress and ionic stress. Preferably, the water stress is drought stress. The term salt stress is not restricted to common salt (NaCl), but may be any stress caused by one or more of: NaCl, KCl, LiCl, MgCl2, CaCl2, amongst others.

Performance of the methods of the invention gives plants having enhanced yield-related traits, preferably enhanced seed yield-related traits, under abiotic stress growth conditions relative to control plants grown in comparable stress conditions. Therefore, according to the present invention, there is provided a method for enhancing yield-related traits, preferably enhancing seed yield-related traits, in plants grown under abiotic stress growth conditions, which method comprises modulating (preferably, increasing) expression in a plant of a nucleic acid sequence encoding a GRP polypeptide. According to one aspect of the invention, the abiotic stress is an osmotic stress, selected from one or more of the following: water stress, salt stress, oxidative stress and ionic stress.

Performance of the methods of the invention gives plants grown under conditions of reduced nutrient availability, particularly under conditions of reduced nitrogen availability, enhanced yield-related traits, preferably enhancing seed yield-related traits, relative to control plants grown under comparable conditions. Therefore, according to the present invention, there is provided a method for enhancing yield-related traits in plants grown under conditions of reduced nutrient availability, which method comprises modulating (preferably, increasing) expression in a plant of a nucleic acid sequence encoding a GRP polypeptide. Reduced nutrient availability may comprise reduced availability of nutrients such as nitrogen, phosphates and other phosphorous-containing compounds, potassium, calcium, cadmium, magnesium, manganese, iron and boron, amongst others.

The present invention encompasses plants, parts thereof (including seeds), or plant cells obtainable by the methods according to the present invention. The plants, parts thereof, or plant cells comprise a nucleic acid transgene encoding a GRP polypeptide as defined above, preferably a basic-helix-loop-helix 4 (bHLH4) polypeptide.

The invention also provides genetic constructs and vectors to facilitate introduction and/or expression in plants of nucleic acid sequences encoding GRP polypeptides. The gene constructs may be inserted into vectors, which may be commercially available, suitable for transforming into plants and suitable for expression of the gene of interest in the transformed cells. The invention also provides use of a gene construct as defined herein in the methods of the invention.

More specifically, the present invention provides a construct comprising:
(a) a nucleic acid sequence encoding a GRP polypeptide, preferably a basic-helix-loop-helix 4 (bHLH4) polypeptide as defined above;
(b) one or more control sequences capable of driving expression of the nucleic acid sequence of (a); and optionally
(c) a transcription termination sequence.

Preferably, the nucleic acid sequence encoding a GRP polypeptide is as defined above. The term “control sequence” and “termination sequence” are as defined herein.

Plants are transformed with a vector comprising any of the nucleic acid sequences described above. The skilled artisan is well aware of the genetic elements that must be present on the vector in order to successfully transform, select and propagate host cells containing the sequence of interest. The sequence of interest is operably linked to one or more control sequences (at least to a promoter).

Advantageously, any type of promoter, whether natural or synthetic, may be used to drive expression of the nucleic acid sequence. A constitutive promoter is particularly useful in the methods of the invention. See the “Definitions” section herein for definitions of the various promoter types.

It should be clear that the applicability of the present invention is not restricted to the GRP polypeptide-encoding nucleic acid sequence represented by SEQ ID NO: 104, nor is the applicability of the invention restricted to expression of a GRP polypeptide-encoding nucleic acid sequence when driven by a constitutive promoter.

The constitutive promoter is preferably a high mobility group B (HMGB) promoter, preferably an HMGB promoter from rice. Further preferably the HMGB promoter is represented by a nucleic acid sequence substantially similar to SEQ ID NO: 106, most preferably the HMGB promoter is as represented by SEQ ID NO: 106. See Table 2 in the “Definitions” section herein for further examples of constitutive promoters.

Optionally, one or more terminator sequences may be used in the construct introduced into a plant. Additional regulatory elements may include transcriptional as well as translational enhancers. Those skilled in the art will be aware of terminator and enhancer sequences that may be suitable for use in performing the invention. An intron sequence may also be added to the 5′ untranslated region (UTR) or in the coding sequence to increase the amount of the mature message that accumulates in the cytosol, as described in the definitions section. Other control sequences (besides promoter, enhancer, silencer, intron sequences, 3′UTR and/or 5′UTR regions) may be protein and/or RNA stabilizing elements. Such sequences would be known or may readily be obtained by a person skilled in the art.

The genetic constructs of the invention may further include an origin of replication sequence that is required for maintenance and/or replication in a specific cell type. One example is when a genetic construct is required to be maintained in a bacterial cell as an episomal genetic element (e.g. plasmid or cosmid molecule). Preferred origins of replication include, but are not limited to, the f1-ori and colE1.

For the detection of the successful transfer of the nucleic acid sequences as used in the methods of the invention and/or selection of transgenic plants comprising these nucleic acid sequences, it is advantageous to use marker genes (or reporter genes). Therefore, the genetic construct may optionally comprise a selectable marker gene. Selectable markers are described in more detail in the “definitions” section herein. The marker genes may be removed or excised from the transgenic cell once they are no longer needed. Techniques for marker removal are known in the art, useful techniques are described above in the definitions section.

The invention also provides a method for the production of transgenic plants having enhanced yield-related traits, preferably enhanced seed yield-related traits, relative to control plants, comprising introduction and expression in a plant of a nucleic acid sequence encoding a GRP polypeptide as defined hereinabove.

More specifically, the present invention provides a method for the production of transgenic plants having enhanced yield-related traits, preferably enhanced seed yield-related traits, which method comprises:
(i) introducing and expressing in a plant, plant part, or plant cell, a nucleic acid sequence encoding a GRP polypeptide; and
(ii) cultivating the plant, plant part or plant cell under conditions promoting plant growth and development.

The nucleic acid sequence of (i) may be any of the nucleic acid sequences capable of encoding a GRP polypeptide as defined herein, in one embodiment having the activity of a basic-helix-loop-helix 4 (bHLH4) polypeptide.

The nucleic acid sequence may be introduced directly into a plant cell or into the plant itself (including introduction into a tissue, organ or any other part of a plant). According to a preferred feature of the present invention, the nucleic acid sequence is preferably introduced into a plant by transformation. The term “transformation” is described in more detail in the “definitions” section herein.

The genetically modified plant cells can be regenerated via all methods with which the skilled worker is familiar. Suitable methods can be found in the abovementioned publications by S. D. Kung and R. Wu, Potrykus or Hofgen and Willmitzer.

Generally after transformation, plant cells or cell groupings are selected for the presence of one or more markers which are encoded by plant-expressible genes co-transferred with the gene of interest, following which the transformed material is regenerated into a whole plant. To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as the ones described above.

Following DNA transfer and regeneration, putatively transformed plants may also be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

The present invention clearly extends to any plant cell or plant produced by any of the methods described herein, and to all plant parts and propagules thereof. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced by the parent in the methods according to the invention.

The invention also includes host cells containing an isolated nucleic acid sequence encoding a GRP polypeptide as defined hereinabove. Preferred host cells according to the invention are plant cells. Host plants for the nucleic acid transgene or the vector used in the method according to the invention, the expression cassette or construct or vector are, in principle, advantageously all plants, which are capable of synthesizing the polypeptides used in the inventive method.

The methods of the invention are advantageously applicable to any plant. Plants that are particularly useful in the methods of the invention include all plants which belong to the superfamily Viridiplantae, in particular monocotyledonous and dicotyledonous plants including fodder or forage legumes, ornamental plants, food crops, trees or shrubs. According to a preferred embodiment of the present invention, the plant is a crop plant. Examples of crop plants include soybean, sunflower, canola, alfalfa, rapeseed, cotton, tomato, potato and tobacco. Further preferably, the plant is a monocotyledonous plant. Examples of monocotyledonous plants include sugarcane. More preferably the plant is a cereal. Examples of cereals include rice, maize, wheat, barley, millet, rye, triticale, sorghum and oats.

The invention also extends to harvestable parts of a plant such as, but not limited to seeds, leaves, fruits, flowers, stems, roots, rhizomes, tubers and bulbs. The invention furthermore relates to products derived, preferably directly derived, from a harvestable part of such a plant, such as dry pellets or powders, oil, fat and fatty acids, starch or proteins.

According to a preferred feature of the invention, the modulated expression is increased expression. Methods for increasing expression of nucleic acid sequences or genes, or gene products, are well documented in the art and examples are provided in the definitions section.

As mentioned above, a preferred method for modulating (preferably, increasing) expression of a nucleic acid sequence encoding a GRP polypeptide is by introducing and expressing in a plant a nucleic acid sequence encoding a GRP polypeptide; however the effects of performing the method, i.e. enhancing yield-related traits, preferably enhancing seed yield-related traits, may also be achieved using other well known techniques, including but not limited to T-DNA activation tagging, TILLING, homologous recombination. A description of these techniques is provided in the definitions section.

The present invention also encompasses use of nucleic acid sequences encoding GRP polypeptides as described herein and use of these GRP polypeptides in enhancing any of the aforementioned yield-related traits, preferably seed yield-related traits, in plants.

Nucleic acid sequences encoding GRP polypeptide described herein, or the GRP polypeptides themselves, may find use in breeding programmes in which a DNA marker is identified which may be genetically linked to a GRP polypeptide-encoding gene. The genes/nucleic acid sequences, or the GRP polypeptides themselves may be used to define a molecular marker. This DNA or protein marker may then be used in breeding programmes to select plants having enhanced yield-related traits, preferably enhanced seed yield-related traits, as defined hereinabove in the methods of the invention.

Allelic variants of a GRP polypeptide-encoding nucleic acid sequence/gene may also find use in marker-assisted breeding programmes. Such breeding programmes sometimes require introduction of allelic variation by mutagenic treatment of the plants, using for example EMS mutagenesis; alternatively, the programme may start with a collection of allelic variants of so called “natural” origin caused unintentionally. Identification of allelic variants then takes place, for example, by PCR. This is followed by a step for selection of superior allelic variants of the sequence in question and which give enhanced yield-related traits, preferably enhanced seed yield-related traits. Selection is typically carried out by monitoring growth performance of plants containing different allelic variants of the sequence in question. Growth performance may be monitored in a greenhouse or in the field. Further optional steps include crossing plants in which the superior allelic variant was identified with another plant. This could be used, for example, to make a combination of interesting phenotypic features.

Nucleic acid sequences encoding GRP polypeptides may also be used as probes for genetically and physically mapping the genes that they are a part of, and as markers for traits linked to those genes. Such information may be useful in plant breeding in order to develop lines with desired phenotypes. Such use of GRP polypeptide-encoding nucleic acid sequences requires only a nucleic acid sequence of at least 15 nucleotides in length. The GRP polypeptide-encoding nucleic acid sequences may be used as restriction fragment length polymorphism (RFLP) markers. Southern blots (Sambrook J, Fritsch E F and Maniatis T (1989) Molecular Cloning, A Laboratory Manual) of restriction-digested plant genomic DNA may be probed with the GRP-encoding nucleic acid sequences. The resulting banding patterns may then be subjected to genetic analyses using computer programs such as MapMaker (Lander et al. (1987) Genomics 1: 174-181) in order to construct a genetic map. In addition, the nucleic acid sequences may be used to probe Southern blots containing restriction endonuclease-treated genomic DNAs of a set of individuals representing parent and progeny of a defined genetic cross. Segregation of the DNA polymorphisms is noted and used to calculate the position of the GRP polypeptide-encoding nucleic acid sequence in the genetic map previously obtained using this population (Botstein et al. (1980) Am. J. Hum. Genet. 32:314-331).

The production and use of plant gene-derived probes for use in genetic mapping is described in Bernatzky and Tanksley (1986) Plant Mol. Biol. Reporter 4: 37-41. Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

The nucleic acid sequence probes may also be used for physical mapping (i.e., placement of sequences on physical maps; see Hoheisel et al. In: Non-mammalian Genomic Analysis: A Practical Guide, Academic press 1996, pp. 319-346, and references cited therein).

In another embodiment, the nucleic acid sequence probes may be used in direct fluorescence in situ hybridisation (FISH) mapping (Trask (1991) Trends Genet. 7:149-154). Although current methods of FISH mapping favour use of large clones (several kb to several hundred kb; see Laan et al. (1995) Genome Res. 5:13-20), improvements in sensitivity may allow performance of FISH mapping using shorter probes.

A variety of nucleic acid sequence amplification-based methods for genetic and physical mapping may be carried out using the nucleic acid sequences. Examples include allele-specific amplification (Kazazian (1989) J. Lab. Clin. Med 11:95-96), polymorphism of PCR-amplified fragments (CAPS; Sheffield et al. (1993) Genomics 16:325-332), allele-specific ligation (Landegren et al. (1988) Science 241:1077-1080), nucleotide extension reactions (Sokolov (1990) Nucleic Acids Res. 18:3671), Radiation Hybrid Mapping (Walter et al. (1997) Nat. Genet. 7:22-28) and Happy Mapping (Dear and Cook (1989) Nucleic Acids Res. 17:6795-6807). For these methods, the sequence of a nucleic acid sequence is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for mapping methods.

The methods according to the present invention result in plants having enhanced yield-related traits, preferably enhanced seed yield-related traits, as described hereinbefore. These traits may also be combined with other economically advantageous traits, such as further yield-enhancing traits, tolerance to other abiotic and biotic stresses, traits modifying various architectural features and/or biochemical and/or physiological features.

The term “table A3” used in this specification is to be taken to specify the content of table A3a and table A3b. The term “table A3a” used in this specification is to be taken to specify the content of table A3a. The term “table A3b” used in this specification is to be taken to specify the content of table A3b. In one preferred embodiment, the term “table A3” means table A3b.

In one embodiment the invention relates to subject mater summarized as follows:

Item 42: A method for enhancing yield-related traits, preferably enhancing seed yield-related traits, in plants relative to control plants, comprising modulating expression (preferably, increasing) in a plant of a nucleic acid sequence encoding a GRP polypeptide, wherein said GRP polypeptide is a basic-helix-loop-helix 4 (bHLH4) polypeptide as represented by SEQ ID NO: 2 or any of the polypeptides given in table A3 or an orthologue, paralogue, or homologue thereof, and optionally selecting for plants having enhanced yield-related traits, preferably enhanced seed yield-related traits.

Item 43: A method according to item 42, wherein said GRP polypeptide as represented by SEQ ID NO: 105 and an orthologue, paralogue, or homologue thereof, have a basic-helix-loop-helix dimerisation region with InterPro entry IPR001092, and helix-loop-helix DNA-binding with InterPro entry IPR011598.

Item 44: Method according to item 42 or 43, wherein said GRP polypeptide has in increasing order of preference at least 50%, 55%, 60%, 65%, 70%, 75%, 80%, 85%, 90%, 95%, 98%, 99% or more amino acid sequence identity to the GRP polypeptide as represented by SEQ ID NO: 105 or to any of the polypeptide sequences given in Table A3 of Example 1.
Item 45: Method according to any preceding item 42 to 44, wherein said nucleic acid sequence encoding a GRP polypeptide is represented by the nucleic acid sequence of SEQ ID NO: 104 or a portion thereof, or a sequence capable of hybridising with the nucleic acid sequence of SEQ ID NO: 104 or a portion thereof or a sequence capable of hybridising with any one of the nucleic acid sequences SEQ ID NOs given in Table A3 of Example 1.
Item 46: Method according to any preceding item 42 to 45 wherein said modulated expression (preferably, increased) is effected by introducing and expressing in a plant a nucleic acid sequence encoding said GRP polypeptide.
Item 47: Method according to any preceding item 42 to 46, wherein said enhanced yield-related traits comprise one or more of: increased early vigour, increased greenness index, increased total seed yield per plant, increased number of filled seeds, increased seed fill rate, and increased harvest index, relative to control plants.
Item 48: Method according to any preceding item 42 to 47, wherein said modulated expression is increased expression.
Item 49: Method according to any preceding item 42 to 48, wherein said nucleic acid sequence is operably linked to a constitutive promoter, preferably to a high mobility group B (HMGB) promoter, most preferably to an HMGB promoter from rice.
Item 50: Method according to any preceding item 42 to 49, wherein said nucleic acid sequence encoding a GRP polypeptide is of plant origin, preferably from a dicotyledonous plant, further preferably from the family Brassicaceae, more preferably from Arabidopsis thaliana.
Item 51: Plant, part thereof (including seeds), or plant cell obtainable by a method according to any preceding item 42 to 45, wherein said plant, part thereof, or plant cell comprises a nucleic acid transgene encoding a GRP polypeptide.
Item 52: An isolated nucleic acid sequence comprising a nucleic acid sequence selected from the group consisting of:
(a) an isolated nucleic acid molecule encoding the polypeptide as depicted in SEQ ID NO: 122, 124, 126, 128 and/or 130 and/or table A3b;
(b) an isolated nucleic acid molecule as depicted in SEQ ID NO:121, 123, 125, 127 and/or 129 and/or table A3b;
(c) an isolated nucleic acid molecule, which, as a result of the degeneracy of the genetic code, can be derived from a polypeptide sequence depicted in table A3 and confers enhanced yield-related traits relative to control plants;
(d) an isolated nucleic acid molecule having at least 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with the nucleic acid molecule sequence of a polynucleotide comprising the nucleic acid molecule depicted in table A3 and confers enhanced yield-related traits relative to control plants;
(e) an isolated nucleic acid molecule encoding a polypeptide having at least 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of the polypeptide encoded by the nucleic acid molecule of (a) to (c) and confers enhanced yield-related traits relative to control plants;
(f) an isolated nucleic acid molecule which hybridizes with a nucleic acid molecule of (a) to (c) under stringent hybridization conditions and confers enhanced yield-related traits relative to control plants;
(g) an isolated nucleic acid molecule encoding a polypeptide comprising the consensus sequence or one or more polypeptide motifs as shown FIG. 10 and preferably having the activity represented by a nucleic acid molecule comprising a polynucleotide as depicted in table A3;
(h) an isolated nucleic acid molecule encoding a polypeptide having the activity represented by a protein as depicted in table A3 and confering enhanced yield-related traits relative to control plants;
and
(i) a nucleic acid molecule which is obtainable by screening a suitable nucleic acid library under stringent hybridization conditions with a probe comprising a complementary sequence of a nucleic acid molecule of (a) or (b) or with a fragment thereof, having at least 15 nt, preferably 20 nt, 30 nt, 50 nt, 100 nt, 200 nt or 500 nt of a nucleic acid molecule complementary to a nucleic acid molecule sequence characterized in (a) to (e) and encoding a polypeptide having the activity represented by a protein comprising a polypeptide as depicted in table A3;
whereby the nucleic acid molecule according to (a) to (i) is at least in one or more nucleotides different from the sequence depicted in table A3a and preferably which encodes a protein which differs at least in one or more amino acids from the protein sequences depicted in table A3a.
   Item 53: Construct comprising:
(a) nucleic acid sequence encoding a GRP polypeptide as defined in item 42 to 44 or 52; one or more control sequences capable of driving expression of the nucleic acid sequence of (a); and optionally
(b) a transcription termination sequence.
   Item 54: Construct according to item 53, wherein one of said control sequences is a constitutive promoter, preferably an HMGB promoter, most preferably an HMGB promoter from rice.
   Item 55: Use of a construct according to any of items 53 to 54 in a method for making plants having enhanced yield-related traits, particularly increased early vigour, increased greenness index, increased total seed yield per plant, increased number of filled seeds, increased seed fill rate, and increased harvest index, relative to control plants.
   Item 56: Plant, plant part or plant cell transformed with a construct according to any of items 53 to 54.
   Item 57: Method for the production of a transgenic plant having enhanced-yield related traits relative to control plants, comprising:
(i) introducing and expressing in a plant, plant part, or plant cell, a nucleic acid sequence encoding a GRP polypeptide as defined in items 42 to 44 and 52; and
(ii) cultivating the plant, plant part, or plant cell under conditions promoting plant growth and development.
   Item 58: Transgenic plant having enhanced yield-related traits, particularly increased early vigour, increased greenness index, increased total seed yield per plant, increased number of filled seeds, increased seed fill rate, and increased harvest index, relative to control plants, resulting from increased expression of a nucleic acid sequence encoding a GRP polypeptide as defined in items 42 to 44 and 52, or a transgenic plant cell derived from said transgenic plant.
   Item 59: Transgenic plant according to items 51, 56 and 58, or a transgenic plant cell derived thereof, wherein said plant is a crop plant or a monocot or a cereal, such as rice, maize, wheat, barley, millet, rye, triticale, sorghum and oats.
   Item 60: Harvestable parts of a plant comprising a nucleic acid sequence encoding a GRP polypeptide according to item 59, wherein said harvestable parts are preferably seeds.
   Item 61: Products derived from a plant according to item 59 and/or from harvestable parts of a plant according to item 60.
   Item 62: Use of a nucleic acid sequence encoding a GRP polypeptide in enhancing yield-related traits in plants, particularly in increasing early vigour, increasing greenness index, increasing total seed yield per plant, increasing number of filled seeds, increasing seed fill rate, and increasing harvest index, relative to control plants.

According to a further embodiment of the present invention, there is also provided an isolated polypeptide comprising a polypeptide sequence selected from the group consisting of:

(i) an amino acid sequence encoded by any one of the nucleic acids represented by Item 52;
(ii) an amino acid sequence having, in increasing order of preference, at least 50%, 55%, 60%, 65%, 70%, 75%, 80%, 85%, 90%, 95%, 96%, 97%, 98%, 99% or more sequence identity to the amino acid sequence of (i);
(iii) derivatives of any of the amino acid sequences given in (i) or (ii) above.

In one embodiment of the invention it has now been found that modulating expression (preferably, increasing) in a plant of a nucleic acid sequence encoding a GRP polypeptide, wherein said GRP polypeptide is an isopentenyl transferase (IPT) polypeptide, gives plants having enhanced yield-related traits relative to control plants. According to a first embodiment, the present invention provides a method for enhancing yield-related traits in plants relative to control plants, comprising modulating expression (preferably, increasing) in a plant of a nucleic acid sequence encoding a GRP polypeptide, wherein said GRP polypeptide is an isopentenyl transferase (IPT) polypeptide.

A preferred method for modulating (preferably, increasing) expression of a nucleic acid sequence encoding a GRP polypeptide is by introducing and expressing in a plant a nucleic acid sequence encoding a GRP polypeptide.

Any reference hereinafter to a “protein useful in the methods of the invention” is taken to mean a GRP polypeptide as defined herein. Any reference hereinafter to a “nucleic acid sequence useful in the methods of the invention” is taken to mean a nucleic acid sequence capable of encoding such a GRP polypeptide. The nucleic acid sequence to be introduced into a plant (and therefore useful in performing the methods of the invention) is any nucleic acid sequence encoding the type of protein which will now be described, hereafter also named “GRP nucleic acid sequence” or “GRP gene”.

A “GRP polypeptide” as defined herein refers the polypeptides represented by SEQ ID NO: 132, and to orthologues, paralogues, and homologues thereof or any of the polypeptide sequences given in Table A4 of Example 1 herein.

Preferably, the orthologues, paralogues, and homologues of SEQ ID NO: 132 belong to the tRNA isopentenyltransferase family with InterPro entries IPR002627 and IPR011593.

Alternatively or additionally, a “GRP polypeptide” as defined herein refers to any polypeptide having in increasing order of preference at least 50%, 55%, 60%, 65%, 70%, 75%, 80%, 85%, 90%, 95%, 98%, 99% or more amino acid sequence identity to the GRP polypeptide as represented by SEQ ID NO: 132 or to any of the polypeptide sequences given in Table A4 of Example 1 herein.

The term “domain” and “motif” is defined in the “definitions” section herein. Specialist databases exist for the identification of domains, for example, SMART (Schultz et al. (1998) Proc. Natl. Acad. Sci. USA 95, 5857-5864; Letunic et al. (2002) Nucleic Acids Res 30, 242-244), InterPro (Mulder et al., (2003) Nucl. Acids. Res. 31, 315-318), Prosite (Bucher and Bairoch (1994), A generalized profile syntax for biomolecular sequences motifs and its function in automatic sequence interpretation. (In) ISMB-94; Proceedings 2nd International Conference on Intelligent Systems for Molecular Biology. Altman R., Brutlag D., Karp P., Lathrop R., Searls D., Eds., pp 53-61, AAAI Press, Menlo Park; Hulo et al., Nucl. Acids. Res. 32:D134-D137, (2004) ), or Pfam (Bateman et al., Nucleic Acids Research 30(1): 276-280 (2002) ). A set of tools for in silico analysis of protein sequences is available on the ExPASy proteomics server (Swiss Institute of Bioinformatics (Gasteiger et al., ExPASy: the proteomics server for in-depth protein knowledge and analysis, Nucleic Acids Res. 31:3784-3788 (2003) ). Domains or motifs may also be identified using routine techniques, such as by sequence alignment.

Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the global (i.e. spanning the complete sequences) alignment of two sequences that maximizes the number of matches and minimizes the number of gaps. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information (NCBI). Homologues may readily be identified using, for example, the ClustalW multiple sequence alignment algorithm (version 1.83), with the default pairwise alignment parameters, and a scoring method in percentage. Global percentages of similarity and identity may also be determined using one of the methods available in the MatGAT software package (Campanella et al., BMC Bioinformatics. 2003 Jul. 10; 4:29. MatGAT: an application that generates similarity/identity matrices using protein or DNA sequences). Minor manual editing may be performed to optimise alignment between conserved motifs, as would be apparent to a person skilled in the art. Furthermore, instead of using full-length sequences for the identification of homologues, specific domains may also be used. The sequence identity values may be determined over the entire nucleic acid sequence or polypeptide sequence or over selected domains or conserved motif(s), using the programs mentioned above using the default parameters.

Cytokinins, which are central regulators of cell division and differentiation in plants, are adenine derivatives carrying an isopentenyl side chain that may be hydroxylated. Plants have two classes of isopentenyltransferases (IPTs) acting on the adenine moiety: ATP/ADP isopentenyltransferases (in Arabidopsis thaliana, AtlPT1, 3, 4-8) and tRNA IPTs (in Arabidopsis, AtlPT2 and 9; Miyawaki et al. (2006) Proc Natl Acad Sci USA. 103(44): 16598-16603.). As described above, isopentenylation of ATP and ADP, by ATP/ADP IPTs, is the key step in the biosynthesis of iP- and tZ-type cytokinins. GRP polypeptides, as far as SEQ ID NO: 2, and its orthologues, paralogues, and homologues are concerned, typically are responsible for the isopentenylation of ATP and ADP in the biosynthesis of iP- and tZ-type cytokinins. Measuring such enzymatic activity is well known in the art (for example, Miyawaki et al. supra).

The present invention is illustrated by transforming plants with the nucleic acid sequence represented by SEQ ID NO: 131, encoding the polypeptide sequence of SEQ ID NO: 132. However, performance of the invention is not restricted to these sequences; the methods of the invention may advantageously be performed using any GRP-encoding nucleic acid sequence or GRP polypeptide as defined herein.

Examples of nucleic acid sequences encoding GRP polypeptides may be found in databases known in the art. Such nucleic acid sequences are useful in performing the methods of the invention. Orthologues and paralogues, the terms “orthologues” and “paralogues” being as defined herein, may readily be identified by performing a so-called reciprocal blast search. Typically, this involves a first BLAST involving BLASTing a query sequence (for example using SEQ ID NO: 132) against any sequence database, such as the publicly available NCBI database. BLASTN or TBLASTX (using standard default values) are generally used when starting from a nucleotide sequence, and BLASTP or TBLASTN (using standard default values) when starting from a protein sequence. The BLAST results may optionally be filtered. The full-length sequences of either the filtered results or non-filtered results are then BLASTed back (second BLAST) against sequences from the organism from which the query sequence is derived (where the query sequence is SEQ ID NO: 131 or SEQ ID NO: 132, the second BLAST would therefore be against Arabidopsis thaliana sequences). The results of the first and second BLASTs are then compared. A paralogue is identified if a high-ranking hit from the first blast is from the same species as from which the query sequence is derived, a BLAST back then ideally results in the query sequence amongst the highest hits; an orthologue is identified if a high-ranking hit in the first BLAST is not from the same species as from which the query sequence is derived, and preferably results upon BLAST back in the query sequence being among the highest hits.

High-ranking hits are those having a low E-value. The lower the E-value, the more significant the score (or in other words the lower the chance that the hit was found by chance). Computation of the E-value is well known in the art. In addition to E-values, comparisons are also scored by percentage identity. Percentage identity refers to the number of identical nucleotides (or amino acids) between the two compared nucleic acid sequence (or polypeptide) sequences over a particular length. In the case of large families, ClustalW may be used, followed by a neighbour joining tree, to help visualize clustering of related genes and to identify orthologues and paralogues.

Nucleic acid sequence variants encoding homologues and derivatives of SEQ ID NO: 132 may also be useful in practising the methods of the invention, the terms “homologue” and “derivative” being as defined herein. Also useful in the methods of the invention are nucleic acid sequences encoding homologues and derivatives of orthologues or paralogues of SEQ ID NO: 132 or of any of the polypeptide sequences given in Table A4 of Example 1. Homologues and derivatives useful in the methods of the present invention have substantially the same biological and functional activity as the unmodified protein from which they are derived.

Further nucleic acid sequence variants useful in practising the methods of the invention include portions of nucleic acid sequences encoding GRP polypeptides, nucleic acid sequences hybridising to nucleic acid sequences encoding GRP polypeptides, splice variants of nucleic acid sequences encoding GRP polypeptides, allelic variants of nucleic acid sequences encoding GRP polypeptides and variants of nucleic acid sequences encoding GRP polypeptides obtained by gene shuffling. The terms hybridising sequence, splice variant, allelic variant and gene shuffling are as described herein.

Nucleic acid sequences encoding GRP polypeptides need not be full-length nucleic acid sequences, since performance of the methods of the invention does not rely on the use of full-length nucleic acid sequences. According to the present invention, there is provided a method for enhancing yield-related traits in plants, comprising introducing and expressing in a plant a portion of SEQ ID NO: 131, or a portion of a nucleic acid sequence encoding an orthologue, paralogue, or homologue of SEQ ID NO: 132.

A portion of a nucleic acid sequence may be prepared, for example, by making one or more deletions to the nucleic acid sequence. The portions may be used in isolated form or they may be fused to other coding (or non-coding) sequences in order to, for example, produce a protein that combines several activities. When fused to other coding sequences, the resultant polypeptide produced upon translation may be bigger than that predicted for the polypeptide portion.

Portions useful in the methods of the invention, encode a GRP polypeptide as defined herein, and have substantially the same biological activity as the polypeptide sequence given in SEQ ID NO: 132 or as any of the polypeptide sequences given in Table A4 of Example 1. Preferably, the portion is a portion of the nucleic acid sequence given in SEQ ID NO: 131, or is a portion of a nucleic acid sequence encoding an orthologue or paralogue of the polypeptide sequence given in SEQ ID NO: 132. Preferably the portion is at least 300, 400, 500, 600, 700, 800, 850, 900, 950, 1000, 1050, or more consecutive nucleotides in length, the consecutive nucleotides being of SEQ ID NO: 131, or of a nucleic acid sequence encoding an orthologue or paralogue of SEQ ID NO: 132. Most preferably the portion is a portion of the nucleic acid sequence of SEQ ID NO: 131.

Another nucleic acid sequence variant useful in the methods of the invention is a nucleic acid sequence capable of hybridising, under reduced stringency conditions, preferably under stringent conditions, with a nucleic acid sequence encoding a GRP polypeptide as defined herein, or with a portion as defined herein.

According to the present invention, there is provided a method for enhancing yield-related traits in plants comprising introducing and expressing in a plant a nucleic acid sequence capable of hybridizing to SEQ ID NO: 131, or comprising introducing and expressing in a plant a nucleic acid sequence capable of hybridising to a nucleic acid sequence encoding an orthologue, paralogue, or homologue of SEQ ID NO: 132 or of any of the polypeptide sequences given in Table A of Example 1.

Hybridising sequences useful in the methods of the invention encode a GRP polypeptide as defined herein, having substantially the same biological activity as the polypeptide sequence given in SEQ ID NO: 132. Preferably, the hybridising sequence is capable of hybridising to SEQ ID NO: 131, or to a portion of this sequence, a portion being as defined above, or the hybridising sequence is capable of hybridising to a nucleic acid sequence encoding an orthologue or paralogue of SEQ ID NO: 132, or to a portion thereof or to any of the polypeptide sequences given in Table A of Example 1.

Another nucleic acid sequence variant useful in the methods of the invention is a splice variant encoding a GRP polypeptide as defined hereinabove, a splice variant being as defined herein.

According to the present invention, there is provided a method for enhancing yield-related traits in plants, comprising introducing and expressing in a plant a splice variant of SEQ ID NO: 131, or a splice variant of a nucleic acid sequence encoding an orthologue, paralogue, or homologue of SEQ ID NO: 132.

Another nucleic acid sequence variant useful in performing the methods of the invention is an allelic variant of a nucleic acid sequence encoding a GRP polypeptide as defined hereinabove, an allelic variant being as defined herein.

According to the present invention, there is provided a method for enhancing yield-related traits in plants comprising introducing and expressing in a plant an allelic variant of SEQ ID NO: 131, or comprising introducing and expressing in a plant an allelic variant of a nucleic acid sequence encoding an orthologue, paralogue, or homologue of the polypeptide sequence represented by SEQ ID NO: 132.

The allelic variants useful in the methods of the present invention have substantially the same biological activity as the GRP polypeptide of SEQ ID NO: 132. Allelic variants exist in nature, and encompassed within the methods of the present invention is the use of these natural alleles. Gene shuffling or directed evolution may also be used to generate variants of nucleic acid sequences encoding GRP polypeptides as defined above; the term “gene shuffling” being as defined herein.

According to the present invention, there is provided a method for enhancing yield-related traits in plants comprising introducing and expressing in a plant a variant nucleic acid sequence of SEQ ID NO: 131, or comprising introducing and expressing in a plant a variant of a nucleic acid sequence encoding an orthologue, paralogue, or homologue of SEQ ID NO: 132, which variant nucleic acid sequence is obtained by gene shuffling.

Furthermore, nucleic acid sequence variants may also be obtained by site-directed mutagenesis. Several methods are available to achieve site-directed mutagenesis, the most common being PCR based methods (Current Protocols in Molecular Biology. Wiley Eds.).

Nucleic acid sequences encoding GRP polypeptides may be derived from any natural or artificial source. The nucleic acid sequence may be modified from its native form in composition and/or genomic environment through deliberate human manipulation. Preferably the GRP polypeptide-encoding nucleic acid sequence is from a plant. In the case of SEQ ID NO: 131, the GRP polypeptide encoding nucleic acid sequence is preferably from a dicotyledonous plant, more preferably from the family Brassicaceae, most preferably the nucleic acid sequence is from Arabidopsis thaliana.

Performance of the methods of the invention gives plants having enhanced yield-related traits, preferably enhanced seed yield-related traits. The terms “yield” and “seed yield” are described in more detail in the “definitions” section herein.

Reference herein to enhanced yield-related traits is taken to mean an increase in early vigour and/or in biomass (weight) of one or more parts of a plant, which may include aboveground (harvestable) parts and/or (harvestable) parts below ground. In particular, such harvestable parts are biomass and/or seeds, and performance of the methods of the invention results in plants having increased early vigour, biomass and/or seed yield relative to the early vigour, biomass or seed yield of control plants.

Taking corn as an example, a yield increase may be manifested as one or more of the following: increase in the number of plants established per hectare or acre, an increase in the number of ears per plant, an increase in the number of rows, number of kernels per row, kernel weight, thousand kernel weight, ear length/diameter, increase in the seed filling rate (which is the number of filled seeds divided by the total number of seeds and multiplied by 100), among others. Taking rice as an example, a yield increase may manifest itself as an increase in one or more of the following: number of plants per hectare or acre, number of panicles per plant, number of spikelets per panicle, number of flowers (florets) per panicle (which is expressed as a ratio of the number of filled seeds over the number of primary panicles), increase in the seed filling rate (which is the number of filled seeds divided by the total number of seeds and multiplied by 100), increase in thousand kernel weight, among others.

The present invention provides a method for enhancing yield-related traits, especially biomass and/or seed yield of plants, relative to control plants, which method comprises modulating expression (preferably, increasing) in a plant of a nucleic acid sequence encoding a GRP polypeptide as defined herein.

Since the transgenic plants according to the present invention have enhanced yield-related traits, it is likely that these plants exhibit an increased growth rate (during at least part of their life cycle), relative to the growth rate of control plants at a corresponding stage in their life cycle.

The increased growth rate may be specific to one or more parts of a plant (including seeds), or may be throughout substantially the whole plant. Plants having an increased growth rate may have a shorter life cycle. The life cycle of a plant may be taken to mean the time needed to grow from a dry mature seed up to the stage where the plant has produced dry mature seeds, similar to the starting material. This life cycle may be influenced by factors such as early vigour, growth rate, greenness index, flowering time and speed of seed maturation. The increase in growth rate may take place at one or more stages in the life cycle of a plant or during substantially the whole plant life cycle. Increased growth rate during the early stages in the life cycle of a plant may reflect enhanced vigour. The increase in growth rate may alter the harvest cycle of a plant allowing plants to be sown later and/or harvested sooner than would otherwise be possible (a similar effect may be obtained with earlier flowering time). If the growth rate is sufficiently increased, it may allow for the further sowing of seeds of the same plant species (for example sowing and harvesting of rice plants followed by sowing and harvesting of further rice plants all within one conventional growing period). Similarly, if the growth rate is sufficiently increased, it may allow for the further sowing of seeds of different plants species (for example the sowing and harvesting of corn plants followed by, for example, the sowing and optional harvesting of soybean, potato or any other suitable plant). Harvesting additional times from the same rootstock in the case of some crop plants may also be possible. Altering the harvest cycle of a plant may lead to an increase in annual biomass production per acre (due to an increase in the number of times (say in a year) that any particular plant may be grown and harvested). An increase in growth rate may also allow for the cultivation of transgenic plants in a wider geographical area than their wild-type counterparts, since the territorial limitations for growing a crop are often determined by adverse environmental conditions either at the time of planting (early season) or at the time of harvesting (late season). Such adverse conditions may be avoided if the harvest cycle is shortened. The growth rate may be determined by deriving various parameters from growth curves, such parameters may be: T-Mid (the time taken for plants to reach 50% of their maximal size) and T-90 (time taken for plants to reach 90% of their maximal size), amongst others.

According to a preferred feature of the present invention, performance of the methods of the invention gives plants having an increased growth rate relative to control plants. Therefore, according to the present invention, there is provided a method for increasing the growth rate of plants, which method comprises modulating expression (preferably, increasing) in a plant of a nucleic acid sequence encoding a GRP polypeptide as defined herein.

An enhancement of yield-related traits (an increase in seed yield and/or growth rate) occurs whether the plant is under non-stress conditions or whether the plant is exposed to various stresses compared to control plants. Plants typically respond to exposure to stress by growing more slowly. In conditions of severe stress, the plant may even stop growing altogether. Mild stress on the other hand is defined herein as being any stress to which a plant is exposed which does not result in the plant ceasing to grow altogether without the capacity to resume growth. Mild stress in the sense of the invention leads to a reduction in the growth of the stressed plants of less than 40%, 35% or 30%, preferably less than 25%, 20% or 15%, more preferably less than 14%, 13%, 12%, 11% or 10% or less in comparison to the control plant under non-stress conditions. Due to advances in agricultural practices (irrigation, fertilization, pesticide treatments) severe stresses are not often encountered in cultivated crop plants. As a consequence, the compromised growth induced by mild stress is often an undesirable feature for agriculture. Mild stresses are the everyday biotic and/or abiotic (environmental) stresses to which a plant is exposed. Abiotic stresses may be due to drought or excess water, anaerobic stress, salt stress, chemical toxicity, oxidative stress and hot, cold or freezing temperatures. The abiotic stress may be an osmotic stress caused by a water stress (particularly due to drought), salt stress, oxidative stress or an ionic stress. Biotic stresses are typically those stresses caused by pathogens, such as bacteria, viruses, nematodes, fungi and insects. The term “non-stress” conditions as used herein are those environmental conditions that allow optimal growth of plants. Persons skilled in the art are aware of normal soil conditions and climatic conditions for a given location.

Performance of the methods of the invention gives plants grown under non-stress conditions or under mild stress conditions having enhanced yield-related traits relative to control plants grown under comparable conditions. Therefore, according to the present invention, there is provided a method for enhancing yield-related traits in plants grown under non-stress conditions or under mild stress conditions, which method comprises modulating (preferably, increasing) expression in a plant of a nucleic acid sequence encoding a GRP polypeptide.

Performance of the methods according to the present invention results in plants grown under abiotic stress conditions having enhanced yield-related traits relative to control plants grown under comparable stress conditions. As reported in Wang et al. (Planta (2003) 218: 1-14), abiotic stress leads to a series of morphological, physiological, biochemical and molecular changes that adversely affect plant growth and productivity. Drought, salinity, extreme temperatures and oxidative stress are known to be interconnected and may induce growth and cellular damage through similar mechanisms. Rabbani et al. (Plant Physiol (2003) 133: 1755-1767) describes a particularly high degree of “cross talk” between drought stress and high-salinity stress. For example, drought and/or salinisation are manifested primarily as osmotic stress, resulting in the disruption of homeostasis and ion distribution in the cell. Oxidative stress, which frequently accompanies high or low temperature, salinity or drought stress, may cause denaturing of functional and structural proteins. As a consequence, these diverse environmental stresses often activate similar cell signalling pathways and cellular responses, such as the production of stress proteins, up-regulation of anti-oxidants, accumulation of compatible solutes and growth arrest. Since diverse environmental stresses activate similar pathways, plants having enhanced yield-related traits, preferably enhanced seed yield-related traits, under one type of abiotic stress growth conditions, relative to control plants grown under comparable abiotic stress conditions, should not be seen as a limitation to that type of abiotic stress growth conditions, but more as a screen to indicate the involvement of GRP polypeptides as defined above, in enhancing yield-related traits in abiotic stress growth conditions in general.

The term “abiotic stress” as defined herein is taken to mean any one or more of: water stress (due to drought or excess water), anaerobic stress, salt stress, temperature stress (due to hot, cold or freezing temperatures), chemical toxicity stress and oxidative stress. According to one aspect of the invention, the abiotic stress is an osmotic stress, selected from water stress, salt stress, oxidative stress and ionic stress. Preferably, the water stress is drought stress. The term salt stress is not restricted to common salt (NaCl), but may be any stress caused by one or more of: NaCl, KCl, LiCl, MgCl2, CaCl2, amongst others.

Performance of the methods of the invention gives plants having enhanced yield-related traits under abiotic stress growth conditions relative to control plants grown in comparable stress conditions. Therefore, according to the present invention, there is provided a method for enhancing yield-related traits in plants grown under abiotic stress growth conditions, which method comprises modulating (preferably, increasing) expression in a plant of a nucleic acid sequence encoding a GRP polypeptide. According to one aspect of the invention, the abiotic stress is an osmotic stress, selected from one or more of the following: water stress, salt stress, oxidative stress and ionic stress.

Performance of the methods of the invention gives plants grown under conditions of reduced nutrient availability, particularly under conditions of reduced nitrogen availability, enhanced yield-related traits relative to control plants grown under comparable conditions. Therefore, according to the present invention, there is provided a method for enhancing yield-related traits in plants grown under conditions of reduced nutrient availability, which method comprises modulating (preferably, increasing) expression in a plant of a nucleic acid sequence encoding a GRP polypeptide. Reduced nutrient availability may comprise reduced availability of nutrients such as nitrogen, phosphates and other phosphorous-containing compounds, potassium, calcium, cadmium, magnesium, manganese, iron and boron, amongst others.

The present invention encompasses plants, parts thereof (including seeds), or plant cells obtainable by the methods according to the present invention. The plants, parts thereof, or plant cells comprise a nucleic acid transgene encoding a GRP polypeptide as defined above, preferably of isopentenyl transferase (IPT) polypeptide.

The invention also provides genetic constructs and vectors to facilitate introduction and/or expression in plants of nucleic acid sequences encoding GRP polypeptides. The gene constructs may be inserted into vectors, which may be commercially available, suitable for transforming into plants and suitable for expression of the gene of interest in the transformed cells. The invention also provides use of a gene construct as defined herein in the methods of the invention.

More specifically, the present invention provides a construct comprising:
(i) a nucleic acid sequence encoding a GRP polypeptide as defined above, preferably of isopentenyl transferase (IPT) polypeptide;
(ii) one or more control sequences capable of driving expression of the nucleic acid sequence of (a); and optionally
(iii) a transcription termination sequence.

Preferably, the nucleic acid sequence encoding a GRP polypeptide is as defined above. The term “control sequence” and “termination sequence” are as defined herein.

Plants are transformed with a vector comprising any of the nucleic acid sequences described above. The skilled artisan is well aware of the genetic elements that must be present on the vector in order to successfully transform, select and propagate host cells containing the sequence of interest. The sequence of interest is operably linked to one or more control sequences (at least to a promoter).

Advantageously, any type of promoter, whether natural or synthetic, may be used to drive expression of the nucleic acid sequence. A seed-specific promoter is particularly useful in the methods of the invention. See the “Definitions” section herein for definitions of the various promoter types.

It should be clear that the applicability of the present invention is not restricted to the GRP polypeptide-encoding nucleic acid sequence represented by SEQ ID NO: 131, nor is the applicability of the invention restricted to expression of a GRP polypeptide-encoding nucleic acid sequence when driven by a seed-specific promoter.

The seed-specific promoter is preferably a prolamin promoter, preferably a prolamin promoter from rice. Further preferably the prolamin promoter is represented by a nucleic acid sequence substantially similar to SEQ ID NO: 133, most preferably the prolamin promoter is as represented by SEQ ID NO: 133. See Table 2 in the “Definitions” section herein for further examples of seed-specific promoters.

Optionally, one or more terminator sequences may be used in the construct introduced into a plant. Additional regulatory elements may include transcriptional as well as translational enhancers. Those skilled in the art will be aware of terminator and enhancer sequences that may be suitable for use in performing the invention. An intron sequence may also be added to the 5′ untranslated region (UTR) or in the coding sequence to increase the amount of the mature message that accumulates in the cytosol, as described in the definitions section. Other control sequences (besides promoter, enhancer, silencer, intron sequences, 3′UTR and/or 5′UTR regions) may be protein and/or RNA stabilizing elements. Such sequences would be known or may readily be obtained by a person skilled in the art.

The genetic constructs of the invention may further include an origin of replication sequence that is required for maintenance and/or replication in a specific cell type. One example is when a genetic construct is required to be maintained in a bacterial cell as an episomal genetic element (e.g. plasmid or cosmid molecule). Preferred origins of replication include, but are not limited to, the f1-ori and colE1.

For the detection of the successful transfer of the nucleic acid sequences as used in the methods of the invention and/or selection of transgenic plants comprising these nucleic acid sequences, it is advantageous to use marker genes (or reporter genes). Therefore, the genetic construct may optionally comprise a selectable marker gene. Selectable markers are described in more detail in the “definitions” section herein. The marker genes may be removed or excised from the transgenic cell once they are no longer needed. Techniques for marker removal are known in the art, useful techniques are described above in the definitions section.

The invention also provides a method for the production of transgenic plants having enhanced yield-related traits relative to control plants, comprising introduction and expression in a plant of a nucleic acid sequence encoding a GRP polypeptide as defined hereinabove, preferably of isopentenyl transferase (IPT) polypeptide.

More specifically, the present invention provides a method for the production of transgenic plants having enhanced yield-related traits, which method comprises:
(i) introducing and expressing in a plant, plant part, or plant cell, a nucleic acid sequence encoding a GRP polypeptide; and
(ii) cultivating the plant, plant part or plant cell under conditions promoting plant growth and development.

The nucleic acid sequence of (i) may be any of the nucleic acid sequences capable of encoding a GRP polypeptide as defined herein.

The nucleic acid sequence may be introduced directly into a plant cell or into the plant itself (including introduction into a tissue, organ or any other part of a plant). According to a preferred feature of the present invention, the nucleic acid sequence is preferably introduced into a plant by transformation. The term “transformation” is described in more detail in the “definitions” section herein.

The genetically modified plant cells can be regenerated via all methods with which the skilled worker is familiar. Suitable methods can be found in the abovementioned publications by S. D. Kung and R. Wu, Potrykus or Hofgen and Willmitzer.

Generally after transformation, plant cells or cell groupings are selected for the presence of one or more markers which are encoded by plant-expressible genes co-transferred with the gene of interest, following which the transformed material is regenerated into a whole plant. To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as the ones described above.

Following DNA transfer and regeneration, putatively transformed plants may also be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

The present invention clearly extends to any plant cell or plant produced by any of the methods described herein, and to all plant parts and propagules thereof. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced by the parent in the methods according to the invention.

The invention also includes host cells containing an isolated nucleic acid sequence encoding a GRP polypeptide as defined hereinabove. Preferred host cells according to the invention are plant cells. Host plants for the nucleic acid transgene or the vector used in the method according to the invention, the expression cassette or construct or vector are, in principle, advantageously all plants, which are capable of synthesizing the polypeptides used in the inventive method.

The methods of the invention are advantageously applicable to any plant. Plants that are particularly useful in the methods of the invention include all plants which belong to the superfamily Viridiplantae, in particular monocotyledonous and dicotyledonous plants including fodder or forage legumes, ornamental plants, food crops, trees or shrubs. According to a preferred embodiment of the present invention, the plant is a crop plant. Examples of crop plants include soybean, sunflower, canola, alfalfa, rapeseed, cotton, tomato, potato and tobacco. Further preferably, the plant is a monocotyledonous plant. Examples of monocotyledonous plants include sugarcane. More preferably the plant is a cereal. Examples of cereals include rice, maize, wheat, barley, millet, rye, triticale, sorghum and oats.

The invention also extends to harvestable parts of a plant such as, but not limited to seeds, leaves, fruits, flowers, stems, roots, rhizomes, tubers and bulbs. The invention furthermore relates to products derived, preferably directly derived, from a harvestable part of such a plant, such as dry pellets or powders, oil, fat and fatty acids, starch or proteins.

According to a preferred feature of the invention, the modulated expression is increased expression. Methods for increasing expression of nucleic acid sequences or genes, or gene products, are well documented in the art and examples are provided in the definitions section.

As mentioned above, a preferred method for modulating (preferably, increasing) expression of a nucleic acid sequence encoding a GRP polypeptide is by introducing and expressing in a plant a nucleic acid sequence encoding a GRP polypeptide; however the effects of performing the method, i.e. enhancing yield-related traits, may also be achieved using other well known techniques, including but not limited to T-DNA activation tagging, TILLING, homologous recombination. A description of these techniques is provided in the definitions section.

The present invention also encompasses use of nucleic acid sequences encoding GRP polypeptides as described herein and use of these GRP polypeptides in enhancing any of the aforementioned yield-related traits in plants.

Nucleic acid sequences encoding GRP polypeptide described herein, or the GRP polypeptides themselves, may find use in breeding programmes in which a DNA marker is identified which may be genetically linked to a GRP polypeptide-encoding gene. The genes/nucleic acid sequences, or the GRP polypeptides themselves may be used to define a molecular marker. This DNA or protein marker may then be used in breeding programmes to select plants having enhanced yield-related traits, as defined hereinabove in the methods of the invention.

Allelic variants of a GRP polypeptide-encoding nucleic acid sequence/gene may also find use in marker-assisted breeding programmes. Such breeding programmes sometimes require introduction of allelic variation by mutagenic treatment of the plants, using for example EMS mutagenesis; alternatively, the programme may start with a collection of allelic variants of so called “natural” origin caused unintentionally. Identification of allelic variants then takes place, for example, by PCR. This is followed by a step for selection of superior allelic variants of the sequence in question and which give enhanced yield-related traits. Selection is typically carried out by monitoring growth performance of plants containing different allelic variants of the sequence in question. Growth performance may be monitored in a greenhouse or in the field. Further optional steps include crossing plants in which the superior allelic variant was identified with another plant. This could be used, for example, to make a combination of interesting phenotypic features.

Nucleic acid sequences encoding GRP polypeptides may also be used as probes for genetically and physically mapping the genes that they are a part of, and as markers for traits linked to those genes. Such information may be useful in plant breeding in order to develop lines with desired phenotypes. Such use of GRP polypeptide-encoding nucleic acid sequences requires only a nucleic acid sequence of at least 15 nucleotides in length. The GRP polypeptide-encoding nucleic acid sequences may be used as restriction fragment length polymorphism (RFLP) markers. Southern blots (Sambrook J, Fritsch E F and Maniatis T (1989) Molecular Cloning, A Laboratory Manual) of restriction-digested plant genomic DNA may be probed with the GRP-encoding nucleic acid sequences. The resulting banding patterns may then be subjected to genetic analyses using computer programs such as MapMaker (Lander et al. (1987) Genomics 1: 174-181) in order to construct a genetic map. In addition, the nucleic acid sequences may be used to probe Southern blots containing restriction endonuclease-treated genomic DNAs of a set of individuals representing parent and progeny of a defined genetic cross. Segregation of the DNA polymorphisms is noted and used to calculate the position of the GRP polypeptide-encoding nucleic acid sequence in the genetic map previously obtained using this population (Botstein et al. (1980) Am. J. Hum. Genet. 32:314-331).

The production and use of plant gene-derived probes for use in genetic mapping is described in Bernatzky and Tanksley (1986) Plant Mol. Biol. Reporter 4: 37-41. Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

The nucleic acid sequence probes may also be used for physical mapping (i.e., placement of sequences on physical maps; see Hoheisel et al. In: Non-mammalian Genomic Analysis: A Practical Guide, Academic press 1996, pp. 319-346, and references cited therein).

In another embodiment, the nucleic acid sequence probes may be used in direct fluorescence in situ hybridisation (FISH) mapping (Trask (1991) Trends Genet. 7:149-154). Although current methods of FISH mapping favour use of large clones (several kb to several hundred kb; see Laan et al. (1995) Genome Res. 5:13-20), improvements in sensitivity may allow performance of FISH mapping using shorter probes.

A variety of nucleic acid sequence amplification-based methods for genetic and physical mapping may be carried out using the nucleic acid sequences. Examples include allele-specific amplification (Kazazian (1989) J. Lab. Clin. Med 11:95-96), polymorphism of PCR-amplified fragments (CAPS; Sheffield et al. (1993) Genomics 16:325-332), allele-specific ligation (Landegren et al. (1988) Science 241:1077-1080), nucleotide extension reactions (Sokolov (1990) Nucleic Acids Res. 18:3671), Radiation Hybrid Mapping (Walter et al. (1997) Nat. Genet. 7:22-28) and Happy Mapping (Dear and Cook (1989) Nucleic Acids Res. 17:6795-6807). For these methods, the sequence of a nucleic acid sequence is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for mapping methods.

The methods according to the present invention result in plants having enhanced yield-related traits, as described hereinbefore. These traits may also be combined with other economically advantageous traits, such as further yield-enhancing traits, tolerance to other abiotic and biotic stresses, traits modifying various architectural features and/or biochemical and/or physiological features.

In one embodiment the invention relates to subject mater summarized as follows:

Item 63: A method for enhancing yield-related traits in plants relative to control plants, comprising modulating expression (preferably, increasing) in a plant of a nucleic acid sequence encoding a GRP polypeptide, wherein said GRP polypeptide is an isopentenyl transferase (IPT) polypeptide as represented by SEQ ID NO: 132 or an orthologue, paralogue, or homologue thereof, and optionally selecting for plants having enhanced yield-related traits.
Item 64: A method according to item 63, wherein said GRP polypeptide as represented by SEQ ID NO: 132 and an orthologue, paralogue, or homologue thereof, belong to the tRNA isopentenyltransferase family with InterPro entries IPR002627 and IPR011593 or comprising a domain or motif according to the consensus sequence as depicted in FIG. 13.
Item 65: Method according to item 63 or 64, wherein said GRP polypeptide has in increasing order of preference at least 50%, 55%, 60%, 65%, 70%, 75%, 80%, 85%, 90%, 95%, 98%, 99% or more amino acid sequence identity to the GRP polypeptide as represented by SEQ ID NO: 132 or to any of the polypeptide sequences given in Table A4 of Example 1.
Item 66: Method according to any preceding item 63 to 65, wherein said nucleic acid sequence encoding a GRP polypeptide is represented by the nucleic acid sequence of SEQ ID NO: 131 or a portion thereof, or a sequence capable of hybridising with the nucleic acid sequence of SEQ ID NO: 131 or a portion thereof or a sequence capable of hybridising with any one of the nucleic acid sequences SEQ ID NOs given in Table A4 of Example 1.
Item 67: Method according to any preceding item 63 to 66 wherein said modulated expression (preferably, increased) is effected by introducing and expressing in a plant a nucleic acid sequence encoding said GRP polypeptide.
Item 68: Method according to any preceding item 63 to 67, wherein said enhanced yield-related traits comprise one or more of: increased total seed yield per plant, increased number of filled seeds, increased total number of seeds, and increased harvest index, relative to control plants.
Item 69: Method according to any preceding item 63 to 68, wherein said modulated expression is increased expression.
Item 70: Method according to any preceding item 63 to 69, wherein said nucleic acid sequence is operably linked to a seed-specific promoter, preferably to a prolamin promoter, most preferably to a prolamin promoter from rice.
Item 71: Method according to any preceding item 63 to 70, wherein said nucleic acid sequence encoding a GRP polypeptide is of plant origin, preferably from a dicotyledonous plant, further preferably from the family Brassicaceae, more preferably from Arabidopsis thaliana.
Item 72: Plant, part thereof (including seeds), or plant cell obtainable by a method according to any preceding item 63 to 71, wherein said plant, part thereof, or plant cell comprises a nucleic acid transgene encoding a GRP polypeptide.

Item 73: Construct comprising:
(a) nucleic acid sequence encoding a GRP polypeptide as defined in item 63 to 65;
(b) one or more control sequences capable of driving expression of the nucleic acid sequence of (a); and optionally
(c) a transcription termination sequence.
   Item 74: Construct according to item 73, wherein one of said control sequences is a seed-specific promoter, preferably a prolamin promoter, most preferably a prolamin promoter from rice.
   Item 75: Use of a construct according to any of items 73 to 75 in a method for making plants having enhanced yield-related traits, particularly increased total seed yield per plant, increased number of filled seeds, increased total number of seeds, and increased harvest index, relative to control plants.
   Item 76: Plant, plant part or plant cell transformed with a construct according to any of items 74 to 75.
   Item 77: Method for the production of a transgenic plant having enhanced-yield related traits relative to control plants, comprising:
(i) introducing and expressing in a plant, plant part, or plant cell, a nucleic acid sequence encoding a GRP polypeptide as defined in item 63 to 65; and
(ii) cultivating the plant, plant part, or plant cell under conditions promoting plant growth and development.
   Item 78: Transgenic plant having enhanced yield-related traits, particularly increased total seed yield per plant, increased number of filled seeds, increased total number of seeds, and increased harvest index, relative to control plants, resulting from increased expression of a nucleic acid sequence encoding a GRP polypeptide as defined in items 63 to 65, or a transgenic plant cell derived from said transgenic plant.
   Item 79: Transgenic plant according to item 72, 76 or 78, or a transgenic plant cell derived thereof, wherein said plant is a crop plant or a monocot or a cereal, such as rice, maize, wheat, barley, millet, rye, triticale, sorghum and oats.
   Item 80: Harvestable parts of a plant comprising a nucleic acid sequence encoding a GRP polypeptide according to item 79, wherein said harvestable parts are preferably seeds.
   Item 81: Products derived from a plant according to item 79 and/or from harvestable parts of a plant according to item 80.
   Item 81: Use of a nucleic acid sequence encoding a GRP polypeptide in enhancing yield-related traits in plants, particularly in increasing total seed yield per plant, increasing number of filled seeds, increasing total number of seeds, and increasing harvest index, relative to control plants.

In one embodiment of the invention it has now been found that modulating expression in a plant of a nucleic acid encoding an STO polypeptide gives plants having enhanced yield-related traits relative to control plants. According to a first embodiment, the present invention provides a method for enhancing yield-related traits in plants relative to control plants, comprising modulating expression in a plant of a nucleic acid encoding an STO polypeptide.

A preferred method for modulating (preferably, increasing) expression of a nucleic acid encoding an STO polypeptide is by introducing and expressing in a plant a nucleic acid encoding an STO polypeptide.

Any reference hereinafter to a “protein useful in the methods of the invention” is taken to mean an STO polypeptide as defined herein. Any reference hereinafter to a “nucleic acid useful in the methods of the invention” is taken to mean a nucleic acid capable of encoding such an STO polypeptide. The nucleic acid to be introduced into a plant (and therefore useful in performing the methods of the invention) is any nucleic acid encoding the type of protein which will now be described, hereinafter also named “STO nucleic acid” or “STO gene”.

An “STO polypeptide” as defined herein refers to any polypeptide comprising at least one B-box domain. The B-box domain is an amino acid domain conserved in proteins of diverse origin including prokaryotic and eukaryotic organism. B-box domain is about 40 amino acids in length comprising conserved cysteine and/or histidine residues at key positions such that the domain can fall into a tertiary structure similar to the zinc fingers involved in the coordination of zinc ions. NMR analysis reveals that the B-box structure comprises two beta-strands, two helical turns and three extended loop regions different from any other zinc binding motif (Borden et al. 1995 EMBO J. 1995 Dec. 1; 14(23):5947-56).

Proteins comprising B-box domains can be found in specific databases such as Pfam and Interpro. The accession number of the B-box domain in the release 15.1 of the Interpro database is IPR000315, and PF00643 in Pfam version 21.0.

Based on sequence comparison of B-box domains found in different proteins it is possible to define consensus sequences representing B-box domains. For example Reymond et al. (EMBO J. 2001 May 1; 20(9):2140-51) were able to define consensus sequences to the two B-box domains present in TRIM proteins of Homo sapiens, as represented by CXX(C/D)X(7-12)CXXCXXXXCXX(C/H)X(3-4)HX(4-9)H for B-box1 and CXXHX(7-9)CXX(C/D/E/H)XXXXCXXCX(3-6)H(X-4)(H/C) for B-box2. A consensus sequence found within the B-box domains of many STO proteins of plant origin is represented by SEQ ID NO: 221 (CX2CX16CX2C). A signature characteristic of all B-box domains is the presence of at least 4 amino acid residues, typically Cysteine and/or Histidine residues with the putative capacity to bind zinc ions.

Preferred STO polypeptides useful in the methods of the invention comprise at least one, more preferably two B B-box domain having the consensus represented by CX2CX16CX2C (SEQ ID NO: 221).

The B-box domains found in SEQ ID NO: 169 are represented by SEQ ID NO: 219 and SEQ ID NO: 220. Further preferred STO polypeptides useful in the methods of the invention comprise at least one, more preferably two B-box domains having at least 50%, 55%, 60%, 65%, 70%, 75%, 80%, 85%, 90%, 95% or more sequence identity to SEQ ID NO: 219 or SEQ ID NO: 220.

Alternatively, a B-box domain in an STO polypeptide may be identified by performing a sequence comparison with known polypeptides comprising a B-box domain and establishing the similarity in the region of the B-box domain. The sequences may be aligned using any of the methods well known in the art such as Blast algorithms. The probability for the alignment to occur with a given sequence is taken as basis for identifying similar polypeptides. A parameter that is typically used to represent such probability is called e-value. The E-value is a measure of the reliability of the S score. The S score is a measure of the similarity of the query to the sequence shown. The e-value describes how often a given S score is expected to occur at random. The e-value cut-off may be as high as 1.0. The typical threshold for a trusted e-value from a BLAST search output using an STO polypeptide as query sequence can is lower than 1.e⁻⁵, 1.e⁻¹⁰, 1.e⁻¹⁵, 1.e⁻²⁰, 1.e⁻²⁵, 1.e⁻⁵⁰, 1.e⁻⁷⁵, 1.e⁻¹⁰⁰, 1.e⁻²⁰⁰, 1.e⁻³⁰⁰, 1.e⁻⁴⁰⁰, 1.e⁻⁵⁰⁰, 1.e⁻⁶⁰⁰, 1.e⁻⁷⁰⁰ and 1.e⁻⁸⁰⁰. Preferred STO polypeptides useful in the methods of the invention comprise at least one, more preferably two B-box domains having a sequence having in increasing order of preference an e-value lower than e⁻⁵(=10⁻⁵), 1.e⁻¹⁰, 1.e⁻¹⁵, 1.e⁻²⁰, 1.e⁻²⁵, 1.e⁻⁵⁰, 1.e⁻⁷⁵, 1.e⁻¹⁰⁰, 1.e⁻²⁰⁰, 1.e⁻³⁰⁰, 1.e⁻⁴⁰⁰, 1.e⁻⁵⁰⁰, 1.e⁻⁶⁰⁰, 1.e⁻⁷⁰⁰ and 1.e⁻⁸⁰⁰ in an alignment with SEQ ID NO: 219 or SEQ ID NO: 220 or any B-box domain as found in a known STO polypeptide, such as those listed in Table A.

Preferably, the STO polypeptide sequence useful in the methods of the invention when used in the construction of a phylogenetic tree, such as the one depicted in FIG. 16, clusters with the group of STO polypeptides comprising the amino acid sequence represented by SEQ ID NO: 169 (OS04g0540200 in the Figure) rather than with any other group.

Alternatively or additionally, a “STO polypeptide” as defined herein refers to any polypeptide having in increasing order of preference at least 50%, 55%, 60%, 65%, 70%, 75%, 80%, 85%, 90%, 95%, 98%, 99% or more polypeptide sequence identity to the STO polypeptide as represented by SEQ ID NO: 169, or by SEQ ID NO: 171, or to any of the polypeptide sequences given in Table A5 of Example 1 herein.

The term “domain” and “motif” is defined in the “definitions” section herein. Specialist databases exist for the identification of domains, for example, SMART (Schultz et al. (1998) Proc. Natl. Acad. Sci. USA 95, 5857-5864; Letunic et al. (2002) Nucleic Acids Res 30, 242-244), InterPro (Mulder et al., (2003) Nucl. Acids. Res. 31, 315-318), Prosite (Bucher and Bairoch (1994), A generalized profile syntax for biomolecular sequences motifs and its function in automatic sequence interpretation. (In) ISMB-94; Proceedings 2nd International Conference on Intelligent Systems for Molecular Biology. Altman R., Brutlag D., Karp P., Lathrop R., Searls D., Eds., pp 53-61, AAAI Press, Menlo Park; Hulo et al., Nucl. Acids. Res. 32:D134-D137, (2004) ), or Pfam (Bateman et al., Nucleic Acids Research 30(1): 276-280 (2002)). A set of tools for in silico analysis of protein sequences is available on the ExPASy proteomics server (Swiss Institute of Bioinformatics (Gasteiger et al., ExPASy: the proteomics server for in-depth protein knowledge and analysis, Nucleic Acids Res. 31:3784-3788 (2003)). Domains or motifs may also be identified using routine techniques, such as by sequence alignment.

Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the global (i.e. spanning the complete sequences) alignment of two sequences that maximizes the number of matches and minimizes the number of gaps. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information (NCBI). Homologues may readily be identified using, for example, the ClustalW multiple sequence alignment algorithm (version 1.83), with the default pairwise alignment parameters, and a scoring method in percentage. Global percentages of similarity and identity may also be determined using one of the methods available in the MatGAT software package (Campanella et al., BMC Bioinformatics. 2003 Jul. 10; 4:29. MatGAT: an application that generates similarity/identity matrices using protein or DNA sequences.). Minor manual editing may be performed to optimise alignment between conserved motifs, as would be apparent to a person skilled in the art. Furthermore, instead of using full-length sequences for the identification of homologues, specific domains may also be used. The sequence identity values may be determined over the entire nucleic acid or amino acid sequence or over selected domains or conserved motif(s), using the programs mentioned above using the default parameters.

Furthermore, STO polypeptides (at least in their native form) typically when expressed in a transgenic plant, preferably rice, under the control of a constitutive promoter result in modulation of flowering time, typically in shortening flowering time. Tools and techniques for producing transgenic plants and measuring flowering time are well known in the art. Further details are provided in the Examples section.

The present invention is illustrated by transforming plants with the nucleic acid sequence represented by SEQ ID NO: 168, encoding the polypeptide sequence of SEQ ID NO: 169. However, performance of the invention is not restricted to these sequences; the methods of the invention may advantageously be performed using any STO-encoding nucleic acid or STO polypeptide as defined herein.

Examples of nucleic acids encoding STO polypeptides are given in Table A5 of Example 1 herein. Such nucleic acids are useful in performing the methods of the invention. The amino acid sequences given in Table A5 of Example 1 are example sequences of orthologues and paralogues of the STO polypeptide represented by SEQ ID NO: 169, the terms “orthologues” and “paralogues” being as defined herein. Further orthologues and paralogues may readily be identified by performing a so-called reciprocal blast search. Typically, this involves a first BLAST involving BLASTing a query sequence (for example using any of the sequences listed in Table A5 of Example 1) against any sequence database, such as the publicly available NCBI database. BLASTN or TBLASTX (using standard default values) are generally used when starting from a nucleotide sequence, and BLASTP or TBLASTN (using standard default values) when starting from a protein sequence. The BLAST results may optionally be filtered. The full-length sequences of either the filtered results or non-filtered results are then BLASTed back (second BLAST) against sequences from the organism from which the query sequence is derived (where the query sequence is SEQ ID NO: 168 or SEQ ID NO: 169, the second BLAST would therefore be against rice sequences). The results of the first and second BLASTs are then compared. A paralogue is identified if a high-ranking hit from the first blast is from the same species as from which the query sequence is derived, a BLAST back then ideally results in the query sequence amongst the highest hits; an orthologue is identified if a high-ranking hit in the first BLAST is not from the same species as from which the query sequence is derived, and preferably results upon BLAST back in the query sequence being among the highest hits.

### Preferred Homologs

High-ranking hits are those having a low E-value. The lower the E-value, the more significant the score (or in other words the lower the chance that the hit was found by chance). Computation of the E-value is well known in the art. In addition to E-values, comparisons are also scored by percentage identity. Percentage identity refers to the number of identical nucleotides (or amino acids) between the two compared nucleic acid (or polypeptide) sequences over a particular length. In the case of large families, ClustalW may be used, followed by a neighbour joining tree, to help visualize clustering of related genes and to identify orthologues and paralogues.

Nucleic acid variants may also be useful in practising the methods of the invention. Examples of such variants include nucleic acids encoding homologues and derivatives of any one of the amino acid sequences given in Table A5 of Example 1, the terms “homologue” and “derivative” being as defined herein. Also useful in the methods of the invention are nucleic acids encoding homologues and derivatives of orthologues or paralogues of any one of the amino acid sequences given in Table A5 of Example 1. Homologues and derivatives useful in the methods of the present invention have substantially the same biological and functional activity as the unmodified protein from which they are derived.

Further nucleic acid variants useful in practising the methods of the invention include portions of nucleic acids encoding STO polypeptides, nucleic acids hybridising to nucleic acids encoding STO polypeptides, splice variants of nucleic acids encoding STO polypeptides, allelic variants of nucleic acids encoding STO polypeptides and variants of nucleic acids encoding STO polypeptides obtained by gene shuffling. The terms hybridising sequence, splice variant, allelic variant and gene shuffling are as described herein.

Nucleic acids encoding STO polypeptides need not be full-length nucleic acids, since performance of the methods of the invention does not rely on the use of full-length nucleic acid sequences. According to the present invention, there is provided a method for enhancing yield-related traits in plants, comprising introducing and expressing in a plant a portion of any one of the nucleic acid sequences given in Table A5 of Example 1, or a portion of a nucleic acid encoding an orthologue, paralogue or homologue of any of the amino acid sequences given in Table A5 of Example 1.

A portion of a nucleic acid may be prepared, for example, by making one or more deletions to the nucleic acid. The portions may be used in isolated form or they may be fused to other coding (or non-coding) sequences in order to, for example, produce a protein that combines several activities. When fused to other coding sequences, the resultant polypeptide produced upon translation may be bigger than that predicted for the protein portion.

Portions useful in the methods of the invention, encode an STO polypeptide as defined herein, and have substantially the same biological activity as the amino acid sequences given in Table A5 of Example 1. Preferably, the portion is a portion of any one of the nucleic acids given in Table A of Example 1, or is a portion of a nucleic acid encoding an orthologue or paralogue of any one of the amino acid sequences given in Table A5 of Example 1. Preferably the portion is at least 100, 200, 300, 400, 500, 550, 600, 650, 700, 750, 800, 850, 900, 950, 1000 consecutive nucleotides in length, the consecutive nucleotides being of any one of the nucleic acid sequences given in Table A5 of Example 1, or of a nucleic acid encoding an orthologue or paralogue of any one of the amino acid sequences given in Table A5 of Example 1. Most preferably the portion is a portion of the nucleic acid of SEQ ID NO: 168. Preferably, the portion encodes an amino acid sequence which, when used in the construction of a phylogenetic tree, such as the one depicted in FIG. 16, Preferably, the STO polypeptide sequence useful in the methods of the invention when used in the construction of a phylogenetic tree, such as the one depicted in FIG. 16, clusters with the group of STO polypeptides comprising the amino acid sequence represented by SEQ ID NO: 169 (OS04g0540200 in the Figure) rather than with any other group.

Another nucleic acid variant useful in the methods of the invention is a nucleic acid capable of hybridising, under reduced stringency conditions, preferably under stringent conditions, with a nucleic acid encoding an STO polypeptide as defined herein, or with a portion as defined herein.

According to the present invention, there is provided a method for enhancing yield-related traits in plants, comprising introducing and expressing in a plant a nucleic acid capable of hybridizing to any one of the nucleic acids given in Table A5 of Example 1, or comprising introducing and expressing in a plant a nucleic acid capable of hybridising to a nucleic acid encoding an orthologue, paralogue or homologue of any of the nucleic acid sequences given in Table A5 of Example 1.

Hybridising sequences useful in the methods of the invention encode an STO polypeptide as defined herein, having substantially the same biological activity as the amino acid sequences given in Table A5 of Example 1. Preferably, the hybridising sequence is capable of hybridising to any one of the nucleic acids given in Table A5 of Example 1, or to a portion of any of these sequences, a portion being as defined above, or the hybridising sequence is capable of hybridising to a nucleic acid encoding an orthologue or paralogue of any one of the amino acid sequences given in Table A5 of Example 1. Most preferably, the hybridising sequence is capable of hybridising to a nucleic acid as represented by SEQ ID NO: 168 or to a portion thereof.

Preferably, the hybridising sequence encodes a polypeptide with an amino acid sequence which, when full-length and used in the construction of a phylogenetic tree, such as the one depicted in FIG. 16, clusters with the group of STO polypeptides comprising the amino acid sequence represented by SEQ ID NO: 169 (OS04g0540200 in the Figure) rather than with any other group.

Another nucleic acid variant useful in the methods of the invention is a splice variant encoding an STO polypeptide as defined hereinabove, a splice variant being as defined herein.

According to the present invention, there is provided a method for enhancing yield-related traits in plants, comprising introducing and expressing in a plant a splice variant of any one of the nucleic acid sequences given in Table A5 of Example 1, or a splice variant of a nucleic acid encoding an orthologue, paralogue or homologue of any of the amino acid sequences given in Table A5 of Example 1.

Preferred splice variants are splice variants of a nucleic acid represented by SEQ ID NO: 168, or a splice variant of a nucleic acid encoding an orthologue or paralogue of SEQ ID NO: 169. Preferably, the amino acid sequence encoded by the splice variant, when used in the construction of a phylogenetic tree, such as the one depicted in FIG. 16, clusters with the group of STO polypeptides comprising the amino acid sequence represented by SEQ ID NO: 169 (OS04g0540200 in the Figure) rather than with any other group.

Another nucleic acid variant useful in performing the methods of the invention is an allelic variant of a nucleic acid encoding an STO polypeptide as defined hereinabove, an allelic variant being as defined herein.

According to the present invention, there is provided a method for enhancing yield-related traits in plants, comprising introducing and expressing in a plant an allelic variant of any one of the nucleic acids given in Table A5 of Example 1, or comprising introducing and expressing in a plant an allelic variant of a nucleic acid encoding an orthologue, paralogue or homologue of any of the amino acid sequences given in Table A5 of Example 1.

The allelic variants useful in the methods of the present invention have substantially the same biological activity as the STO polypeptide of SEQ ID NO: 169 and any of the amino acids depicted in Table A of Example 1. Allelic variants exist in nature, and encompassed within the methods of the present invention is the use of these natural alleles. Preferably, the allelic variant is an allelic variant of SEQ ID NO: 168 or an allelic variant of a nucleic acid encoding an orthologue or paralogue of SEQ ID NO: 169. Preferably, the amino acid sequence encoded by the allelic variant, when used in the construction of a phylogenetic tree, such as the one depicted in FIG. 16, clusters with the group of STO polypeptides comprising the amino acid sequence represented by SEQ ID NO: 169 (OS04g0540200 in the Figure) rather than with any other group.

Gene shuffling or directed evolution may also be used to generate variants of nucleic acids encoding STO polypeptides as defined above; the term “gene shuffling” being as defined herein.

According to the present invention, there is provided a method for enhancing yield-related traits in plants, comprising introducing and expressing in a plant a variant of any one of the nucleic acid sequences given in Table A5 of Example 1, or comprising introducing and expressing in a plant a variant of a nucleic acid encoding an orthologue, paralogue or homologue of any of the amino acid sequences given in Table A5 of Example 1, which variant nucleic acid is obtained by gene shuffling.

Preferably, the amino acid sequence encoded by the variant nucleic acid directly or indirectly obtained by gene shuffling, when used in the construction of a phylogenetic tree such as the one depicted in FIG. 16, clusters with the group of STO polypeptides comprising the amino acid sequence represented by SEQ ID NO: 169 (OS04g0540200 in the Figure) rather than with any other group.

Furthermore, nucleic acid variants may also be obtained by site-directed mutagenesis. Several methods are available to achieve site-directed mutagenesis, the most common being PCR based methods (Current Protocols in Molecular Biology. Wiley Eds.).

Nucleic acids encoding STO polypeptides may be derived from any natural or artificial source. The nucleic acid may be modified from its native form in composition and/or genomic environment through deliberate human manipulation. Preferably the STO polypeptide-encoding nucleic acid is from a plant, further preferably from a monocotyledonous plant, more preferably from the family Poaceae, most preferably the nucleic acid is from Oryza sativa.

Performance of the methods of the invention gives plants having enhanced yield-related traits. In particular performance of the methods of the invention gives plants having increased yield, especially increased seed yield relative to control plants. The terms “yield” and “seed yield” are described in more detail in the “definitions” section herein.

Reference herein to enhanced yield-related traits is taken to mean an increase in early (seedling) vigour and altered flowering time more particular shorter of flowering time relative to control plants. Preferably the shortening of the flowering time is at least of 1, 2, 3, 4, 5, 6, 7, 8, 9, 10, 11, 12, 13, 14, 15, 16, 17, 18, 19, 20, 21, 22, 23, 24 or 25 days or at least by 5%, 10%, 15%, 20%, or 25% of the time compared to relative to control plants

Taking corn as an example, a yield increase may be manifested as one or more of the following: increase in the number of plants established per hectare or acre, an increase in the number of ears per plant, an increase in the number of rows, number of kernels per row, kernel weight, thousand kernel weight, ear length/diameter, increase in the seed filling rate (which is the number of filled seeds divided by the total number of seeds and multiplied by 100), among others. Taking rice as an example, a yield increase may manifest itself as an increase in one or more of the following: number of plants per hectare or acre, number of panicles per plant, number of spikelets per panicle, number of flowers (florets) per panicle (which is expressed as a ratio of the number of filled seeds over the number of primary panicles), increase in the seed filling rate (which is the number of filled seeds divided by the total number of seeds and multiplied by 100), increase in thousand kernel weight, among others.

The present invention provides a method for increasing early (seedling) vigour and altering flowering time, particularly shortening of flowering time of plants, relative to control plants which method comprises modulating expression, preferably increasing expression, in a plant of a nucleic acid encoding an STO polypeptide as defined herein.

Since the transgenic plants according to the present invention have increased yield, it is likely that these plants exhibit an increased growth rate (during at least part of their life cycle), relative to the growth rate of control plants at a corresponding stage in their life cycle.

The increased growth rate may be specific to one or more parts of a plant (including seeds), or may be throughout substantially the whole plant. Plants having an increased growth rate may have a shorter life cycle. The life cycle of a plant may be taken to mean the time needed to grow from a dry mature seed up to the stage where the plant has produced dry mature seeds, similar to the starting material. This life cycle may be influenced by factors such as early vigour, growth rate, greenness index, flowering time and speed of seed maturation. The increase in growth rate may take place at one or more stages in the life cycle of a plant or during substantially the whole plant life cycle. Increased growth rate during the early stages in the life cycle of a plant may reflect enhanced or increased vigour. The increase in growth rate may alter the harvest cycle of a plant allowing plants to be sown later and/or harvested sooner than would otherwise be possible (a similar effect may be obtained with earlier flowering time). If the growth rate is sufficiently increased, it may allow for the further sowing of seeds of the same plant species (for example sowing and harvesting of rice plants followed by sowing and harvesting of further rice plants all within one conventional growing period). Similarly, if the growth rate is sufficiently increased, it may allow for the further sowing of seeds of different plants species (for example the sowing and harvesting of corn plants followed by, for example, the sowing and optional harvesting of soybean, potato or any other suitable plant). Harvesting additional times from the same rootstock in the case of some crop plants may also be possible. Altering the harvest cycle of a plant may lead to an increase in annual biomass production per acre (due to an increase in the number of times (say in a year) that any particular plant may be grown and harvested). An increase in growth rate may also allow for the cultivation of transgenic plants in a wider geographical area than their wild-type counterparts, since the territorial limitations for growing a crop are often determined by adverse environmental conditions either at the time of planting (early season) or at the time of harvesting (late season). Such adverse conditions may be avoided if the harvest cycle is shortened. The growth rate may be determined by deriving various parameters from growth curves, such parameters may be: T-Mid (the time taken for plants to reach 50% of their maximal size) and T-90 (time taken for plants to reach 90% of their maximal size), amongst others.

According to a preferred feature of the present invention, performance of the methods of the invention gives plants having an increased growth rate relative to control plants. Therefore, according to the present invention, there is provided a method for increasing the growth rate of plants, which method comprises modulating expression, preferably increasing expression, in a plant of a nucleic acid encoding an STO polypeptide as defined herein.

An increase in yield and/or growth rate occurs whether the plant is under non-stress conditions or whether the plant is exposed to various stresses compared to control plants. Plants typically respond to exposure to stress by growing more slowly. In conditions of severe stress, the plant may even stop growing altogether. Mild stress on the other hand is defined herein as being any stress to which a plant is exposed which does not result in the plant ceasing to grow altogether without the capacity to resume growth. Mild stress in the sense of the invention leads to a reduction in the growth of the stressed plants of less than 40%, 35% or 30%, preferably less than 25%, 20% or 15%, more preferably less than 14%, 13%, 12%, 11% or 10% or less in comparison to the control plant under non-stress conditions. Due to advances in agricultural practices (irrigation, fertilization, pesticide treatments) severe stresses are not often encountered in cultivated crop plants. As a consequence, the compromised growth induced by mild stress is often an undesirable feature for agriculture. Mild stresses are the everyday biotic and/or abiotic (environmental) stresses to which a plant is exposed. Abiotic stresses may be due to drought or excess water, anaerobic stress, salt stress, chemical toxicity, oxidative stress and hot, cold or freezing temperatures. The abiotic stress may be an osmotic stress caused by a water stress (particularly due to drought), salt stress, oxidative stress or an ionic stress. Biotic stresses are typically those stresses caused by pathogens, such as bacteria, viruses, fungi and insects.

In particular, the methods of the present invention may be performed under non-stress conditions or under conditions of mild drought to give plants having increased yield relative to control plants. As reported in Wang et al. (Planta (2003) 218: 1-14), abiotic stress leads to a series of morphological, physiological, biochemical and molecular changes that adversely affect plant growth and productivity. Drought, salinity, extreme temperatures and oxidative stress are known to be interconnected and may induce growth and cellular damage through similar mechanisms. Rabbani et al. (Plant Physiol (2003) 133: 1755-1767) describes a particularly high degree of “cross talk” between drought stress and high-salinity stress. For example, drought and/or salinisation are manifested primarily as osmotic stress, resulting in the disruption of homeostasis and ion distribution in the cell. Oxidative stress, which frequently accompanies high or low temperature, salinity or drought stress, may cause denaturing of functional and structural proteins. As a consequence, these diverse environmental stresses often activate similar cell signalling pathways and cellular responses, such as the production of stress proteins, up-regulation of anti-oxidants, accumulation of compatible solutes and growth arrest. The term “non-stress” conditions as used herein are those environmental conditions that allow optimal growth of plants. Persons skilled in the art are aware of normal soil conditions and climatic conditions for a given location.

Performance of the methods of the invention gives plants grown under non-stress conditions or under mild drought conditions increased yield relative to control plants grown under comparable conditions. Therefore, according to the present invention, there is provided a method for increasing yield in plants grown under non-stress conditions or under mild drought conditions, which method comprises increasing expression in a plant of a nucleic acid encoding an STO polypeptide.

Performance of the methods of the invention gives plants grown under conditions of nutrient deficiency, particularly under conditions of nitrogen deficiency, increased yield relative to control plants grown under comparable conditions. Therefore, according to the present invention, there is provided a method for increasing yield in plants grown under conditions of nutrient deficiency, which method comprises increasing expression in a plant of a nucleic acid encoding an STO polypeptide. Nutrient deficiency may result from a lack of nutrients such as nitrogen, phosphates and other phosphorous-containing compounds, potassium, calcium, cadmium, magnesium, manganese, iron and boron, amongst others.

The present invention encompasses plants or parts thereof (including seeds) obtainable by the methods according to the present invention. The plants or parts thereof comprise a nucleic acid transgene encoding an STO polypeptide as defined above.

The invention also provides genetic constructs and vectors to facilitate introduction and/or expression in plants of nucleic acids encoding STO polypeptides. The gene constructs may be inserted into vectors, which may be commercially available, suitable for transforming into plants and suitable for expression of the gene of interest in the transformed cells. The invention also provides use of a gene construct as defined herein in the methods of the invention.

More specifically, the present invention provides a construct comprising:
(a) a nucleic acid encoding an STO polypeptide as defined above;
(b) one or more control sequences capable of driving expression of the nucleic acid sequence of (a); and optionally
(c) a transcription termination sequence.

Preferably, the nucleic acid encoding an STO polypeptide is as defined above. The term “control sequence” and “termination sequence” are as defined herein.

Plants are transformed with a vector comprising any of the nucleic acids described above. The skilled artisan is well aware of the genetic elements that must be present on the vector in order to successfully transform, select and propagate host cells containing the sequence of interest. The sequence of interest is operably linked to one or more control sequences (at least to a promoter).

Advantageously, any type of promoter, whether natural or synthetic, may be used to drive expression of the nucleic acid sequence. A constitutive promoter is particularly useful in the methods. See the “Definitions” section herein for definitions of the various promoter types.

It should be clear that the applicability of the present invention is not restricted to the STO polypeptide-encoding nucleic acid represented by SEQ ID NO: 168, nor is the applicability of the invention restricted to expression of an STO polypeptide-encoding nucleic acid when driven by a constitutive promoter.

The constitutive promoter is preferably a GOS2 promoter, preferably a GOS2 promoter from rice. Further preferably the constitutive promoter is represented by a nucleic acid sequence substantially similar to SEQ ID NO: 218, most preferably the constitutive promoter is as represented by SEQ ID NO: 218. See Table 2 in the “Definitions” section herein for further examples of constitutive promoters.

Optionally, one or more terminator sequences may be used in the construct introduced into a plant. Additional regulatory elements may include transcriptional as well as translational enhancers. Those skilled in the art will be aware of terminator and enhancer sequences that may be suitable for use in performing the invention. An intron sequence may also be added to the 5′ untranslated region (UTR) or in the coding sequence to increase the amount of the mature message that accumulates in the cytosol, as described in the definitions section. Other control sequences (besides promoter, enhancer, silencer, intron sequences, 3′UTR and/or 5′UTR regions) may be protein and/or RNA stabilizing elements. Such sequences would be known or may readily be obtained by a person skilled in the art.

The genetic constructs of the invention may further include an origin of replication sequence that is required for maintenance and/or replication in a specific cell type. One example is when a genetic construct is required to be maintained in a bacterial cell as an episomal genetic element (e.g. plasmid or cosmid molecule). Preferred origins of replication include, but are not limited to, the f1-ori and colE1.

For the detection of the successful transfer of the nucleic acid sequences as used in the methods of the invention and/or selection of transgenic plants comprising these nucleic acids, it is advantageous to use marker genes (or reporter genes). Therefore, the genetic construct may optionally comprise a selectable marker gene. Selectable markers are described in more detail in the “definitions” section herein. The marker genes may be removed or excised from the transgenic cell once they are no longer needed. Techniques for marker removal are known in the art, useful techniques are described above in the definitions section.

The invention also provides a method for the production of transgenic plants having enhanced yield-related traits relative to control plants, comprising introduction and expression in a plant of any nucleic acid encoding an STO polypeptide as defined hereinabove.

More specifically, the present invention provides a method for the production of transgenic plants having increased enhanced yield-related traits, particularly increased (early) seedling vigour and/or altered flowering time more particular shorter flowering time of plants, which method comprises:

(i) introducing and expressing in a plant or plant cell an STO polypeptide-encoding nucleic acid; and
(ii) cultivating the plant cell under conditions promoting plant growth and development.

The nucleic acid of (i) may be any of the nucleic acids capable of encoding an STO polypeptide as defined herein.

The nucleic acid may be introduced directly into a plant cell or into the plant itself (including introduction into a tissue, organ or any other part of a plant). According to a preferred feature of the present invention, the nucleic acid is preferably introduced into a plant by transformation. The term “transformation” is described in more detail in the “definitions” section herein.

The genetically modified plant cells can be regenerated via all methods with which the skilled worker is familiar. Suitable methods can be found in the abovementioned publications by S. D. Kung and R. Wu, Potrykus or Hofgen and Willmitzer.

Generally after transformation, plant cells or cell groupings are selected for the presence of one or more markers which are encoded by plant-expressible genes co-transferred with the gene of interest, following which the transformed material is regenerated into a whole plant. To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as the ones described above.

Following DNA transfer and regeneration, putatively transformed plants may also be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

The present invention clearly extends to any plant cell or plant produced by any of the methods described herein, and to all plant parts and propagules thereof. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced by the parent in the methods according to the invention.

The invention also includes host cells containing an isolated nucleic acid encoding an STO polypeptide as defined hereinabove. Preferred host cells according to the invention are plant cells. Host plants for the nucleic acids or the vector used in the method according to the invention, the expression cassette or construct or vector are, in principle, advantageously all plants, which are capable of synthesizing the polypeptides used in the inventive method.

The methods of the invention are advantageously applicable to any plant. Plants that are particularly useful in the methods of the invention include all plants which belong to the superfamily Viridiplantae, in particular monocotyledonous and dicotyledonous plants including fodder or forage legumes, ornamental plants, food crops, trees or shrubs. According to a preferred embodiment of the present invention, the plant is a crop plant. Examples of crop plants include soybean, sunflower, canola, alfalfa, rapeseed, cotton, tomato, potato and tobacco. Further preferably, the plant is a monocotyledonous plant. Examples of monocotyledonous plants include sugarcane. More preferably the plant is a cereal. Examples of cereals include rice, maize, wheat, barley, millet, rye, triticale, sorghum and oats.

The invention also extends to harvestable parts of a plant such as, but not limited to seeds, leaves, fruits, flowers, stems, roots, rhizomes, tubers and bulbs. The invention furthermore relates to products derived, preferably directly derived, from a harvestable part of such a plant, such as dry pellets or powders, oil, fat and fatty acids, starch or proteins.

According to a preferred feature of the invention, the modulated expression is increased expression. Methods for increasing expression of nucleic acids or genes, or gene products, are well documented in the art and examples are provided in the definitions section.

As mentioned above, a preferred method for modulating (preferably, increasing) expression of a nucleic acid encoding an STO polypeptide is by introducing and expressing in a plant a nucleic acid encoding an STO polypeptide; however the effects of performing the method, i.e. enhancing yield-related traits may also be achieved using other well known techniques, including, but not limited to, T-DNA activation tagging, TILLING, homologous recombination. A description of these techniques is provided in the definitions section.

The present invention also encompasses use of nucleic acids encoding STO polypeptides as described herein and use of these STO polypeptides in enhancing any of the aforementioned yield-related traits in plants.

Nucleic acids encoding STO polypeptide described herein, or the STO polypeptides themselves, may find use in breeding programmes in which a DNA marker is identified, which may be genetically linked to an STO polypeptide-encoding gene. The nucleic acids/genes, or the STO polypeptides themselves may be used to define a molecular marker. This DNA or protein marker may then be used in breeding programmes to select plants having enhanced yield-related traits as defined hereinabove in the methods of the invention.

Allelic variants of an STO polypeptide-encoding nucleic acid/gene may also find use in marker-assisted breeding programmes. Such breeding programmes sometimes require introduction of allelic variation by mutagenic treatment of the plants, using for example EMS mutagenesis; alternatively, the programme may start with a collection of allelic variants of so called “natural” origin caused unintentionally. Identification of allelic variants then takes place, for example, by PCR. This is followed by a step for selection of superior allelic variants of the sequence in question and which give increased yield. Selection is typically carried out by monitoring growth performance of plants containing different allelic variants of the sequence in question. Growth performance may be monitored in a greenhouse or in the field. Further optional steps include crossing plants in which the superior allelic variant was identified with another plant. This could be used, for example, to make a combination of interesting phenotypic features.

Nucleic acids encoding STO polypeptides may also be used as probes for genetically and physically mapping the genes that they are a part of, and as markers for traits linked to those genes. Such information may be useful in plant breeding in order to develop lines with desired phenotypes. Such use of STO polypeptide-encoding nucleic acids requires only a nucleic acid sequence of at least 15 nucleotides in length. The STO polypeptide-encoding nucleic acids may be used as restriction fragment length polymorphism (RFLP) markers. Southern blots (Sambrook J, Fritsch E F and Maniatis T (1989) Molecular Cloning, A Laboratory Manual) of restriction-digested plant genomic DNA may be probed with the STO-encoding nucleic acids. The resulting banding patterns may then be subjected to genetic analyses using computer programs such as MapMaker (Lander et al. (1987) Genomics 1: 174-181) in order to construct a genetic map. In addition, the nucleic acids may be used to probe Southern blots containing restriction endonuclease-treated genomic DNAs of a set of individuals representing parent and progeny of a defined genetic cross. Segregation of the DNA polymorphisms is noted and used to calculate the position of the STO polypeptide-encoding nucleic acid in the genetic map previously obtained using this population (Botstein et al. (1980) Am. J. Hum. Genet. 32:314-331).

The production and use of plant gene-derived probes for use in genetic mapping is described in Bernatzky and Tanksley (1986) Plant Mol. Biol. Reporter 4: 37-41. Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

The nucleic acid probes may also be used for physical mapping (i.e., placement of sequences on physical maps; see Hoheisel et al. In: Non-mammalian Genomic Analysis: A Practical Guide, Academic press 1996, pp. 319-346, and references cited therein).

In another embodiment, the nucleic acid probes may be used in direct fluorescence in situ hybridisation (FISH) mapping (Trask (1991) Trends Genet. 7:149-154). Although current methods of FISH mapping favour use of large clones (several kb to several hundred kb; see Laan et al. (1995) Genome Res. 5:13-20), improvements in sensitivity may allow performance of FISH mapping using shorter probes.

A variety of nucleic acid amplification-based methods for genetic and physical mapping may be carried out using the nucleic acids. Examples include allele-specific amplification (Kazazian (1989) J. Lab. Clin. Med 11:95-96), polymorphism of PCR-amplified fragments (CAPS; Sheffield et al. (1993) Genomics 16:325-332), allele-specific ligation (Landegren et al. (1988) Science 241:1077-1080), nucleotide extension reactions (Sokolov (1990) Nucleic Acid Res. 18:3671), Radiation Hybrid Mapping (Walter et al. (1997) Nat. Genet. 7:22-28) and Happy Mapping (Dear and Cook (1989) Nucleic Acid Res. 17:6795-6807). For these methods, the sequence of a nucleic acid is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for mapping methods.

The methods according to the present invention result in plants having enhanced yield-related traits, as described hereinbefore. These traits may also be combined with other economically advantageous traits, such as further yield-enhancing traits, tolerance to other abiotic and biotic stresses, traits modifying various architectural features and/or biochemical and/or physiological features.

The term “table A5” used in this specification is to be taken to specify the content of table A5a and table A5b. The term “table A5a” used in this specification is to be taken to specify the content of table A5a. The term “table A5b” used in this specification is to be taken to specify the content of table A5b. In one preferred embodiment, the term “table A5” means table A5b.

In one embodiment the invention relates to subject mater summarized as follows:

Item 82: A method for enhancing yield-related traits in plants relative to control plants, comprising modulating expression in a plant of a nucleic acid encoding an STO polypeptide, wherein said STO polypeptide comprises a B-box domain.
Item 83: Method according to item 82, wherein said B-box domain comprises a consensus sequences as represented by SEQ ID NO: 221.
Item 84: Method according to items 82 or 83, wherein said STO polypeptides comprises a B-box domain having at least 50%, 55%, 60%, 65%, 70%, 75%, 80%, 85%, 90%, 95% or more sequence identity to SEQ ID NO: 219 or SEQ ID NO: 220.
Item 85: Method according to any of items 82 to 84, wherein said modulated expression is effected by introducing and expressing in a plant a nucleic acid encoding an STO polypeptide.
Item 86: Method according to any of items 82 to 85, wherein said nucleic acid encoding an STO polypeptide encodes any one of the proteins listed in Table A5 or is a portion of such a nucleic acid, or a nucleic acid capable of hybridising with such a nucleic acid.
Item 87: Method according to any of items 82 to 86, wherein said nucleic acid sequence encodes an orthologue or paralogue of any of the proteins given in Table A5.
Item 88: Method according to any of items 82 to 87, wherein said enhanced yield-related traits comprise increased seedling vigour and/or altered flowering time preferably shorter flowering time relative to control plants.
Item 89: Method according to any of items 82 to 88, wherein said enhanced yield-related traits are obtained under non-stress conditions.
Item 90: Method according to any of items 85 to 89, wherein said nucleic acid is operably linked to a constitutive promoter, preferably to a GOS2 promoter, most preferably to a GOS2 promoter from rice.
Item 91: Method according to any of items 82 to 90, wherein said nucleic acid encoding an STO polypeptide is of plant origin, preferably from a monocotyledonous plant, further preferably from the family Poaceae, more preferably from the genus Oryza, most preferably from Oryza sativa.
Item 92: Plant or part thereof, including seeds, obtainable by a method according to any preceding claim, wherein said plant or part thereof comprises a recombinant nucleic acid encoding an STO polypeptide.

Item 93: An isolated nucleic acid sequence comprising a nucleic acid sequence selected from the group consisting of:
(a) an isolated nucleic acid molecule encoding the polypeptide as depicted in SEQ ID NO: 223 and/or table A5b;
(b) an isolated nucleic acid molecule as depicted in SEQ ID NO: 222 and/or table A5b;
(c) an isolated nucleic acid molecule, which, as a result of the degeneracy of the genetic code, can be derived from a polypeptide sequence depicted in table A5 and confers enhanced yield-related traits relative to control plants;
(d) an isolated nucleic acid molecule having at least 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with the nucleic acid molecule sequence of a polynucleotide comprising the nucleic acid molecule depicted in table A5 and confers enhanced yield-related traits relative to control plants;
(e) an isolated nucleic acid molecule encoding a polypeptide having at least 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of the polypeptide encoded by the nucleic acid molecule of (a) to (c) and confers enhanced yield-related traits relative to control plants;
(f) an isolated nucleic acid molecule which hybridizes with a nucleic acid molecule of (a) to (c) under stringent hybridization conditions and confers enhanced yield-related traits relative to control plants;
(g) an isolated nucleic acid molecule encoding a polypeptide comprising the consensus sequence or one or more polypeptide motifs as shown FIG. 15 and preferably having the activity represented by a nucleic acid molecule comprising a polynucleotide as depicted in table A5;
(h) an isolated nucleic acid molecule encoding a polypeptide having the activity represented by a protein as depicted in table A5 and confering enhanced yield-related traits relative to control plants; and
(i) a nucleic acid molecule which is obtainable by screening a suitable nucleic acid library under stringent hybridization conditions with a probe comprising a complementary sequence of a nucleic acid molecule of (a) or (b) or with a fragment thereof, having at least 15 nt, preferably 20 nt, 30 nt, 50 nt, 100 nt, 200 nt or 500 nt of a nucleic acid molecule complementary to a nucleic acid molecule sequence characterized in (a) to (e) and encoding a polypeptide having the activity represented by a protein comprising a polypeptide as depicted in table A5;
whereby the nucleic acid molecule according to (a) to (i) is at least in one or more nucleotides different from the sequence depicted in table A5a and preferably which encodes a protein which differs at least in one or more amino acids from the protein sequences depicted in table A5a.
   Item 94: Construct comprising:
   (a) nucleic acid encoding an STO polypeptide as defined in any of items 82 to 84 and 93;
   (b) one or more control sequences capable of driving expression of the nucleic acid sequence of (a); and optionally
   (c) a transcription termination sequence.
   Item 95: Construct according to item 94, wherein one of said control sequences is a constitutive promoter, preferably a GOS2 promoter, most preferably a GOS2 promoter from rice.
   Item 96:Use of a construct according to any of items 94 to 95 in a method for making plants having enhanced yield-related traits, preferably increased seedling vigour and/or altered flowering time, particularly shorter flowering time relative to control plants.
   Item 97: Plant, plant part or plant cell transformed with a construct according to any of items 94 to 95.
   Item 98: Method for the production of a transgenic plant having enhanced yield-related traits, preferably increased seedling vigour and/or altered flowering time, particularly shorter flowering time relative to control plants, comprising:
   (i) introducing and expressing in a plant a nucleic acid encoding an STO polypeptide as defined in any of items 82 to 84 and 93; and
   (ii) cultivating the plant cell under conditions promoting plant growth and development.
   Item 99: Transgenic plant having enhanced yield-related traits, preferably increased seedling vigour and/or altered flowering time, particularly shorter flowering time relative to control plants, resulting from increased expression of a nucleic acid encoding an STO polypeptide as defined in any of items 82 to 84 and 93, or a transgenic plant cell derived from said transgenic plant.
   Item 100: Transgenic plant according to item 92, 97 or 99, or a transgenic plant cell derived thereof, wherein said plant is a crop plant or a monocot or a cereal, such as rice, maize, wheat, barley, millet, rye, triticale, sorghum and oats.
   Item 101: Harvestable parts of a plant according to item 100, wherein said harvestable parts are preferably shoot biomass and/or seeds.
   Item 102: Products derived from a plant according to item 100 and/or from harvestable parts of a plant according to item 101.
   Item 103: Use of a nucleic acid encoding an STO polypeptide in increasing seedling vigour and/or altering flowering time, particularly shortening flowering time relative to control plants.

According to a further embodiment of the present invention, there is also provided an isolated polypeptide comprising a polypeptide sequence selected from the group consisting of:

(i) an amino acid sequence encoded by any one of the nucleic acids represented by Item 93;
(ii) an amino acid sequence having, in increasing order of preference, at least 50%, 55%, 60%, 65%, 70%, 75%, 80%, 85%, 90%, 95%, 96%, 97%, 98%, 99% or more sequence identity to the amino acid sequence of (i);
(iii) derivatives of any of the amino acid sequences given in (i) or (ii) above.

In a further embodiment of the invention it has now been found that modulating expression in a plant of a nucleic acid encoding a UGE polypeptide gives plants having enhanced yield-related traits relative to control plants. According to a first embodiment, the present invention provides a method for enhancing yield-related traits in plants relative to control plants, comprising modulating expression in a plant of a nucleic acid encoding a UGE polypeptide.

A preferred method for modulating (preferably, increasing) expression of a nucleic acid encoding a UGE polypeptide is by introducing and expressing in a plant a nucleic acid encoding a UGE polypeptide.

Any reference hereinafter to a “protein useful in the methods of the invention” is taken to mean a UGE polypeptide. Any reference hereinafter to a “nucleic acid useful in the methods of the invention” is taken to mean a nucleic acid capable of encoding such a UGE polypeptide. The nucleic acid to be introduced into a plant (and therefore useful in performing the methods of the invention) is any nucleic acid encoding the type of protein which will now be described, hereinafter also named “UGE nucleic acid” or “UGE gene”. UGE polypeptides and UGE nucleic acids have been described before (Maxwell and Robichon-Szulmajster (1960); J. Biol. Chem. 235 (1960) 308-312; Wilson, D. B. and Hogness, D. S. (1964). J. Biol. Chem. 239 2469-2481; reviewed by Frey FASEB J. 1996 March; 10(4):461-70; Thoden et al. (1997). Biochemistry 36:6294-6304; Dormann and Benning 1998; Shaw et al. 2003. Mol. Biochem. Parasitol. 126, 173-180; Barber et al. 2006).

A “UGE polypeptide” comprises an epimerase domain and typically has UDP-Glucose-4-epimerase activity. Epimerase domain refers to a sequence of varying length, in average around 200 amino acids long, conserved amongst epimerases (InterPro reference IPR005886; Pfam reference: PF01370).

UGE polypeptides can be found by searching in specialized databases such as Pfam, (Finn et al. Nucleic Acids Research (2006) Database Issue 34:D247-D251). Pfam compiles a large collection of multiple sequence alignments and hidden Markov models (HMM) covering many common protein domains and families and is available through the Sanger Institute in the United Kingdom. Trusted matches as considered in the Pfam database are those sequences scoring higher than the gathering cut-off threshold. The gathering cutoff threshold of the epimerase domain in the Pfam HMM_fs method is −46.3 and in the Pfam HMM_fs method is 16.7. However potential matches, comprising true epimerase domains, may still fall under the gathering cut-off. Preferably a UGE polypeptide useful in the methods of the invention is a protein having one or more domains in their sequence that exceed the gathering cutoff of the Pfam protein domain family PF01370, known as epimerase or NAD dependent epimerase/dehydratase family domain.

Alternatively, an epimerase domain in a polypeptide may be identified by performing a sequence comparison with known polypeptides comprising a epimerase domain and establishing the similarity in the region of the epimerase domain. The sequences may be aligned using any of the methods well known in the art such as Blast algorithms. The probability for the alignment to occur with a given sequence is taken as basis for identifying similar polypeptides. A parameter that is typically used to represent such probability is called e-value. The E-value is a measure of the reliability of the S score. The S score is a measure of the similarity of the query to the sequence shown. The e-value describes how often a given S score is expected to occur at random. The e-value cut-off may be as high as 1.0. The typical threshold for a trusted e-value from a BLAST search output using an epimerase polypeptide as query sequence is lower than e5(=10-5), 1.e-10, 1.e-15, 1.e-20, 1.e-25, 1.e-50, 1.e-75, 1.e-100, 1.e-200, 1.e-300, 1.e-400, 1.e-500, 1.e-600, 1.e-700 and 1.e-800. Preferably UGE polypeptides useful in the methods of the invention comprise a sequence having in increasing order of preference an e-value lower than e-5(=10-5), 1.e-10, 1.e-15, 1.e-20, 1.e-25, 1.e-50, 1.e-75, 1.e-100, 1.e-200, 1.e-300, 1.e-400, 1.e-500, 1.e-600, 1.e-700 and 1.e-800 in an alignment with an epimerase domain found in a known UGE polypeptide, such as for example SEQ ID NO: 225.

A preferred UGE polypeptide useful in the methods of the invention refers to a polypeptide comprising an epimerase domain, such domain having in increasing order of preference at least 70%, 75%, 80%, 85%, 90%, 95%, 97% or more sequence identity to SEQ ID NO: 275 (sequence representing the epimerase domain comprised in SEQ ID NO: 225).

Examples of UGE polypeptides useful in the methods of the invention are given in Table A6. The amino acid coordinates of the epimerase domain in the representative UGE protein as represented by SEQ ID NO: 225 are given in Table C. The sequence of the epimerase domain comprised in SEQ ID NO: 225 is given in SEQ ID NO: 275).

Preferred UGE polypeptides useful in the methods of the invention are those having in increasing order of preference at least 70%, 75%, 80%, 85%, 90%, 95%, 96%; 98% or more sequence identity to any of the polypeptides given in Table A6.

UGE polypeptides typically comprise conserved motifs relevant to their enzymatic activity such as i) Motif 8 involved in binding of the UGE protein to the cofactor NAD+ and represented by GXXGXXG (SEQ ID NO: 276) and ii) Motif 9 located in the active site as represented by YGRT/S (SEQ ID NO: 277), wherein the G can be substituted by any other non polar amino acid and the R by any other polar amino acid.

UGE polypeptides may comprise in addition to the epimerase domain one or more of the following conserved motifs: (i) Motif 10 as represented by SEQ ID NO: 278, which comprises the NAD+ binding motif, (ii) Motif 11 as represented by SEQ ID NO: 279 and iii) Motif 12 as represented by SEQ ID: 280. FIG. 19 shows the conserved motifs Motif 8 to Motif 12 and their relative position in the UGE polypeptide sequence represented by SEQ ID NO: 225.

Therefore, preferred UGE polypeptides useful in the method of the invention, comprise in addition to the epimerase domain any one or more of the following conserved motifs:
(i) Motif 8 as represented by SEQ ID NO: 276;
(ii) Motif 9 as represented by SEQ ID NO: 277 wherein the G can be substituted by any other non polar amino acid and the R by any other polar amino acid;
(iii) Motif 10 as represented by SEQ ID NO: 278, wherein 1, 2, 3 or 4 mismatches are allowed;
(iv) Motif 11 as represented by SEQ ID NO: 279,
(v) Motif 12 as represented by SEQ ID NO: 280, wherein 1, 2, 3 or 4 mismatches are allowed.

The polarity of the 20 essential amino acids in given in Table 3. Most frequently the amino acids comprised in polypeptides are alpha amino acids having an amine and a carboxyl group attached to the same carbon, the alpha carbon, which amino acid molecules often comprise a side chain attached to the alpha carbon. Table 3 shows the classification of amino acids based on the physical and biochemical properties of the side chain.

TABLE 3
Classification of amino acids according to the side chain properties.

| | | | | Side chain | |
|---|---|---|---|---|---|
| | | | Side chain | acidity | Hydropathy |
| Amino Acid | 3-Letter | 1-Letter | polarity | or basicity | index |
| Arginine | Arg | R | polar | basic | −4.5 |
| Asparagine | Asn | N | polar | neutral | −3.5 |
| Aspartic acid | Asp | D | polar | acidic | −3.5 |
| Cysteine | Cys | C | polar | neutral | 2.5 |
| Glutamic acid | Glu | E | polar | acidic | −3.5 |
| Glutamine | Gln | Q | polar | neutral | −3.5 |
| Histidine | His | H | polar | basic | −3.2 |
| Lysine | Lys | K | polar | basic | −3.9 |
| Serine | Ser | S | polar | neutral | −0.8 |
| Threonine | Thr | T | polar | neutral | −0.7 |
| Tyrosine | Tyr | Y | polar | neutral | −1.3 |
| Alanine | Ala | A | nonpolar | neutral | 1.8 |
| Glycine | Gly | G | nonpolar | neutral | −0.4 |
| Isoleucine | Ile | I | nonpolar | neutral | 4.5 |
| Leucine | Leu | L | nonpolar | neutral | 3.8 |
| Methionine | Met | M | nonpolar | neutral | 1.9 |
| Phenylalanine | Phe | F | nonpolar | neutral | 2.8 |
| Proline | Pro | P | nonpolar | neutral | −1.6 |
| Tryptophan | Trp | W | nonpolar | neutral | −0.9 |
| Valine | Val | V | nonpolar | neutral | 4.2 |

Examples of UGE polypeptides comprising one or more of the conserved motifs Motif 8 to Motif 12 are given in Example 1. FIG. 20 shows the position of the conserved motifs in those UGE polypeptides.

Additionally UGE polypeptides may comprise a signal polypeptide functional in subcellular localisation of the protein in a cell. For example SEQ ID NO: 225 comprises a putative secretory pathway signal peptide located between amino acid 13 and 37, with a putative cleavage site between amino acid 36 and 37.

UGE polypeptides may be modified further in the cell, for example, by cleavage of signal peptides to produce a so-called, mature UGE polypeptide. A preferred derivative of a UGE polypeptide useful in the methods of the invention is that resulting from removal of the signalling peptide and corresponding to the mature UGE polypeptide.

Preferably, the polypeptide sequence which when used in the construction of a phylogenetic tree, such as the one depicted in FIG. 21, clusters with the group comprising the amino acid sequence represented by SEQ ID NO: 225 (Os_UGE2) or alternatively, when in a tree such as that of FIG. 1 in Rosti et al. 2007 (The Plant Cell 19:1565-1579) clusters with group comprising AtUGE2.

The term “domain” and “motif' is defined in the “definitions” section herein. Specialist databases exist for the identification of domains, for example, SMART (Schultz et al. (1998) Proc. Natl. Acad. Sci. USA 95, 5857-5864; Letunic et al. (2002) Nucleic Acids Res 30, 242-244), InterPro (Mulder et al., (2003) Nucl. Acids. Res. 31, 315-318), Prosite (Bucher and Bairoch (1994), A generalized profile syntax for biomolecular sequences motifs and its function in automatic sequence interpretation. (In) ISMB-94; Proceedings 2nd International Conference on Intelligent Systems for Molecular Biology. Altman R., Brutlag D., Karp P., Lathrop R., Searls D., Eds., pp 53-61, AAAI Press, Menlo Park; Hulo et al., Nucl. Acids. Res. 32:D134-D137, (2004)), or Pfam (Bateman et al., Nucleic Acids Research 30(1): 276-280 (2002)). A set of tools for in silico analysis of protein sequences is available on the ExPASy proteomics server (Swiss Institute of Bioinformatics (Gasteiger et al., ExPASy: the proteomics server for in-depth protein knowledge and analysis, Nucleic Acids Res. 31:3784-3788 (2003)). Domains or motifs may also be identified using routine techniques, such as by sequence alignment.

Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the global (i.e. spanning the complete sequences) alignment of two sequences that maximizes the number of matches and minimizes the number of gaps. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information (NCBI). Homologues may readily be identified using, for example, the ClustalW multiple sequence alignment algorithm (version 1.83), with the default pairwise alignment parameters, and a scoring method in percentage. Global percentages of similarity and identity may also be determined using one of the methods available in the MatGAT software package (Campanella et al., BMC Bioinformatics. 2003 Jul. 10; 4:29. MatGAT: an application that generates similarity/identity matrices using protein or DNA sequences.). Minor manual editing may be performed to optimise alignment between conserved motifs, as would be apparent to a person skilled in the art. Furthermore, instead of using full-length sequences for the identification of homologues, specific domains may also be used. The sequence identity values may be determined over the entire nucleic acid or amino acid sequence or over selected domains or conserved motif(s), using the programs mentioned above using the default parameters.

Furthermore, UGE polypeptides (at least in their native form) typically have UDP-Glucose 4-epimerase or UDP-Galactose 4-epimerase activity. In addition UGE polypeptides useful in the methods of the invention may also be enzymatically inactive. Tools and techniques for measuring UDP_Glucose 4-epimerase and UDP-Galactose 4-epimerase activity are well known in the art. Further details on the reaction properties or activity as well as examples of UGE polypeptides are well known in the art and can be found in specialized databases such as BRENDA (The Comprehensive Enzyme Information System) developed and maintained at the University of Cologne (Germany). In vivo assays include the complementation of defective UDP_Glucose 4-epimerase activity in microorganisms carrying mutations in genes encoding UGE polypeptides, e.g. the Scharomyces cerevisiae strain carrying a deficiency in the gene encoding the GAL10 protein (Dormann and Benning. 1996. Arch Biochem Biophys. 327(1):27-34). Methods to purify UGE polypeptides and to measure the epimearase activity in vitro have also been described (Barber 2006).

The present invention is illustrated by transforming plants with the nucleic acid sequence represented by SEQ ID NO: 224, encoding the polypeptide sequence of SEQ ID NO: 225. However, performance of the invention is not restricted to these sequences; the methods of the invention may advantageously be performed using any UGE-encoding nucleic acid or UGE polypeptide as defined herein.

Examples of nucleic acids encoding UGE polypeptides are given in Table A6 of Example 1 herein. Such nucleic acids are useful in performing the methods of the invention. The amino acid sequences given in Table A6 of Example 1 are example sequences of orthologues and paralogues of the UGE polypeptide represented by SEQ ID NO: 225, the terms “orthologues” and “paralogues” being as defined herein. Further orthologues and paralogues may readily be identified by performing a so-called reciprocal blast search. Typically, this involves a first BLAST involving BLASTing a query sequence (for example using any of the sequences listed in Table A6 of Example 1) against any sequence database, such as the publicly available NCBI database. BLASTN or TBLASTX (using standard default values) are generally used when starting from a nucleotide sequence, and BLASTP or TBLASTN (using standard default values) when starting from a protein sequence. The BLAST results may optionally be filtered. The full-length sequences of either the filtered results or non-filtered results are then BLASTed back (second BLAST) against sequences from the organism from which the query sequence is derived (where the query sequence is SEQ ID NO: 224 or SEQ ID NO: 225, the second BLAST would therefore be against rice sequences). The results of the first and second BLASTs are then compared. A paralogue is identified if a high-ranking hit from the first blast is from the same species as from which the query sequence is derived, a BLAST back then ideally results in the query sequence amongst the highest hits; an orthologue is identified if a high-ranking hit in the first BLAST is not from the same species as from which the query sequence is derived, and preferably results upon BLAST back in the query sequence being among the highest hits.

High-ranking hits are those having a low E-value. The lower the E-value, the more significant the score (or in other words the lower the chance that the hit was found by chance). Computation of the E-value is well known in the art. In addition to E-values, comparisons are also scored by percentage identity. Percentage identity refers to the number of identical nucleotides (or amino acids) between the two compared nucleic acid (or polypeptide) sequences over a particular length. In the case of large families, ClustalW may be used, followed by a neighbour joining tree, to help visualize clustering of related genes and to identify orthologues and paralogues.

Nucleic acid variants may also be useful in practising the methods of the invention. Examples of such variants include nucleic acids encoding homologues and derivatives of any one of the amino acid sequences given in Table A6 of Example 1, the terms “homologue” and “derivative” being as defined herein. Also useful in the methods of the invention are nucleic acids encoding homologues and derivatives of orthologues or paralogues of any one of the amino acid sequences given in Table A6 of Example 1. Homologues and derivatives useful in the methods of the present invention have substantially the same biological and functional activity as the unmodified protein from which they are derived.

Further nucleic acid variants useful in practising the methods of the invention include portions of nucleic acids encoding UGE polypeptides, nucleic acids hybridising to nucleic acids encoding UGE polypeptides, splice variants of nucleic acids encoding UGE polypeptides, allelic variants of nucleic acids encoding UGE polypeptides and variants of nucleic acids encoding UGE polypeptides obtained by gene shuffling. The terms hybridising sequence, splice variant, allelic variant and gene shuffling are as described herein.

Nucleic acids encoding UGE polypeptides need not be full-length nucleic acids, since performance of the methods of the invention does not rely on the use of full-length nucleic acid sequences. According to the present invention, there is provided a method for enhancing yield-related traits in plants, comprising introducing and expressing in a plant a portion of any one of the nucleic acid sequences given in Table A6 of Example 1, or a portion of a nucleic acid encoding an orthologue, paralogue or homologue of any of the amino acid sequences given in Table A6 of Example 1.

A portion of a nucleic acid may be prepared, for example, by making one or more deletions to the nucleic acid. The portions may be used in isolated form or they may be fused to other coding (or non-coding) sequences in order to, for example, produce a protein that combines several activities. When fused to other coding sequences, the resultant polypeptide produced upon translation may be bigger than that predicted for the protein portion.

Portions useful in the methods of the invention, encode a UGE polypeptide as defined herein, and have substantially the same biological activity as the amino acid sequences given in Table A6 of Example 1. Preferably, the portion is a portion of any one of the nucleic acids given in Table A of Example 1, or is a portion of a nucleic acid encoding an orthologue or paralogue of any one of the amino acid sequences given in Table A6 of Example 1. Preferably the portion is at least 250, 500, 550, 600, 650, 700, 750, 800, 850, 900, 950, 1000 consecutive nucleotides in length, the consecutive nucleotides being of any one of the nucleic acid sequences given in Table A6 of Example 1, or of a nucleic acid encoding an orthologue or paralogue of any one of the amino acid sequences given in Table A6 of Example 1. Most preferably the portion is a portion of the nucleic acid of SEQ ID NO: 224. Preferably, the portion encodes an amino acid sequence which when used in the construction of a phylogenetic tree, such as the one depicted in FIG. 21, clusters with the group comprising the amino acid sequence represented by SEQ ID NO: 225 (Os_UGE2) or alternatively, when in a tree such as that of FIG. 1 in Rosti et al. 2007 (The Plant Cell 19:1565-1579) clusters with group comprising AtUGE2.

Another nucleic acid variant useful in the methods of the invention is a nucleic acid capable of hybridising, under reduced stringency conditions, preferably under stringent conditions, with a nucleic acid encoding a UGE polypeptide as defined herein, or with a portion as defined herein.

According to the present invention, there is provided a method for enhancing yield-related traits in plants, comprising introducing and expressing in a plant a nucleic acid capable of hybridizing to any one of the nucleic acids given in Table A6 of Example 1, or comprising introducing and expressing in a plant a nucleic acid capable of hybridising to a nucleic acid encoding an orthologue, paralogue or homologue of any of the nucleic acid sequences given in Table A6 of Example 1.

Hybridising sequences useful in the methods of the invention encode a UGE polypeptide as defined herein, having substantially the same biological activity as the amino acid sequences given in Table A6 of Example 1. Preferably, the hybridising sequence is capable of hybridising to any one of the nucleic acids given in Table A6 of Example 1, or to a portion of any of these sequences, a portion being as defined above, or the hybridising sequence is capable of hybridising to a nucleic acid encoding an orthologue or paralogue of any one of the amino acid sequences given in Table A6 of Example 1. Most preferably, the hybridising sequence is capable of hybridising to a nucleic acid as represented by SEQ ID NO: 224 or to a portion thereof.

Preferably, the hybridising sequence encodes a polypeptide with an amino acid sequence which when used in the construction of a phylogenetic tree, such as the one depicted in FIG. 21, clusters with the group comprising the amino acid sequence represented by SEQ ID NO: 225 (Os_UGE2) or alternatively when used in the construction of a phylogenetic tree such as that of FIG. 1 in Rosti et al. 2007 (The Plant Cell 19:1565-1579) clusters with group comprising AtUGE2.

Another nucleic acid variant useful in the methods of the invention is a splice variant encoding a UGE polypeptide as defined hereinabove, a splice variant being as defined herein.

According to the present invention, there is provided a method for enhancing yield-related traits in plants, comprising introducing and expressing in a plant a splice variant of any one of the nucleic acid sequences given in Table A6 of Example 1, or a splice variant of a nucleic acid encoding an orthologue, paralogue or homologue of any of the amino acid sequences given in Table A6 of Example 1.

Preferred splice variants are splice variants of a nucleic acid represented by SEQ ID NO: 224, or a splice variant of a nucleic acid encoding an orthologue or paralogue of SEQ ID NO: 225. Preferably, the amino acid sequence encoded by the splice variant, when used in the construction of a phylogenetic tree, such as the one depicted in FIG. 21, clusters with the group comprising the amino acid sequence represented by SEQ ID NO: 225 (Os_UGE2) or alternatively when used in the construction of a phylogenetic tree such as that of FIG. 1 in Rosti et al. 2007 (The Plant Cell 19:1565-1579) clusters with group comprising AtUGE2.

Another nucleic acid variant useful in performing the methods of the invention is an allelic variant of a nucleic acid encoding a UGE polypeptide as defined hereinabove, an allelic variant being as defined herein.

According to the present invention, there is provided a method for enhancing yield-related traits in plants, comprising introducing and expressing in a plant an allelic variant of any one of the nucleic acids given in Table A6 of Example 1, or comprising introducing and expressing in a plant an allelic variant of a nucleic acid encoding an orthologue, paralogue or homologue of any of the amino acid sequences given in Table A6 of Example 1.

The allelic variants useful in the methods of the present invention have substantially the same biological activity as the UGE polypeptide of SEQ ID NO: 225 and any of the amino acids depicted in Table A6 of Example 1. Allelic variants exist in nature, and encompassed within the methods of the present invention is the use of these natural alleles. Preferably, the allelic variant is an allelic variant of SEQ ID NO: 224 or an allelic variant of a nucleic acid encoding an orthologue or paralogue of SEQ ID NO: 225. Preferably, the amino acid sequence encoded by the allelic variant, when used in the construction of a phylogenetic tree, such as the one depicted in FIG. 21, clusters with the group comprising the amino acid sequence represented by SEQ ID NO: 225 (Os_UGE2) or alternatively when used in the construction of a phylogenetic tree such as that of FIG. 1 in Rosti et al. 2007 (The Plant Cell 19:1565-1579) clusters with group comprising AtUGE2.

Gene shuffling or directed evolution may also be used to generate variants of nucleic acids encoding UGE polypeptides as defined above; the term “gene shuffling” being as defined herein.

According to the present invention, there is provided a method for enhancing yield-related traits in plants, comprising introducing and expressing in a plant a variant of any one of the nucleic acid sequences given in Table A6 of Example 1, or comprising introducing and expressing in a plant a variant of a nucleic acid encoding an orthologue, paralogue or homologue of any of the amino acid sequences given in Table A6 of Example 1, which variant nucleic acid is obtained by gene shuffling.

Preferably, the amino acid sequence encoded by the variant nucleic acid obtained by gene shuffling, when used in the construction of a phylogenetic tree such as the one depicted in FIG. 21, clusters with the group comprising the amino acid sequence represented by SEQ ID NO: 225 (Os_UGE2) or alternatively when used in the construction of a phylogenetic tree such as that of FIG. 1 in Rosti et al. 2007 (The Plant Cell 19:1565-1579) clusters with group comprising AtUGE2.

Furthermore, nucleic acid variants may also be obtained by site-directed mutagenesis. Several methods are available to achieve site-directed mutagenesis, the most common being PCR based methods (Current Protocols in Molecular Biology. Wiley Eds.).

Nucleic acids encoding UGE polypeptides may be derived from any natural or artificial source. The nucleic acid may be modified from its native form in composition and/or genomic environment through deliberate human manipulation. Preferably the UGE polypeptide-encoding nucleic acid is from a plant, further preferably from a monocotyledonous plant, more preferably from the family Poaceae, most preferably the nucleic acid is from Oryza sativa.

Performance of the methods of the invention gives plants having enhanced yield-related traits. In particular performance of the methods of the invention gives plants having increased yield, especially increased seed yield relative to control plants. The terms “yield” and “seed yield” are described in more detail in the “definitions” section herein.

Reference herein to enhanced yield-related traits is taken to mean an increase in biomass (weight) of one or more parts of a plant, which may include aboveground (harvestable) parts and/or (harvestable) parts below ground. In particular, such harvestable parts are seeds, and performance of the methods of the invention results in plants having increased seed yield relative to the seed yield of control plants.

Taking corn as an example, a yield increase may be manifested as one or more of the following: increase in the number of plants established per hectare or acre, an increase in the number of ears per plant, an increase in the number of rows, number of kernels per row, kernel weight, thousand kernel weight, ear length/diameter, increase in the seed filling rate (which is the number of filled seeds divided by the total number of seeds and multiplied by 100), among others. Taking rice as an example, a yield increase may manifest itself as an increase in one or more of the following: number of plants per hectare or acre, number of panicles per plant, number of spikelets per panicle, number of flowers (florets) per panicle (which is expressed as a ratio of the number of filled seeds over the number of primary panicles), increase in the seed filling rate (which is the number of filled seeds divided by the total number of seeds and multiplied by 100), increase in thousand kernel weight, among others.

The present invention provides a method for increasing yield, especially seed yield of plants, relative to control plants, which method comprises modulating expression, preferably increasing expression, in a plant of a nucleic acid encoding a UGE polypeptide as defined herein.

Since the transgenic plants according to the present invention have increased yield, it is likely that these plants exhibit an increased growth rate (during at least part of their life cycle), relative to the growth rate of control plants at a corresponding stage in their life cycle.

The increased growth rate may be specific to one or more parts of a plant (including seeds), or may be throughout substantially the whole plant. Plants having an increased growth rate may have a shorter life cycle. The life cycle of a plant may be taken to mean the time needed to grow from a dry mature seed up to the stage where the plant has produced dry mature seeds, similar to the starting material. This life cycle may be influenced by factors such as early vigour, growth rate, greenness index, flowering time and speed of seed maturation. The increase in growth rate may take place at one or more stages in the life cycle of a plant or during substantially the whole plant life cycle. Increased growth rate during the early stages in the life cycle of a plant may reflect enhanced vigour. The increase in growth rate may alter the harvest cycle of a plant allowing plants to be sown later and/or harvested sooner than would otherwise be possible (a similar effect may be obtained with earlier flowering time). If the growth rate is sufficiently increased, it may allow for the further sowing of seeds of the same plant species (for example sowing and harvesting of rice plants followed by sowing and harvesting of further rice plants all within one conventional growing period). Similarly, if the growth rate is sufficiently increased, it may allow for the further sowing of seeds of different plants species (for example the sowing and harvesting of corn plants followed by, for example, the sowing and optional harvesting of soybean, potato or any other suitable plant). Harvesting additional times from the same rootstock in the case of some crop plants may also be possible. Altering the harvest cycle of a plant may lead to an increase in annual biomass production per acre (due to an increase in the number of times (say in a year) that any particular plant may be grown and harvested). An increase in growth rate may also allow for the cultivation of transgenic plants in a wider geographical area than their wild-type counterparts, since the territorial limitations for growing a crop are often determined by adverse environmental conditions either at the time of planting (early season) or at the time of harvesting (late season). Such adverse conditions may be avoided if the harvest cycle is shortened. The growth rate may be determined by deriving various parameters from growth curves, such parameters may be: T-Mid (the time taken for plants to reach 50% of their maximal size) and T-90 (time taken for plants to reach 90% of their maximal size), amongst others.

According to a preferred feature of the present invention, performance of the methods of the invention gives plants having an increased growth rate relative to control plants. Therefore, according to the present invention, there is provided a method for increasing the growth rate of plants, which method comprises modulating expression, preferably increasing expression, in a plant of a nucleic acid encoding a UGE polypeptide as defined herein.

An increase in yield and/or growth rate occurs whether the plant is under non-stress conditions or whether the plant is exposed to various stresses compared to control plants. Plants typically respond to exposure to stress by growing more slowly. In conditions of severe stress, the plant may even stop growing altogether. Mild stress on the other hand is defined herein as being any stress to which a plant is exposed which does not result in the plant ceasing to grow altogether without the capacity to resume growth. Mild stress in the sense of the invention leads to a reduction in the growth of the stressed plants of less than 40%, 35% or 30%, preferably less than 25%, 20% or 15%, more preferably less than 14%, 13%, 12%, 11% or 10% or less in comparison to the control plant under non-stress conditions. Due to advances in agricultural practices (irrigation, fertilization, pesticide treatments) severe stresses are not often encountered in cultivated crop plants. As a consequence, the compromised growth induced by mild stress is often an undesirable feature for agriculture. Mild stresses are the everyday biotic and/or abiotic (environmental) stresses to which a plant is exposed. Abiotic stresses may be due to drought or excess water, anaerobic stress, salt stress, chemical toxicity, oxidative stress and hot, cold or freezing temperatures. The abiotic stress may be an osmotic stress caused by a water stress (particularly due to drought), salt stress, oxidative stress or an ionic stress. Biotic stresses are typically those stresses caused by pathogens, such as bacteria, viruses, fungi and insects.

In particular, the methods of the present invention may be performed under non-stress conditions or under conditions of mild drought to give plants having increased yield relative to control plants. As reported in Wang et al. (Planta (2003) 218: 1-14), abiotic stress leads to a series of morphological, physiological, biochemical and molecular changes that adversely affect plant growth and productivity. Drought, salinity, extreme temperatures and oxidative stress are known to be interconnected and may induce growth and cellular damage through similar mechanisms. Rabbani et al. (Plant Physiol (2003) 133: 1755-1767) describes a particularly high degree of “cross talk” between drought stress and high-salinity stress. For example, drought and/or salinisation are manifested primarily as osmotic stress, resulting in the disruption of homeostasis and ion distribution in the cell. Oxidative stress, which frequently accompanies high or low temperature, salinity or drought stress, may cause denaturing of functional and structural proteins. As a consequence, these diverse environmental stresses often activate similar cell signalling pathways and cellular responses, such as the production of stress proteins, up-regulation of anti-oxidants, accumulation of compatible solutes and growth arrest. The term “non-stress” conditions as used herein are those environmental conditions that allow optimal growth of plants. Persons skilled in the art are aware of normal soil conditions and climatic conditions for a given location.

Performance of the methods of the invention gives plants grown under non-stress conditions or under mild drought conditions increased yield relative to control plants grown under comparable conditions. Therefore, according to the present invention, there is provided a method for increasing yield in plants grown under non-stress conditions or under mild drought conditions, which method comprises increasing expression in a plant of a nucleic acid encoding a UGE polypeptide.

Performance of the methods of the invention gives plants grown under conditions of nutrient deficiency, particularly under conditions of nitrogen deficiency, increased yield relative to control plants grown under comparable conditions. Therefore, according to the present invention, there is provided a method for increasing yield in plants grown under conditions of nutrient deficiency, which method comprises increasing expression in a plant of a nucleic acid encoding a UGE polypeptide. Nutrient deficiency may result from a lack of nutrients such as nitrogen, phosphates and other phosphorous-containing compounds, potassium, calcium, cadmium, magnesium, manganese, iron and boron, amongst others.

The present invention encompasses plants or parts thereof (including seeds) obtainable by the methods according to the present invention. The plants or parts thereof comprise a nucleic acid transgene encoding a UGE polypeptide as defined above.

The invention also provides genetic constructs and vectors to facilitate introduction and/or expression in plants of nucleic acids encoding UGE polypeptides. The gene constructs may be inserted into vectors, which may be commercially available, suitable for transforming into plants and suitable for expression of the gene of interest in the transformed cells. The invention also provides use of a gene construct as defined herein in the methods of the invention.

More specifically, the present invention provides a construct comprising:

(a) a nucleic acid encoding a UGE polypeptide as defined above;
(b) one or more control sequences capable of driving expression of the nucleic acid sequence of (a); and optionally
(c) a transcription termination sequence.

Preferably, the nucleic acid encoding a UGE polypeptide is as defined above. The term “control sequence” and “termination sequence” are as defined herein.

Plants are transformed with a vector comprising any of the nucleic acids described above. The skilled artisan is well aware of the genetic elements that must be present on the vector in order to successfully transform, select and propagate host cells containing the sequence of interest. The sequence of interest is operably linked to one or more control sequences (at least to a promoter).

Advantageously, any type of promoter, whether natural or synthetic, may be used to drive expression of the nucleic acid sequence. A constitutive promoter is particularly useful in the methods. See the “Definitions” section herein for definitions of the various promoter types.

It should be clear that the applicability of the present invention is not restricted to the UGE polypeptide-encoding nucleic acid represented by SEQ ID NO: 1, nor is the applicability of the invention restricted to expression of a UGE polypeptide-encoding nucleic acid when driven by a constitutive promoter, or when driven by a root-specific promoter.

The constitutive promoter is preferably a GOS2 promoter, preferably a GOS2 promoter from rice. Further preferably the constitutive promoter is represented by a nucleic acid sequence substantially similar to SEQ ID NO: 270, most preferably the constitutive promoter is as represented by SEQ ID NO: 270. See Table 2 in the “Definitions” section herein for further examples of constitutive promoters.

Optionally, one or more terminator sequences may be used in the construct introduced into a plant. Additional regulatory elements may include transcriptional as well as translational enhancers. Those skilled in the art will be aware of terminator and enhancer sequences that may be suitable for use in performing the invention. An intron sequence may also be added to the 5′ untranslated region (UTR) or in the coding sequence to increase the amount of the mature message that accumulates in the cytosol, as described in the definitions section. Other control sequences (besides promoter, enhancer, silencer, intron sequences, 3′UTR and/or 5′UTR regions) may be protein and/or RNA stabilizing elements. Such sequences would be known or may readily be obtained by a person skilled in the art.

The genetic constructs of the invention may further include an origin of replication sequence that is required for maintenance and/or replication in a specific cell type. One example is when a genetic construct is required to be maintained in a bacterial cell as an episomal genetic element (e.g. plasmid or cosmid molecule). Preferred origins of replication include, but are not limited to, the f1-ori and colE1.

For the detection of the successful transfer of the nucleic acid sequences as used in the methods of the invention and/or selection of transgenic plants comprising these nucleic acids, it is advantageous to use marker genes (or reporter genes). Therefore, the genetic construct may optionally comprise a selectable marker gene. Selectable markers are described in more detail in the “definitions” section herein. The marker genes may be removed or excised from the transgenic cell once they are no longer needed. Techniques for marker removal are known in the art, useful techniques are described above in the definitions section.

The invention also provides a method for the production of transgenic plants having enhanced yield-related traits relative to control plants, comprising introduction and expression in a plant of any nucleic acid encoding a UGE polypeptide as defined hereinabove.

More specifically, the present invention provides a method for the production of transgenic plants having increased enhanced yield-related traits, particularly increased (seed) yield, which method comprises:
(i) introducing and expressing in a plant or plant cell a UGE polypeptide-encoding nucleic acid; and
(ii) cultivating the plant cell under conditions promoting plant growth and development.

The nucleic acid of (i) may be any of the nucleic acids capable of encoding a UGE polypeptide as defined herein.

The nucleic acid may be introduced directly into a plant cell or into the plant itself (including introduction into a tissue, organ or any other part of a plant). According to a preferred feature of the present invention, the nucleic acid is preferably introduced into a plant by transformation. The term “transformation” is described in more detail in the “definitions” section herein.

The genetically modified plant cells can be regenerated via all methods with which the skilled worker is familiar. Suitable methods can be found in the abovementioned publications by S. D. Kung and R. Wu, Potrykus or Högen and Willmitzer.

Generally after transformation, plant cells or cell groupings are selected for the presence of one or more markers which are encoded by plant-expressible genes co-transferred with the gene of interest, following which the transformed material is regenerated into a whole plant.

To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as the ones described above.

Following DNA transfer and regeneration, putatively transformed plants may also be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

The present invention clearly extends to any plant cell or plant produced by any of the methods described herein, and to all plant parts and propagules thereof. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced by the parent in the methods according to the invention.

The invention also includes host cells containing an isolated nucleic acid encoding a UGE polypeptide as defined hereinabove. Preferred host cells according to the invention are plant cells. Host plants for the nucleic acids or the vector used in the method according to the invention, the expression cassette or construct or vector are, in principle, advantageously all plants, which are capable of synthesizing the polypeptides used in the inventive method.

The methods of the invention are advantageously applicable to any plant. Plants that are particularly useful in the methods of the invention include all plants which belong to the superfamily Viridiplantae, in particular monocotyledonous and dicotyledonous plants including fodder or forage legumes, ornamental plants, food crops, trees or shrubs. According to a preferred embodiment of the present invention, the plant is a crop plant. Examples of crop plants include soybean, sunflower, canola, alfalfa, rapeseed, cotton, tomato, potato and tobacco. Further preferably, the plant is a monocotyledonous plant. Examples of monocotyledonous plants include sugarcane. More preferably the plant is a cereal. Examples of cereals include rice, maize, wheat, barley, millet, rye, triticale, sorghum and oats.

The invention also extends to harvestable parts of a plant such as, but not limited to seeds, leaves, fruits, flowers, stems, roots, rhizomes, tubers and bulbs. The invention furthermore relates to products derived, preferably directly derived, from a harvestable part of such a plant, such as dry pellets or powders, oil, fat and fatty acids, starch or proteins.

According to a preferred feature of the invention, the modulated expression is increased expression. Methods for increasing expression of nucleic acids or genes, or gene products, are well documented in the art and examples are provided in the definitions section.

As mentioned above, a preferred method for modulating (preferably, increasing) expression of a nucleic acid encoding a UGE polypeptide is by introducing and expressing in a plant a nucleic acid encoding a UGE polypeptide; however the effects of performing the method, i.e. enhancing yield-related traits may also be achieved using other well known techniques, including but not limited to T-DNA activation tagging, TILLING, homologous recombination. A description of these techniques is provided in the definitions section.

The present invention also encompasses use of nucleic acids encoding UGE polypeptides as described herein and use of these UGE polypeptides in enhancing any of the aforementioned yield-related traits in plants.

Nucleic acids encoding UGE polypeptide described herein, or the UGE polypeptides themselves, may find use in breeding programmes in which a DNA marker is identified which may be genetically linked to a UGE polypeptide-encoding gene. The nucleic acids/genes, or the UGE polypeptides themselves may be used to define a molecular marker. This DNA or protein marker may then be used in breeding programmes to select plants having enhanced yield-related traits as defined hereinabove in the methods of the invention.

Allelic variants of a UGE polypeptide-encoding nucleic acid/gene may also find use in marker-assisted breeding programmes. Such breeding programmes sometimes require introduction of allelic variation by mutagenic treatment of the plants, using for example EMS mutagenesis; alternatively, the programme may start with a collection of allelic variants of so called “natural” origin caused unintentionally. Identification of allelic variants then takes place, for example, by PCR. This is followed by a step for selection of superior allelic variants of the sequence in question and which give increased yield. Selection is typically carried out by monitoring growth performance of plants containing different allelic variants of the sequence in question. Growth performance may be monitored in a greenhouse or in the field. Further optional steps include crossing plants in which the superior allelic variant was identified with another plant. This could be used, for example, to make a combination of interesting phenotypic features.

Nucleic acids encoding UGE polypeptides may also be used as probes for genetically and physically mapping the genes that they are a part of, and as markers for traits linked to those genes. Such information may be useful in plant breeding in order to develop lines with desired phenotypes. Such use of UGE polypeptide-encoding nucleic acids requires only a nucleic acid sequence of at least 15 nucleotides in length. The UGE polypeptide-encoding nucleic acids may be used as restriction fragment length polymorphism (RFLP) markers. Southern blots (Sambrook J, Fritsch E F and Maniatis T (1989) Molecular Cloning, A Laboratory Manual) of restriction-digested plant genomic DNA may be probed with the UGE-encoding nucleic acids. The resulting banding patterns may then be subjected to genetic analyses using computer programs such as MapMaker (Lander et al. (1987) Genomics 1: 174-181) in order to construct a genetic map. In addition, the nucleic acids may be used to probe Southern blots containing restriction endonuclease-treated genomic DNAs of a set of individuals representing parent and progeny of a defined genetic cross. Segregation of the DNA polymorphisms is noted and used to calculate the position of the UGE polypeptide-encoding nucleic acid in the genetic map previously obtained using this population (Botstein et al. (1980) Am. J. Hum. Genet. 32:314-331).

The production and use of plant gene-derived probes for use in genetic mapping is described in Bernatzky and Tanksley (1986) Plant Mol. Biol. Reporter 4: 37-41. Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

The nucleic acid probes may also be used for physical mapping (i.e., placement of sequences on physical maps; see Hoheisel et al. In: Non-mammalian Genomic Analysis: A Practical Guide, Academic press 1996, pp. 319-346, and references cited therein).

In another embodiment, the nucleic acid probes may be used in direct fluorescence in situ hybridisation (FISH) mapping (Trask (1991) Trends Genet. 7:149-154). Although current methods of FISH mapping favour use of large clones (several kb to several hundred kb; see Laan et al. (1995) Genome Res. 5:13-20), improvements in sensitivity may allow performance of FISH mapping using shorter probes.

A variety of nucleic acid amplification-based methods for genetic and physical mapping may be carried out using the nucleic acids. Examples include allele-specific amplification (Kazazian (1989) J. Lab. Clin. Med 11:95-96), polymorphism of PCR-amplified fragments (CAPS; Sheffield et al. (1993) Genomics 16:325-332), allele-specific ligation (Landegren et al. (1988) Science 241:1077-1080), nucleotide extension reactions (Sokolov (1990) Nucleic Acid Res. 18:3671), Radiation Hybrid Mapping (Walter et al. (1997) Nat. Genet. 7:22-28) and Happy Mapping (Dear and Cook (1989) Nucleic Acid Res. 17:6795-6807). For these methods, the sequence of a nucleic acid is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for mapping methods.

The methods according to the present invention result in plants having enhanced yield-related traits, as described hereinbefore. These traits may also be combined with other economically advantageous traits, such as further yield-enhancing traits, tolerance to other abiotic and biotic stresses, traits modifying various architectural features and/or biochemical and/or physiological features.

The term “table A6” used in this specification is to be taken to specify the content of table A6a and table A6b. The term “table A6a” used in this specification is to be taken to specify the content of table A6a. The term “table A6b” used in this specification is to be taken to specify the content of table A6b. In one preferred embodiment, the term “table A6” means table A6b.

In one embodiment the invention relates to subject mater summarized as follows:

Item 104: A method for enhancing yield-related traits in plants relative to control plants, comprising modulating expression in a plant of a nucleic acid encoding a UGE polypeptide.
Item 105: Method according to item 104, wherein said UGE polypeptide comprises an epimerase domain having in increasing order of preference at least 70%, 75%, 80%, 85%, 90%, 95%, 97% or more sequence identity to SEQ ID NO: 275.
Item 106: Method according to item 104 or 105, wherein said UGE polypeptide comprises one or more of the following motifs:
Motif 8 as represented by SEQ ID NO: 276;
Motif 9 as represented by SEQ ID NO: 277 wherein the G can be substituted by any other non polar amino acid and the R by any other polar amino acid;
Motif 10 as represented by SEQ ID NO: 278, wherein 1, 2, 3 or 4 mismatches are allowed;
Motif 11 as represented by SEQ ID NO: 279,
Motif 12 as represented by SEQ ID NO: 280, wherein 1, 2, 3 or 4 mismatches are allowed.
Item 107: Method according to item 104 to 106, wherein said modulated expression is effected by introducing and expressing in a plant a nucleic acid encoding a UGE polypeptide.
Item 108: Method according to any of the items 104 to 107, wherein said nucleic acid encoding a UGE polypeptide encodes any one of the proteins listed in Table A6 or is a portion of such a nucleic acid, or a nucleic acid capable of hybridising with such a nucleic acid.
Item 109: Method according to any of the items 104 to 108, wherein said nucleic acid sequence encodes an orthologue or paralogue of any of the proteins given in Table A6.
Item 110: Method according to any of the items 104 to 109, wherein said enhanced yield-related traits comprise increased yield, preferably increased seed yield relative to control plants.
Item 111: Method according to any of the items 104 to 110, wherein said enhanced yield-related traits are obtained under non-stress conditions.
Item 112: Method according to any of the items 104 to 111, wherein said enhanced yield-related traits are obtained under conditions of drought stress.
Item 113: Method according to any of the items 104 to 112, wherein said nucleic acid is operably linked to a constitutive promoter, preferably to a GOS2 promoter, most preferably to a GOS2 promoter from rice.
Item 114: Method according to any of the items 104 to 113, wherein said nucleic acid encoding a UGE polypeptide is of plant origin, preferably from a monocotyledonous plant, further preferably from the family Poaceae, more preferably from the genus Oryza, most preferably from Oryza sativa.
Item 115: Plant or part thereof, including seeds, obtainable by a method according to any of the items 104 to 114, wherein said plant or part thereof comprises a recombinant nucleic acid encoding a UGE polypeptide.
Item 116: An isolated nucleic acid sequence comprising a nucleic acid sequence selected from the group consisting of:
(a) an isolated nucleic acid molecule encoding the polypeptide as depicted in SEQ ID NO: 282, 284, 286, 288, 290, 292, 294, 296, 298, 300, 302, 304, 306, 308, 310, 312, 314, 316, 318 and/or 320 and/or table A6b;
(b) an isolated nucleic acid molecule as depicted in SEQ ID NO: 281, 283, 285, 287, 289, 291, 293, 295, 297, 299, 301, 303, 305, 307, 309, 311, 313, 315, 317 and/or 319 and/or table A6b;
(c) an isolated nucleic acid molecule, which, as a result of the degeneracy of the genetic code, can be derived from a polypeptide sequence depicted in table A6 and confers enhanced yield-related traits relative to control plants;
(d) an isolated nucleic acid molecule having at least 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with the nucleic acid molecule sequence of a polynucleotide comprising the nucleic acid molecule depicted in table A6 and confers enhanced yield-related traits relative to control plants;
(e) an isolated nucleic acid molecule encoding a polypeptide having at least 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of the polypeptide encoded by the nucleic acid molecule of (a) to (c) and confers enhanced yield-related traits relative to control plants;
(f) an isolated nucleic acid molecule which hybridizes with a nucleic acid molecule of (a) to (c) under stringent hybridization conditions and confers enhanced yield-related traits relative to control plants;
(g) an isolated nucleic acid molecule encoding a polypeptide comprising the consensus sequence or one or more polypeptide motifs as shown FIG. 20 and preferably having the activity represented by a nucleic acid molecule comprising a polynucleotide as depicted in table A6;
(h) an isolated nucleic acid molecule encoding a polypeptide having the activity represented by a protein as depicted in table A6 and confering enhanced yield-related traits relative to control plants;
and
(i) a nucleic acid molecule which is obtainable by screening a suitable nucleic acid library under stringent hybridization conditions with a probe comprising a complementary sequence of a nucleic acid molecule of (a) or (b) or with a fragment thereof, having at least 15 nt, preferably 20 nt, 30 nt, 50 nt, 100 nt, 200 nt or 500 nt of a nucleic acid molecule complementary to a nucleic acid molecule sequence characterized in (a) to (e) and encoding a polypeptide having the activity represented by a protein comprising a polypeptide as depicted in table A6;
whereby the nucleic acid molecule according to (a) to (i) is at least in one or more nucleotides different from the sequence depicted in table A6a and preferably which encodes a protein which differs at least in one or more amino acids from the protein sequences depicted in table A6a.
   Item 117: Construct comprising:
(a) nucleic acid encoding a UGE polypeptide as defined in items 104 to 106 or 116;
(b) one or more control sequences capable of driving expression of the nucleic acid sequence of (a); and optionally
(c) a transcription termination sequence.
   Item 118: Construct according to item 117, wherein one of said control sequences is a constitutive promoter, preferably a GOS2 promoter, most preferably a GOS2 promoter from rice.
   Item 119: Use of a construct according to any of the items 117 to 118 in a method for making plants having enhanced yield-related traits preferably increased yield, more preferably increased seed yield relative to control plants.
   Item 120: Plant, plant part or plant cell transformed with a construct according to item 117 or 118.
   Item 121: Method for the production of a transgenic plant having increased yield, particularly increased seed yield relative to control plants, comprising:
   (i) introducing and expressing in a plant a nucleic acid encoding a UGE polypeptide as defined in items 104 to 106 or 116; and
   (ii) cultivating the plant cell under conditions promoting plant growth and development.
   Item 122: Transgenic plant having increased yield, particularly increased seed yield, relative to control plants, resulting from increased expression of a nucleic acid encoding a UGE polypeptide as defined in items 104 to 106 or 116, or a transgenic plant cell derived from said transgenic plant.
   Item 123: Transgenic plant according to item 115, 120 or 122, or a transgenic plant cell derived thereof, wherein said plant is a crop plant or a monocot or a cereal, such as rice, maize, wheat, barley, millet, rye, triticale, sorghum and oats.
   Item 124: Harvestable parts of a plant according to item 123, wherein said harvestable parts are preferably shoot biomass and/or seeds.
   Item 125: Products derived from a plant according to item 123 and/or from harvestable parts of a plant according to item 124.
   Item 126: Use of a nucleic acid encoding a UGE polypeptide enhancing yield-related traits preferably increased yield, more preferably increased seed yield relative to control plants.

According to a further embodiment of the present invention, there is also provided an isolated polypeptide comprising a polypeptide sequence selected from the group consisting of:

(i) an amino acid sequence encoded by any one of the nucleic acids represented by Item 116;
(ii) an amino acid sequence having, in increasing order of preference, at least 50%, 55%, 60%, 65%, 70%, 75%, 80%, 85%, 90%, 95%, 96%, 97%, 98%, 99% or more sequence identity to the amino acid sequence of (i);
(iii) derivatives of any of the amino acid sequences given in (i) or (ii) above.

### DESCRIPTION OF FIGURES

The present invention will now be described with reference to the following figures in which:

FIG. 1 represents the domain structure of SEQ ID NO: 2 with the conserved motifs indicated by underlining and their number. The HLH domain as determined by SMART is shown in bold underlined.

FIG. 2 represents a multiple alignment of a number of bHLH6-like proteins. A dot indicates conserved residues, a colon indicates highly conserved residues and an asterisk stands for perfectly conserved residues. The highest degree of sequence conservation is found in the region of the bHLH domain and in the more N-terminal part of the protein sequence

FIG. 3 represents the binary vector for increased expression in Oryza sativa of a bHLH6-like-encoding nucleic acid under the control of a rice GOS2 promoter (pGOS2)

FIG. 4 details examples of sequences useful in performing the methods according to the present invention.

FIG. 5 represents a CLUSTAL W (1; 83) multiple sequence alignment of RrmJ/FtsJ polypeptides from Table A2.

FIG. 6 represents the binary vector for increased expression in Oryza sativa of a GRP-encoding nucleic acid sequence (wherein said GRP polypeptide is an RrmJ/FtsJ polypeptide) under the control of a rice oleosin promoter (pOleo::GRP)

FIG. 7 details examples of sequences useful in performing the methods according to the present invention.

FIG. 8 represents the binary vector for increased expression in Oryza sativa of a GRP-encoding nucleic acid sequence (wherein said GRP polypeptide is a a basic-helix-loop-helix 4 (bHLH4) polypeptide) under the control of a rice HMGB promoter (pHMGB::GRP)

FIG. 9 details examples of sequences useful in performing the methods according to the present invention.

FIG. 10 represents a multiple sequence alignment of GRP-polypeptides (wherein said GRP polypeptide is a basic-helix-loop-helix 4 (bHLH4) polypeptide). A consensus sequence, domains and motifs representative of bHLH4-polypeptides are given. In the consensus sequence the highly conserved amino acids are provided; empty blank spaces in between represent any amino acid.

FIG. 11 represents the binary vector for increased expression in Oryza sativa of a GRP-encoding nucleic acid sequence (wherein said GRP polypeptide is an isopentenyl transferase (IPT) polypeptide under the control of a rice prolamine promoter (pProl::GRP)

FIG. 12 details examples of sequences useful in performing the methods according to the present invention.

FIG. 13 represents a multiple sequence alignment of GRP-polypeptides (wherein said GRP polypeptide is a isopentenyl transferase (IPT) polypeptide). A consensus sequence, domains and motifs representative of IPT-polypeptides are given. In the consensus sequence the highly conserved amino acids are provided; empty blank spaces in between represent any amino acid.

FIG. 14 represents the amino acid sequence and domain structure of SEQ ID NO: 169. The two conserved B-box domains in SEQ ID NO: 169 are indicated by dotted line (B-box domain 1) and by a continuous line (B-box domain 2). The four cysteine residues in each B-box domain with putative potential to bind zinc ions are indicated by boxes.

FIG. 15 represents a multiple sequence alignment of STO polypeptides. Position of the conserved amino acid residues and domains corresponding to those described in FIG. 14 are indicated. A consensus sequence representative of STO polypeptides is given. In the consensus sequence the highly conserved amino acids are provided; empty blank spaces in between represent any amino acid.

FIG. 16 shows phylogenetic tree of STO polypeptides. In the phylogenetic tree SEQ ID NO: 169 is represented by Os04g0540200.

FIG. 17 represents the binary vector for increased expression in Oryza sativa of an STO-encoding nucleic acid under the control of a rice GOS2 promoter (pGOS2)

FIG. 18 details examples of sequences useful in performing the methods according to the present invention.

FIG. 19 represents the amino acid sequence of SEQ ID NO: 225 wherein relevant functional domains and conserved motifs are indicated. Epimerase domain (boxed region), Motif 8 (capital letters in bold, underlined with an interrupted line), Motif 9 (small letters in bold), Motif 10 (capital letter in bold), Motif 11 (capital letters underlined by dots) and Motif 12 (capital letters underlined with a continuous line) are indicated.

FIG. 20 represents a multiple protein alignment of the UGE polypeptides of Table A6. A consensus sequences is given. Position of the conserved domain and motifs is indicated.

FIG. 21 shows a phylogenetic tree of the of the UGE polypeptides of Table A6.

FIG. 22 represents the binary vector for increased expression in Oryza sativa of a UGE-encoding nucleic acid under the control of a rice GOS2 promoter (pGOS2).

FIG. 23 details examples of sequences useful in performing the methods according to the present invention.

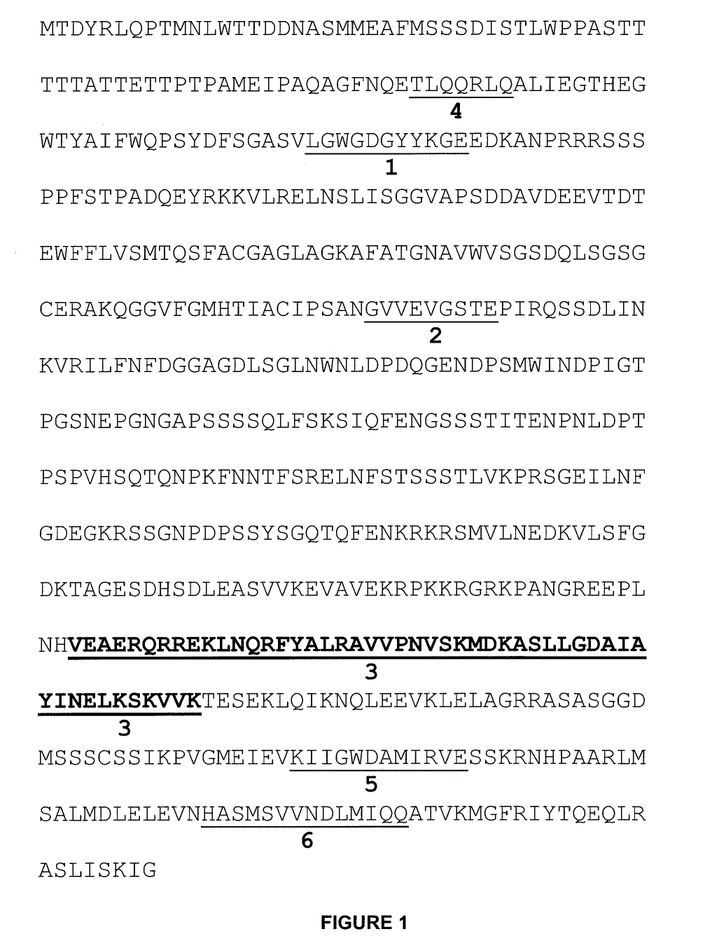

### EXAMPLES

The present invention will now be described with reference to the following examples, which are by way of illustration alone. The following examples are not intended to completely define or otherwise limit the scope of the invention.

DNA manipulation: unless otherwise stated, recombinant DNA techniques are performed according to standard protocols described in (Sambrook (2001) Molecular Cloning: a laboratory manual, 3rd Edition Cold Spring Harbor Laboratory Press, CSH, New York) or in Volumes 1 and 2 of Ausubel et al. (1994), Current Protocols in Molecular Biology, Current Protocols. Standard materials and methods for plant molecular work are described in Plant Molecular Biology Labfax (1993) by R. D. D. Croy, published by BIOS Scientific Publications Ltd (UK) and Blackwell Scientific Publications (UK).

### Example 1

### Identification of Sequences Related to the Nucleic Acid Sequence Used in the Methods of the Invention

Sequences (full length cDNA, ESTs or genomic) related to the nucleic acid sequence used in the methods of the present invention were identified amongst those maintained in the Entrez Nucleotides database at the National Center for Biotechnology Information (NCBI) using database sequence search tools, such as the Basic Local Alignment Tool (BLAST) (Altschul et al. (1990) J. Mol. Biol. 215:403-410; and Altschul et al. (1997) Nucleic Acids Res. 25:3389-3402). The program is used to find regions of local similarity between sequences by comparing nucleic acid or polypeptide sequences to sequence databases and by calculating the statistical significance of matches. For example, the polypeptide encoded by the nucleic acid used in the present invention was used for the TBLASTN algorithm, with default settings and the filter to ignore low complexity sequences set off. The output of the analysis was viewed by pairwise comparison, and ranked according to the probability score (E-value), where the score reflect the probability that a particular alignment occurs by chance (the lower the E-value, the more significant the hit). In addition to E-values, comparisons were also scored by percentage identity. Percentage identity refers to the number of identical nucleotides (or amino acids) between the two compared nucleic acid (or polypeptide) sequences over a particular length. In some instances, the default parameters may be adjusted to modify the stringency of the search. For example the E-value may be increased to show less stringent matches. This way, short nearly exact matches may be identified.

Table A provides a list of nucleic acid sequences related to the nucleic acid sequence used in the methods of the present invention.

TABLE A1
Examples of bHLH6-like polypeptides:

| | Nucleic acid | Protein |
|---|---|---|
| Plant Source | SEQ ID NO: | SEQ ID NO: |
| Arabidopsis thaliana | 1 | 2 |
| Phaseolus vulgaris | 13 | 14 |
| Catharanthus roseus | 15 | 16 |
| Solanum tuberosum | 17 | 18 |
| Pisum sativum | 19 | 20 |
| Brassica oleracea | 21 | 22 |
| Oryza sativa | 23 | 24 |
| Rheum australe | 25 | 26 |
| Zea mays | 27 | 28 |
| Physcomitrella patens | 29 | 30 |
| Lycopersicon esculentum | 31 | 32 |
| Populus trichocarpa | 33 | 34 |
| Triticum aestivum | 35 | 36 |
| Populus sp | 37 | 38 |
| Vitis vinifera | 39 | 40 |
| Oryza sativa | | 41 |

TABLE A2a
Examples of GRP polypeptides, wherein the GRP polypeptides are RrmJ/FtsJ
polypeptides:

| | | SEQ ID NO | SEQ ID NO | | Public database |
|---|---|---|---|---|---|
| | Source | nucleic acid | polypeptide | | accession |
| Name | organism | sequence | sequence | Status | number |
| Nicta_RrmJ/ | Nicotiana | 57 | 58 | Partial | |
| FtsJ partial | tabacum | | | | |
| Nicta_RrmJ/ | Nicotiana | 59 | 60 | Full | EB446219, |
| FtsJ | tabacum | | | length | EB448450.1 |
| Arath_RrmJ/ | Arabidopsis | 61 | 62 | Full | AY087952.1 |
| FtsJ | thaliana | | | length | |
| Lyces_RrmJ/ | Lycopersicon | 63 | 64 | Full | BT013964 |
| FtsJ | esculentum | | | length | |
| Medtr_RrmJ/ | Medicago | 65 | 66 | Full | AC149135.2 |
| FtsJ | truncatula | | | length | |
| Orysa_RrmJ/ | Oryza sativa | 67 | 68 | Full | AK103054 |
| FtsJ | | | | length | |
| Osta_RrmJ/FtsJ | Ostreococcus | 69 | 70 | Full | XM_001418019 |
| | lucimarinus | | | length | |
| | CCE9901 | | | | |
| Poptr_RrmJ/ | Populus | 71 | 72 | Full | contig of |
| FtsJ | tremuloides | | | length | CX183550.1 |
| | | | | | DT504907.1 |
| Sacof_RrmJ/ | Saccharum | 73 | 74 | Full | contig of |
| FtsJ | officinarum | | | length | CA279558.1 |
| | | | | | CF576902.1 |
| Triae_RrmJ/ | Triticum aestivum | 75 | 76 | Full | DR740715 |
| FtsJ | | | | length | |
| Apime_RrmJ/ | Apis mellifera | 77 | 78 | Full | XM_392223 |
| FtsJ | | | | length | |
| Caeel_RrmJ/ | Caenorhabditis | 79 | 80 | Full | NM_065442 |
| FtsJ | elegans | | | length | |
| Danre_RrmJ/ | Danio rerio | 81 | 82 | Full | BC085449 |
| FtsJ | | | | length | |
| Homsa_RrmJ/ | Homo sapiens | 83 | 84 | Full | AK024023 |
| FtsJ | | | | length | |
| Horvu_FtsJ | Hordeum vulgare | 92 | 93 | Full | |
| | | | | length | |
| Sorbi_FtsJ | Sorghum bicolor | 94 | 95 | Full | |
| | | | | length | |

TABLE A2b
Examples of GRP polypeptides, wherein the GRP polypeptides are
RrmJ/FtsJ polypeptides:

| | | SEQ ID NO | SEQ ID NO | |
|---|---|---|---|---|
| | Source | nucleic acid | polypeptide | |
| Name | organism | sequence | sequence | Status |
| Brana_FtsJ | Brassica | 96 | 97 | Full length |
| | napus | | | |
| Glyma_FtsJ | Glycine | 98 | 99 | Full length |
| | max | | | |
| Zeama_FtsJ | Zea mays | 100 | 101 | Full length |
| Tager_FtsJ_partial | Tagetes | | | Partial |
| | erecta | | | |

TABLE A3a
Examples of GRP polypeptides, wherein the GRP polypeptides are bHLH4
polypeptides:

| | | SEQ ID | SEQ ID NO | |
|---|---|---|---|---|
| | | NO nucleic acid | polypeptide | Public database |
| Name | Source organism | sequence | sequence | accession number |
| Arath_bHLH4 | Arabidopsis | 104 | 105 | |
| | thaliana | | | |
| Arath_bHLH4 | Arabidopsis | 109 | 110 | |
| II | thaliana | | | |
| Brana_bHLH4 | Brassica napus | 111 | 112 | |
| II | | | | |
| Lotja_bHLH4 | Lotus japonicus | 113 | 114 | |
| Lyces_bHLH4 | Lycopersicon | 115 | 116 | |
| | esculentum | | | |
| Vitvi_bHLH4 | Vitis vinifera | 117 | 118 | contig like |
| | | | | VV78X118066.5 |
| | | | | AM475703.2 |
| Zeama_bHLH4 | Zea mays | 119 | 120 | full length nucleic |
| | | | | acid sequence |
| | | | | contig of |
| | | | | AF061107 and |
| | | | | EE190449 |

TABLE A3b
Examples of GRP polypeptides, wherein the GRP polypeptides are
bHLH4 polypeptides:

| | | | | Public |
|---|---|---|---|---|
| | | SEQ ID NO | SEQ ID NO | database |
| | Source | nucleic acid | polypeptide | accession |
| Name | organism | sequence | sequence | number |
| Brana_bHLH4 | Brassica | 121 | 122 | |
| | napus | | | |
| Glyma_bHLH4 | Glycine max | 123 | 124 | |
| Glyma_bHLH4 | Glycine max | 125 | 126 | |
| II | | | | |
| Horvu_bHLH4 | Hordeum | 127 | 128 | |
| | vulgare | | | |
| Zeama_bHLH4 | Zea mays | 129 | 130 | |
| II | | | | |

TABLE A4
Examples of GRP polypeptides, wherein the GRP polypeptides are IPT
polypeptides:

| | | SEQ ID NO | SEQ ID NO |
|---|---|---|---|
| | Source | nucleic acid | polypeptide |
| Name | organism | sequence | sequence |
| Arath_IPT | Arabidopsis | 131 | 132 |
| | thaliana | | |
| Arath_IPT4 | Arabidopsis | 136 | 137 |
| | thaliana | | |
| Arath_IPT6 | Arabidopsis | 138 | 139 |
| | thaliana | | |
| Humlu_IPT | Humulus lupunus | 140 | 141 |
| like | | | |
| Lotja_IPT | Lotus japonica | 142 | 143 |
| like | | | |
| Medtr_IPT | Medicago | 144 | 145 |
| like | truncatula | | |
| Moral_IPT | Morus alba | 146 | 147 |
| like | | | |
| Orysa_IPT | Oryza sativa | 148 | 149 |
| like | | | |
| Pethy_IPT | Petunia hybrida | 150 | 151 |
| like | | | |
| Poptr_IPT | Populus | 152 | 153 |
| like | tremuloides | | |
| Poptr_IPT | Populus | 154 | 155 |
| like II | tremuloides | | |
| Poptr_IPT | Populus | 156 | 157 |
| like III | tremuloides | | |
| Poptr_IPT | Populus | 158 | 159 |
| like IV | tremuloides | | |
| Poptr_IPT | Populus | 160 | 161 |
| like V | tremuloides | | |
| Poptr_IPT | Populus | 162 | 163 |
| like VI | tremuloides | | |
| Vitvi_IPT like | Vitis vinifera | 164 | 165 |
| Zeama_IPT | Zea mays | 166 | 167 |
| like | | | |

TABLE A5a
Examples of STO nucleic acids a and polypeptides:

| | | Nucleic acid | Protein |
|---|---|---|---|
| Name | Plant Source | SEQ ID NO: | SEQ ID NO: |
| Os04g0540200 | Oryza sativa | 168 | 169 |
| cds3887 | | | |
| Os01g0202500 | Oryza sativa | 170 | 171 |
| Os02g0606200 | Oryza sativa | 172 | 173 |
| Os02g0646200 | Oryza sativa | 174 | 175 |
| Os04g049300 | Oryza sativa | 176 | 177 |
| Os05g0204600 | Oryza sativa | 178 | 179 |
| Os06g0152200 | Oryza sativa | 180 | 181 |
| Os06g071300 | Oryza sativa | 182 | 183 |
| Os09g0527900 | Oryza sativa | 184 | 185 |
| Os12g0209200 | Oryza sativa | 186 | 187 |
| SO_STO | Saccharum officinarum | 188 | 189 |
| AT1G06040_spliced variant1 | Arabidopsis thaliana | 190 | 191 |
| AT1G06040_spliced variant2 | Arabidopsis thaliana | 192 | 193 |
| AT1G75540 | Arabidopsis thaliana | 194 | 195 |
| AT1G78600 | Arabidopsis thaliana | 196 | 197 |
| AT2G31380 | Arabidopsis thaliana | 198 | 199 |
| AT4G10240 | Arabidopsis thaliana | 200 | 201 |
| AT4G39070 | Arabidopsis thaliana | 202 | 203 |
| Mt_ABO84497 | Medicago truncatula | 204 | 205 |
| LE_gi\|45544865 | Lycopersicul esculentum | 206 | 207 |
| ST_gi\|76160970 | Solanum tuberosum | 208 | 209 |
| PP_STO LIKE | populus trichoparcca | 210 | 211 |
| VV_CAN72879 | Vitis vinifera | 212 | 213 |
| GM_gi\|78173056 | Glycine max | 214 | 215 |

TABLE A5b
Examples of STO nucleic acids and polypeptides:

| | | Nucleic acid | Protein SEQ |
|---|---|---|---|
| Name | Plant Source | SEQ ID NO: | ID NO: |
| Zeama_STO | Zea mays | 222 | 223 |

TABLE A6
Examples of UGE nucleic acids and polypeptides:

| | | Nucleic | Protein |
|---|---|---|---|
| | | acid | SEQ |
| Name | Source organism | SEQ ID NO: | ID NO: |
| OS_UGE2 | Oryza sativa | 224 | 225 |
| Os04g0618200 | Oryza sativa | 226 | 227 |
| Os05g0595100 | Oryza sativa | 228 | 229 |
| Os07g0139400 | Oryza sativa | 230 | 231 |
| Os08g0374800 | Oryza sativa | 232 | 233 |
| Os09g0323000 | Oryza sativa | 234 | 235 |
| Os09g0504000 | Oryza sativa | 236 | 237 |
| Os09g0526700 | Oryza sativa | 238 | 239 |
| AT1G12780_UGE1 | Arabidopsis thaliana | 240 | 241 |
| AT1G63180_UGE3 | Arabidopsis thaliana | 242 | 243 |
| AT1G64440_UGE4 | Arabidopsis thaliana | 244 | 245 |
| AT4G10960_UGE5 | Arabidopsis thaliana | 246 | 247 |
| AT4G23920_UGE2 | Arabidopsis thaliana | 248 | 249 |
| Pt_lcl\|scaff_XVI.140 | Populus trichocarpa | 250 | 251 |
| Pt_lcl\|scaff_XI.1329 | Populus trichocarpa | 252 | 253 |
| Pt_lcl\|scaff_VI.203 | Populus trichocarpa | 254 | 255 |
| Pt_lcl\|scaff_III.961 | Populus trichocarpa | 256 | 257 |
| Pt_lcl\|scaff_III.1133 | Populus trichocarpa | 258 | 259 |
| Pt_lcl\|scaff_I.773 | Populus trichocarpa | 260 | 261 |
| Pt_lcl\|scaff_I.2996 | Populus trichocarpa | 262 | 263 |
| Pt_lcl\|scaff_5422 | Populus trichocarpa | 264 | 265 |
| Ot08g015500 | Ostreococcus tauri | 266 | 267 |
| SC_GAL10_P04397 | Ostreococcus tauri | 268 | 269 |
| Ec_GALE_AAO37702 | Escherichia coli | 270 | 271 |

TABLE A6b
Examples of UGE nucleic acids and polypeptides:

| | | Nucleic acid | Protein SEQ |
|---|---|---|---|
| Name | Source organism | SEQ ID NO: | ID NO: |
| Brana_UGE I | Brassica napus | 281 | 282 |
| Brana_UGE II | Brassica napus | 283 | 284 |
| Zeama_UGE I | Zea mays | 285 | 286 |
| Zeama_UGE II | Zea mays | 287 | 288 |
| Zeama_UGE III | Zea mays | 289 | 290 |
| Zeama_UGE IV | Zea mays | 291 | 292 |
| Zeama_UGE V | Zea mays | 293 | 294 |
| Zeama_UGE VI | Zea mays | 295 | 296 |
| Zeama_UGE VII | Zea mays | 297 | 298 |
| Zeama_UGE VIII | Zea mays | 299 | 300 |
| Orysa_UGE | Oryza sativa | 301 | 302 |
| Glyma_UGE I | Glycine max | 303 | 304 |
| Glyma_UGE II | Glycine max | 305 | 306 |
| Glyma_UGE III | Glycine max | 307 | 308 |
| Glyma_UGE IV | Glycine max | 309 | 310 |
| Glyma_UGE V | Glycine max | 311 | 312 |
| Helan_UGE | Helianthus annuus | 313 | 314 |
| Zeama_UGE IX | Zea mays | 315 | 316 |
| Zeama_UGE X | Zea mays | 317 | 318 |
| Glyma_UGE VI | Glycine max | 319 | 320 |

In addition, in some instances, related sequences have tentatively been assembled and publicly disclosed by research institutions, such as The Institute for Genomic Research (TIGR). The Eukaryotic Gene Orthologs (EGO) database may be used to identify such related sequences, either by keyword search or by using the BLAST algorithm with the nucleic acid or polypeptide sequence of interest. Some of the listed sequences are partial; preferably full length sequences or functionally equivalent fragments are used in the methods of the present invention. Where only partial sequences are available from the databases, standard cloning techniques can be used by the skilled in the art for obtaining a full length gene sequence.

### Example 2

### Alignment of Polypeptide Sequences of the Invention

Alignment of polypeptide sequences was performed using the AlignX programme from the Vector NTI (Invitrogen) which is based on the popular Clustal W algorithm of progressive alignment (Thompson et al. (1997) Nucleic Acids Res 25:4876-4882; Chenna et al. (2003). Nucleic Acids Res 31:3497-3500). Default values were for the gap open penalty of 10, for the gap extension penalty of 0,1 and the selected weight matrix was Blosum 62 (if polypeptides are aligned). Minor manual editing was done to further optimise the alignment.

Alignment of bHLH6-Like Polypeptide Sequences

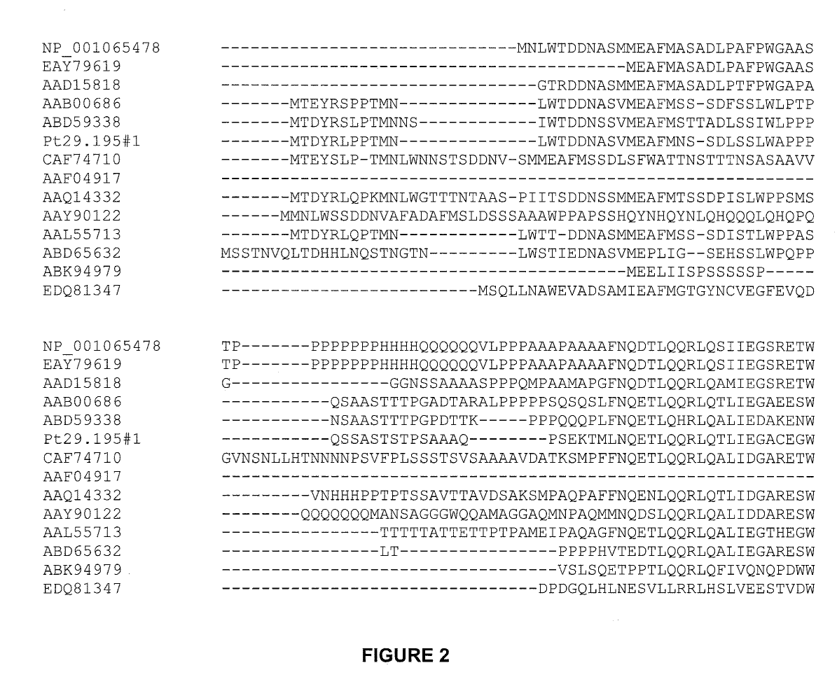

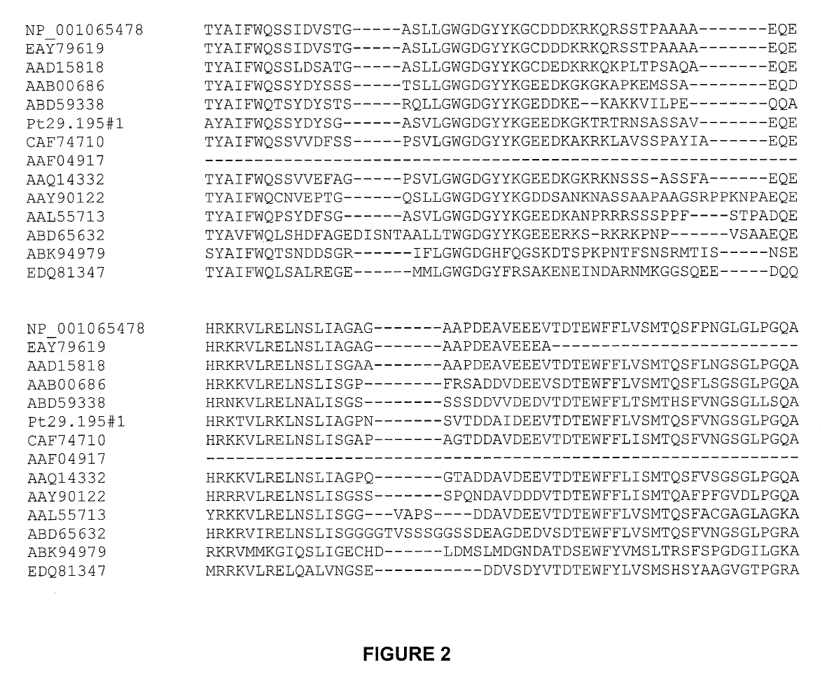

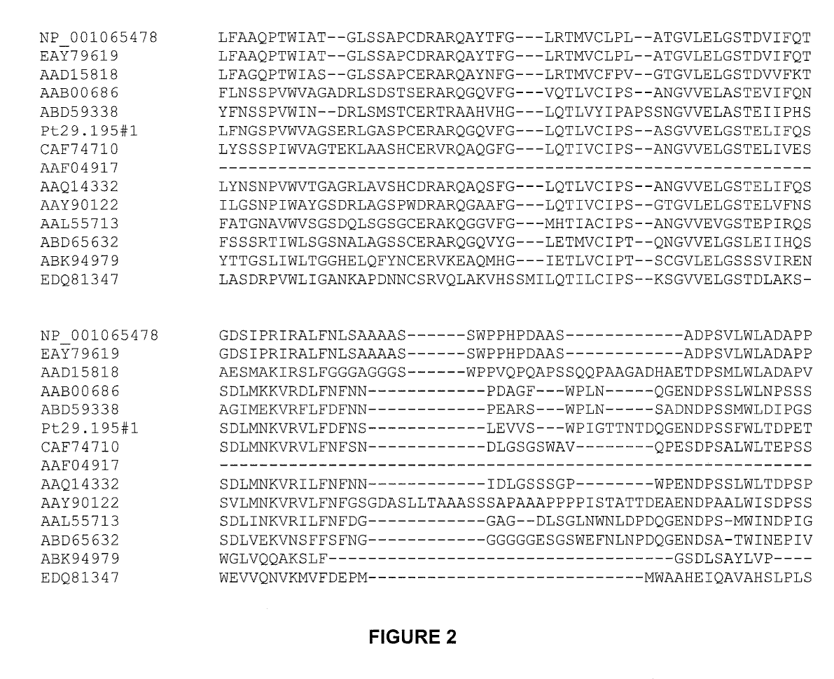

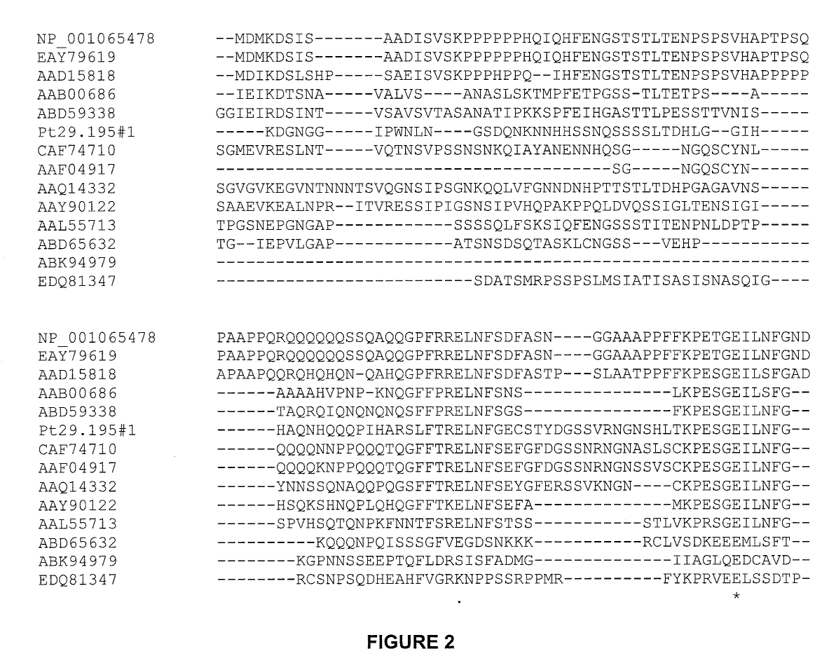

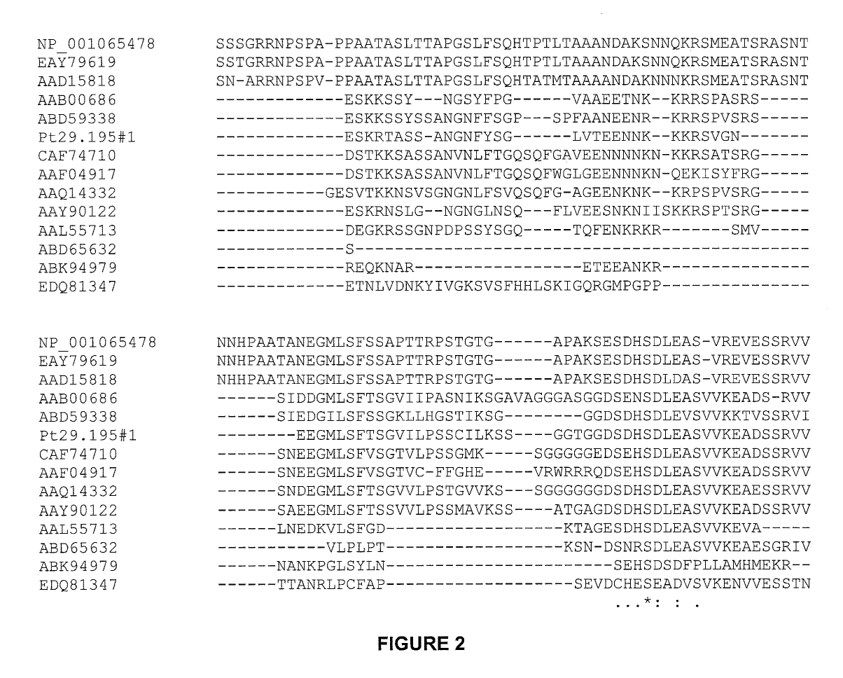

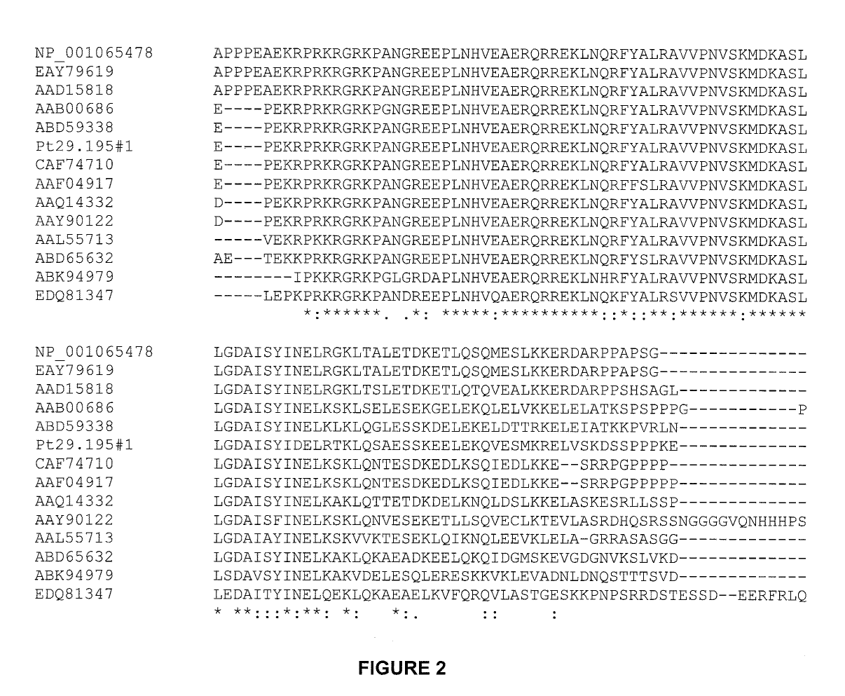

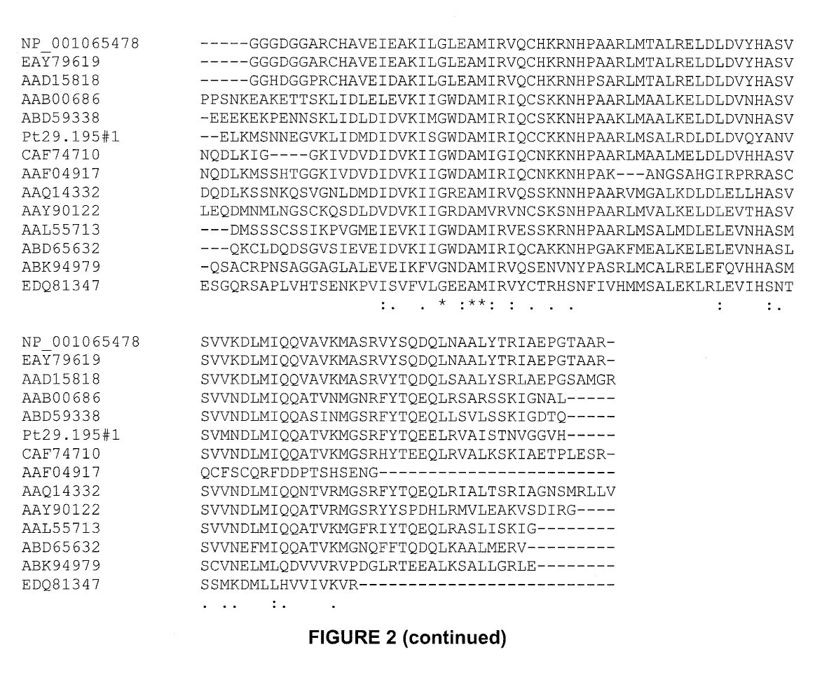

Sequence conservation among bHLH6-like polypeptides is essentially in the N-terminal domain and the C-terminal bHLH domain of the polypeptides, the central part usually being more variable in sequence length and composition. The bHLH6-like polypeptides are aligned in FIG. 2.

### Alignment of RrmJ/FtsJ Polypeptide Sequences

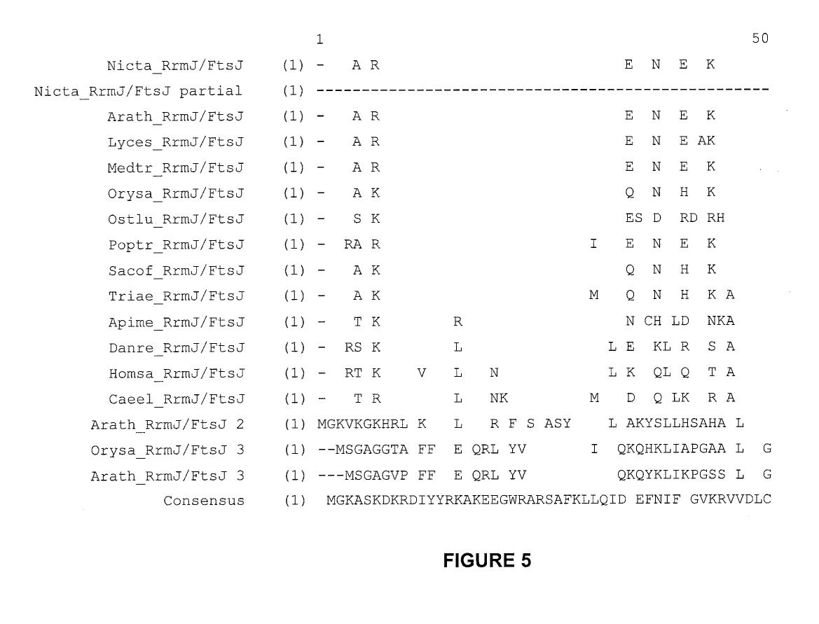

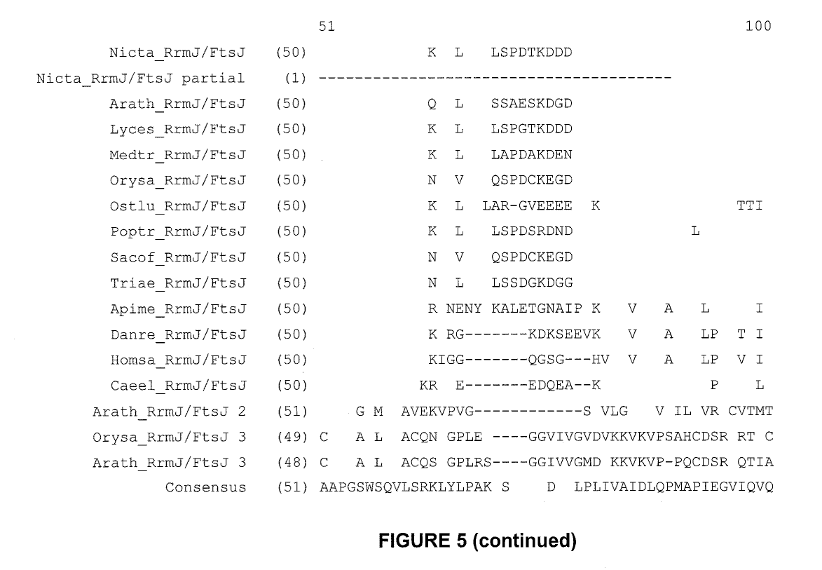

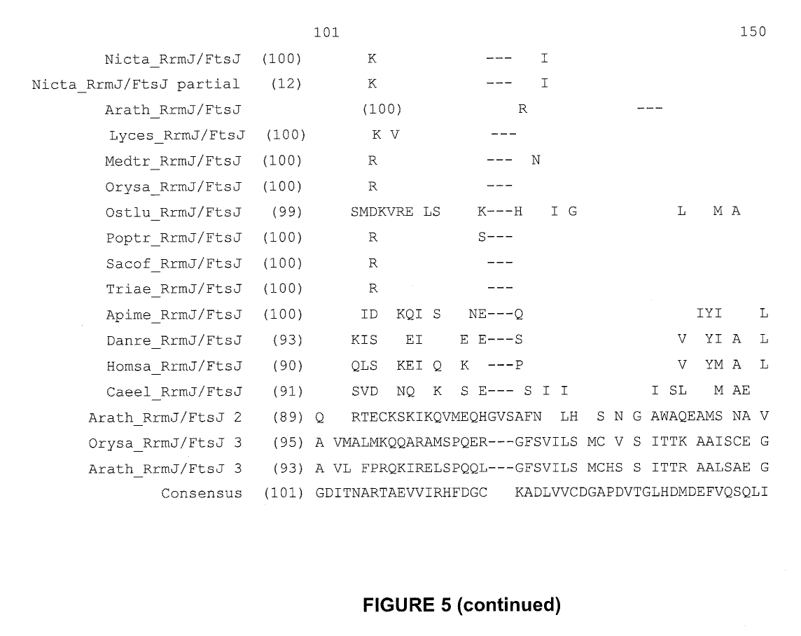

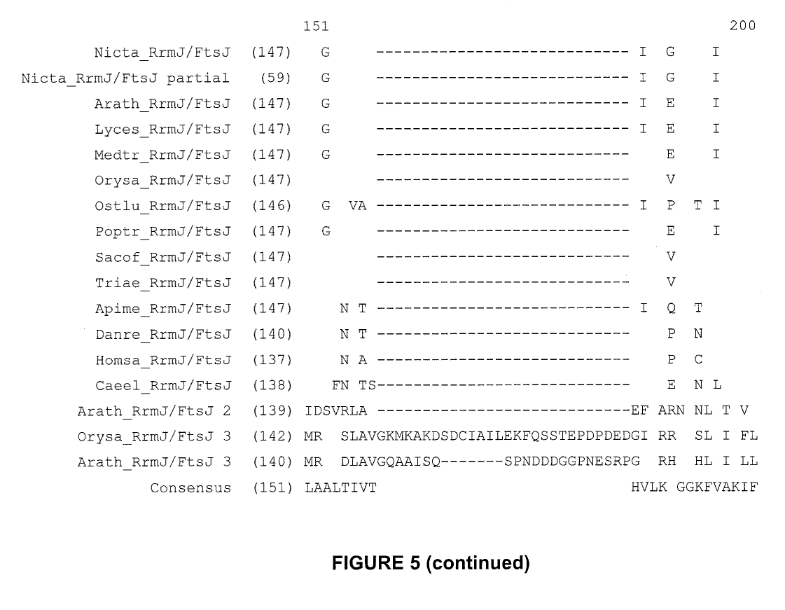

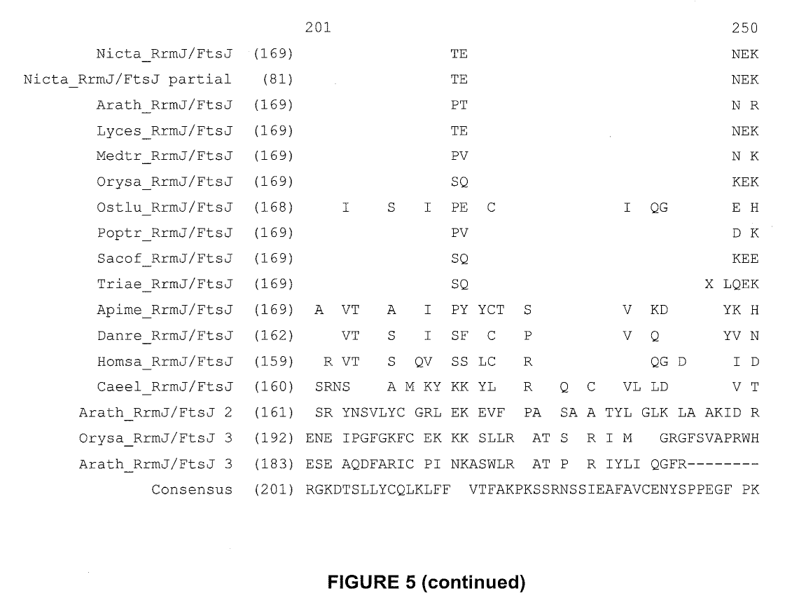

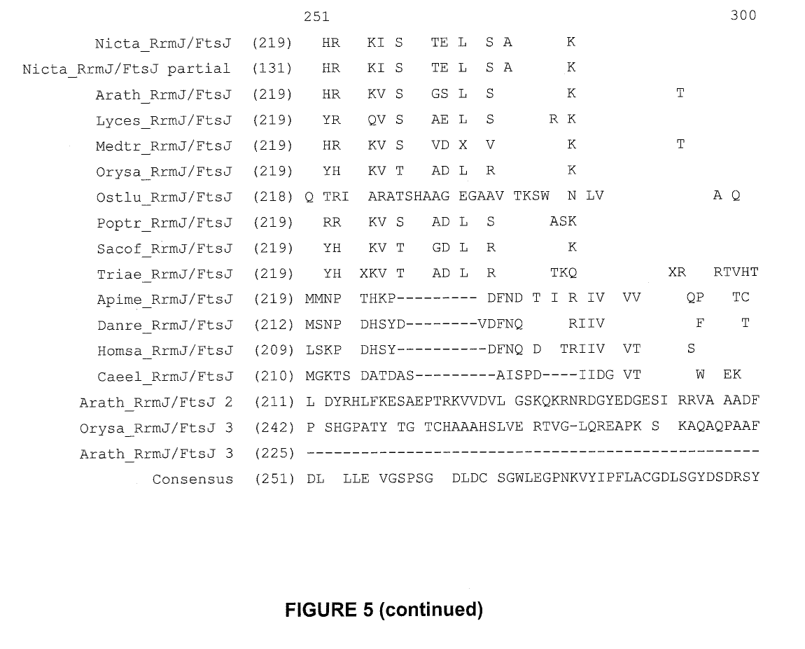

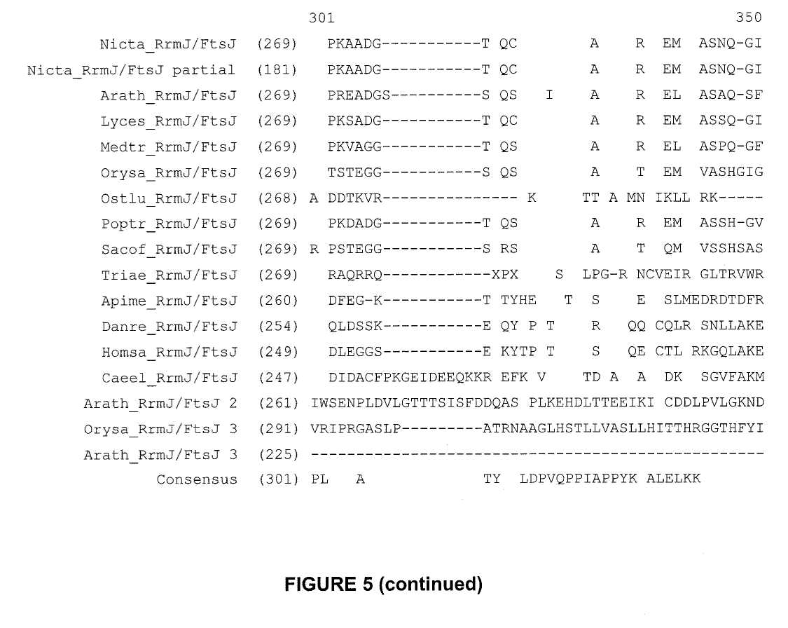

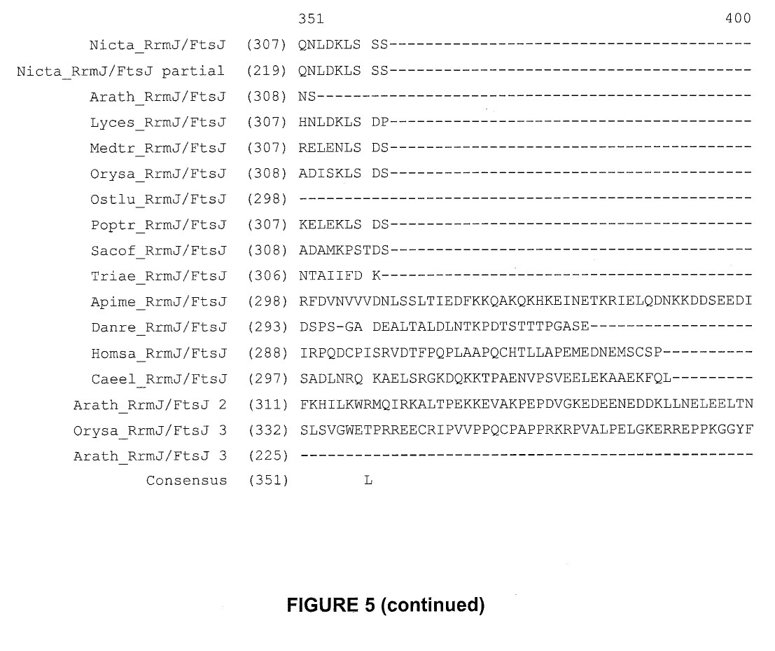

A CLUSTAL W (1; 83) multiple sequence alignment of RrmJ/FtsJ polypeptides from Table A2 is shown in FIG. 5.

A consensus sequence of bHLH4 polypeptides comprising highly conserved representative amino acids at each position is given; blanks in between given amino acids in the consensus sequence represent any amino acid.

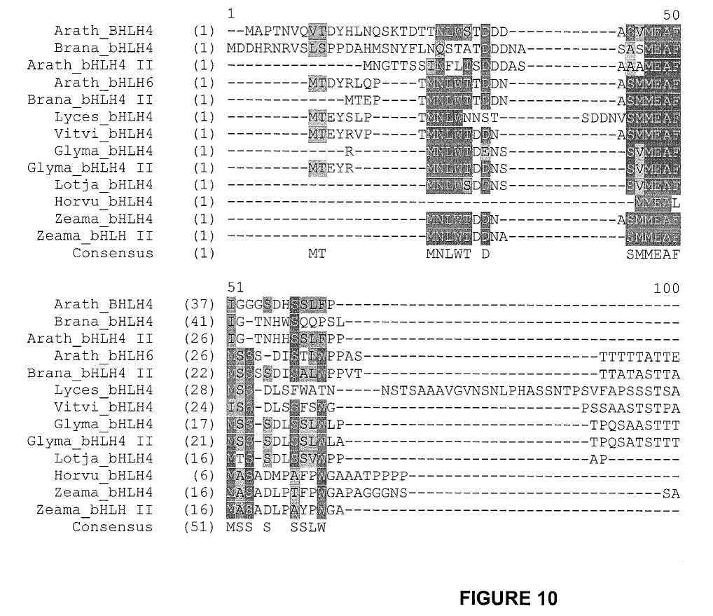

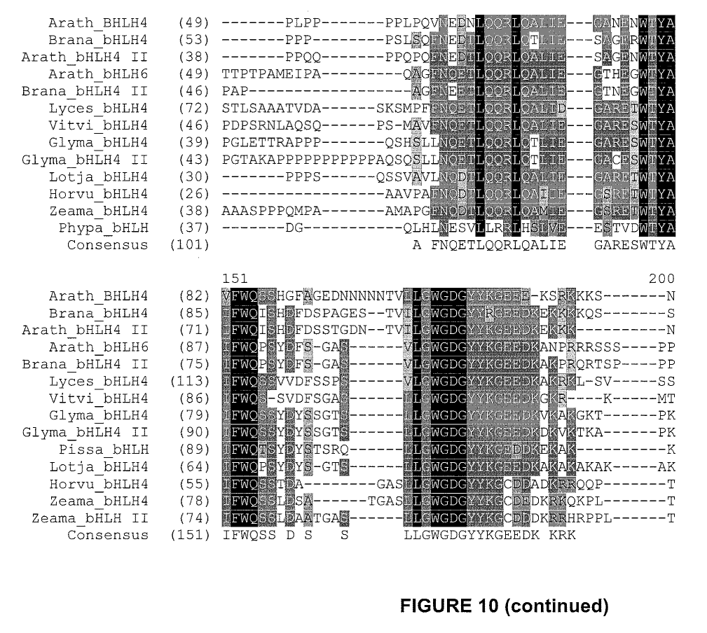

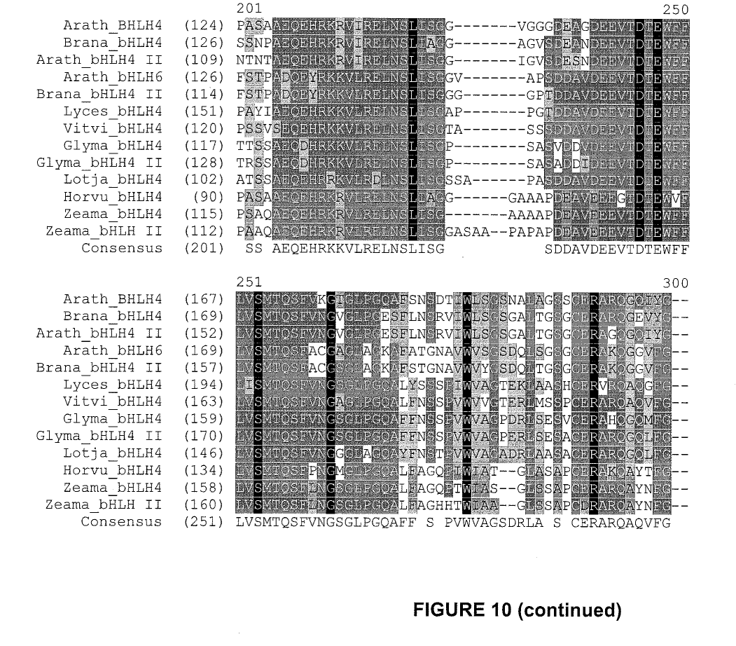

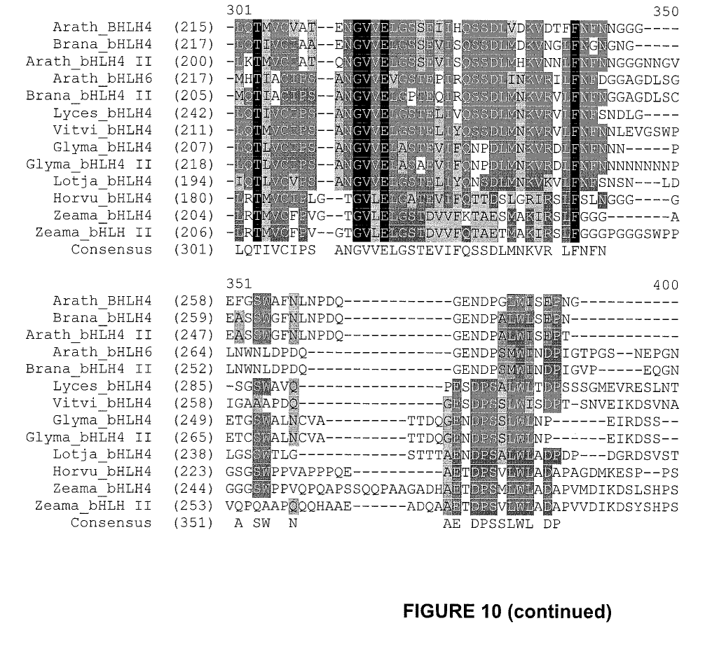

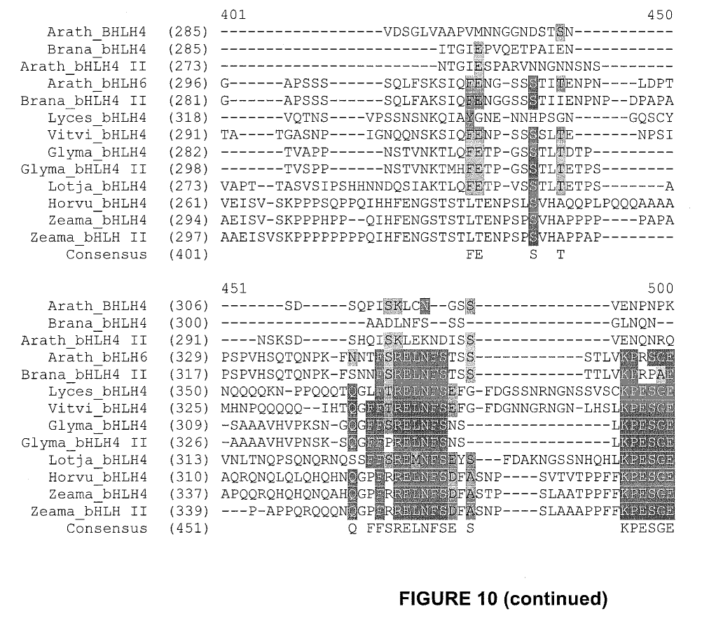

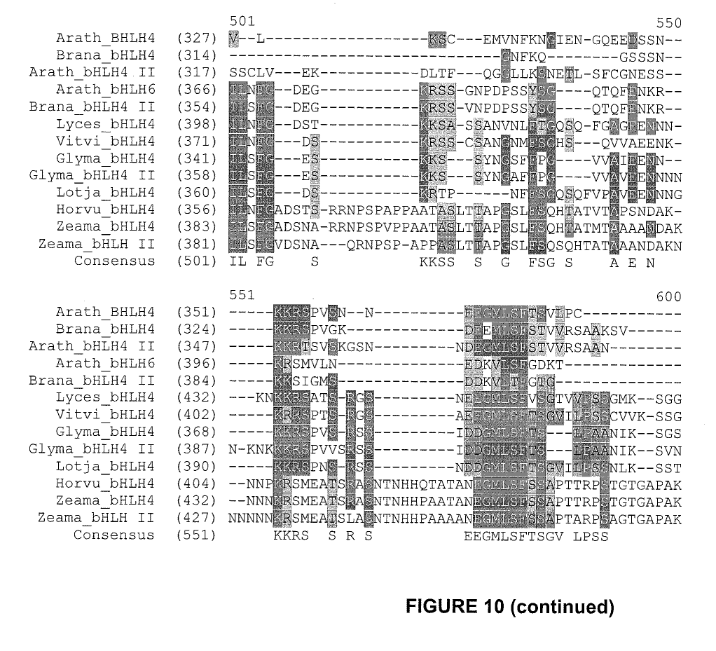

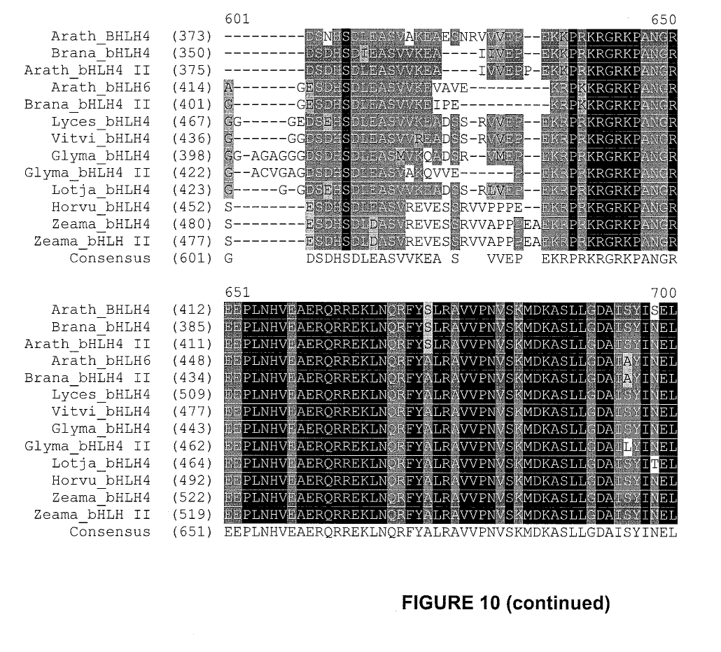

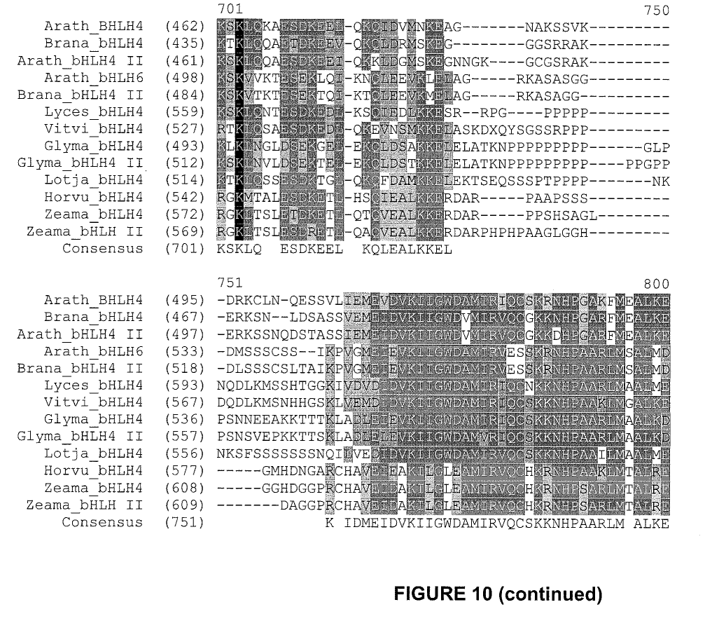

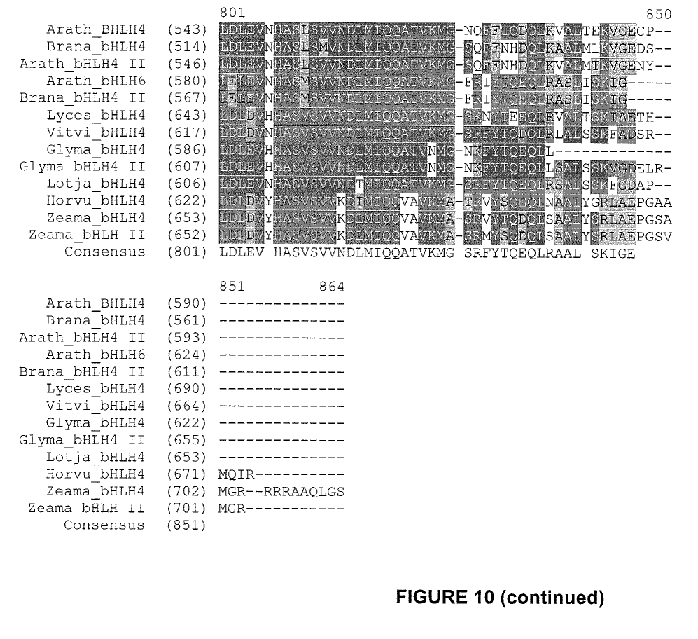

The bHLH4 polypeptides are aligned in FIG. 10.

Sequence conservation among bHLH4 polypeptides was found essentially along the bHLH domain. The consensus sequence represents highly conserved amino acid residues in bHLH4 polypeptides; blanks in the consensus sequence represent highly variable regions.

### Alignment of IPT Polypeptide Sequences

A consensus sequence of IPT polypeptides comprising highly conserved representative amino acids at each position is given; blanks in between given amino acids in the consensus sequence represent any amino acid.

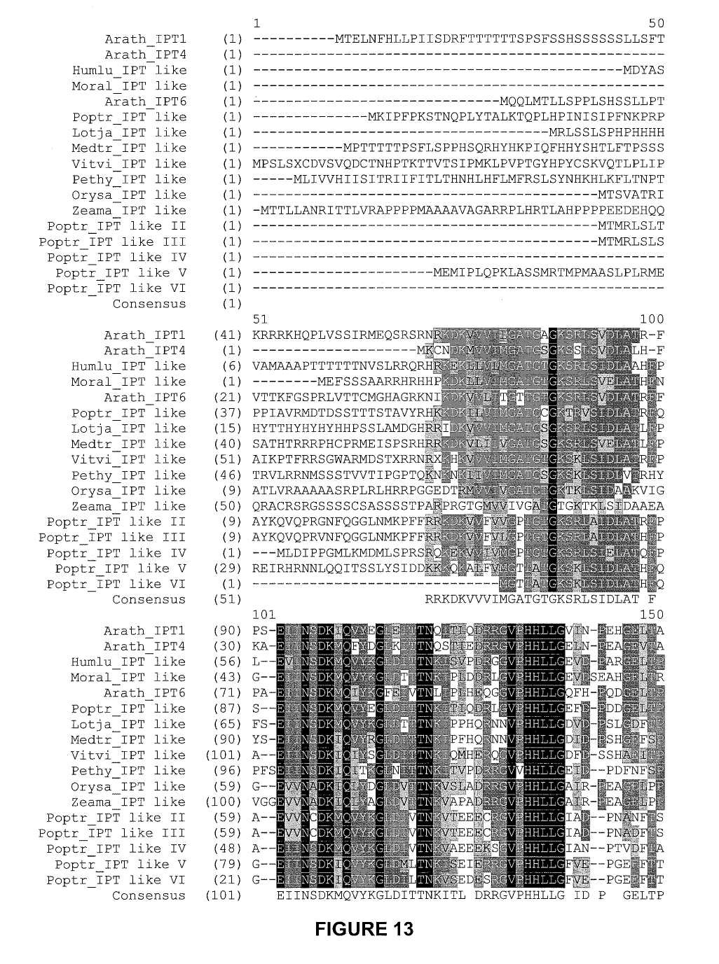

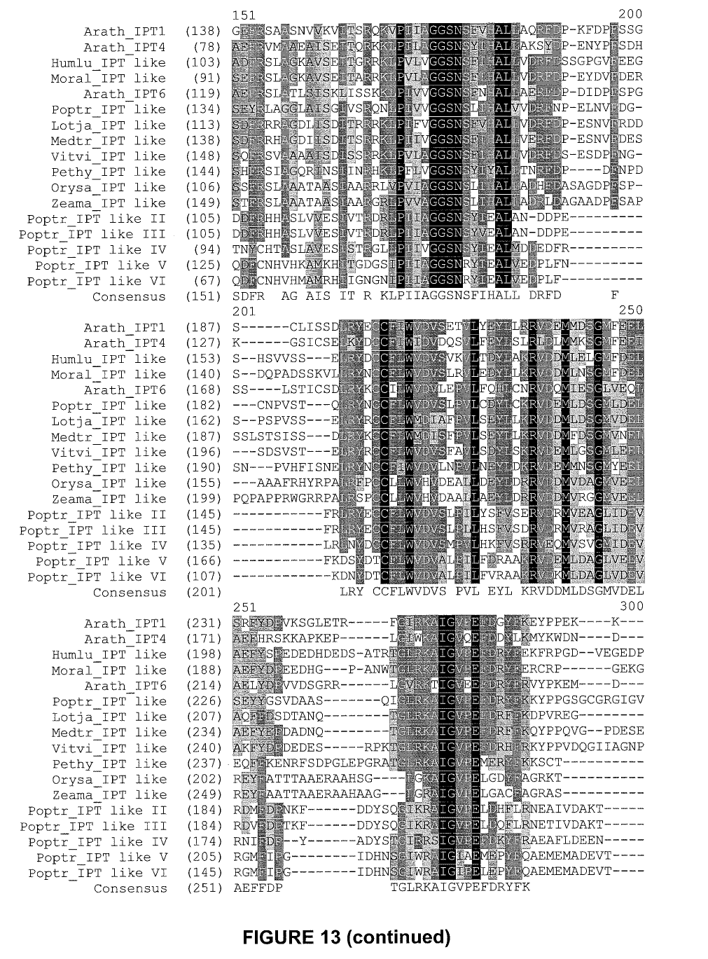

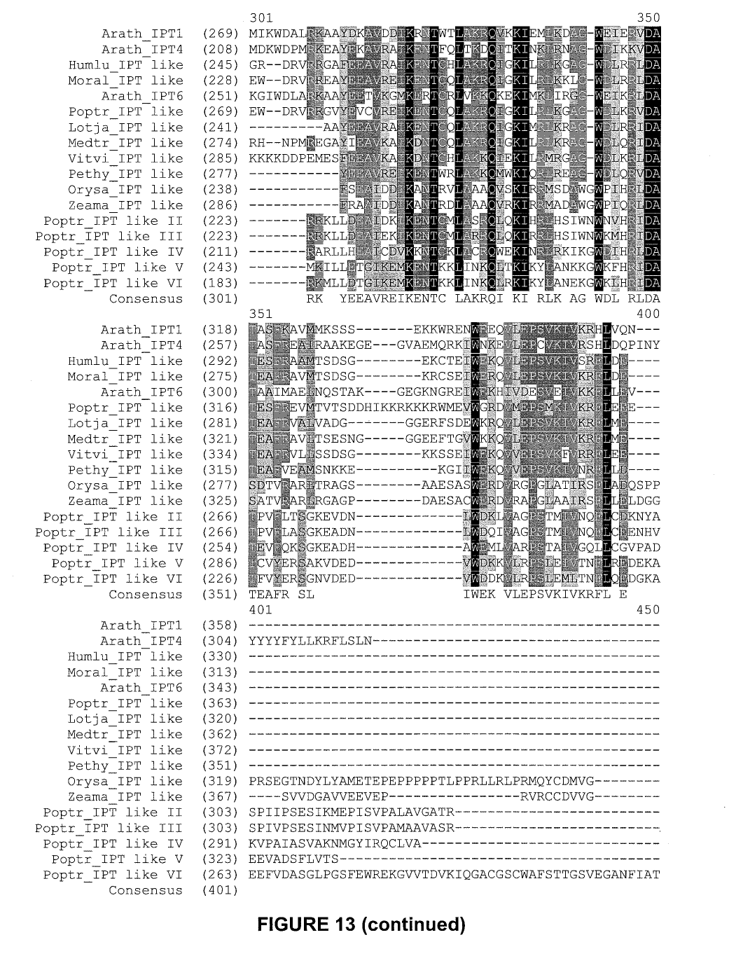

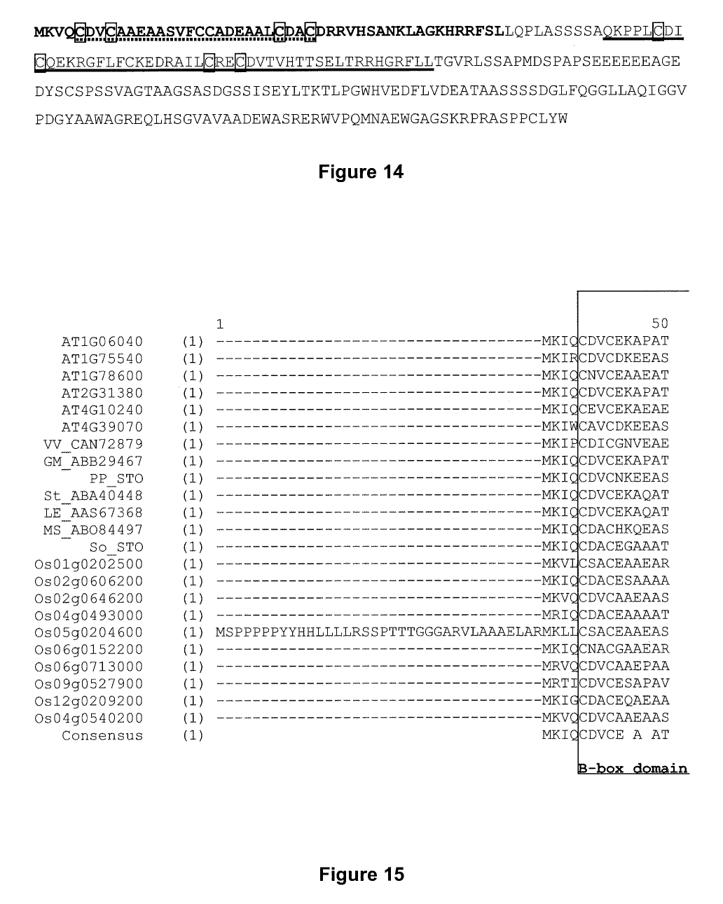

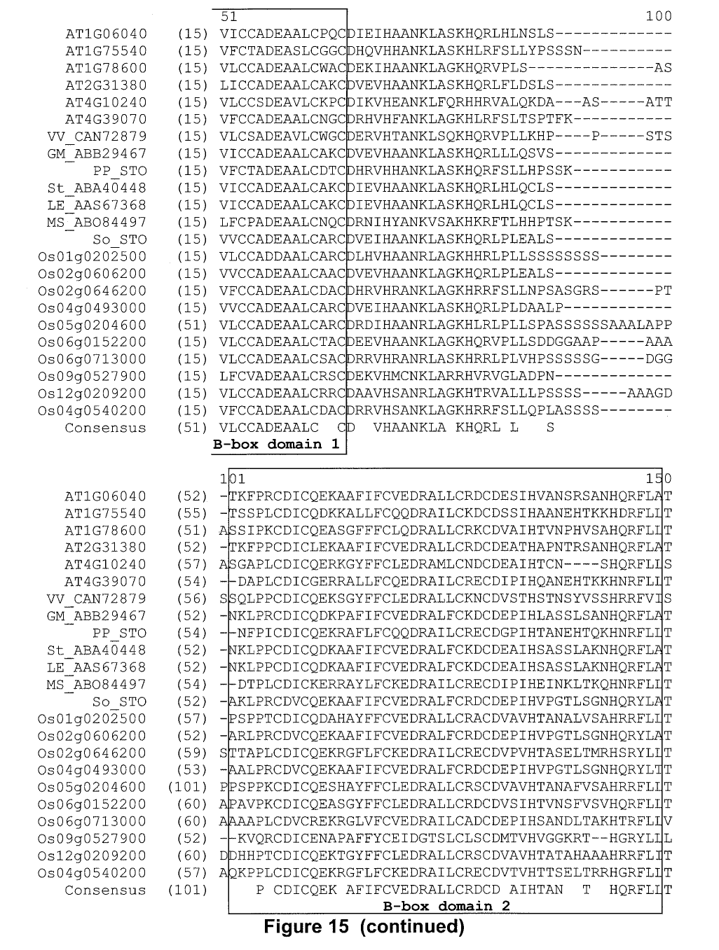

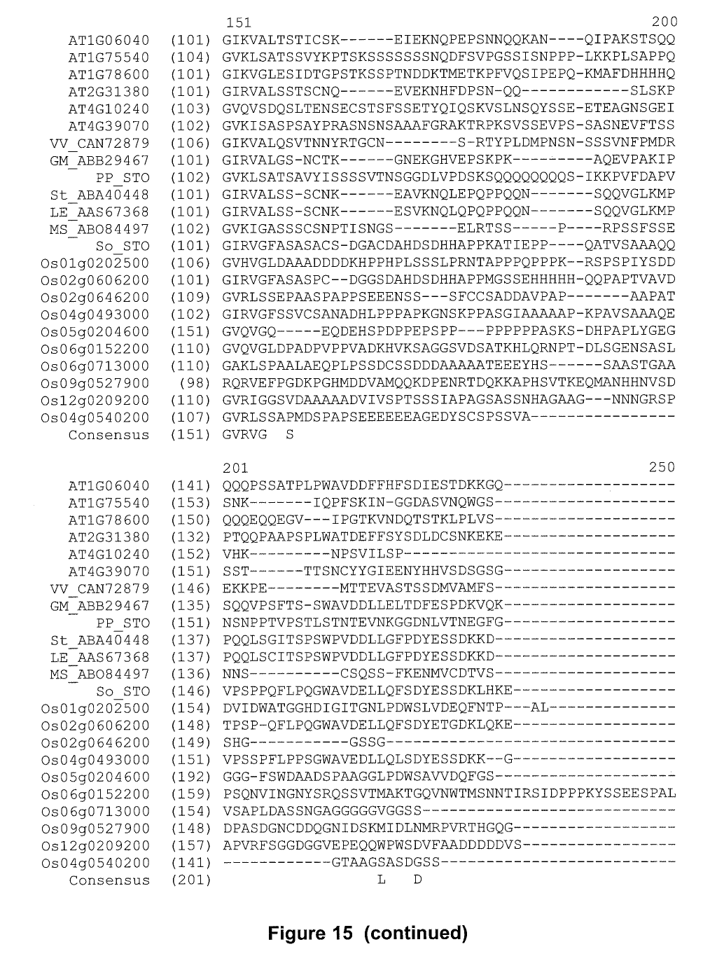

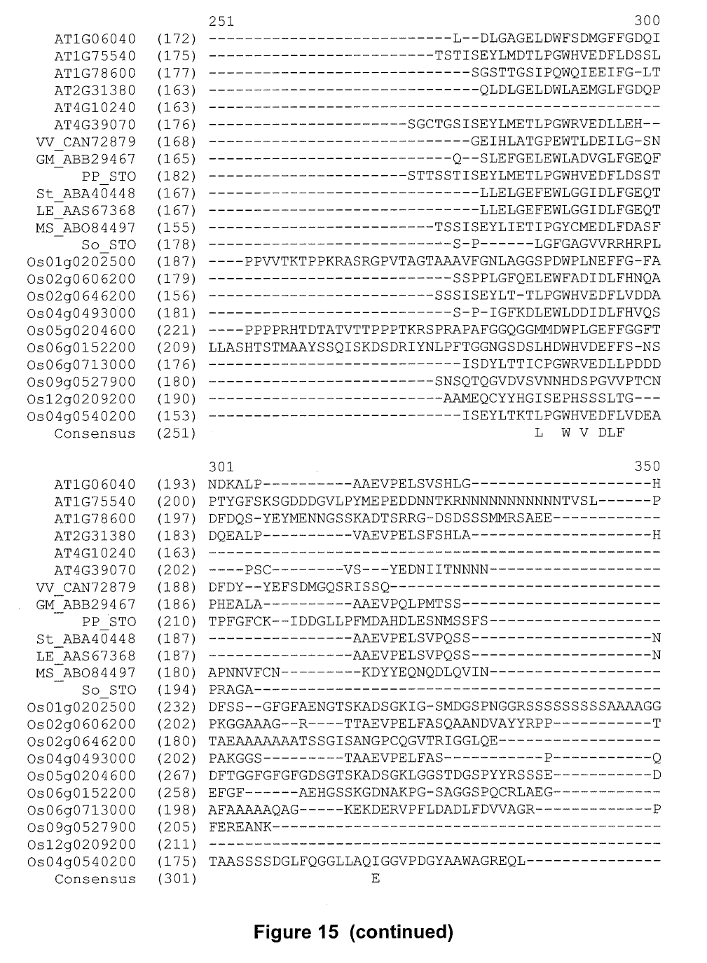

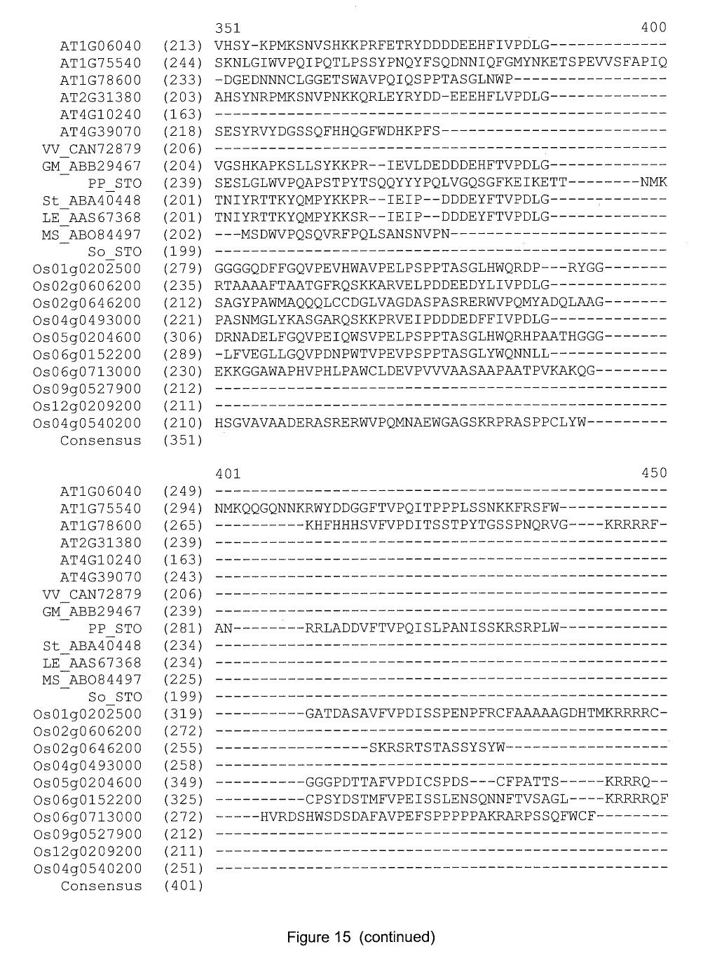

The IPT polypeptides are aligned in FIG. 13.

The consensus sequence represents highly conserved amino acid residues in IPT polypeptides; blanks in the consensus sequence represent highly variable regions.

A phylogenetic tree of GRP polypeptides is constructed using a neighbour-joining clustering algorithm as provided in the AlignX programme from Vector NTI (Invitrogen).

Sequence conservation among STO polypeptides is essentially in the N-terminal region comprising the two B-box domains. The two B-box domains are separated by a linker region of variable length, typically between 2 and 20 amino acids.

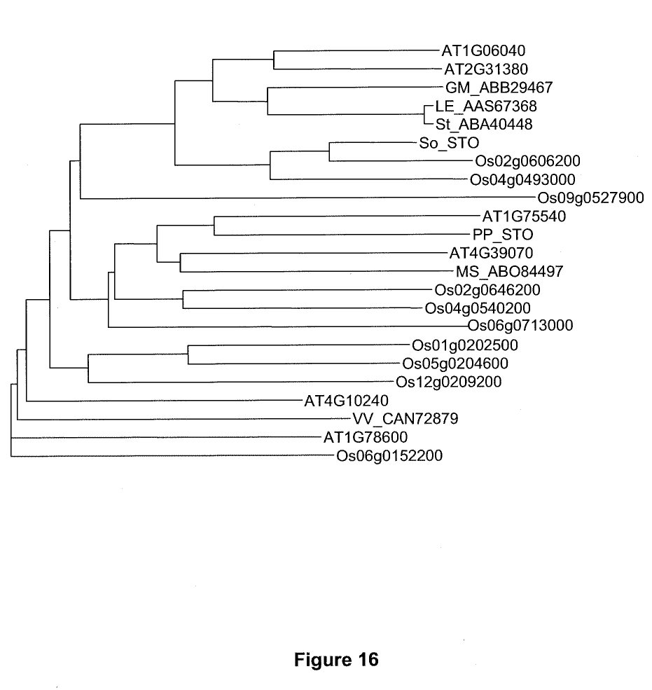

A phylogenetic tree of STO polypeptides (FIG. 16) was constructed using a neighbour-joining clustering algorithm as provided in the AlignX programme from the Vector NTI (Invitrogen).

A consensus sequence of UGE polypeptides comprising highly conserved representative amino acids at each position is given; blanks in between given amino acids in the consensus sequence represent any amino acid.

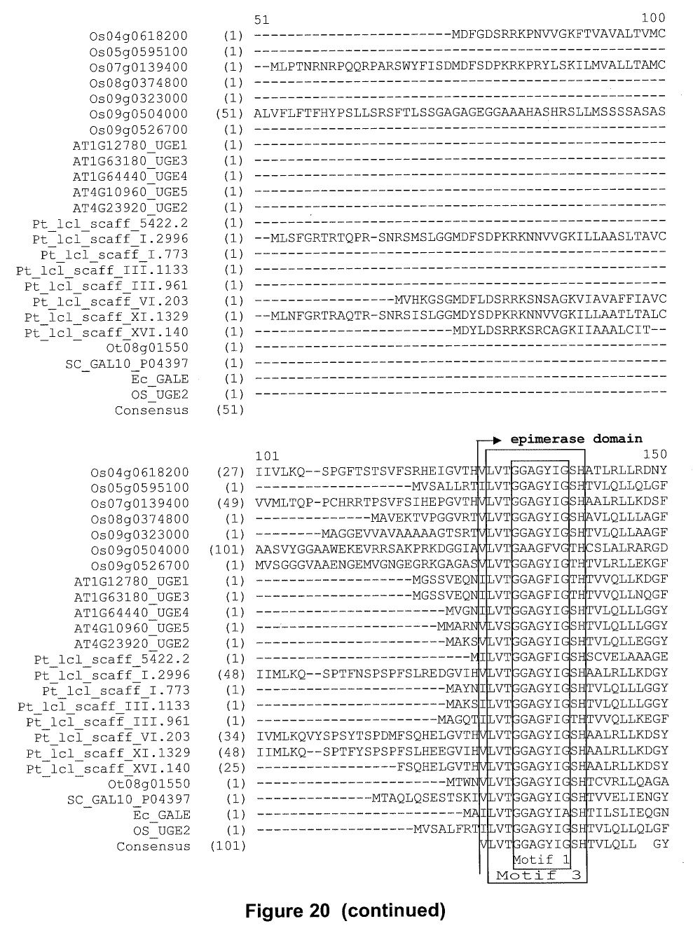

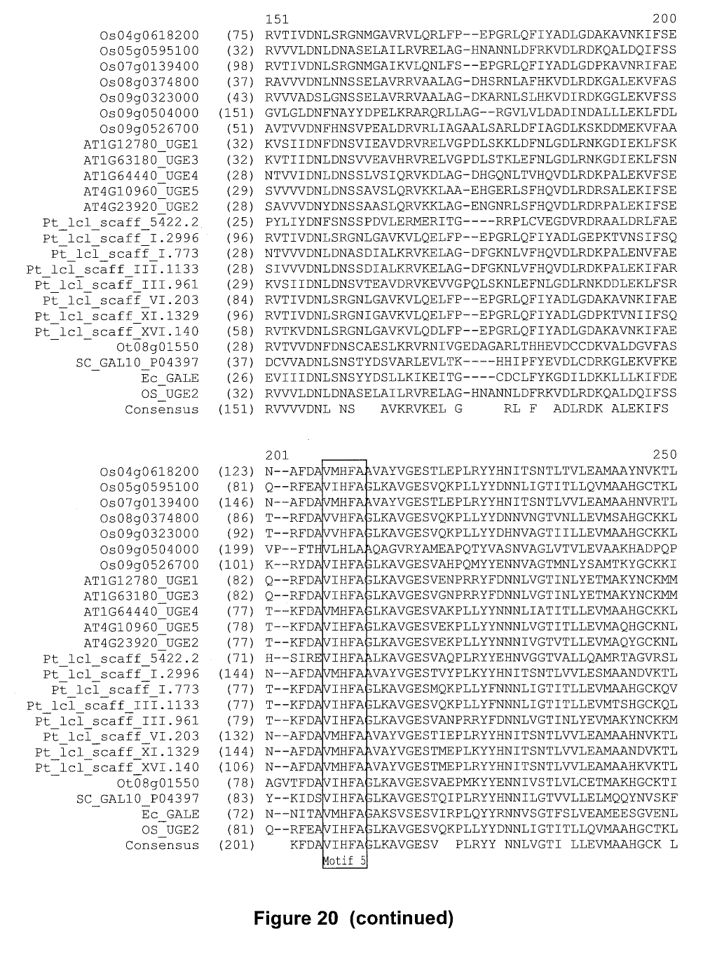

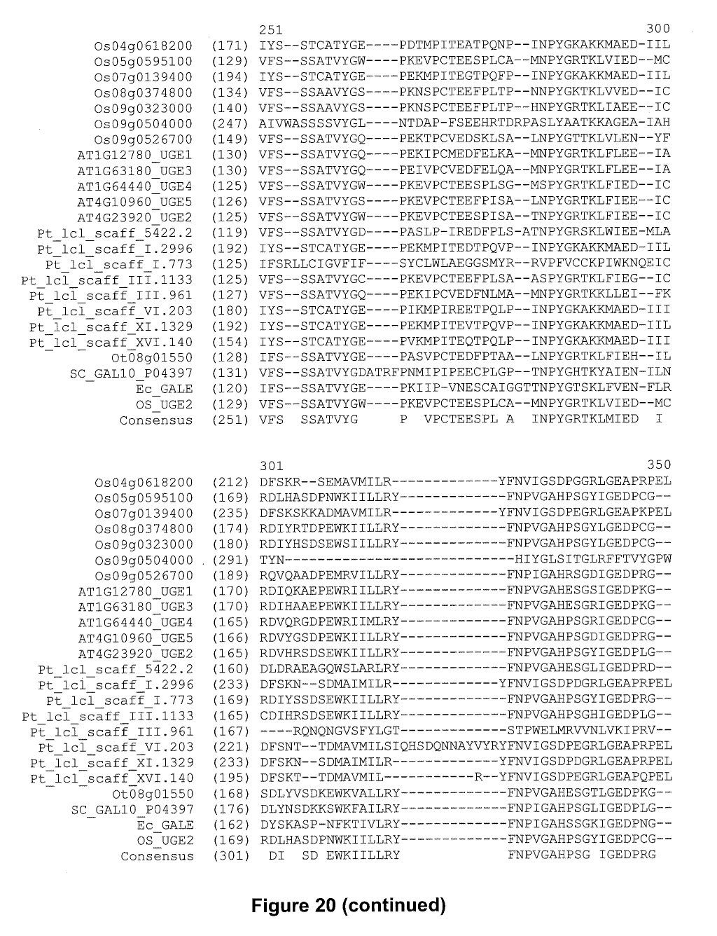

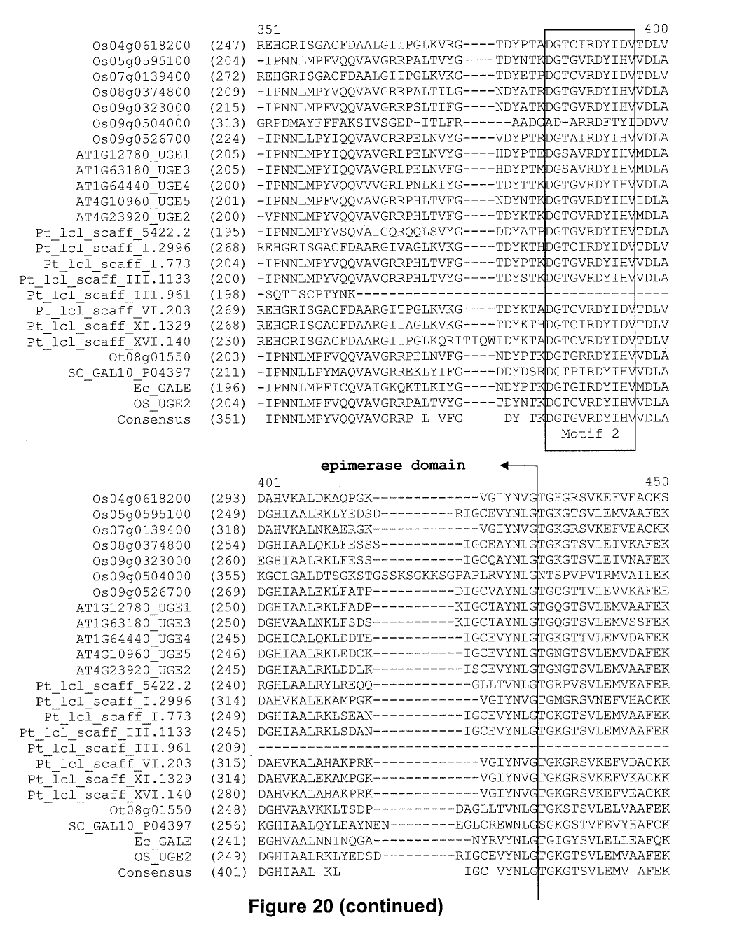

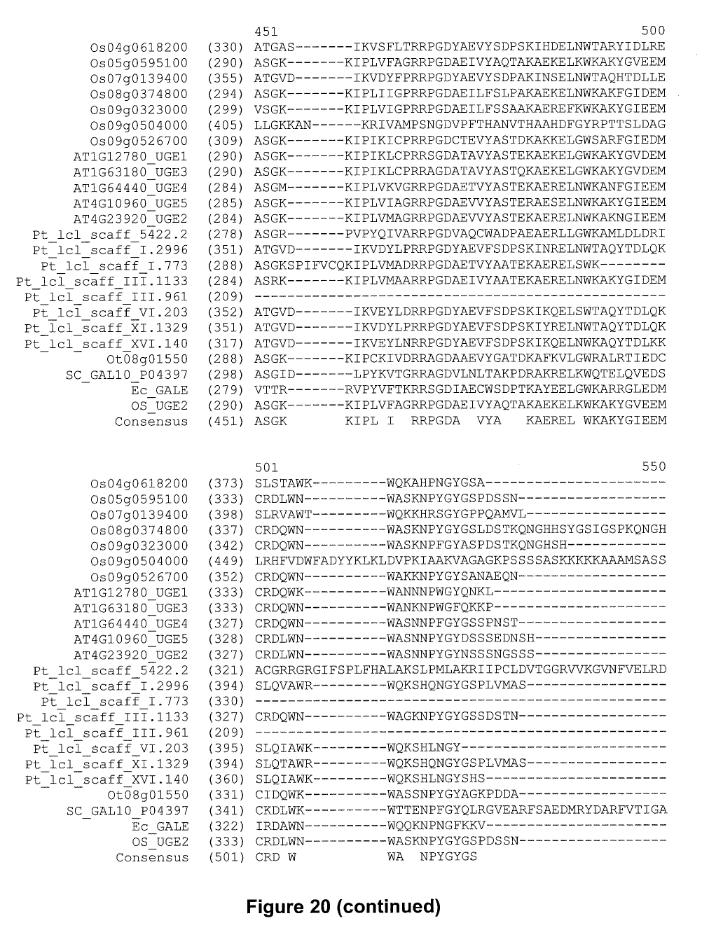

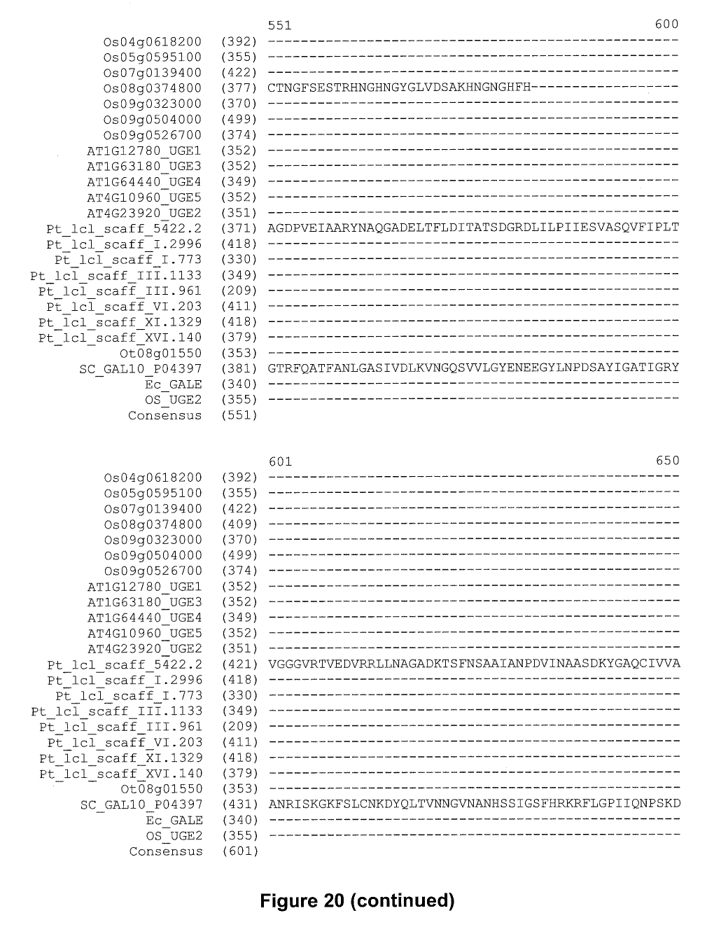

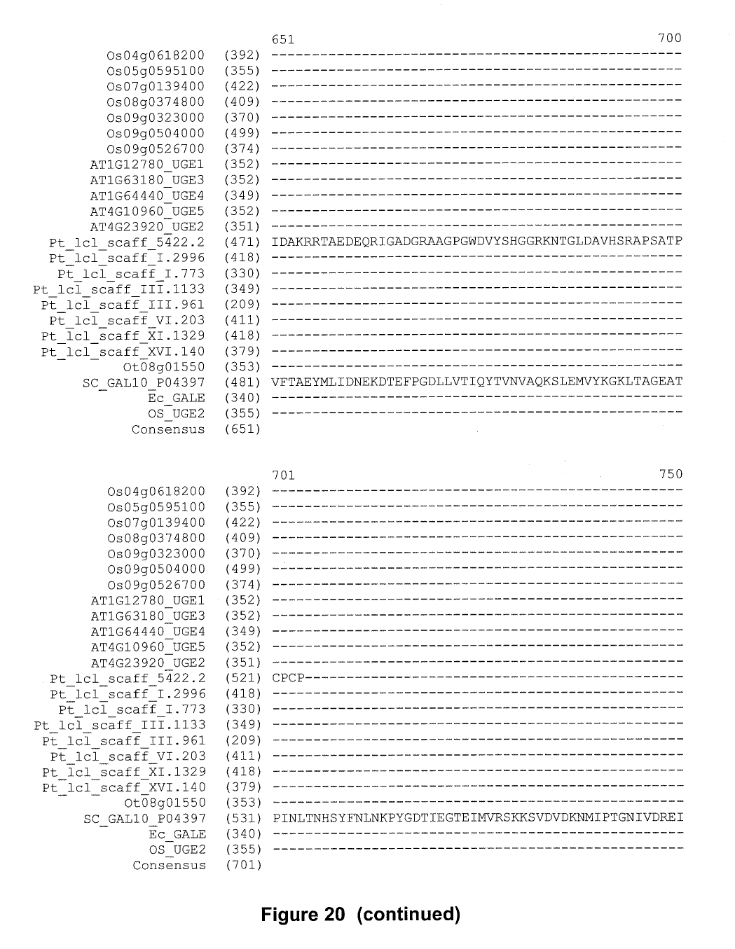

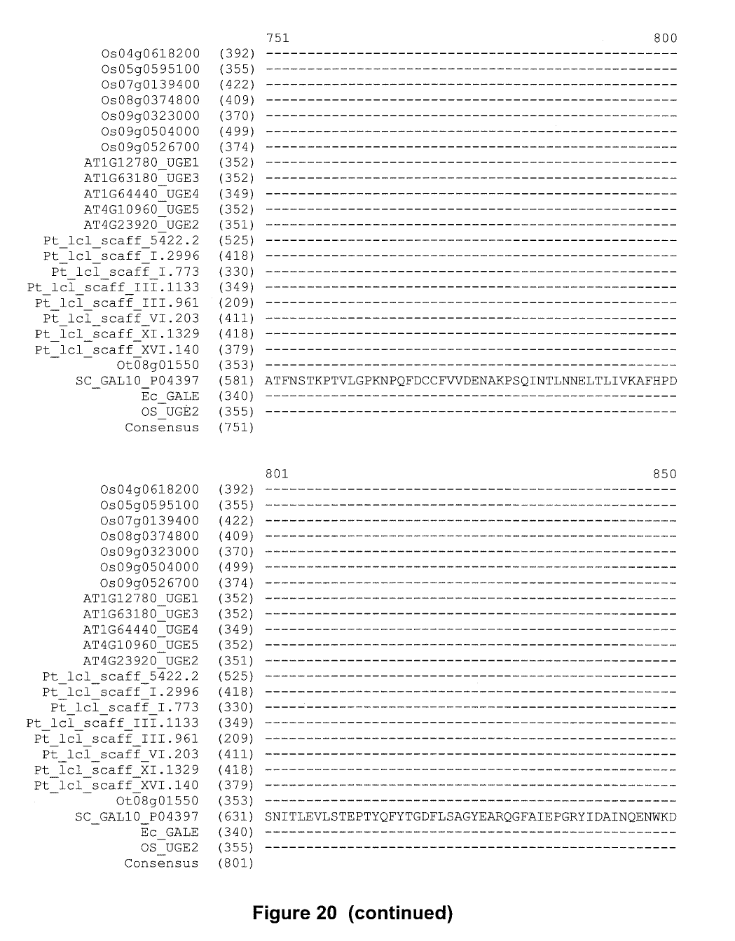

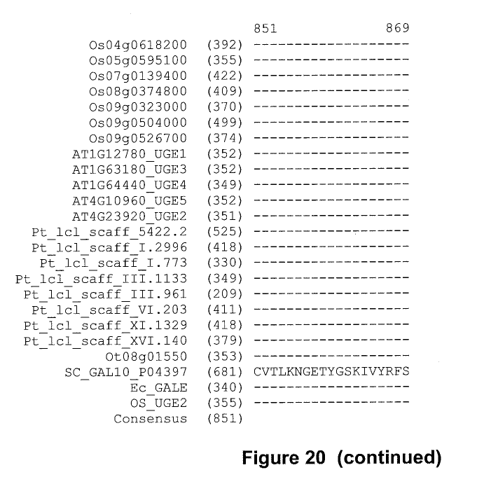

The UGE polypeptides are aligned in FIG. 20.

Sequence conservation among UGE polypeptides is higher in the region located at the N-terminal of the epimerase domain. Regions wherein in the consensus sequence there are defined amino acids given represent the regions with high similarity; blanks in the consensus sequence represent highly variable regions.

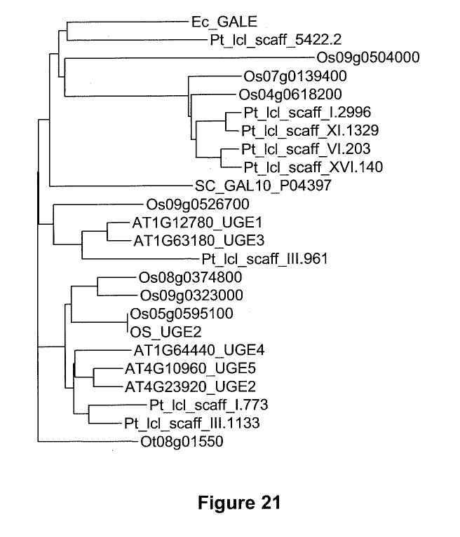

A phylogenetic tree of UGE polypeptides (FIG. 21) was constructed using a neighbour-joining clustering algorithm as provided in the AlignX programme from the Vector NTI (Invitrogen).

### Example 3

### Calculation of Global Percentage Identity Between Polypeptide Sequences Useful in Performing the Methods of the Invention

Global percentages of similarity and identity between full length polypeptide sequences useful in performing the methods of the invention were determined using one of the methods available in the art, the MatGAT (Matrix Global Alignment Tool) software (BMC Bioinformatics. 2003 4:29. MatGAT: an application that generates similarity/identity matrices using protein or DNA sequences. Campanella J J, Bitincka L, Smalley J; software hosted by Ledion Bitincka). MatGAT software generates similarity/identity matrices for DNA or protein sequences without needing pre-alignment of the data. The program performs a series of pair-wise alignments using the Myers and Miller global alignment algorithm (with a gap opening penalty of 12, and a gap extension penalty of 2), calculates similarity and identity using for example Blosum 62 (for polypeptides), and then places the results in a distance matrix. Sequence similarity is shown in the bottom half of the dividing line and sequence identity is shown in the top half of the diagonal dividing line.

Parameters used in the comparison were:

| Scoring matrix: | Blosum62 |
|---|---|
| First Gap: | 12 |
| Extending gap: | 2 |

Results of the software analysis are shown in Table B1 for the global similarity and identity over the full length of the polypeptide sequences. Percentage identity is given above the diagonal in bold and percentage similarity is given below the diagonal (normal face).

The percentage identity between the bHLH6-like polypeptide sequences useful in performing the methods of the invention can be as low as 30% amino acid identity compared to SEQ ID NO: 2 (AAL55713). However, when the alignment is made over the bHLH domain, the sequence identity becomes much higher among the different bHLH6-like proteins. In Table B2, values for the global similarity and identity between sequences that align with SEQ ID NO: 9, (comprising the bHLH domains of bHLH6-like polypeptide sequences) are listed. The sequence identity is about 80% or higher. The used peptide sequences are: SEQ ID NO: 42, NP_{—}001065478; SEQ ID NO: 43, EAY79619; SEQ ID NO: 44, AAD15818; SEQ ID NO: 45, AAB00686; SEQ ID NO: 46, ABD59338; SEQ ID NO: 47, Pt29.195#1; SEQ ID NO: 48, CAF74710; SEQ ID NO: 49, AAF04917; SEQ ID NO: 50, AAQ14332; SEQ ID NO: 51, AAY90122; SEQ ID NO: 52, AAL55713; SEQ ID NO: 53, ABD65632; SEQ ID NO: 54, ABK94979; SEQ ID NO: 55, EDQ81347.

TABLE B1
MatGAT results for global similarity and identity over the full length of the
polypeptide sequences.

| | 1 | 2 | 3 | 4 | 5 | 6 | 7 | 8 | 9 | 10 | 11 | 12 | 13 | 14 |
|---|---|---|---|---|---|---|---|---|---|---|---|---|---|---|
| 1. AAB00686 | | 66.5 | 59.1 | 56.6 | 58.3 | 28.2 | 49.1 | 48.4 | 48.0 | 43.7 | 47.8 | 31.9 | 30.5 | 62.6 |
| 2. ABD59338 | 79.1 | | 54.1 | 50.1 | 55.1 | 27.9 | 46.6 | 43.8 | 45.8 | 41.7 | 45.5 | 29.5 | 31.7 | 57.4 |
| 3. AAQ14332 | 71.1 | 67.7 | | 53.5 | 64.0 | 28.8 | 52.1 | 45.4 | 47.8 | 43.6 | 46.2 | 29.3 | 30.6 | 60.5 |
| 4. AAL55713 | 70.7 | 68.3 | 67.2 | | 52.0 | 24.7 | 46.3 | 47.6 | 47.2 | 42.6 | 45.8 | 33.6 | 33.2 | 55.4 |
| 5. CAF74710 | 70.2 | 68.8 | 78.4 | 66.8 | | 37.3 | 52.9 | 46.4 | 49.7 | 45.5 | 49.2 | 30.0 | 30.0 | 60.5 |
| 6. AAF04917 | 34.9 | 34.4 | 35.1 | 32.4 | 40.9 | | 26.0 | 27.8 | 23.3 | 24.4 | 24.2 | 23.2 | 20.7 | 29.5 |
| 7. AAY90122 | 63.6 | 62.1 | 68.1 | 59.6 | 67.6 | 30.7 | | 41.1 | 45.1 | 42.4 | 44.2 | 27.0 | 29.8 | 49.7 |
| 8. ABD65632 | 66.8 | 61.8 | 60.2 | 66.8 | 59.1 | 34.3 | 54.2 | | 39.3 | 36.8 | 40.0 | 33.5 | 32.2 | 49.2 |
| 9. NP001065478 | 63.8 | 63.5 | 64.8 | 60.8 | 65.5 | 30.9 | 61.4 | 53.5 | | 94.7 | 79.2 | 28.7 | 29.9 | 48.3 |
| 10. EAY79619 | 62.0 | 61.2 | 59.8 | 58.7 | 61.4 | 32.3 | 57.9 | 52.6 | 94.8 | | 75.3 | 27.9 | 27.9 | 43.2 |
| 11. AAD15818 | 62.3 | 62.4 | 63.4 | 59.0 | 65.7 | 31.1 | 62.1 | 54.1 | 87.6 | 83.3 | | 28.5 | 31.1 | 47.0 |
| 12. ABK94979 | 48.6 | 45.4 | 45.2 | 52.6 | 44.9 | 35.8 | 42.6 | 52.2 | 42.9 | 42.8 | 42.7 | | 27.3 | 30.4 |
| 13. EDQ81347 | 50.3 | 49.4 | 47.1 | 50.6 | 48.7 | 31.4 | 46.7 | 53.2 | 45.9 | 44.6 | 45.6 | 48.1 | | 32.2 |
| 14. Pt29.195#1 | 76.8 | 72.6 | 74.5 | 71.6 | 73.4 | 36.9 | 64.0 | 66.2 | 63.7 | 61.5 | 61.8 | 47.9 | 48.8 | |

TABLE B2
MatGAT results for global similarity and identity over the peptide sequences
corresponding to Motif 7.

| | 1 | 2 | 3 | 4 | 5 | 6 | 7 | 8 | 9 | 10 | 11 | 12 | 13 | 14 |
|---|---|---|---|---|---|---|---|---|---|---|---|---|---|---|
| 1. AAL55713 | | 88.6 | 88.6 | 88.6 | 90.0 | 90.0 | 87.1 | 92.9 | 90.0 | 91.4 | 90.0 | 90.0 | 81.4 | 82.9 |
| 2. NP_001065478 | 94.3 | | 100.0 | 98.6 | 92.9 | 94.3 | 92.9 | 92.9 | 90.0 | 92.9 | 91.4 | 91.4 | 80.0 | 84.3 |
| 3. EAY79619 | 94.3 | 100.0 | | 98.6 | 92.9 | 94.3 | 92.9 | 92.9 | 90.0 | 92.9 | 91.4 | 91.4 | 80.0 | 84.3 |
| 4. AAD15818 | 94.3 | 100.0 | 100.0 | | 92.9 | 94.3 | 94.3 | 92.9 | 90.0 | 92.9 | 91.4 | 91.4 | 80.0 | 84.3 |
| 5. AAB00686 | 95.7 | 95.7 | 95.7 | 95.7 | | 94.3 | 90.0 | 94.3 | 91.4 | 92.9 | 92.9 | 91.4 | 84.3 | 82.9 |
| 6. ABD59338 | 94.3 | 95.7 | 95.7 | 95.7 | 94.3 | | 92.9 | 95.7 | 92.9 | 95.7 | 94.3 | 94.3 | 81.4 | 85.7 |
| 7. Pt29.195#1 | 95.7 | 95.7 | 95.7 | 95.7 | 94.3 | 95.7 | | 92.9 | 90.0 | 92.9 | 91.4 | 92.9 | 77.1 | 85.7 |
| 8. CAF74710 | 97.1 | 94.3 | 94.3 | 95.7 | 94.3 | 95.7 | 98.6 | | 97.1 | 97.1 | 97.1 | 94.3 | 80.0 | 85.7 |
| 9. AAF04917 | 97.1 | 94.3 | 94.3 | 95.7 | 94.3 | 95.7 | 98.6 | 100.0 | | 94.3 | 94.3 | 94.3 | 77.1 | 82.9 |
| 10. AAQ14332 | 97.1 | 94.3 | 94.3 | 95.7 | 94.3 | 95.7 | 97.1 | 98.6 | 98.6 | | 94.3 | 95.7 | 81.4 | 85.7 |
| 11. AAY90122 | 95.7 | 95.7 | 95.7 | 97.1 | 95.7 | 97.1 | 98.6 | 98.6 | 98.6 | 97.1 | | 92.9 | 78.6 | 84.3 |
| 12. ABD65632 | 97.1 | 94.3 | 94.3 | 94.3 | 95.7 | 95.7 | 97.1 | 97.1 | 97.1 | 97.1 | 97.1 | | 80.0 | 87.1 |
| 13. ABK94979 | 90.0 | 88.6 | 88.6 | 88.6 | 92.9 | 88.6 | 87.1 | 88.6 | 88.6 | 88.6 | 90.0 | 90.0 | | 71.4 |
| 14. EDQ81347 | 91.4 | 91.4 | 91.4 | 91.4 | 91.4 | 92.9 | 94.3 | 92.9 | 92.9 | 92.9 | 92.9 | 95.7 | 87.1 | |

The percentage identity between the STO polypeptide sequences useful in performing the methods of the invention can be as low as 15% amino acid identity compared to SEQ ID NO: 2 (Os04g0540200)

TABLE B3
MatGAT results for global similarity and identity over the full length of the
polypeptide sequence amongst rice STO proteins.

| | 1 | 2 | 3 | 4 | 5 | 6 | 7 | 8 | 9 | 10 |
|---|---|---|---|---|---|---|---|---|---|---|
| 1. Os04g0540200 | | 29.4 | 32.3 | 63.1 | 33.8 | 28 | 29.2 | 39.3 | 28 | 37.2 |
| 2. Os01g0202500 | 39.5 | | 27.1 | 27.4 | 26.5 | 50.1 | 39.1 | 28.6 | 17.9 | 30.9 |
| 3. Os02g0606200 | 49.1 | 38.4 | | 30.3 | 61.2 | 26.4 | 29.6 | 31.8 | 26.6 | 29.6 |
| 4. Os02g0646200 | 71.7 | 39.8 | 45.8 | | 30.5 | 27 | 29.1 | 41.1 | 25.3 | 33.3 |
| 5. Os04g0493000 | 48.2 | 37.5 | 71.6 | 43.9 | | 28.2 | 30.4 | 34.6 | 25.7 | 31.7 |
| 6. Os05g0204600 | 36.2 | 63 | 36.2 | 36 | 36 | | 37.9 | 26.2 | 17.6 | 29.4 |
| 7. Os06g0152200 | 40 | 54.4 | 42.5 | 39.2 | 40.3 | 54.2 | | 28.8 | 21.1 | 28 |
| 8. Os06g0713000 | 52.3 | 39.8 | 43.8 | 51.9 | 45.5 | 37 | 41.4 | | 21.4 | 29.9 |
| 9. Os09g0527900 | 39.2 | 27.5 | 34.3 | 35.3 | 41.6 | 25.7 | 30.3 | 32.5 | | 25.1 |
| 10. Os12g0209200 | 46.8 | 37.8 | 42.4 | 44.6 | 43.6 | 34.1 | 36.1 | 38.6 | 38.4 | |

TABLE B4
MatGAT results for global similarity and identity over the full length of the
polypeptide sequence amongst orthologous sequences to SEQ IC NO: 2

| | 1 | 2 | 3 | 4 | 5 | 6 | 7 | 8 | 9 | 10 | 11 | 12 | 13 |
|---|---|---|---|---|---|---|---|---|---|---|---|---|---|
| 1. AT1G06040_SplicedVariant1 | | 68.5 | 29.5 | 32 | 69.2 | 29.1 | 31.5 | 31.6 | 58.9 | 59.7 | 32.3 | 30 | 62.5 |
| 2. AT1G06040_SplicedVariant2 | 69 | | 26.2 | 30.1 | 47.4 | 38.3 | 30.6 | 32.9 | 49.4 | 49.8 | 30 | 39.3 | 48.6 |
| 3. AT1G75540 | 43.2 | 35.6 | | 29.5 | 28.4 | 23.4 | 35.6 | 32.3 | 30.5 | 30.2 | 51.5 | 25.5 | 29 |
| 4. AT1G78600 | 48.2 | 40.1 | 45.9 | | 32.1 | 28.2 | 29.7 | 28.9 | 32.1 | 32.1 | 30.9 | 30.5 | 30.6 |
| 5. AT2G31380 | 82.7 | 60.9 | 40.5 | 44.5 | | 29.5 | 34.6 | 32.6 | 58.2 | 58.2 | 32.2 | 29.6 | 58.3 |
| 6. AT4G10240 | 41.9 | 57.6 | 32.9 | 36.5 | 42 | | 30 | 31 | 31.4 | 29.3 | 23.8 | 39.9 | 30.3 |
| 7. AT4G39070 | 50.4 | 43 | 47.7 | 43.5 | 48.3 | 40.9 | | 46.8 | 30 | 30.4 | 38.7 | 29.6 | 30.9 |
| 8. MS_ABO84497 | 49.2 | 49.1 | 46.5 | 41.8 | 49.2 | 44.6 | 60.7 | | 32.6 | 30.8 | 37.5 | 35.1 | 30.6 |
| 9. LE_AAS67368 | 69.4 | 57.1 | 41.4 | 45.2 | 70.6 | 45.9 | 47.1 | 51.9 | | 98.3 | 33.4 | 33.2 | 65.8 |
| 10. ST_ABA40448.1 | 69.8 | 57.1 | 40.8 | 45.5 | 71 | 45.1 | 47.1 | 48.5 | 99.1 | | 33.1 | 32.8 | 66.7 |
| 11. PP_STO | 43.5 | 37.1 | 65.3 | 43.5 | 42.6 | 33.9 | 50.3 | 53.2 | 46.1 | 46.1 | | 27.3 | 30.8 |
| 12. VV-CAN72879.1 | 45.6 | 53.2 | 35.6 | 45.2 | 49.2 | 54.6 | 44.2 | 47.3 | 49.8 | 48.9 | 37.1 | | 30.9 |
| 13. GM_ABB29467 | 74.6 | 57.1 | 40.2 | 44.1 | 73.5 | 43.7 | 45 | 50 | 76.5 | 76.5 | 42.3 | 45.4 | |

The percentage identity between the UGE polypeptide sequences useful in performing the methods of the invention can be as low as 15% amino acid identity compared to SEQ ID NO: 222.

TABLE B5
MatGAT results for global similarity and identity over the full length of the
polypeptide sequences representing UGE isoforms in rice.

| UGE protein Name | 1 | 2 | 3 | 4 | 5 | 6 | 7 | 8 |
|---|---|---|---|---|---|---|---|---|
| 1. Os04g0618200 | | 36.7 | 74.3 | 35.9 | 37 | 21.4 | 36.5 | 36.7 |
| 2. Os05g0595100 | 51.7 | | 36.2 | 65.8 | 72.2 | 21.1 | 58.6 | 99.7 |
| 3. Os07g0139400 | 81.7 | 50.6 | | 34.7 | 34.8 | 21.5 | 33.7 | 36 |
| 4. Os08g0374800 | 51 | 75.7 | 50.6 | | 74.4 | 21.7 | 53.4 | 65.8 |
| 5. Os09g0323000 | 51.9 | 82.4 | 49.6 | 82.1 | | 22.6 | 56.3 | 72.8 |
| 6. Os09g0504000 | 36.7 | 33.1 | 38.8 | 34.5 | 35.7 | | 21.4 | 21.6 |
| 7. Os09g0526700 | 51.4 | 74 | 48.2 | 67.6 | 72.9 | 34.5 | | 58.6 |
| 8. OS_UGE2 | 51.7 | 100 | 50.6 | 75.5 | 82.9 | 33.5 | 73.5 | |

TABLE B6
MatGAT results for global similarity and identity over the full length of the
polypeptide sequences representing UGE isoforms originating form different organisms.

| UGE protein Name | 1 | 2 | 3 | 4 | 5 | 6 | 7 | 8 | 9 |
|---|---|---|---|---|---|---|---|---|---|
| 1. AT1G12780_UGE | | 90.6 | 63.3 | 64.6 | 64.3 | 33.9 | 33.1 | 32.9 | 43.6 |
| 2. AT1G63180_UGE | 95.4 | | 62.1 | 63.8 | 63.5 | 32.7 | 31.8 | 32.1 | 42.7 |
| 3. AT1G64440_UGE | 78.3 | 77.5 | | 79.2 | 79.4 | 37.6 | 34.6 | 34.2 | 32.3 |
| 4. AT4G10960_UGE | 81.5 | 80.1 | 88 | | 88.3 | 37.4 | 34.9 | 34.6 | 31 |
| 5. AT4G23920_UGE | 80.1 | 77.8 | 88 | 92.9 | | 37.1 | 35.3 | 34.8 | 31.3 |
| 6. Pt_lcl\|scaff_XVI.140 | 53.2 | 51.3 | 55 | 54.5 | 54 | | 75.3 | 82.7 | 21.1 |
| 7. Pt_lcl\|scaff_XI.1329 | 49.4 | 47.5 | 51.1 | 49.2 | 49.2 | 81.5 | | 76.2 | 18.9 |
| 8. Pt_lcl\|scaff_VI.203 | 49.5 | 48 | 50 | 50.2 | 48.8 | 87.6 | 85.9 | | 20.4 |
| 9. Pt_lcl\|scaff_III.961 | 47.9 | 46.4 | 42 | 41.9 | 42.6 | 33.3 | 30.9 | 31.7 | |
| 10. Pt_lcl\|scaff_III.1133 | 79.8 | 78.3 | 88.2 | 90.3 | 90.3 | 52.9 | 48.7 | 47.8 | 43.4 |
| 11. Pt_lcl\|scaff_I.773 | 69.2 | 68.7 | 76.1 | 76.1 | 77.4 | 49.2 | 44.1 | 45.9 | 41.9 |
| 12. Pt_lcl\|scaff_I.2996 | 49.4 | 47.5 | 50.8 | 49.4 | 48.9 | 80.6 | 97.1 | 85.1 | 30.9 |
| 13. Pt_lcl\|scaff_5422 | 45 | 44.5 | 45.8 | 48.1 | 47.3 | 39.3 | 38.4 | 39.5 | 25 |
| 14. Ot08g015500 | 34.9 | 34.5 | 35.5 | 37.1 | 36.8 | 28.9 | 29.8 | 29.8 | 20 |
| 15. SC_GAL10_P04397 | 77.8 | 76.4 | 73.6 | 75.6 | 75.3 | 52.1 | 47 | 47.3 | 40.9 |
| 16. Ec_GALE_AAO37702 | 67.5 | 67.8 | 65.5 | 66.7 | 67.4 | 51.1 | 46.5 | 46.1 | 39.5 |
| 17. OS_UGE2 | 80.2 | 79.9 | 83.9 | 87.3 | 86.7 | 55.6 | 50.6 | 50.2 | 42.1 |

| UGE protein Name | 10 | 11 | 12 | 13 | 14 | 15 | 16 | 17 |
|---|---|---|---|---|---|---|---|---|
| 1. AT1G12780_UGE | 65.8 | 54.3 | 33.1 | 33.5 | 24.5 | 59.6 | 47.7 | 65.1 |
| 2. AT1G63180_UGE | 65.5 | 53.7 | 32 | 33.1 | 24 | 60.1 | 49.1 | 65.4 |
| 3. AT1G64440_UGE | 79.6 | 64.6 | 34.4 | 33.9 | 26.9 | 59.9 | 49.3 | 74.9 |
| 4. AT4G10960_UGE | 81.2 | 64.6 | 34.7 | 35.9 | 28.3 | 64.2 | 50.3 | 76.4 |
| 5. AT4G23920_UGE | 83.1 | 65.9 | 35.3 | 35.6 | 28.5 | 62.4 | 50.4 | 75.8 |
| 6. Pt_lcl\|scaff_XVI.140 | 36.8 | 32.9 | 73.2 | 22.5 | 19.6 | 36.1 | 35.2 | 36.1 |
| 7. Pt_lcl\|scaff_XI.1329 | 34.7 | 31 | 94.5 | 21.1 | 19.9 | 33.3 | 33.1 | 34.3 |
| 8. Pt_lcl\|scaff_VI.203 | 34.9 | 32.2 | 75.5 | 22 | 19.4 | 34.2 | 33 | 34.3 |
| 9. Pt_lcl\|scaff_III.961 | 33.1 | 29.9 | 19.6 | 18.2 | 15.2 | 30 | 25.4 | 31.8 |
| 10. Pt_lcl\|scaff_III.1133 | | 73.1 | 34.4 | 34.7 | 27.2 | 62.5 | 50 | 78.2 |
| 11. Pt_lcl\|scaff_I.773 | 80.5 | | 30.8 | 28.6 | 23.7 | 52 | 41.8 | 62.5 |
| 12. Pt_lcl\|scaff_I.2996 | 48.2 | 44.1 | | 20.8 | 19.4 | 33.3 | 32.4 | 34 |
| 13. Pt_lcl\|scaff_5422 | 46.4 | 40.1 | 38.2 | | 32.5 | 35.5 | 32.9 | 35 |
| 14. Ot08g015500 | 36.3 | 32.2 | 29.8 | 45.8 | | 26.2 | 24.5 | 26.9 |
| 15. SC_GAL10_P04397 | 73.9 | 65.1 | 47.2 | 48.7 | 34.6 | | 47.3 | 61.7 |
| 16. Ec_GALE_AAO37702 | 67.5 | 61.9 | 46.3 | 44.5 | 34.2 | 63.6 | | 50 |
| 17. OS_UGE2 | 87.6 | 74.3 | 50.4 | 45 | 37.5 | 73.2 | 67.8 | |

A MATGAT table for local alignment of a specific domain, or data on % identity/similarity between specific domains may also be generated for the other polypeptides (or part thereof) of the invention.

### Example 4

### Identification of Domains Comprised in Polypeptide Sequences Useful in Performing the Methods of the Invention

The Integrated Resource of Protein Families, Domains and Sites (InterPro) database is an integrated interface for the commonly used signature databases for text- and sequence-based searches. The InterPro database combines these databases, which use different methodologies and varying degrees of biological information about well-characterized proteins to derive protein signatures. Collaborating databases include SWISS-PROT, PROSITE, TrEMBL, PRINTS, ProDom and Pfam, Smart and TIGRFAMs. Pfam is a large collection of multiple sequence alignments and hidden Markov models covering many common protein domains and families. Pfam is hosted at the Sanger Institute server in the United Kingdom. Interpro is hosted at the European Bioinformatics Institute in the United Kingdom.

The results of the InterPro scan of the polypeptide sequence as represented by SEQ ID NO: 2 are presented in Table C1. The detected domains classify within the Interpro families IPR001092 (Basic helix-loop-helix dimerisation region bHLH) and IPR001092 (Basic helix-loop-helix dimerisation region bHLH).

TABLE C1
InterPro scan results (major accession numbers) of the
polypeptide sequence as represented by SEQ ID NO: 2.

| | | | Amino acid |
|---|---|---|---|
| | Accession | Accession | coordinates on |
| Database | number | name | SEQ ID NO 2 |
| HMMPfam | PF00010 | HLH | 449-498 |
| ProfileScan | PS50888 | HLH | 438-498 |
| superfamily | SSF47459 | HLH_basic | 451-522 |

The protein sequences representing the GRP (RrmJ/FtsJ polypeptide) are used as query to search the InterPro database. GRP polypeptides useful in performing the methods of the invention have a ribosomal RNA methyltransferase RrmJ/FtsJ domain (InterPro entries IPR002877 and IPR015507; Pfam entry PF01728; PANTHER entry PTHR10920).

The GRP polypeptide (bHLH4 polypeptide) sequences are used as query to search the InterPro database. GRP polypeptides useful in performing the methods of the invention match two InterPro entries: (i) IPR001092 basic helix-loop-helix dimerisation region bHLH; and (ii) IPR011598 helix-loop-helix DNA-binding.

TABLE C2

| | | Integrated | Integrated |
|---|---|---|---|
| | Integrated | database | database |
| InterPro accession | database | accession | accession |
| number | name | number | name |
| IPR001092 | Pfam | PF00010 | HLH |
| Basic helix-loop-helix | | | |
| dimerisation region | | | |
| bHLH | | | |
| IPR001092 | SMART | SM00353 | HLH |
| Basic helix-loop-helix | | | |
| dimerisation region | | | |
| bHLH | | | |
| IPR001092 | Prosite | PS50888 | HLH |
| Basic helix-loop-helix | | | |
| dimerisation region | | | |
| bHLH | | | |
| IPR011598 | | SSF47459 | HLH_basic |
| Helix-loop-helix DNA- | | | |
| binding | | | |
| unintegrated | Panther | PTHR12565 | |
| | Panther | PTHR12565 | SF7 |

The GRP polypeptide (IPT polypeptide) sequences are used as query to search the InterPro database. GRP polypeptides useful in performing the methods of the invention match InterPro entries: (i) IPR002627; and (ii) IPR011593, as shown in the table below:

TABLE C3

| InterPro accession | Integrated database | Integrated database | Integrated database |
|---|---|---|---|
| number | name | accession number | accession name |
| IPR002627 | ProDom | PD004674 | Q6GHD2_STAAR_Q6GHD2 |
| Family tRNA | | | |
| isopentenyltransferase | | | |
| | Panther | PTHR11088 | TRNA DELTA(2)- |
| | | | ISOPENTENYLPYROPHOSPHATE |
| | | | TRANSFERASE-RELATED |
| | Pfam | PF01715 | IPPT |
| IPR011593 | ProDom | PD005388 | Q9CA35_ARATH_Q9CA35 |
| Domain isopentenyl | | | |
| transferase-like | | | |
| No IPR integrated | Panther | PTHR11088:SF20 | CYTOKININ SYNTHASE |

The results of the InterPro scan of the polypeptide sequence as represented by SEQ ID NO: 166 are presented in Table C4.

TABLE C4
InterPro scan results (major accession numbers) of the polypeptide sequence as
represented by SEQ ID NO: 166.

| | | | | Amino acid | | Amino acid | |
|---|---|---|---|---|---|---|---|
| | | | | coordinates | | coordinates | |
| | | | Name | of the B- | | of the B- | |
| Interpro | Accession | Accession | Accession | box1 | | box2 | |
| hit | number | number | number | domain | E-value | domain | E-value |
| IPR000315 | PFAM | PF00643 | zf-B_box | [1-47] | 1.20E−10 | [58-105] | 8.00E−12 |
| | SMART | SM00336 | BBOX | [1-47] | 3.00E−11 | [58-105] | 4.80E−13 |
| | PROFILE | PS50119 | ZF_BBOX | [1-47] | 9.84 | [58-105] | 10.04 |

TABLE C5
Parameters used to build the HMMs for the B-box domain in pfam.

| | Pfam_ls [Download HMM] | Pfam_fs [Download HMM] |
|---|---|---|
| Gathering cutoff | 15.3 15.3; | 21.0 21.0 |
| Trusted cutoff | 15.5 15.5; | 21.0 21.0 |
| Noise cutoff | 15.2 15.2; | 20.8 20.8 |
| Build method of HMM | hmmbuild-F HMM_ls SEED | hmmbuild-f-F HMM_fs SEED |
| | hmmcalibrate --seed 0 HMM_ls | hmmcalibrate --seed 0 HMM_fs |

The results of the InterPro scan of the polypeptide sequence as represented by SEQ ID NO: 222 are presented in Table C6.

TABLE C6
InterPro scan results (major accession numbers) of the
polypeptide sequence as represented by SEQ ID NO: 222.

| | | | Amino acid | |
|---|---|---|---|---|
| | | | coordinates | |
| | Accession | | on SEQ | |
| Database | number | Accession name | ID NO 2 | e-value |
| INTERPRO | IPR001509 | NAD-dependent | [9-273] | 4.20E−74 |
| | | epimerase/ | | |
| | | dehydratase | | |
| PFAM | PF01370 | Epimerase | [9-273] | 4.20E−74 |

### Example 5

### Topology Prediction of the Polypeptide Sequences Useful in Performing the Methods of the Invention

TargetP 1.1 predicts the subcellular location of eukaryotic proteins. The location assignment is based on the predicted presence of any of the N-terminal pre-sequences: chloroplast transit peptide (cTP), mitochondrial targeting peptide (mTP) or secretory pathway signal peptide (SP). Scores on which the final prediction is based are not really probabilities, and they do not necessarily add to one. However, the location with the highest score is the most likely according to TargetP, and the relationship between the scores (the reliability class) may be an indication of how certain the prediction is. The reliability class (RC) ranges from 1 to 5, where 1 indicates the strongest prediction. TargetP is maintained at the server of the Technical University of Denmark.

For the sequences predicted to contain an N-terminal presequence a potential cleavage site can also be predicted.

A number of parameters were selected, such as organism group (non-plant or plant), cutoff sets (none, predefined set of cutoffs, or user-specified set of cutoffs), and the calculation of prediction of cleavage sites (yes or no).

The results of TargetP 1.1 analysis of the polypeptide sequence as represented by SEQ ID NO: 2 are presented Table D. The “plant” organism group has been selected, no cutoffs defined, and the predicted length of the transit peptide requested. The subcellular localization of the polypeptide sequence as represented by SEQ ID NO: 2 may be the cytoplasm or nucleus, no transit peptide is predicted.

TABLE D1
TargetP 1.1 analysis of the polypeptide sequence as
represented by SEQ ID NO: 2

| | |
|---|---|
| Length (AA) | 623 |
| Chloroplastic transit peptide | 0.155 |
| Mitochondrial transit peptide | 0.143 |
| Secretory pathway signal peptide | 0.099 |
| Other subcellular targeting | 0.816 |
| Predicted Location | / |
| Reliability class | 2 |
| Predicted transit peptide length | / |

When analysed with the PredictNLS algorithm, a nuclear localisation is predicted for SEQ ID NO: 2 with a probability of 98%:

| NLS | From | To | signal | Type |
|---|---|---|---|---|
| [KR][KR] × [KR][KR][KR] × [KR][KR] | 433 | 441 | KRPKKRGRK | Potential |

| NumWithNLS | % NucProt |
|---|---|
| 54 | 98.14 |

Many other algorithms can be used to perform such analyses, including:
ChloroP 1.1 hosted on the server of the Technical University of Denmark;
Protein Prowler Subcellular Localisation Predictor version 1.2 hosted on the server of the Institute for Molecular Bioscience, University of Queensland, Brisbane, Australia;
PENCE Proteome Analyst PA-GOSUB 2.5 hosted on the server of the University of Alberta, Edmonton, Alberta, Canada;
TMHMM, hosted on the server of the Technical University of Denmark

The protein sequences representing the GRP are used to query TargetP 1.1. The “plant” organism group is selected, no cutoffs defined, and the predicted length of the transit peptide requested.

The results of TargetP 1.1 analysis of the polypeptide sequence as represented by SEQ ID NO: 225 are presented Table D. The “plant” organism group has been selected, no cutoffs defined, and the predicted length of the transit peptide requested. The polypeptide sequence as represented by SEQ ID NO: 225 may be channelled through the secretory pathway to its final subcellular localization.

TABLE D2
TargetP 1.1 analysis of the polypeptide sequence as
represented by SEQ ID NO: 222

| | |
|---|---|
| Length (AA) | 354 |
| Chloroplastic transit peptide | / |
| Mitochondrial transit peptide | 0.218 |
| Secretory pathway signal peptide | 0.451 |
| Other subcellular targeting | 0.268 |
| Predicted Location | S* |
| Reliability class | 5 |
| Predicted transit peptide length | / |

*S: secretory pathway.

### Example 6

### Functional Assay for the bHLH6-like Polypeptide

A DNA-binding assay for AtbHLH6 is provided in Dombrecht et al. (2007). Briefly, a 1000-bp fragment of the AtbHLH6 coding sequence encompassing codons 285 to 623 was amplified from genomic DNA and cloned into the Nhel-BamHI-digested pTacLCELD6·His vector (Xue, Plant J. 41, 638-649, 2005). The resulting construct encoded the last 338 amino acids of AtbHLH6 (including the bHLH region) in-frame with the reporter protein CelD and a 6×His tag. Correct amplification and cloning were verified by DNA sequencing.

Determination of the consensus sequence of the MYC2 DNA binding motif and the relative binding affinity of these sites was done according to Xue (2005), wherein a fusion of a DNA-binding protein (DBP) to 6•His-tagged cellulase D (CELD) serves both as a means for affinity purification of DBP-DNA complex in the selection of binding sites from a pool of biotinylated random-sequence oligonucleotides and as a reporter for measurement of DNA-binding activity. For the selection of binding-sites using cellulose paper as an affinity matrix, DBP-CELD was incubated at room temperature for 1 h with 20 ng of a biotin-labelled Bio-RS-Oligo in 40 μl of binding/washing buffer (described above) containing 1 mM EDTA, 0.25 μg μl⁻¹ poly d(AC-TG), 1 mg ml⁻¹ BSA and 10% glycerol. An appropriate amount of crude DBP-CELD used for binding-site selection was that achieving 20-30% relative cellulose binding efficiency (percentage cellulose activity bound to cellulose after washing). DBP-CELD/Bio-RS-Oligo mixtures were transferred to 96-well microplate wells containing Whatman 1 filter paper (4 mm²) which was pre-soaked with 10 μl blocking solution [binding/washing buffer containing 0.5 μg μl⁻¹ poly d(AC-TG) and 10% glycerol]. After incubation at 0° C. for 1 hr with gentle shaking, the cellulose paper was washed six times with binding/washing buffer containing 1 mM EDTA and 0.1 mg ml⁻¹ BSA. The use of extensive washing in the target site selection was based on observations of the relatively high stability of DBP-oligonucleotide complex immobilized on solid matrix. DBP-CELD carrying target binding sites were eluted at 40° C. for 15 min with 40 μl of cellulase eluting buffer [10 mM HEPES, pH7, 50 mM KCl, 0.2 mM EDTA, 4 mM cellobiose, 0.05 mg ml⁻¹ BSA and 10 μg ml⁻¹ of a sense sequence-specific primer SP-S]. The eluate was used for PCR amplification of selected oligonucleotides. The PCR product (0.1 μl) without purification was used for the next round of site selection.

Alternatively, 6•His tagged DBP-CELD (10-25 μg crude protein) was incubated in 60 μl of PNT buffer (50 mM sodium phosphate, pH8.0, 300 mM NaCl and 0.05% Tween 20) containing 10 mM imidazole and 350 μg Ni-NTA magnetic agarose beads (Qiagen) at room temperature for 45-60 min with gentle shaking in a micro-tube mixer (Tomy Seiko Co., Tokyo, Japan). The Ni-NTA magnetic beads were collected at the side of the tubes by placing tubes in a 12-tube magnet (Qiagen). Unbound proteins were removed by washing twice with 150-200 μl of PNT containing 20 mM imidazole and once with 60 μl of binding/washing buffer containing 2 mM MgCl₂, 1 mg ml⁻¹ BSA and 10% glycerol. Washed beads were suspended in 40 μl of binding/washing buffer containing 2 mM MgCl₂, 0.25 μg μl⁻¹ poly d(AC-TG), 1 mg ml⁻¹ BSA, 0.0025% Nonidet P-40, 10% glycerol and biotin-labelled oligonucleotides (50 ng Bio-RS-Oligo or 1 μl of PCR product amplified from previously selected oligonucleotides). The suspension was incubated at room temperature for 1.5 h with gentle shaking in the micro-tube mixer. After washing four times (3-4 min each washing) with 150-200 μl of binding/washing buffer containing 2 mM MgCl₂, 0.1 mg ml⁻¹ BSA and 0.0025% Nonidet P-40, the beads carrying target site-bound BDP-CELD were resuspended in 8 μl of 5 mM Tris-Cl (pH 8.0)/0.5 mM EDTA containing 5 μg ml⁻¹ of a sense sequence-specific primer (SP-S) and the suspension was transferred to a clean tube and used for PCR amplification of selected oligonucleotides. The PCR product (1 μl) without purification was used for the next round of site selection. For EMSA assays, 6•His-tagged DBP-CELD proteins were purified using Ni-NTA magnetic agarose beads using a high stringent binding buffer (50 mMsodium phosphate, pH8.0, 1 MNaCl, 10% glycerol, 1% Tween 20 and 10 mM imidazole) and the rest of the procedure followed the manufacturer's instruction. Double-stranded synthetic oligonucleotides (30-45 fmol) labelled with digoxigenin at the 3′-end were incubated with a purified DBP (30-75 ng) in 15 μl of binding buffer [25 mM HEPES/KOH, pH 7.0, 50 mM KCl, 0.5 mM DTT, 2 mM MgCl₂, 0.2 μg μl⁻¹ poly d(AC-TG), 0.3 mg ml⁻¹ BSA and 10% glycerol]. After incubation at room temperature for 30 min, DBP/DNA complexes were separated from free probes on a 6% polyacrylamide gel in a 40-mM Tris-acetate buffer (pH 7.5) containing 5 mM Na acetate, 0.5 mM EDTA and 5% glycerol. The DBP/DNA complexes and free probes in the gels after electrophoresis were transferred to a Hybond N [symbol] membrane. Alkaline phosphatase-conjugated anti-digoxigenin antibody and a chemiluminescent substrate, CDP-Star (Roche Diagnostics) was used for detection of digoxigenin according to the manufacturer's instructions. Further details are provided in Xue (2005).

### Example 7

### Cloning of the Nucleic Acid Sequence Used in the Methods of the Invention

The nucleic acid sequence used in the methods of the invention was amplified by PCR using as template a custom-made Arabidopsis thaliana seedlings cDNA library (in pCMV Sport 6.0; Invitrogen, Paisley, UK). PCR was performed using Hifi Taq DNA polymerase in standard conditions, using 200 ng of template in a 50 μl PCR mix. The primers used were prm06515 (SEQ ID NO: 10; sense, start codon in bold):

5′-ggggacaagtttgtacaaaaaagcaggcttaaacaatgactgattac
cggctacaa-3′

and prm06516 (SEQ ID NO: 11; reverse, complementary):

5′-ggggaccactttgtacaagaaagctgggtacacccttttaaccga
ttttt-3′,

which include the AttB sites for Gateway recombination. The amplified PCR fragment was purified also using standard methods. The first step of the Gateway procedure, the BP reaction, was then performed, during which the PCR fragment recombines in vivo with the pDONR201 plasmid to produce, according to the Gateway terminology, an “entry clone”, pbHLH6-like. Plasmid pDONR201 was purchased from Invitrogen, as part of the Gateway® technology.

The entry clone comprising SEQ ID NO: 1 was then used in an LR reaction with a destination vector used for Oryza sativa transformation. This vector contained as functional elements within the T-DNA borders: a plant selectable marker; a screenable marker expression cassette; and a Gateway cassette intended for LR in vivo recombination with the nucleic acid sequence of interest already cloned in the entry clone. A rice GOS2 promoter (SEQ ID NO: 12) for root specific expression was located upstream of this Gateway cassette.

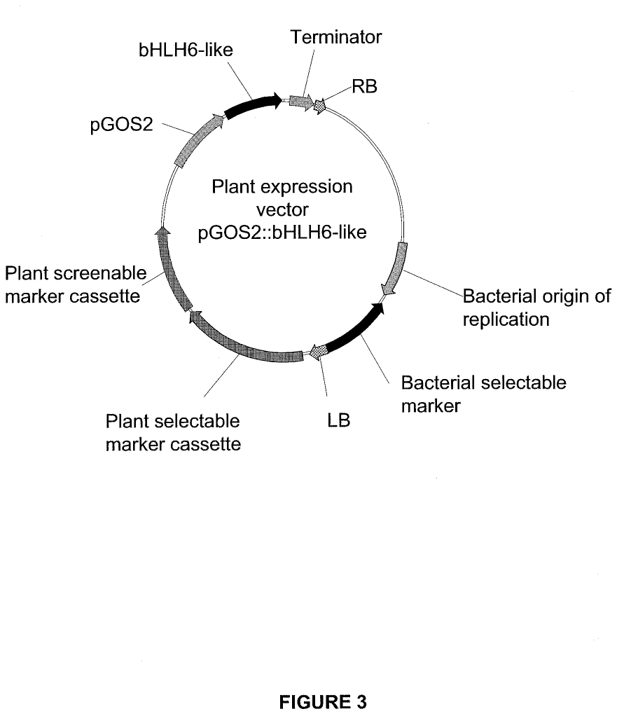

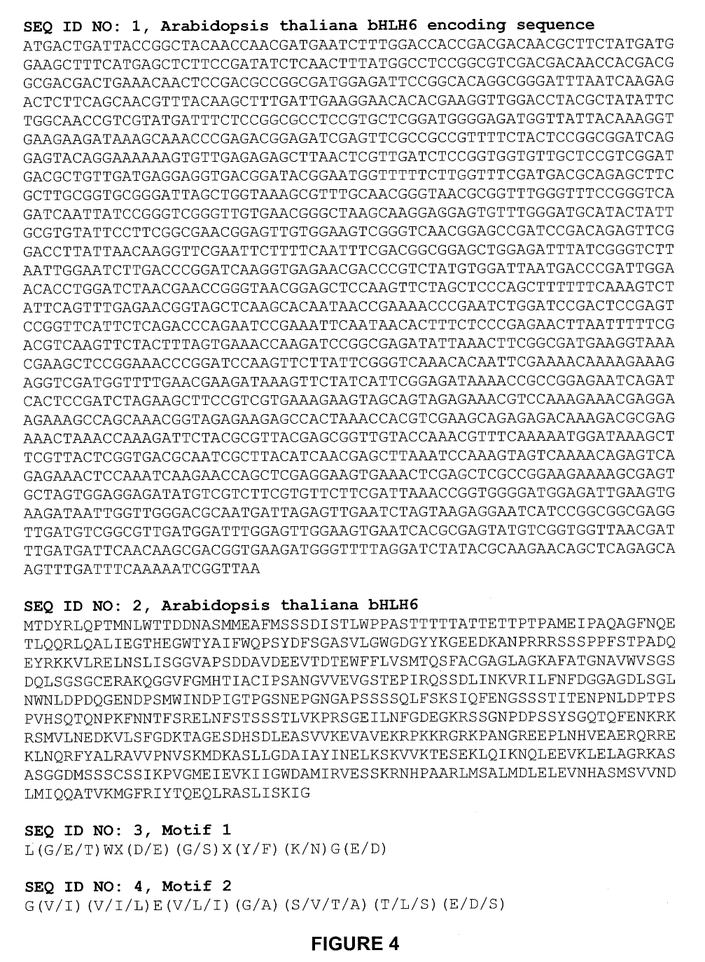

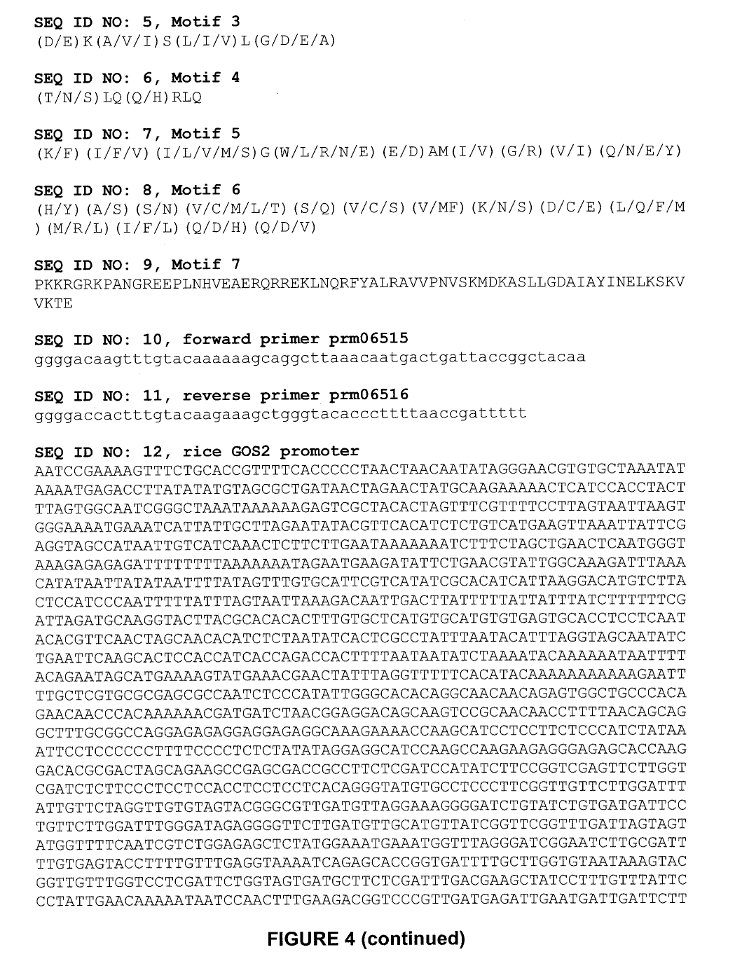

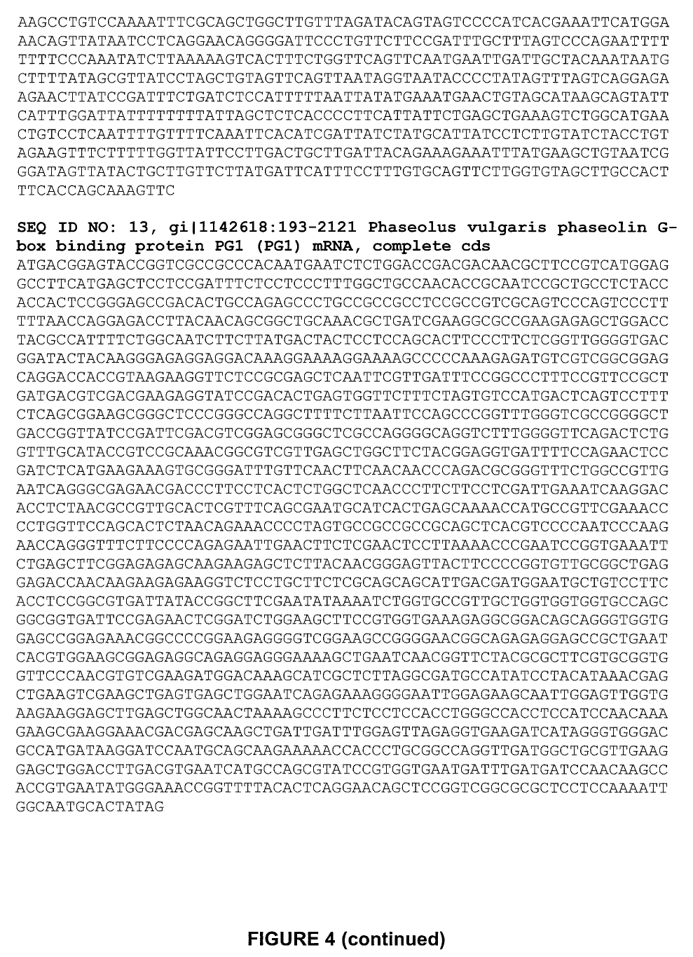

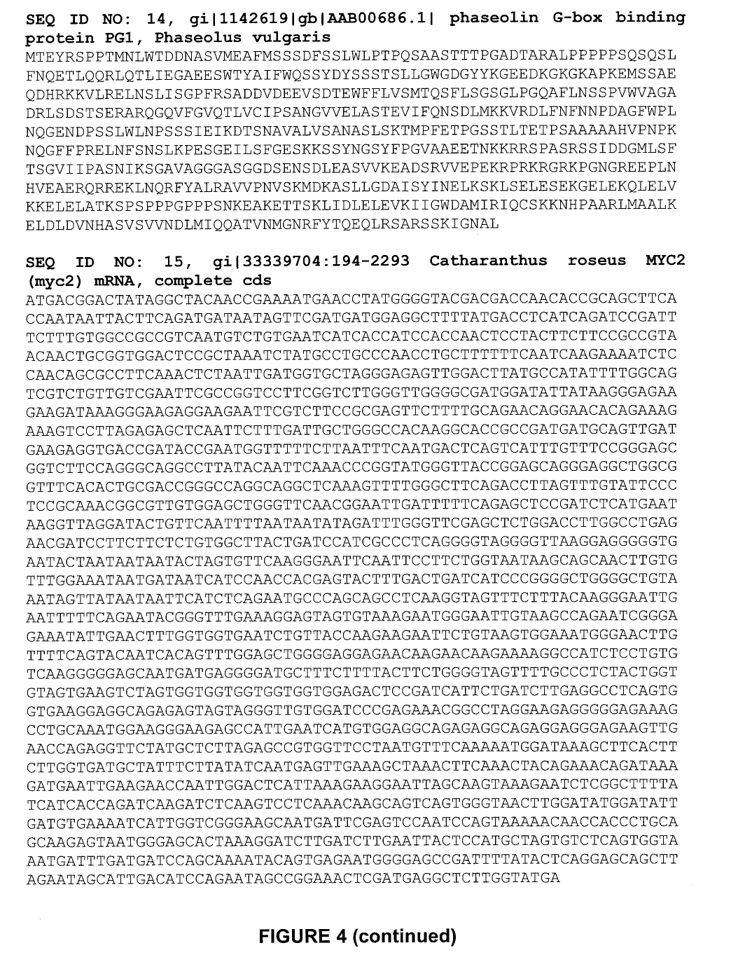

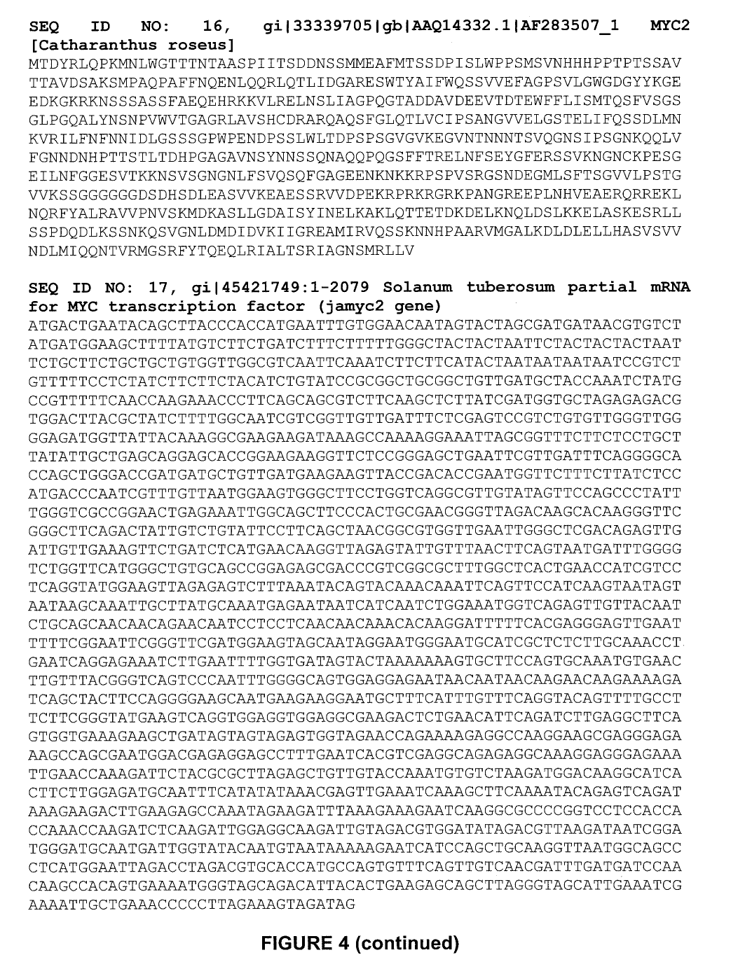

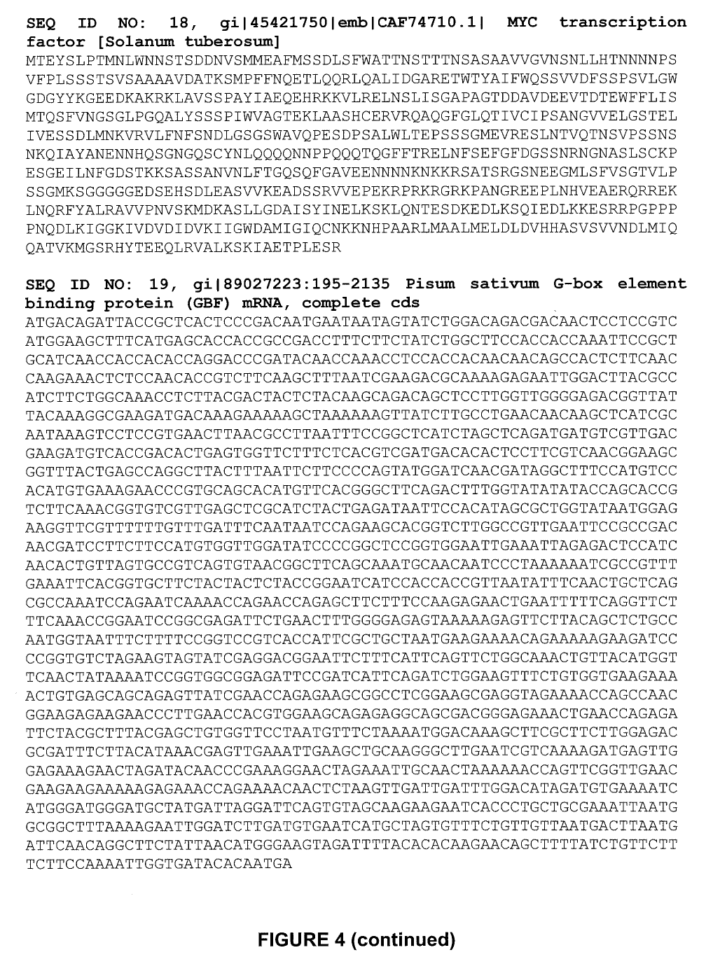

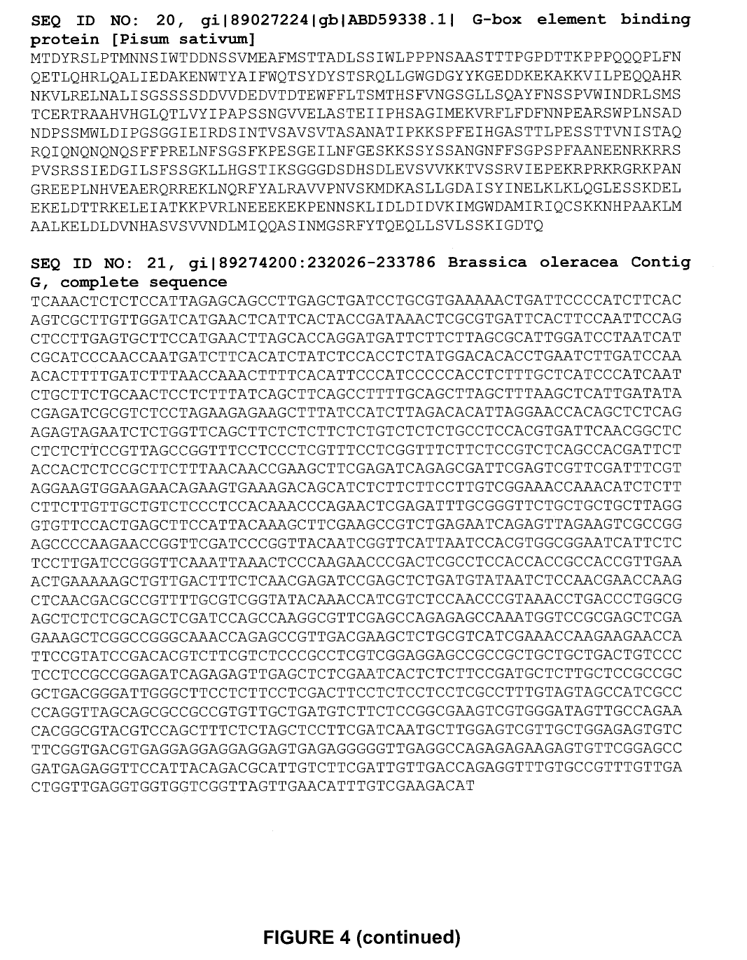

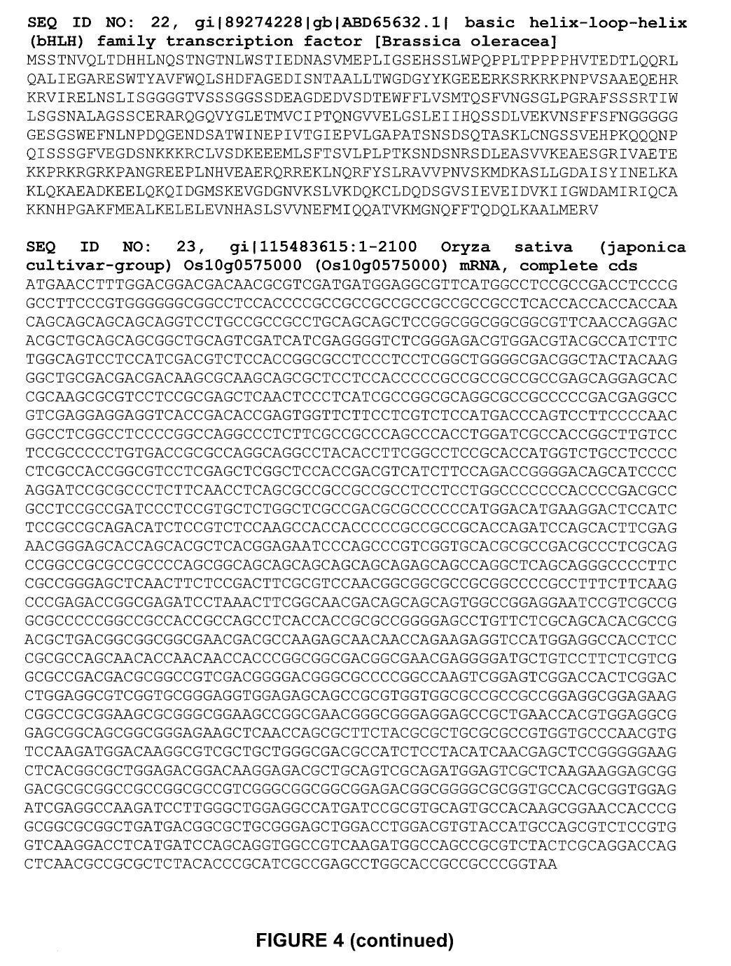

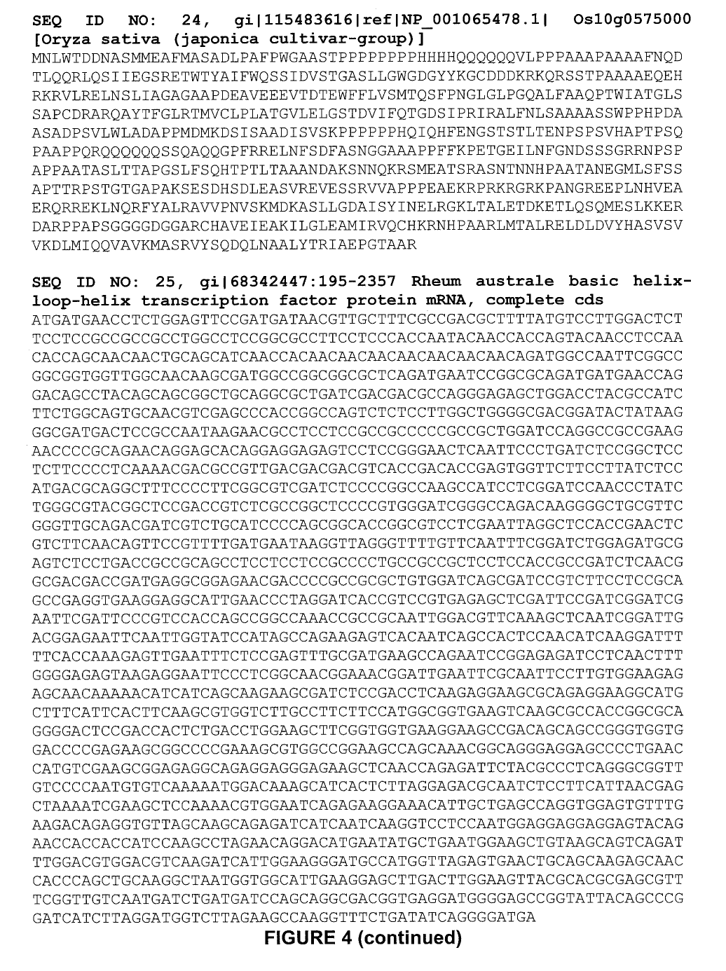

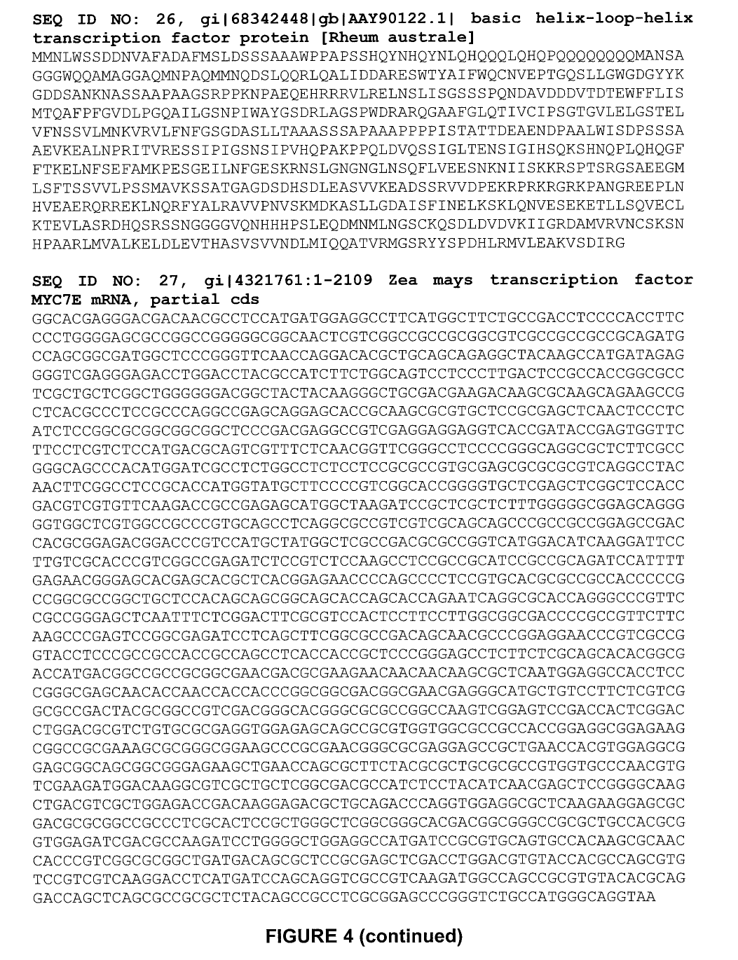

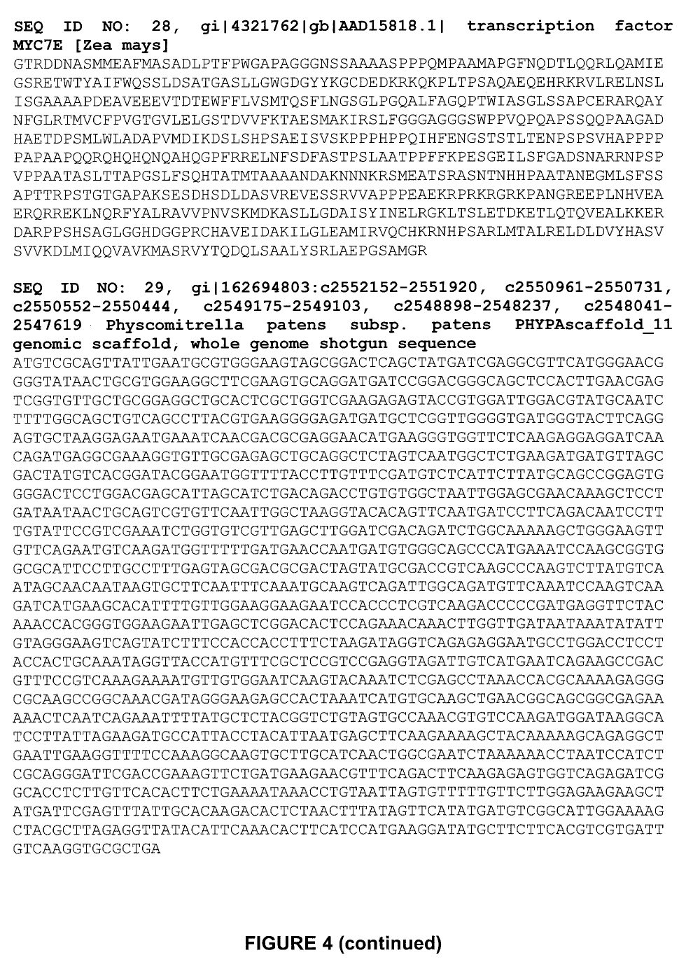

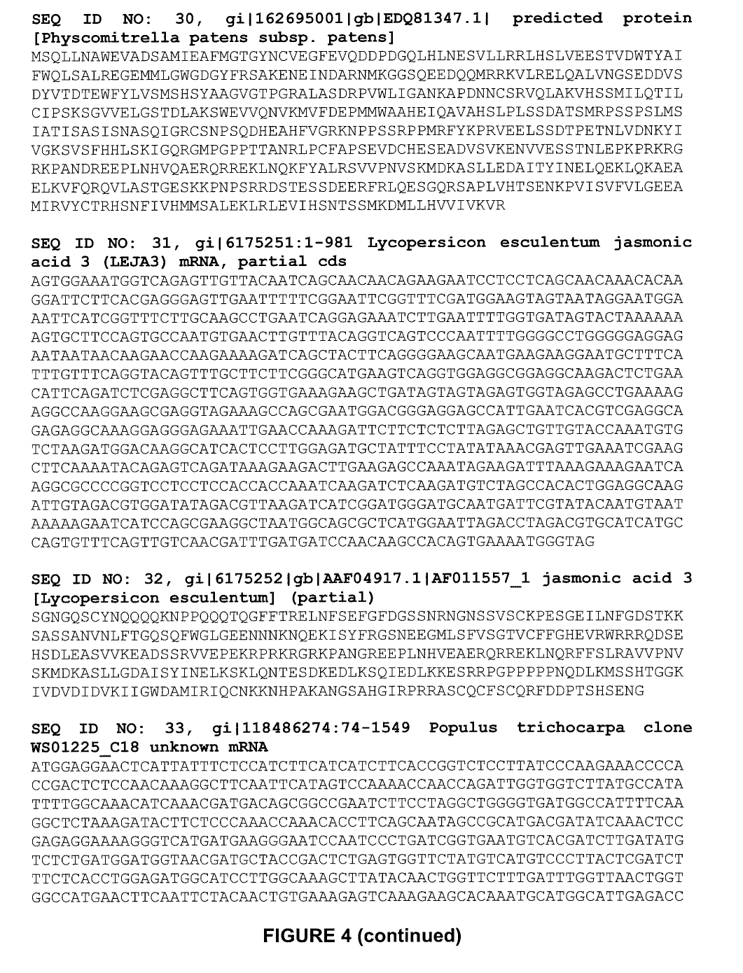

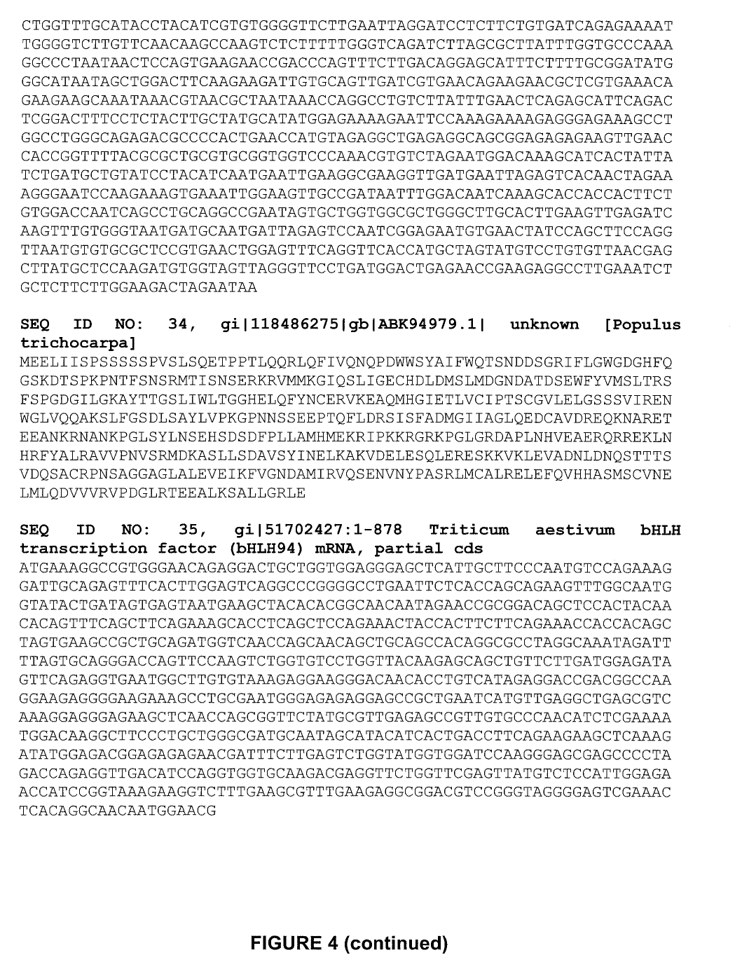

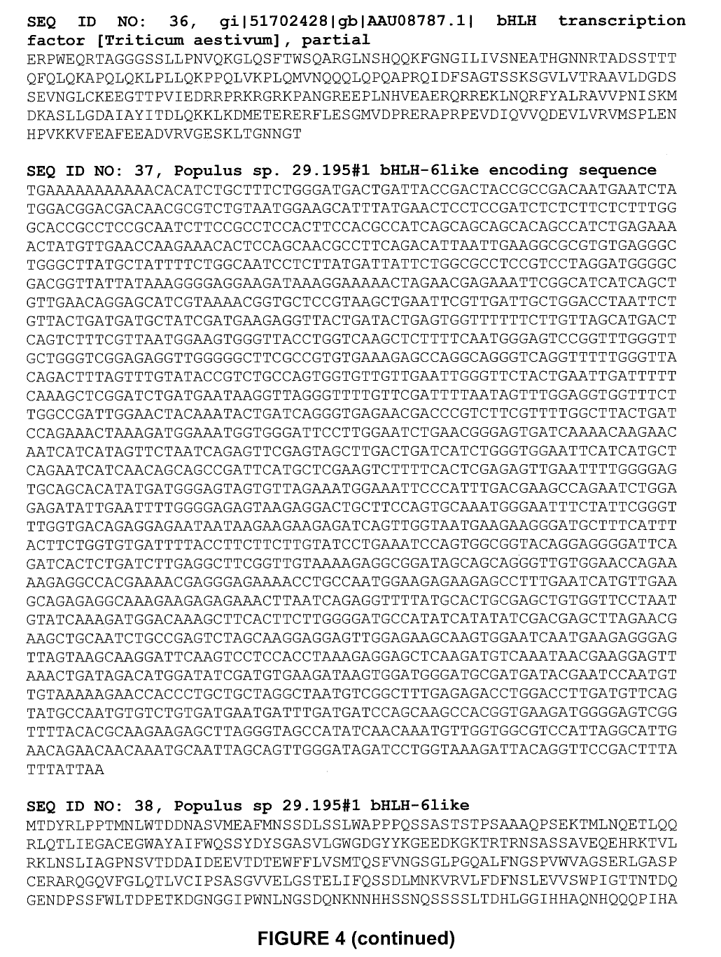

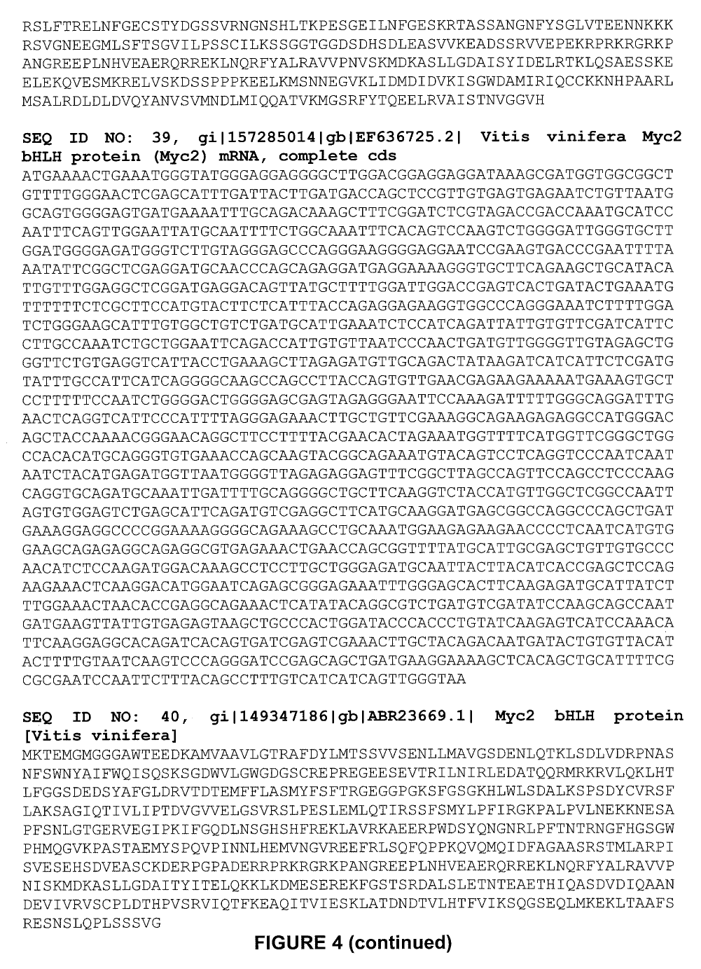

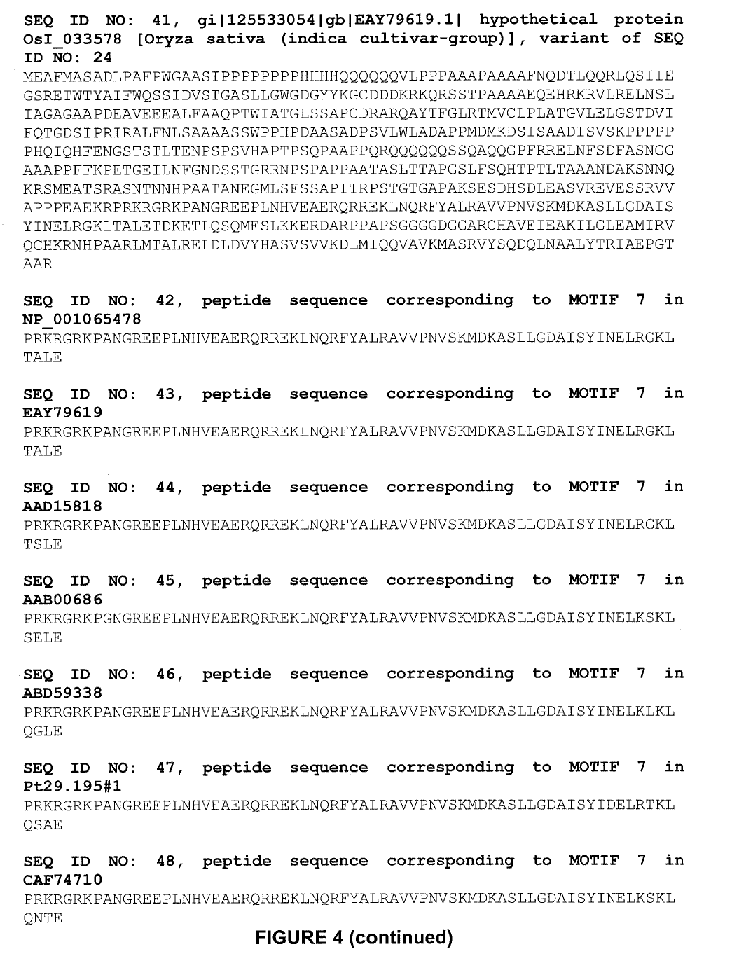

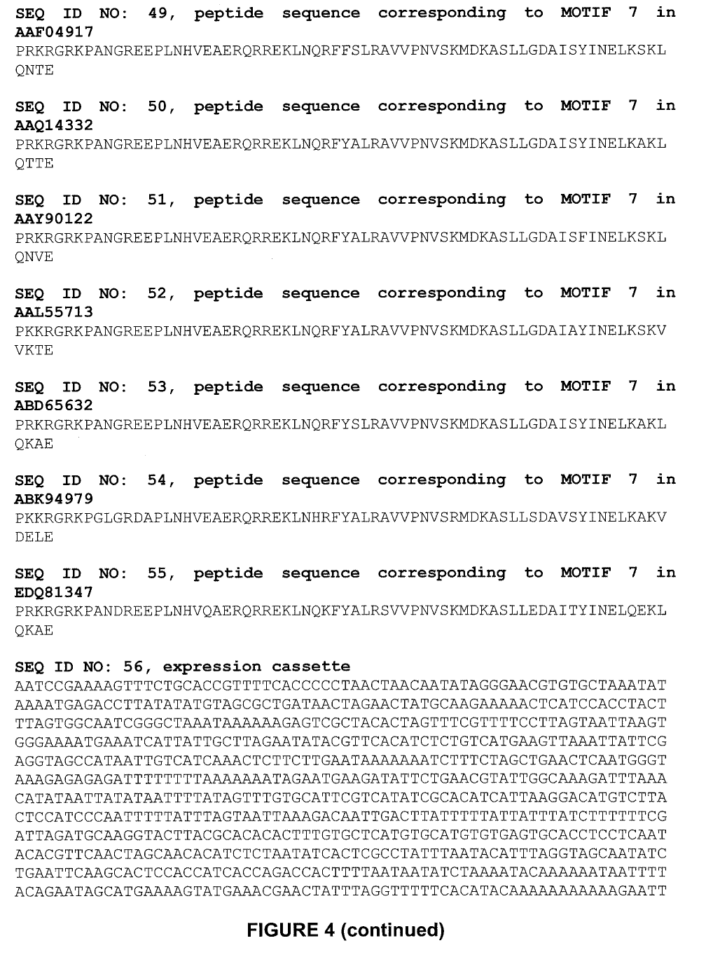

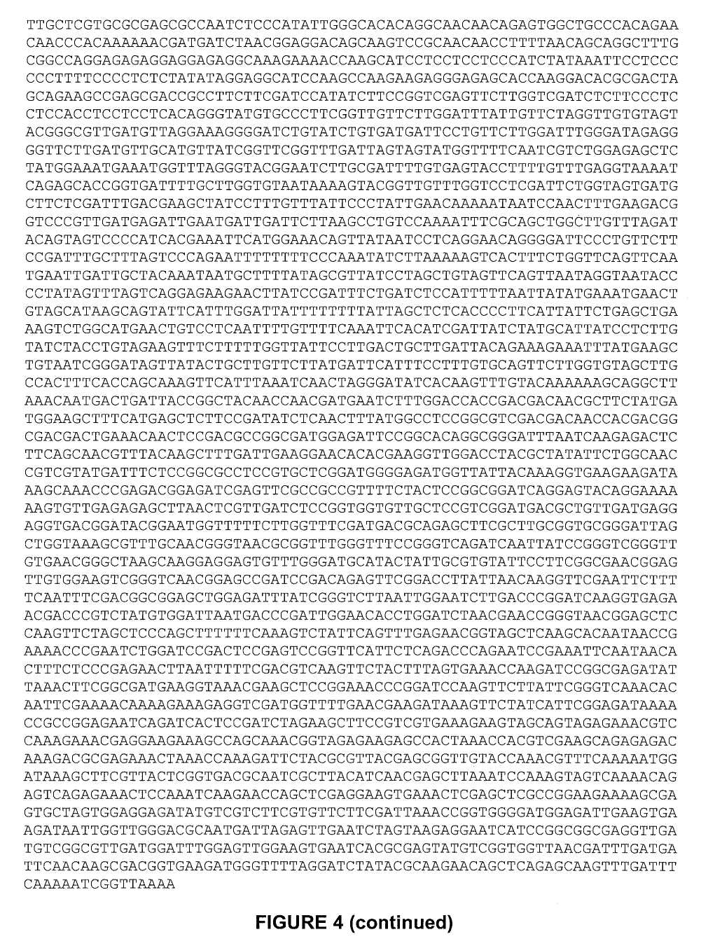

After the LR recombination step, the resulting expression vector pGOS2::bHLH6-like (FIG. 3) was transformed into Agrobacterium strain LBA4044 according to methods well known in the art.

### Example 8

### Plant Transformation

### Rice Transformation

The Agrobacterium containing the expression vector was used to transform Oryza sativa plants. Mature dry seeds of the rice japonica cultivar Nipponbare were dehusked. Sterilization was carried out by incubating for one minute in 70% ethanol, followed by 30 minutes in 0.2% HgCl₂, followed by a 6 times 15 minutes wash with sterile distilled water. The sterile seeds were then germinated on a medium containing 2,4-D (callus induction medium). After incubation in the dark for four weeks, embryogenic, scutellum-derived calli were excised and propagated on the same medium. After two weeks, the calli were multiplied or propagated by subculture on the same medium for another 2 weeks. Embryogenic callus pieces were sub-cultured on fresh medium 3 days before co-cultivation (to boost cell division activity).

Agrobacterium strain LBA4404 containing the expression vector was used for co-cultivation. Agrobacterium was inoculated on AB medium with the appropriate antibiotics and cultured for 3 days at 28° C. The bacteria were then collected and suspended in liquid co-cultivation medium to a density (OD₆₀₀) of about 1. The suspension was then transferred to a Petri dish and the calli immersed in the suspension for 15 minutes. The callus tissues were then blotted dry on a filter paper and transferred to solidified, co-cultivation medium and incubated for 3 days in the dark at 25° C. Co-cultivated calli were grown on 2,4-D-containing medium for 4 weeks in the dark at 28° C. in the presence of a selection agent. During this period, rapidly growing resistant callus islands developed. After transfer of this material to a regeneration medium and incubation in the light, the embryogenic potential was released and shoots developed in the next four to five weeks. Shoots were excised from the calli and incubated for 2 to 3 weeks on an auxin-containing medium from which they were transferred to soil. Hardened shoots were grown under high humidity and short days in a greenhouse.

Approximately 35 independent T0 rice transformants were generated for one construct. The primary transformants were transferred from a tissue culture chamber to a greenhouse. After a quantitative PCR analysis to verify copy number of the T-DNA insert, only single copy transgenic plants that exhibit tolerance to the selection agent were kept for harvest of T1 seed. Seeds were then harvested three to five months after transplanting. The method yielded single locus transformants at a rate of over 50% (Aldemita and Hodges1996, Chan et al. 1993, Hiei et al. 1994).

### Corn Transformation

Transformation of maize (Zea mays) is performed with a modification of the method described by Ishida et al. (1996) Nature Biotech 14(6): 745-50. Transformation is genotype-dependent in corn and only specific genotypes are amenable to transformation and regeneration. The inbred line A188 (University of Minnesota) or hybrids with A188 as a parent are good sources of donor material for transformation, but other genotypes can be used successfully as well. Ears are harvested from corn plant approximately 11 days after pollination (DAP) when the length of the immature embryo is about 1 to 1.2 mm. Immature embryos are cocultivated with Agrobacterium tumefaciens containing the expression vector, and transgenic plants are recovered through organogenesis. Excised embryos are grown on callus induction medium, then maize regeneration medium, containing the selection agent (for example imidazolinone but various selection markers can be used). The Petri plates are incubated in the light at 25° C. for 2-3 weeks, or until shoots develop. The green shoots are transferred from each embryo to maize rooting medium and incubated at 25° C. for 2-3 weeks, until roots develop. The rooted shoots are transplanted to soil in the greenhouse. T1 seeds are produced from plants that exhibit tolerance to the selection agent and that contain a single copy of the T-DNA insert.

### Wheat Transformation

Transformation of wheat is performed with the method described by Ishida et al. (1996) Nature

Biotech 14(6): 745-50. The cultivar Bobwhite (available from CIMMYT, Mexico) is commonly used in transformation. Immature embryos are co-cultivated with Agrobacterium tumefaciens containing the expression vector, and transgenic plants are recovered through organogenesis. After incubation with Agrobacterium, the embryos are grown in vitro on callus induction medium, then regeneration medium, containing the selection agent (for example imidazolinone but various selection markers can be used). The Petri plates are incubated in the light at 25° C. for 2-3 weeks, or until shoots develop. The green shoots are transferred from each embryo to rooting medium and incubated at 25° C. for 2-3 weeks, until roots develop. The rooted shoots are transplanted to soil in the greenhouse. T1 seeds are produced from plants that exhibit tolerance to the selection agent and that contain a single copy of the T-DNA insert.

### Soybean Transformation

Soybean is transformed according to a modification of the method described in the Texas A&M U.S. Pat. No. 5,164,310. Several commercial soybean varieties are amenable to transformation by this method. The cultivar Jack (available from the Illinois Seed foundation) is commonly used for transformation. Soybean seeds are sterilised for in vitro sowing. The hypocotyl, the radicle and one cotyledon are excised from seven-day old young seedlings. The epicotyl and the remaining cotyledon are further grown to develop axillary nodes. These axillary nodes are excised and incubated with Agrobacterium tumefaciens containing the expression vector. After the cocultivation treatment, the explants are washed and transferred to selection media. Regenerated shoots are excised and placed on a shoot elongation medium. Shoots no longer than 1 cm are placed on rooting medium until roots develop. The rooted shoots are transplanted to soil in the greenhouse. T1 seeds are produced from plants that exhibit tolerance to the selection agent and that contain a single copy of the T-DNA insert.

### Rapeseed/Canola Transformation

Cotyledonary petioles and hypocotyls of 5-6 day old young seedling are used as explants for tissue culture and transformed according to Babic et al. (1998, Plant Cell Rep 17: 183-188). The commercial cultivar Westar (Agriculture Canada) is the standard variety used for transformation, but other varieties can also be used. Canola seeds are surface-sterilized for in vitro sowing. The cotyledon petiole explants with the cotyledon attached are excised from the in vitro seedlings, and inoculated with Agrobacterium (containing the expression vector) by dipping the cut end of the petiole explant into the bacterial suspension. The explants are then cultured for 2 days on MSBAP-3 medium containing 3 mg/l BAP, 3% sucrose, 0.7% Phytagar at 23° C., 16 hr light. After two days of co-cultivation with Agrobacterium, the petiole explants are transferred to MSBAP-3 medium containing 3 mg/l BAP, cefotaxime, carbenicillin, or timentin (300 mg/l) for 7 days, and then cultured on MSBAP-3 medium with cefotaxime, carbenicillin, or timentin and selection agent until shoot regeneration. When the shoots are 5-10 mm in length, they are cut and transferred to shoot elongation medium (MSBAP-0.5, containing 0.5 mg/l BAP). Shoots of about 2 cm in length are transferred to the rooting medium (MS0) for root induction. The rooted shoots are transplanted to soil in the greenhouse. T1 seeds are produced from plants that exhibit tolerance to the selection agent and that contain a single copy of the T-DNA insert.

### Alfalfa Transformation

A regenerating clone of alfalfa (Medicago sativa) is transformed using the method of (McKersie et al., 1999 Plant Physiol 119: 839-847). Regeneration and transformation of alfalfa is genotype dependent and therefore a regenerating plant is required. Methods to obtain regenerating plants have been described. For example, these can be selected from the cultivar Rangelander (Agriculture Canada) or any other commercial alfalfa variety as described by Brown DCW and A Atanassov (1985. Plant Cell Tissue Organ Culture 4: 111-112). Alternatively, the RA3 variety (University of Wisconsin) has been selected for use in tissue culture (Walker et al., 1978 Am J Bot 65:654-659). Petiole explants are cocultivated with an overnight culture of Agrobacterium tumefaciens C58C1 pMP90 (McKersie et al., 1999 Plant Physiol 119: 839-847) or LBA4404 containing the expression vector. The explants are cocultivated for 3 d in the dark on SH induction medium containing 288 mg/L Pro, 53 mg/L thioproline, 4.35 g/L K2SO4, and 100 μm acetosyringinone. The explants are washed in half-strength Murashige-Skoog medium (Murashige and Skoog, 1962) and plated on the same SH induction medium without acetosyringinone but with a suitable selection agent and suitable antibiotic to inhibit Agrobacterium growth. After several weeks, somatic embryos are transferred to BOi2Y development medium containing no growth regulators, no antibiotics, and 50 g/L sucrose. Somatic embryos are subsequently germinated on half-strength Murashige-Skoog medium. Rooted seedlings were transplanted into pots and grown in a greenhouse. T1 seeds are produced from plants that exhibit tolerance to the selection agent and that contain a single copy of the T-DNA insert.

### Cotton Transformation

Cotton is transformed using Agrobacterium tumefaciens according to the method described in U.S. Pat. No. 5,159,135. Cotton seeds are surface sterilised in 3% sodium hypochlorite solution during 20 minutes and washed in distilled water with 500 μg/ml cefotaxime. The seeds are then transferred to SH-medium with 50 μg/ml benomyl for germination. Hypocotyls of 4 to 6 days old seedlings are removed, cut into 0.5 cm pieces and are placed on 0.8% agar. An Agrobacterium suspension (approx. 108 cells per ml, diluted from an overnight culture transformed with the gene of interest and suitable selection markers) is used for inoculation of the hypocotyl explants. After 3 days at room temperature and lighting, the tissues are transferred to a solid medium (1.6 g/l Gelrite) with Murashige and Skoog salts with B5 vitamins (Gamborg et al., Exp. Cell Res. 50:151-158 (1968)), 0.1 mg/l 2,4-D, 0.1 mg/l 6-furfurylaminopurine and 750 μg/ml MgCL2, and with 50 to 100 μg/ml cefotaxime and 400-500 μg/ml carbenicillin to kill residual bacteria. Individual cell lines are isolated after two to three months (with subcultures every four to six weeks) and are further cultivated on selective medium for tissue amplification (30° C., 16 hr photoperiod). Transformed tissues are subsequently further cultivated on non-selective medium during 2 to 3 months to give rise to somatic embryos. Healthy looking embryos of at least 4 mm length are transferred to tubes with SH medium in fine vermiculite, supplemented with 0.1 mg/l indole acetic acid, 6 furfurylaminopurine and gibberellic acid. The embryos are cultivated at 30° C. with a photoperiod of 16 hrs, and plantlets at the 2 to 3 leaf stage are transferred to pots with vermiculite and nutrients. The plants are hardened and subsequently moved to the greenhouse for further cultivation.

### Example 9

### Phenotypic Evaluation Procedure

### 9.1 Evaluation Setup

Approximately 35 independent T0 rice transformants were generated. The primary transformants were transferred from a tissue culture chamber to a greenhouse for growing and harvest of T1 seed. Six events, of which the T1 progeny segregated 3:1 for presence/absence of the transgene, were retained. For each of these events, approximately 10 T1 seedlings containing the transgene (hetero- and homo-zygotes) and approximately 10 T1 seedlings lacking the transgene (nullizygotes) were selected by monitoring visual marker expression. The transgenic plants and the corresponding nullizygotes were grown side-by-side at random positions. Greenhouse conditions were of shorts days (12 hours light), 28° C. in the light and 22° C. in the dark, and a relative humidity of 70%.

Four T1 events were further evaluated in the T2 generation following the same evaluation procedure as for the T1 generation but with more individuals per event. From the stage of sowing until the stage of maturity the plants were passed several times through a digital imaging cabinet. At each time point digital images (2048×1536 pixels, 16 million colours) were taken of each plant from at least 6 different angles.

### Drought Screen

Plants from T2 seeds are grown in potting soil under normal conditions until they approached the heading stage. They are then transferred to a “dry” section where irrigation is withheld. Humidity probes are inserted in randomly chosen pots to monitor the soil water content (SWC). When SWC goes below certain thresholds, the plants are automatically re-watered continuously until a normal level is reached again. The plants are then re-transferred again to normal conditions. The rest of the cultivation (plant maturation, seed harvest) is the same as for plants not grown under abiotic stress conditions. Growth and yield parameters are recorded as detailed for growth under normal conditions.

### Nitrogen Use Efficiency Screen

Rice plants from T2 seeds are grown in potting soil under normal conditions except for the nutrient solution. The pots are watered from transplantation to maturation with a specific nutrient solution containing reduced N nitrogen (N) content, usually between 7 to 8 times less. The rest of the cultivation (plant maturation, seed harvest) is the same as for plants not grown under abiotic stress. Growth and yield parameters are recorded as detailed for growth under normal conditions.

### Salt Stress Screen

Plants are grown on a substrate made of coco fibers and argex (3 to 1 ratio). A normal nutrient solution is used during the first two weeks after transplanting the plantlets in the greenhouse. After the first two weeks, 25 mM of salt (NaCl) is added to the nutrient solution, until the plants were harvested. Seed-related parameters are then measured.

### 9.2 Statistical Analysis: F Test

A two factor ANOVA (analysis of variants) was used as a statistical model for the overall evaluation of plant phenotypic characteristics. An F test was carried out on all the parameters measured of all the plants of all the events transformed with the gene of the present invention. The F test was carried out to check for an effect of the gene over all the transformation events and to verify for an overall effect of the gene, also known as a global gene effect. The threshold for significance for a true global gene effect was set at a 5% probability level for the F test. A significant F test value points to a gene effect, meaning that it is not only the mere presence or position of the gene that is causing the differences in phenotype.

Because two experiments with overlapping events were carried out, a combined analysis was performed. This is useful to check consistency of the effects over the two experiments, and if this is the case, to accumulate evidence from both experiments in order to increase confidence in the conclusion. The method used was a mixed-model approach that takes into account the multilevel structure of the data (i.e. experiment-event-segregants). P values were obtained by comparing likelihood ratio test to chi square distributions.

### 9.3 Parameters Measured

### Biomass-Related Parameter Measurement

From the stage of sowing until the stage of maturity the plants were passed several times through a digital imaging cabinet. At each time point digital images (2048×1536 pixels, 16 million colours) were taken of each plant from at least 6 different angles.

The plant aboveground area (or leafy biomass) was determined by counting the total number of pixels on the digital images from aboveground plant parts discriminated from the background. This value was averaged for the pictures taken on the same time point from the different angles and was converted to a physical surface value expressed in square mm by calibration. Experiments show that the aboveground plant area measured this way correlates with the biomass of plant parts above ground. The above ground area is the area measured at the time point at which the plant had reached its maximal leafy biomass. The early vigour is the plant (seedling) aboveground area three weeks post-germination.

Early vigour was determined by counting the total number of pixels from aboveground plant parts discriminated from the background. This value was averaged for the pictures taken on the same time point from different angles and was converted to a physical surface value expressed in square mm by calibration. The results described below are for plants three weeks post-germination.

### Seed-Related Parameter Measurements

The mature primary panicles were harvested, counted, bagged, barcode-labelled and then dried for three days in an oven at 37° C. The panicles were then threshed and all the seeds were collected and counted. The filled husks were separated from the empty ones using an air-blowing device. The empty husks were discarded and the remaining fraction was counted again. The filled husks were weighed on an analytical balance. The number of filled seeds was determined by counting the number of filled husks that remained after the separation step. The total seed yield was measured by weighing all filled husks harvested from a plant. Total seed number per plant was measured by counting the number of husks harvested from a plant. Thousand Kernel Weight (TKW) is extrapolated from the number of filled seeds counted and their total weight. The Harvest Index (HI) in the present invention is defined as the ratio between the total seed yield and the above ground area (mm²), multiplied by a factor 10⁶. The total number of flowers per panicle as defined in the present invention is the ratio between the total number of seeds and the number of mature primary panicles. The seed fill rate as defined in the present invention is the proportion (expressed as a %) of the number of filled seeds over the total number of seeds (or florets). Number of flowers per panicle was calculated from the number of total seeds and the number of first panicles and is an estimation of the average number of florets per panicle on a plant.

### Example 10

### Results of the Phenotypic Evaluation of the Transgenic Plants

Transgenic rice plants expressing the bHLH6-like protein of SEQ ID NO: 2 grown under normal growth conditions showed compared to the control plants, an increase in emergence vigour and in seed yield (as demonstrated by number of flowers per panicle, see table below). These increases were measured in 2 experiments with T1 and T2 generation plants and were statistically significant.

| | T1 generation | T2 generation |
|---|---|---|
| parameter | % increase | % increase |
| Emergence vigour | 11.9 | 2.8 |
| number of flowers per panicle | 7.4 | 6.2 |

Furthermore positive effects were observed for total weight of seeds, number of filled seeds and harvest index, in the T1 generation as well as in T2 generation.

### Example 11

### Cloning of the Nucleic Acid Sequence Used in the Methods of the Invention

### Cloning of SEQ ID NO: 57 (Coding for RrmJ/FtsJ Methyltransferase):

A cDNA-AFLP experiment performed on a synchronized tobacco BY2 cell culture (Nicotiana tabacum L. cv. Bright Yellow-2), and BY2 expressed sequence tags that were cell cycle modulated could be identified and were elected for further cloning. The expressed sequence tags were used to screen a tobacco cDNA library (in a Gateway compatible vector; Invitrogen, Paisley, UK) to isolate the full-length cDNA of interest, namely one coding for RrmJ/FtsJ methyltransferase nucleic acid sequence as represented by SEQ ID NO: 57.

The isolated plasmid comprising SEQ ID NO: 57 was then used in an LR reaction with a destination vector used for Oryza sativa transformation. This vector contained as functional elements within the T-DNA borders: a plant selectable marker; a screenable marker expression cassette; and a Gateway cassette intended for LR in vivo recombination with the nucleic acid sequence of interest already cloned in the entry clone. A rice oleosin promoter (SEQ ID NO: 91) for seed specific expression was located upstream of this Gateway cassette.

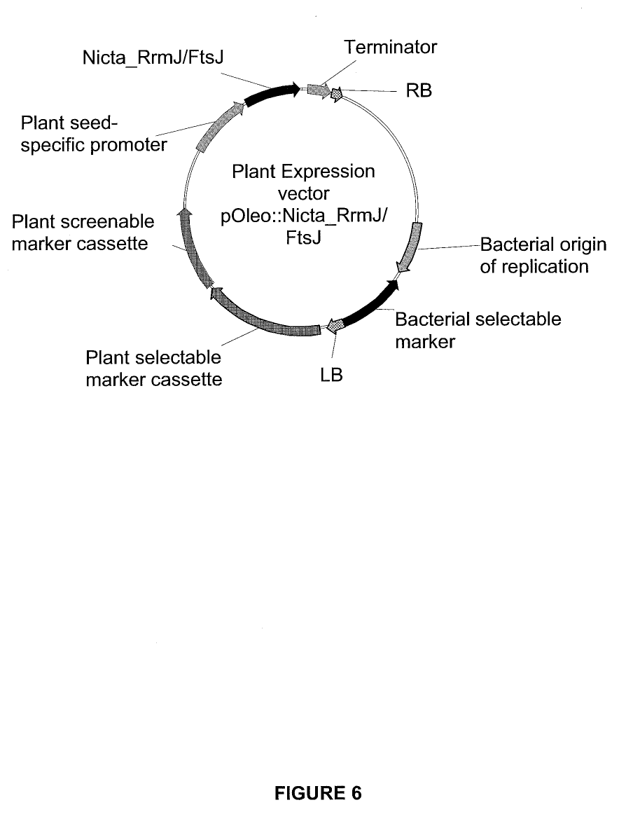

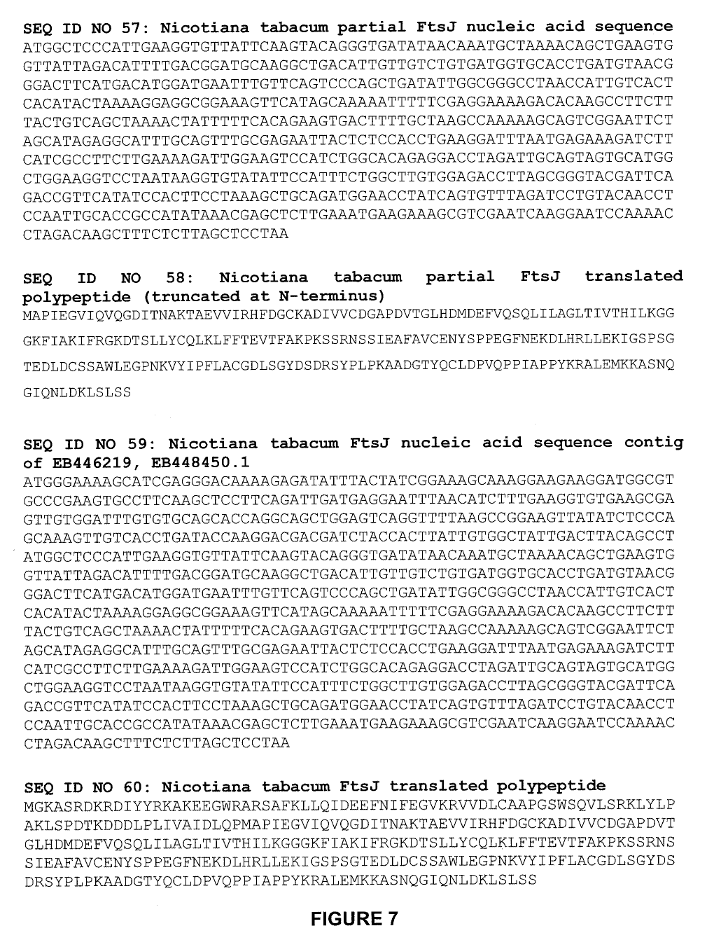

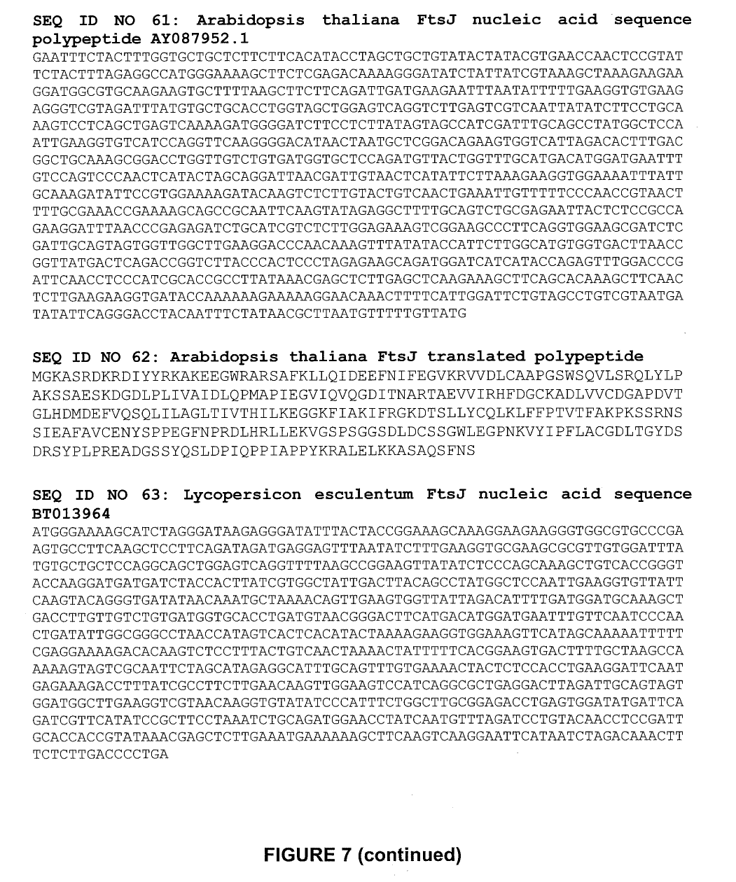

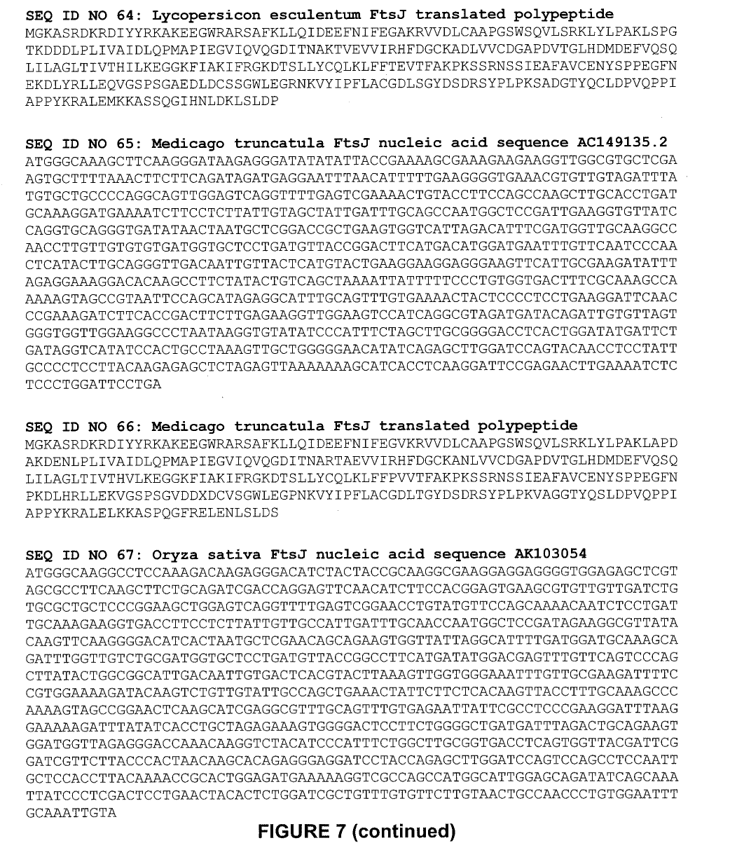

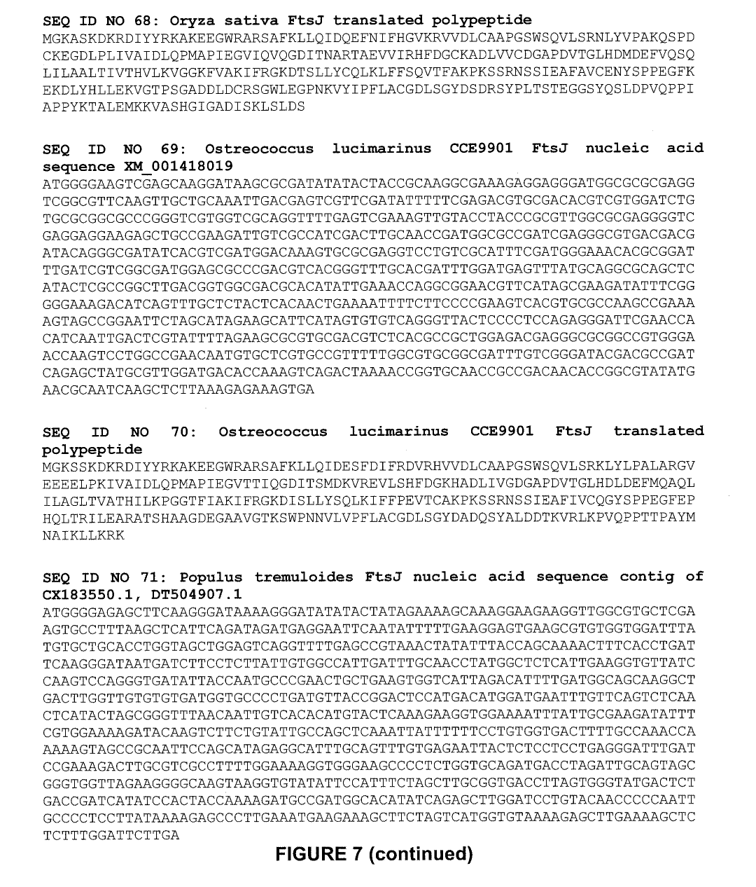

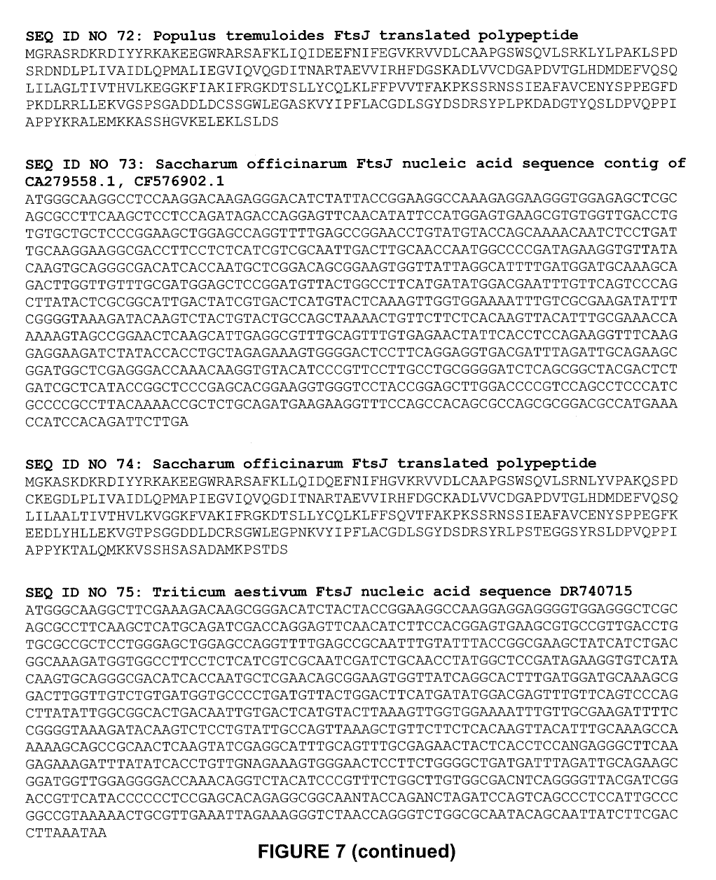

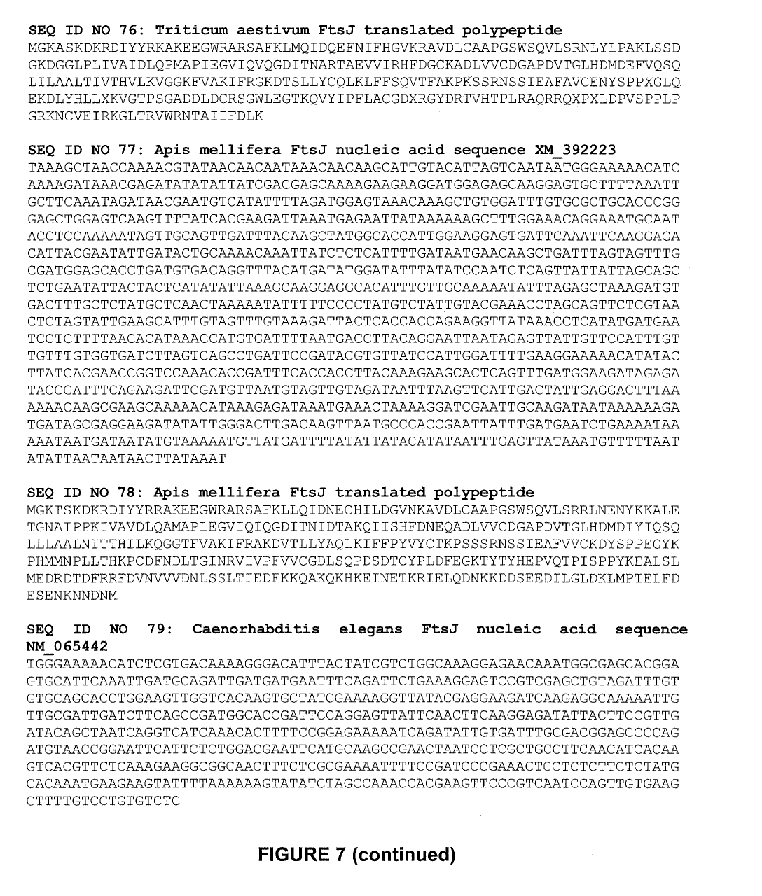

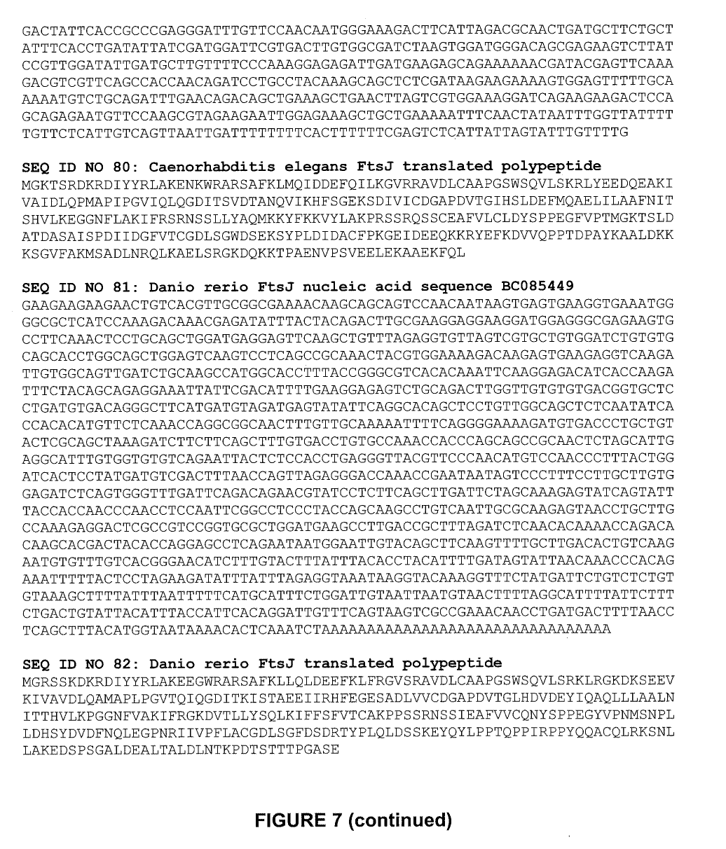

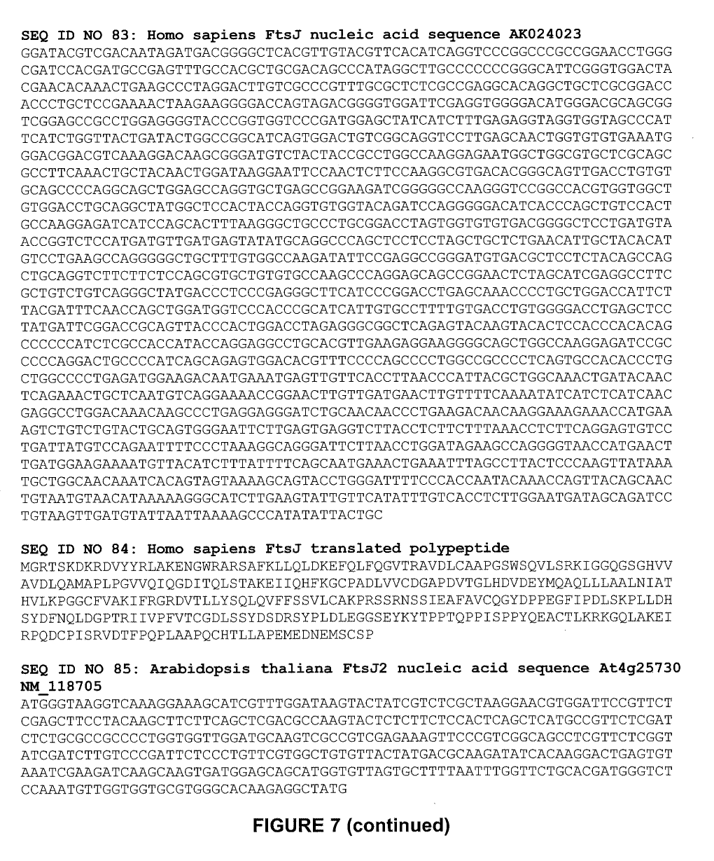

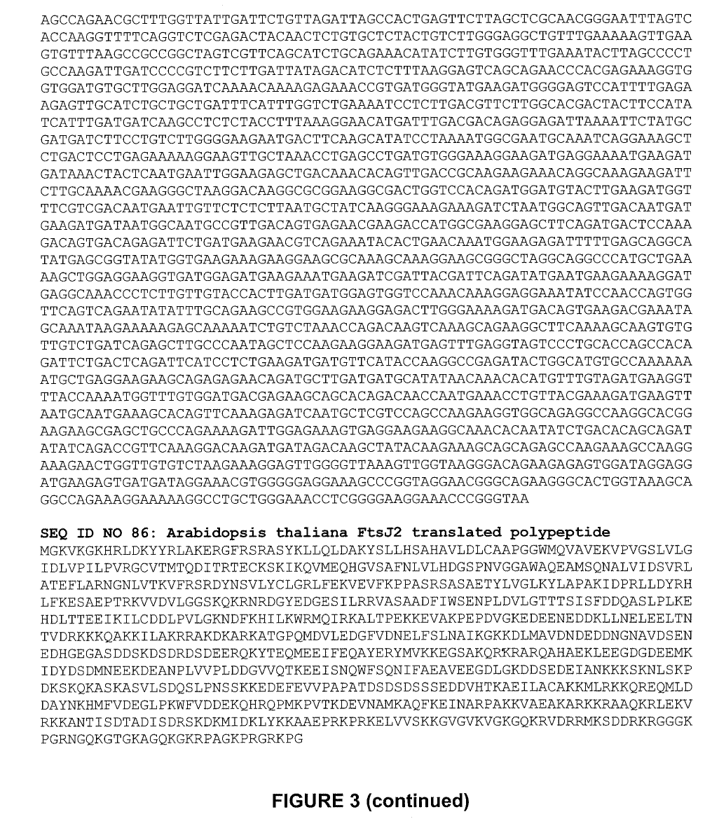

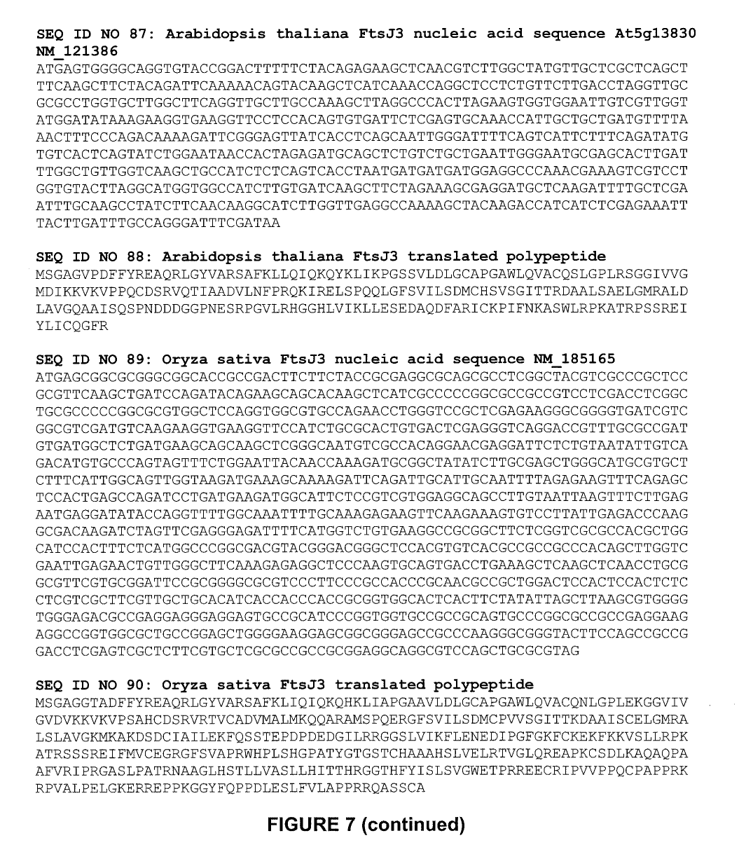

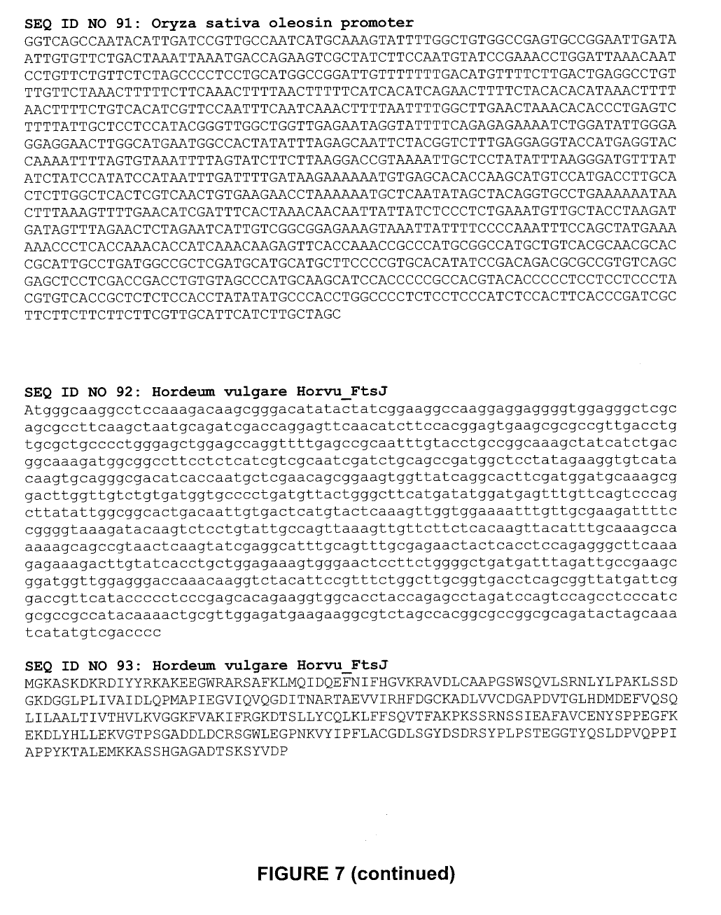

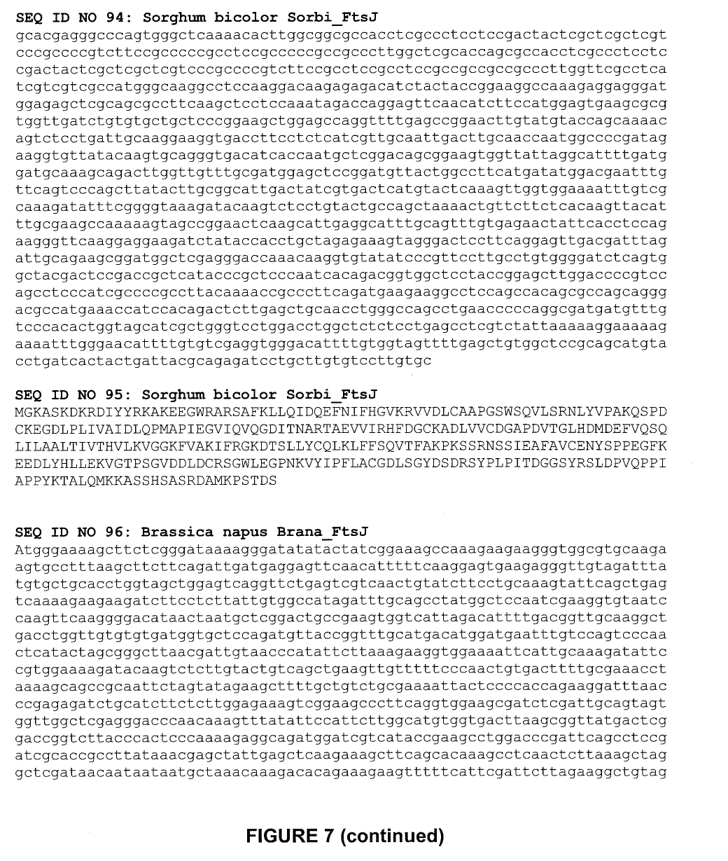

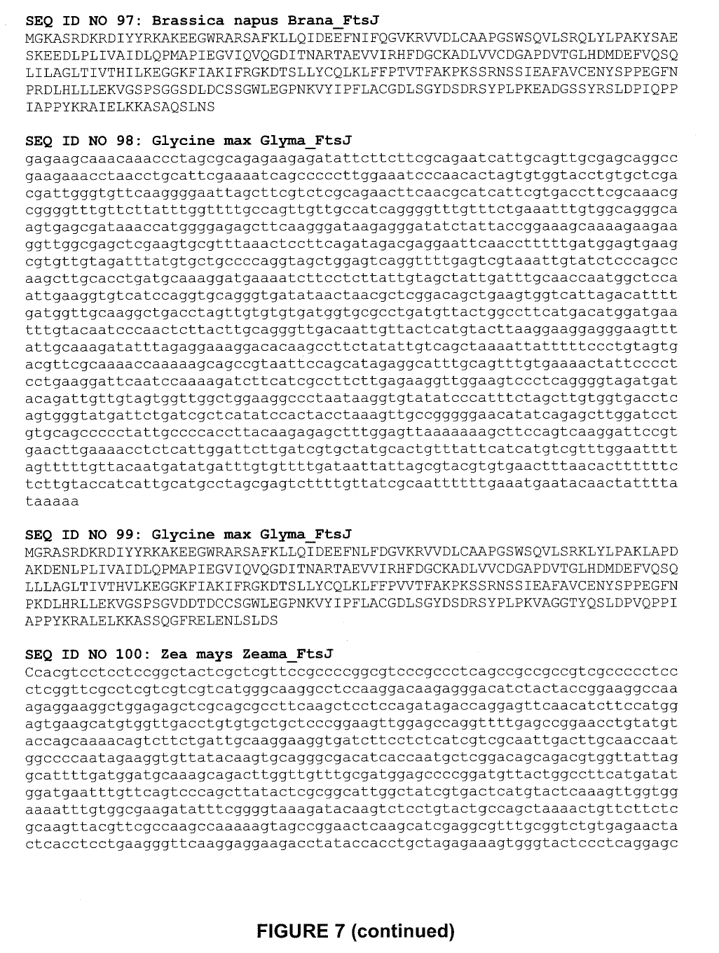

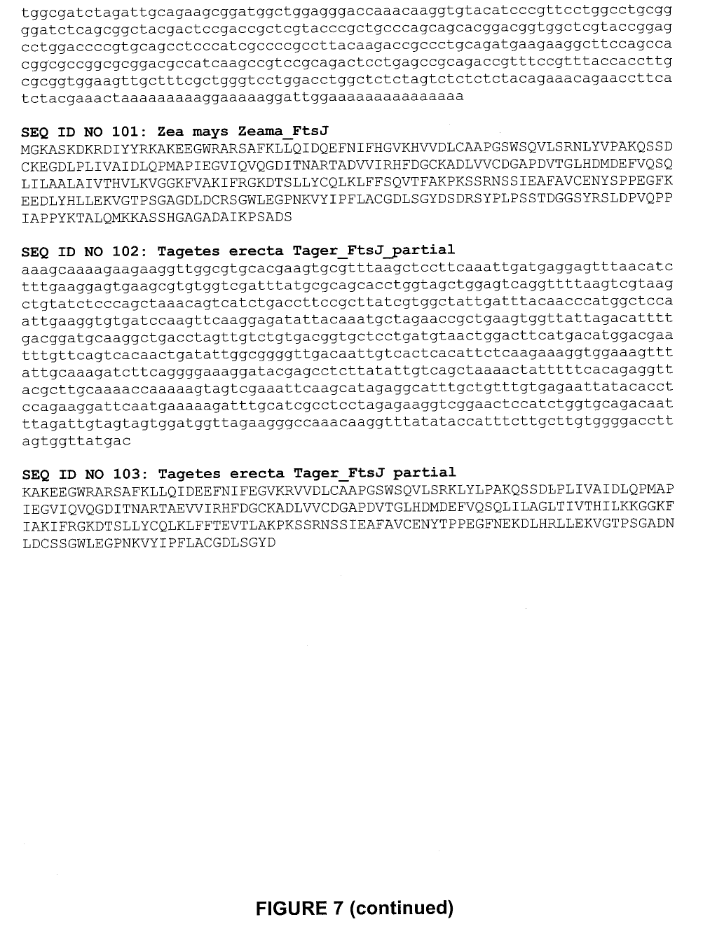

After the LR recombination step, the resulting expression vector pOleo::GRP (FIG. 6) was transformed into Agrobacterium strain LBA4044 according to methods well known in the art.

### Example 12

### Plant Transformation

### Rice Transformation

The Agrobacterium containing the expression vector was used to transform Oryza sativa plants. Mature dry seeds of the rice japonica cultivar Nipponbare were dehusked. Sterilization was carried out by incubating for one minute in 70% ethanol, followed by 30 minutes in 0.2% HgCl₂, followed by a 6 times 15 minutes wash with sterile distilled water. The sterile seeds were then germinated on a medium containing 2,4-D (callus induction medium). After incubation in the dark for four weeks, embryogenic, scutellum-derived calli were excised and propagated on the same medium. After two weeks, the calli were multiplied or propagated by subculture on the same medium for another 2 weeks. Embryogenic callus pieces were sub-cultured on fresh medium 3 days before co-cultivation (to boost cell division activity).

Agrobacterium strain LBA4404 containing the expression vector was used for co-cultivation. Agrobacterium was inoculated on AB medium with the appropriate antibiotics and cultured for 3 days at 28° C. The bacteria were then collected and suspended in liquid co-cultivation medium to a density (OD₆₀₀) of about 1. The suspension was then transferred to a Petri dish and the calli immersed in the suspension for 15 minutes. The callus tissues were then blotted dry on a filter paper and transferred to solidified, co-cultivation medium and incubated for 3 days in the dark at 25° C. Co-cultivated calli were grown on 2,4-D-containing medium for 4 weeks in the dark at 28° C. in the presence of a selection agent. During this period, rapidly growing resistant callus islands developed. After transfer of this material to a regeneration medium and incubation in the light, the embryogenic potential was released and shoots developed in the next four to five weeks. Shoots were excised from the calli and incubated for 2 to 3 weeks on an auxin-containing medium from which they were transferred to soil. Hardened shoots were grown under high humidity and short days in a greenhouse.

Approximately 35 independent T0 rice transformants were generated for one construct. The primary transformants were transferred from a tissue culture chamber to a greenhouse. After a quantitative PCR analysis to verify copy number of the T-DNA insert, only single copy transgenic plants that exhibit tolerance to the selection agent were kept for harvest of T1 seed. Seeds were then harvested three to five months after transplanting. The method yielded single locus transformants at a rate of over 50% (Aldemita and Hodges1996, Chan et al. 1993, Hiei et al. 1994).

### Example 13

### Phenotypic Evaluation Procedure

### 13.1 Evaluation Setup

Approximately 35 independent T0 rice transformants were generated. The primary transformants were transferred from a tissue culture chamber to a greenhouse for growing and harvest of T1 seed. Six events, of which the T1 progeny segregated 3:1 for presence/absence of the transgene, were retained. For each of these events, approximately 10 T1 seedlings containing the transgene (hetero- and homo-zygotes) and approximately 10 T1 seedlings lacking the transgene (nullizygotes) were selected by monitoring visual marker expression. The transgenic plants and the corresponding nullizygotes were grown side-by-side at random positions. Greenhouse conditions were of shorts days (12 hours light), 28° C. in the light and 22° C. in the dark, and a relative humidity of 70%.

Four T1 events were further evaluated in the T2 generation following the same evaluation procedure as for the T1 generation but with more individuals per event.

From the stage of sowing until the stage of maturity the plants were passed several times through a digital imaging cabinet. At each time point digital images (2048×1536 pixels, 16 million colours) were taken of each plant from at least 6 different angles.

### Drought Screen

Plants from T2 seeds are grown in potting soil under normal conditions until they approached the heading stage. They are then transferred to a “dry” section where irrigation is withheld. Humidity probes are inserted in randomly chosen pots to monitor the soil water content (SWC). When SWC goes below certain thresholds, the plants are automatically re-watered continuously until a normal level is reached again. The plants are then re-transferred again to normal conditions. The rest of the cultivation (plant maturation, seed harvest) is the same as for plants not grown under abiotic stress conditions. Growth and yield parameters are recorded as detailed for growth under normal conditions.

Nitrogen Use Efficiency Screen

Rice plants from T2 seeds are grown in potting soil under normal conditions except for the nutrient solution. The pots are watered from transplantation to maturation with a specific nutrient solution containing reduced N nitrogen (N) content, usually between 7 to 8 times less. The rest of the cultivation (plant maturation, seed harvest) is the same as for plants not grown under abiotic stress. Growth and yield parameters are recorded as detailed for growth under normal conditions.

### 13.2 Statistical Analysis: F Test

A two factor ANOVA (analysis of variants) was used as a statistical model for the overall evaluation of plant phenotypic characteristics. An F test was carried out on all the parameters measured of all the plants of all the events transformed with the gene of the present invention. The F test was carried out to check for an effect of the gene over all the transformation events and to verify for an overall effect of the gene, also known as a global gene effect. The threshold for significance for a true global gene effect was set at a 5% probability level for the F test. A significant F test value points to a gene effect, meaning that it is not only the mere presence or position of the gene that is causing the differences in phenotype.

Because two experiments with overlapping events were carried out, a combined analysis is performed. This is useful to check consistency of the effects over the two experiments, and if this is the case, to accumulate evidence from both experiments in order to increase confidence in the conclusion. The method used is a mixed-model approach that takes into account the multilevel structure of the data (i.e. experiment-event-segregants). P values were obtained by comparing likelihood ratio test to chi square distributions.

### 13.3 Parameters Measured

### Biomass-Related Parameter Measurement

The plant aboveground area (or leafy biomass) was determined by counting the total number of pixels on the digital images from aboveground plant parts discriminated from the background. This value was averaged for the pictures taken on the same time point from the different angles and was converted to a physical surface value expressed in square mm by calibration. Experiments show that the aboveground plant area measured this way correlates with the biomass of plant parts above ground. The above ground area is the area measured at the time point at which the plant had reached its maximal leafy biomass.

### Seed-Related Parameter Measurements

The mature primary panicles were harvested, counted, bagged, barcode-labelled and then dried for three days in an oven at 37° C. The panicles were then threshed and all the seeds were collected and counted. The filled husks were separated from the empty ones using an air-blowing device. The empty husks were discarded and the remaining fraction was counted again. The filled husks were weighed on an analytical balance. The number of filled seeds was determined by counting the number of filled husks that remained after the separation step. The total seed yield was measured by weighing all filled husks harvested from a plant. Total seed number per plant was measured by counting the number of husks harvested from a plant. Thousand Kernel Weight (TKW) is extrapolated from the number of filled seeds counted and their total weight. The Harvest Index (HI) in the present invention is defined as the ratio between the total seed yield and the above ground area (mm²), multiplied by a factor 10⁶. The total number of flowers per panicle as defined in the present invention is the ratio between the total number of seeds and the number of mature primary panicles. The seed fill rate as defined in the present invention is the proportion (expressed as a %) of the number of filled seeds over the total number of seeds (or florets).

### Example 14

### Results of the Phenotypic Evaluation of the Transgenic Plants

The transgenic rice plants expressing the GRP nucleic acid sequence represented by SEQ ID NO: 57 under control of the oleosin promoter showed an increase of more than 5% for total weight of seeds, number of filled seeds, fill rate, and harvest index.

| | Average % increase | Average % increase |
|---|---|---|
| | in 2 events in the T1 | in 2 events in the T2 |
| | generation | generation |
| Total seed yield per plant | 48% | 46% |
| Total number of filled seeds | 46% | 44% |
| Fill rate | 39% | 45% |
| Harvest index | 40% | 40% |

### Example 15

### Other Crop Transformation

### Corn Transformation

Transformation of maize (Zea mays) is performed with a modification of the method described by Ishida et al. (1996) Nature Biotech 14(6): 745-50. Transformation is genotype-dependent in corn and only specific genotypes are amenable to transformation and regeneration. The inbred line A188 (University of Minnesota) or hybrids with A188 as a parent are good sources of donor material for transformation, but other genotypes can be used successfully as well. Ears are harvested from corn plant approximately 11 days after pollination (DAP) when the length of the immature embryo is about 1 to 1.2 mm. Immature embryos are cocultivated with Agrobacterium tumefaciens containing the expression vector, and transgenic plants are recovered through organogenesis. Excised embryos are grown on callus induction medium, then maize regeneration medium, containing the selection agent (for example imidazolinone but various selection markers can be used). The Petri plates are incubated in the light at 25° C. for 2-3 weeks, or until shoots develop. The green shoots are transferred from each embryo to maize rooting medium and incubated at 25° C. for 2-3 weeks, until roots develop. The rooted shoots are transplanted to soil in the greenhouse. T1 seeds are produced from plants that exhibit tolerance to the selection agent and that contain a single copy of the T-DNA insert.

### Wheat Transformation

Transformation of wheat is performed with the method described by Ishida et al. (1996) Nature Biotech 14(6): 745-50. The cultivar Bobwhite (available from CIMMYT, Mexico) is commonly used in transformation. Immature embryos are co-cultivated with Agrobacterium tumefaciens containing the expression vector, and transgenic plants are recovered through organogenesis. After incubation with Agrobacterium, the embryos are grown in vitro on callus induction medium, then regeneration medium, containing the selection agent (for example imidazolinone but various selection markers can be used). The Petri plates are incubated in the light at 25° C. for 2-3 weeks, or until shoots develop. The green shoots are transferred from each embryo to rooting medium and incubated at 25° C. for 2-3 weeks, until roots develop. The rooted shoots are transplanted to soil in the greenhouse. T1 seeds are produced from plants that exhibit tolerance to the selection agent and that contain a single copy of the T-DNA insert.

### Soybean Transformation

Soybean is transformed according to a modification of the method described in the Texas A&M U.S. Pat. No. 5,164,310. Several commercial soybean varieties are amenable to transformation by this method. The cultivar Jack (available from the Illinois Seed foundation) is commonly used for transformation. Soybean seeds are sterilised for in vitro sowing. The hypocotyl, the radicle and one cotyledon are excised from seven-day old young seedlings. The epicotyl and the remaining cotyledon are further grown to develop axillary nodes. These axillary nodes are excised and incubated with Agrobacterium tumefaciens containing the expression vector. After the cocultivation treatment, the explants are washed and transferred to selection media. Regenerated shoots are excised and placed on a shoot elongation medium. Shoots no longer than 1 cm are placed on rooting medium until roots develop. The rooted shoots are transplanted to soil in the greenhouse. T1 seeds are produced from plants that exhibit tolerance to the selection agent and that contain a single copy of the T-DNA insert.

### Rapeseed/Canola Transformation

Cotyledonary petioles and hypocotyls of 5-6 day old young seedling are used as explants for tissue culture and transformed according to Babic et al. (1998, Plant Cell Rep 17: 183-188). The commercial cultivar Westar (Agriculture Canada) is the standard variety used for transformation, but other varieties can also be used. Canola seeds are surface-sterilized for in vitro sowing. The cotyledon petiole explants with the cotyledon attached are excised from the in vitro seedlings, and inoculated with Agrobacterium (containing the expression vector) by dipping the cut end of the petiole explant into the bacterial suspension. The explants are then cultured for 2 days on MSBAP-3 medium containing 3 mg/l BAP, 3% sucrose, 0.7% Phytagar at 23° C., 16 hr light. After two days of co-cultivation with Agrobacterium, the petiole explants are transferred to MSBAP-3 medium containing 3 mg/l BAP, cefotaxime, carbenicillin, or timentin (300 mg/l) for 7 days, and then cultured on MSBAP-3 medium with cefotaxime, carbenicillin, or timentin and selection agent until shoot regeneration. When the shoots are 5-10 mm in length, they are cut and transferred to shoot elongation medium (MSBAP-0.5, containing 0.5 mg/l BAP). Shoots of about 2 cm in length are transferred to the rooting medium (MS0) for root induction. The rooted shoots are transplanted to soil in the greenhouse. T1 seeds are produced from plants that exhibit tolerance to the selection agent and that contain a single copy of the T-DNA insert.

### Alfalfa Transformation

A regenerating clone of alfalfa (Medicago sativa) is transformed using the method of (McKersie et al., 1999 Plant Physiol 119: 839-847). Regeneration and transformation of alfalfa is genotype dependent and therefore a regenerating plant is required. Methods to obtain regenerating plants have been described. For example, these can be selected from the cultivar Rangelander (Agriculture Canada) or any other commercial alfalfa variety as described by Brown DCW and A Atanassov (1985. Plant Cell Tissue Organ Culture 4: 111-112). Alternatively, the RA3 variety (University of Wisconsin) has been selected for use in tissue culture (Walker et al., 1978 Am J Bot 65:654-659). Petiole explants are cocultivated with an overnight culture of Agrobacterium tumefaciens C58C1 pMP90 (McKersie et al., 1999 Plant Physiol 119: 839-847) or LBA4404 containing the expression vector. The explants are cocultivated for 3 d in the dark on SH induction medium containing 288 mg/L Pro, 53 mg/L thioproline, 4.35 g/L K2SO4, and 100 μm acetosyringinone. The explants are washed in half-strength Murashige-Skoog medium (Murashige and Skoog, 1962) and plated on the same SH induction medium without acetosyringinone but with a suitable selection agent and suitable antibiotic to inhibit Agrobacterium growth. After several weeks, somatic embryos are transferred to BOi2Y development medium containing no growth regulators, no antibiotics, and 50 g/L sucrose. Somatic embryos are subsequently germinated on half-strength Murashige-Skoog medium. Rooted seedlings were transplanted into pots and grown in a greenhouse. T1 seeds are produced from plants that exhibit tolerance to the selection agent and that contain a single copy of the T-DNA insert.

### Cotton Transformation

Cotton (Gossypium hirsutum L.) transformation is performed using Agrobacterium tumefaciens, on hypocotyls explants. The commercial cultivars such as Coker 130 or Coker 312 (SeedCo, Lubbock, Tex.) are standard varieties used for transformation, but other varieties can also be used. The seeds are surface sterilized and germinated in the dark. Hypocotyl explants are cut from the germinated seedlings to lengths of about 1-1.5 centimeter. The hypotocyl explant is submersed in the Agrobacterium tumefaciens inoculum containing the expression vector, for 5 minutes then co-cultivated for about 48 hours on MS+1.8 mg/l KNO3+2% glucose at 24° C., in the dark. The explants are transferred the same medium containing appropriate bacterial and plant selectable markers (renewed several times), until embryogenic calli is seen. The calli are separated and subcultured until somatic embryos appear. Plantlets derived from the somatic embryos are matured on rooting medium until roots develop. The rooted shoots are transplanted to potting soil in the greenhouse. T1 seeds are produced from plants that exhibit tolerance to the selection agent and that contain a single copy of the T-DNA insert.

### Example 16

### Cloning of the Nucleic Acid Sequence Used in the Methods of the Invention

Cloning of SEQ ID NO: 104 (Encoding a GRP, Wherein Said GRP Polypeptide is a Basic-Helix-Loop-Helix 4 (bHLH4) Polypeptide):

The nucleic acid sequence SEQ ID NO: 104 used in the methods of the invention was amplified by PCR using as template a custom-made Arabidopsis thaliana mixed tissues cDNA library (in pCMV Sport 6.0; Invitrogen, Paisley, UK). PCR was performed using Hifi Taq DNA polymerase in standard conditions, using 200 ng of template in a 50 μl PCR mix. The primers used were prm06763 (SEQ ID NO: 107; sense:

5′ ggggacaagtttgtacaaaaaagcaggcttaaacaatggctccga
cgaatgtt 3′

and prm06764 (SEQ ID NO: 108; reverse, complementary):

5′ ggggaccactttgtacaagaaagctgggttgctgacttcaattcat
ggac 3′,

which include the AttB sites for Gateway recombination. The amplified PCR fragment was purified also using standard methods. The first step of the Gateway procedure, the BP reaction, was then performed, during which the PCR fragment recombines in vivo with the pDONR201 plasmid to produce, according to the Gateway terminology, an “entry clone”. Plasmid pDONR201 was purchased from Invitrogen, as part of the Gateway® technology.

The entry clone comprising SEQ ID NO: 104 was then used in an LR reaction with a destination vector used for Oryza sativa transformation. This vector contained as functional elements within the T-DNA borders: a plant selectable marker; a screenable marker expression cassette; and a Gateway cassette intended for LR in vivo recombination with the nucleic acid sequence of interest already cloned in the entry clone. A rice HMGB promoter (SEQ ID NO: 106) for constitutive expression was located upstream of this Gateway cassette.

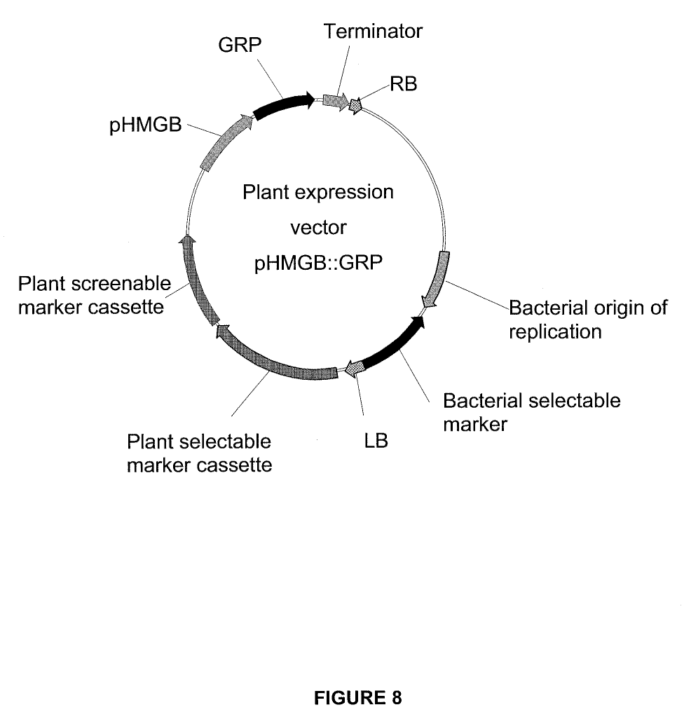

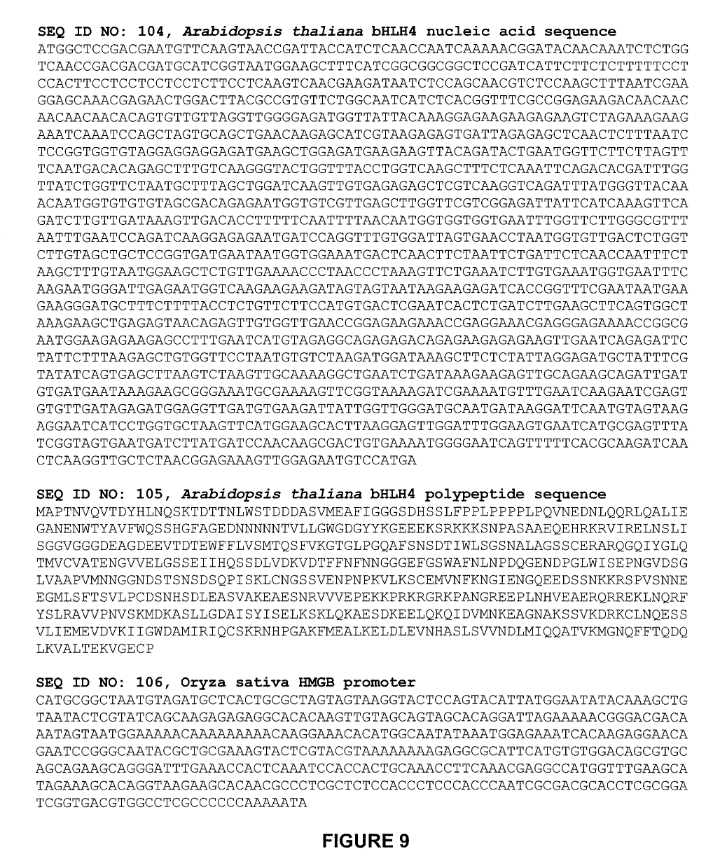

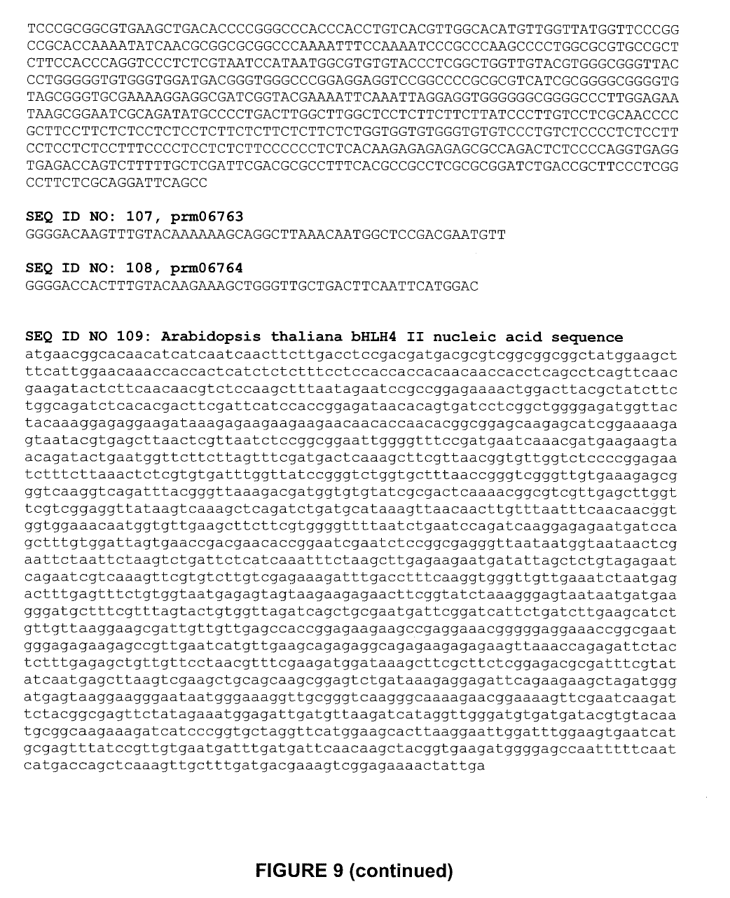

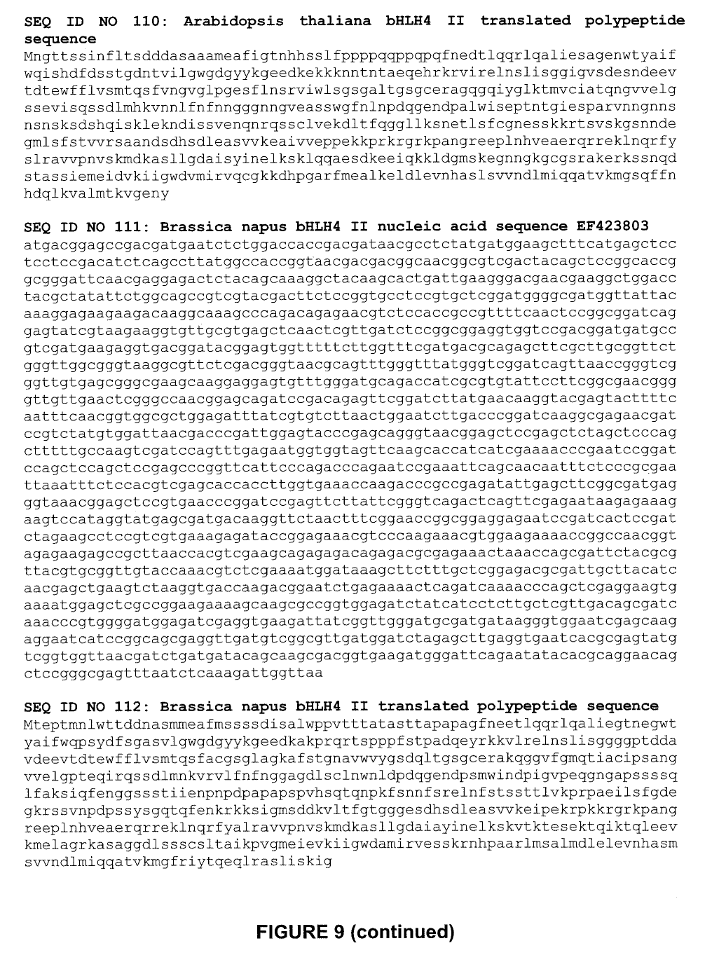

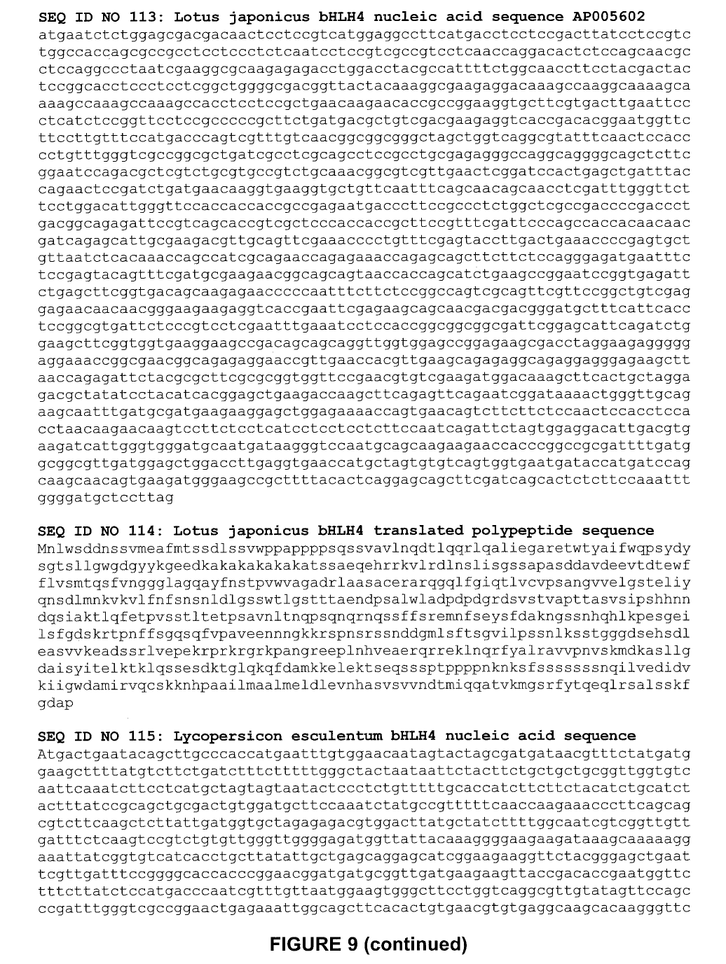

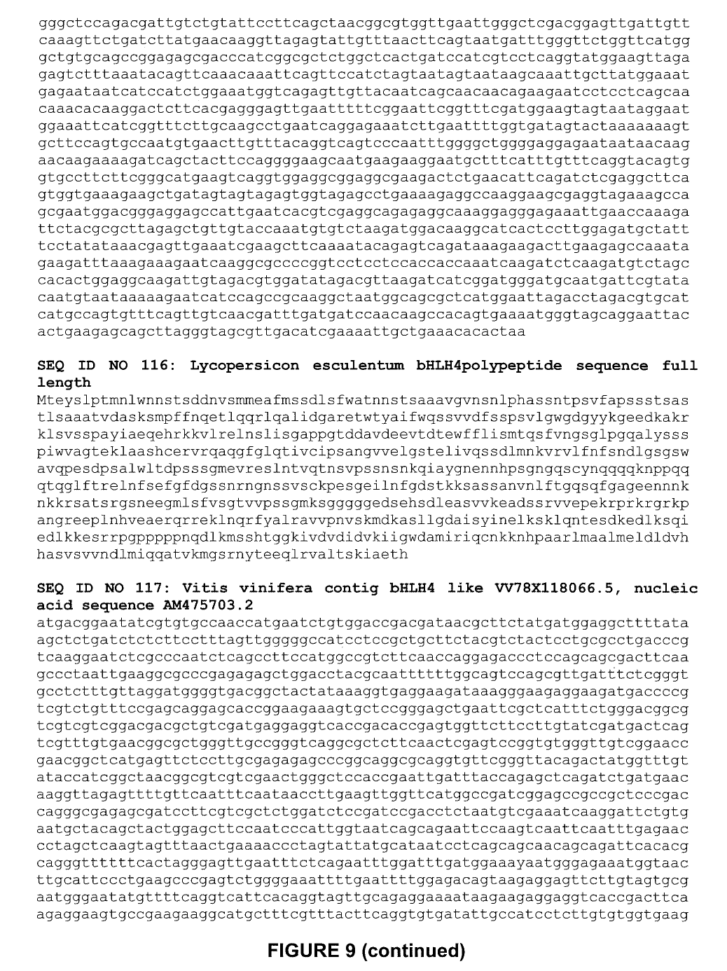

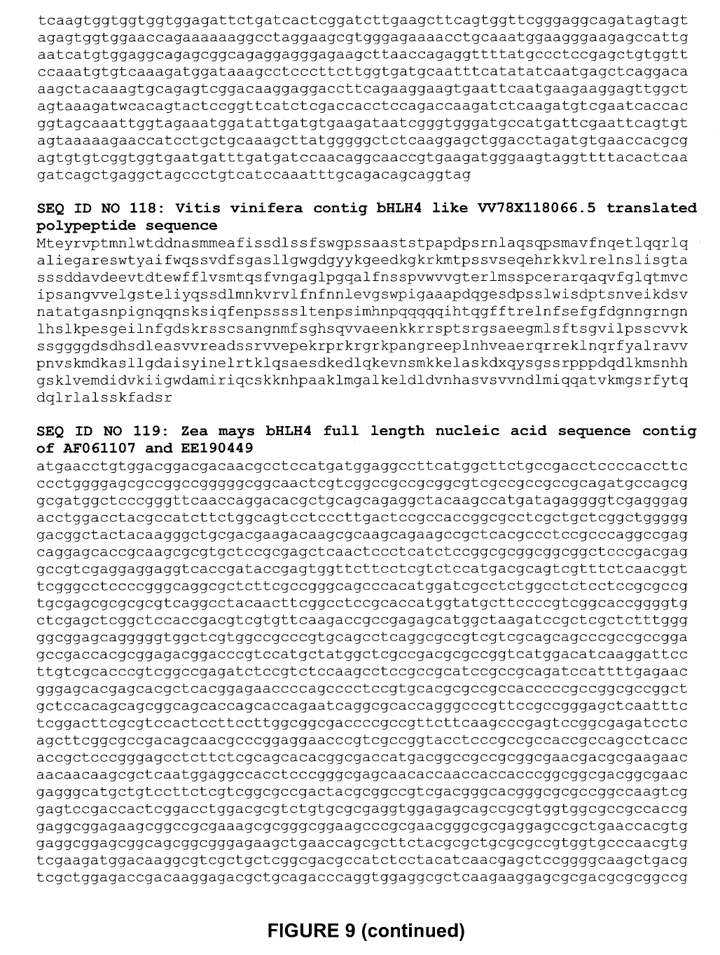

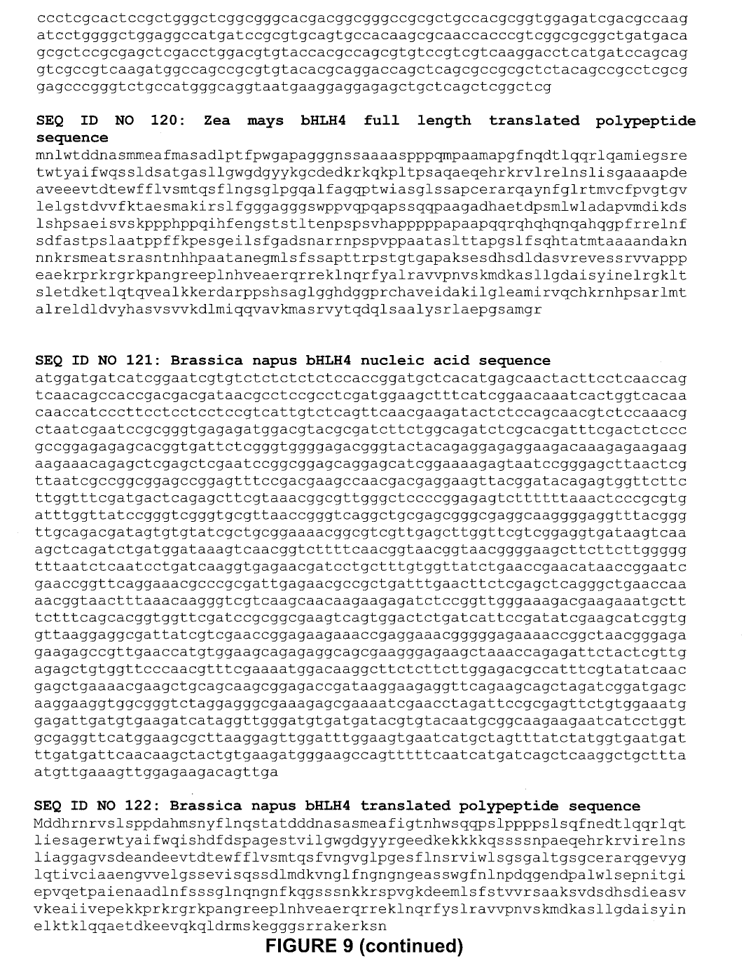

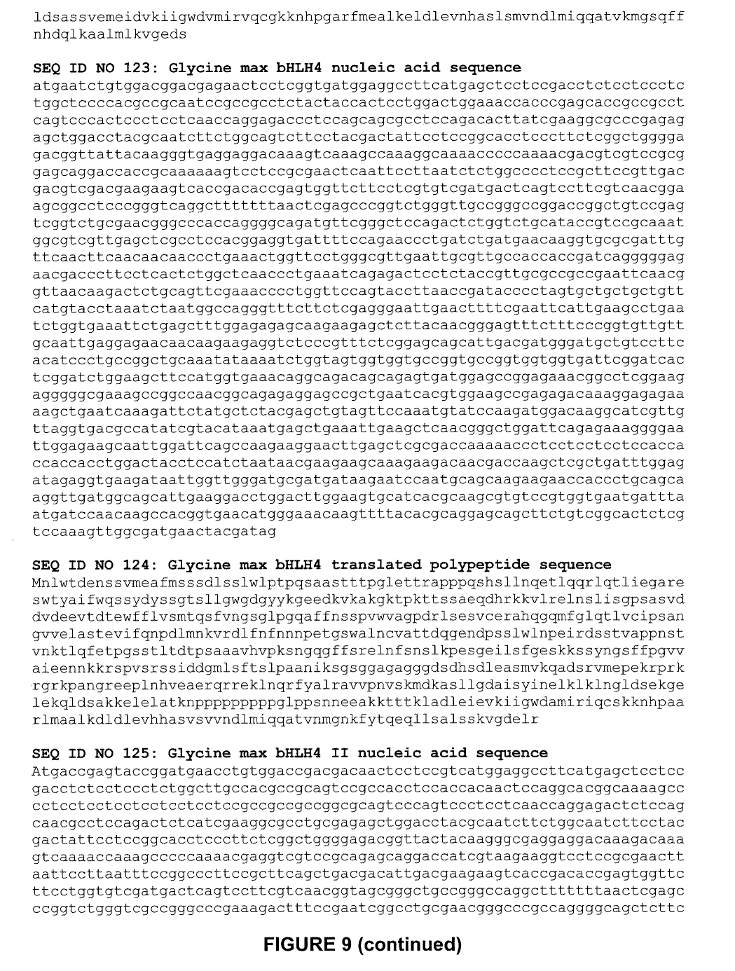

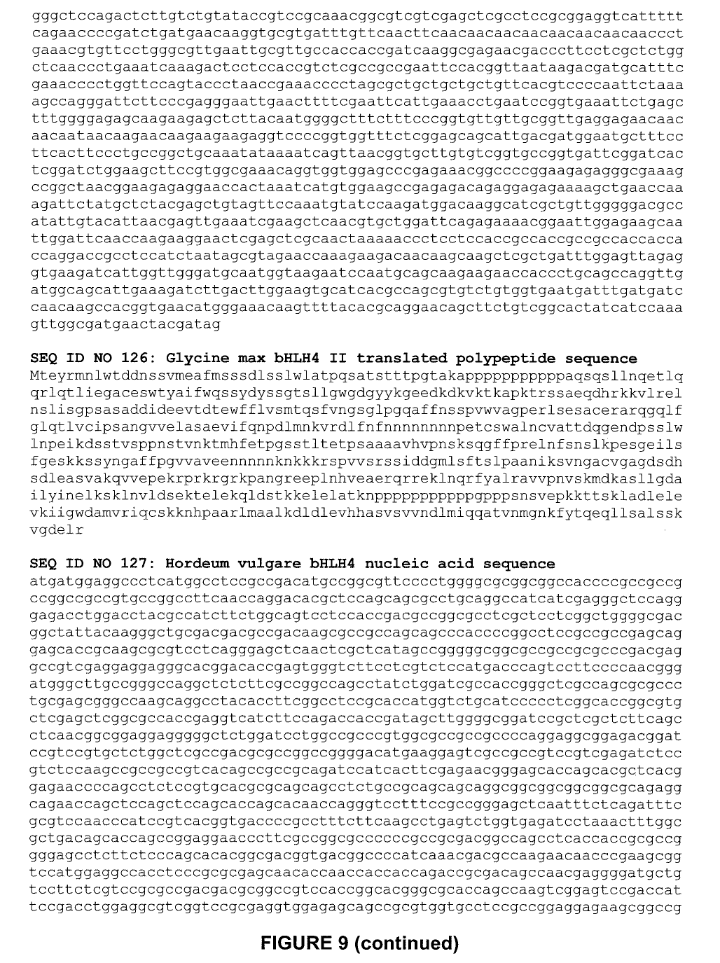

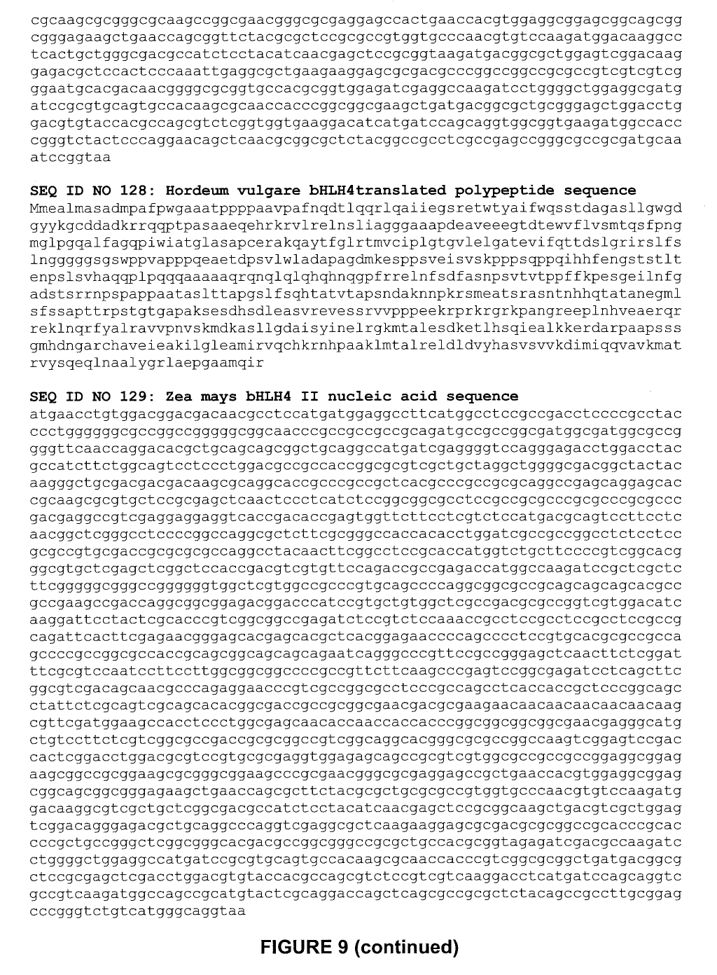

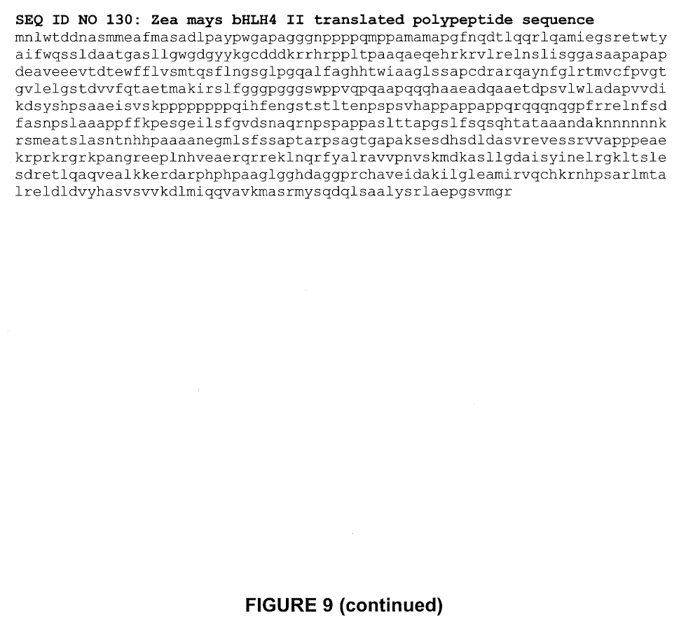

After the LR recombination step, the resulting expression vector pHMGB::GRP (FIG. 8) was transformed into Agrobacterium strain LBA4044 according to methods well known in the art.

### Example 17

### Plant Transformation

### Rice Transformation

The Agrobacterium containing the expression vector was used to transform Oryza sativa plants. Mature dry seeds of the rice japonica cultivar Nipponbare were dehusked. Sterilization was carried out by incubating for one minute in 70% ethanol, followed by 30 minutes in 0.2% HgCl₂, followed by a 6 times 15 minutes wash with sterile distilled water. The sterile seeds were then germinated on a medium containing 2,4-D (callus induction medium). After incubation in the dark for four weeks, embryogenic, scutellum-derived calli were excised and propagated on the same medium. After two weeks, the calli were multiplied or propagated by subculture on the same medium for another 2 weeks. Embryogenic callus pieces were sub-cultured on fresh medium 3 days before co-cultivatin (to boost cell division activity).

Agrobacterium strain LBA4404 containing the expression vector was used for co-cultivation. Agrobacterium was inoculated on AB medium with the appropriate antibiotics and cultured for 3 days at 28° C. The bacteria were then collected and suspended in liquid co-cultivation medium to a density (OD₆₀₀) of about 1. The suspension was then transferred to a Petri dish and the calli immersed in the suspension for 15 minutes. The callus tissues were then blotted dry on a filter paper and transferred to solidified, co-cultivation medium and incubated for 3 days in the dark at 25° C. Co-cultivated calli were grown on 2,4-D-containing medium for 4 weeks in the dark at 28° C. in the presence of a selection agent. During this period, rapidly growing resistant callus islands developed. After transfer of this material to a regeneration medium and incubation in the light, the embryogenic potential was released and shoots developed in the next four to five weeks. Shoots were excised from the calli and incubated for 2 to 3 weeks on an auxin-containing medium from which they were transferred to soil. Hardened shoots were grown under high humidity and short days in a greenhouse.

Approximately 35 independent T0 rice transformants were generated for one construct. The primary transformants were transferred from a tissue culture chamber to a greenhouse. After a quantitative PCR analysis to verify copy number of the T-DNA insert, only single copy transgenic plants that exhibit tolerance to the selection agent were kept for harvest of T1 seed. Seeds were then harvested three to five months after transplanting. The method yielded single locus transformants at a rate of over 50% (Aldemita and Hodges1996, Chan et al. 1993, Hiei et al. 1994).

### Example 18

### Phenotypic Evaluation Procedure

### 18.1 Evaluation Setup

Approximately 35 independent T0 rice transformants were generated. The primary transformants were transferred from a tissue culture chamber to a greenhouse for growing and harvest of T1 seed. Five events, of which the T1 progeny segregated 3:1 for presence/absence of the transgene, were retained. For each of these events, approximately 10 T1 seedlings containing the transgene (hetero- and homo-zygotes) and approximately 10 T1 seedlings lacking the transgene (nullizygotes) were selected by monitoring visual marker expression. The transgenic plants and the corresponding nullizygotes were grown side-by-side at random positions. Greenhouse conditions were of shorts days (12 hours light), 28° C. in the light and 22° C. in the dark, and a relative humidity of 70%.

Four T1 events were further evaluated in the T2 generation following the same evaluation procedure as for the T1 generation but with more individuals per event.

From the stage of sowing until the stage of maturity the plants were passed several times through a digital imaging cabinet. At each time point digital images (2048×1536 pixels, 16 million colours) were taken of each plant from at least 6 different angles.

### 18.2 Statistical Analysis: F Test

A two factor ANOVA (analysis of variants) was used as a statistical model for the overall evaluation of plant phenotypic characteristics. An F test was carried out on all the parameters measured of all the plants of all the events transformed with the gene of the present invention. The F test was carried out to check for an effect of the gene over all the transformation events and to verify for an overall effect of the gene, also known as a global gene effect. The threshold for significance for a true global gene effect was set at a 5% probability level for the F test. A significant F test value points to a gene effect, meaning that it is not only the mere presence or position of the gene that is causing the differences in phenotype.

Because two experiments with overlapping events were carried out, a combined analysis is performed. This is useful to check consistency of the effects over the two experiments, and if this is the case, to accumulate evidence from both experiments in order to increase confidence in the conclusion. The method used is a mixed-model approach that takes into account the multilevel structure of the data (i.e. experiment-event-segregants). P values were obtained by comparing likelihood ratio test to chi square distributions.

### 18.3 Parameters Measured

### Biomass-Related Parameter Measurement

The plant aboveground area (or leafy biomass) was determined by counting the total number of pixels on the digital images from aboveground plant parts discriminated from the background. This value was averaged for the pictures taken on the same time point from the different angles and was converted to a physical surface value expressed in square mm by calibration. Experiments show that the aboveground plant area measured this way correlates with the biomass of plant parts above ground. The above ground area is the area measured at the time point at which the plant had reached its maximal leafy biomass.

### Seed-Related Parameter Measurements

The mature primary panicles were harvested, counted, bagged, barcode-labelled and then dried for three days in an oven at 37° C. The panicles were then threshed and all the seeds were collected and counted. The filled husks were separated from the empty ones using an air-blowing device. The empty husks were discarded and the remaining fraction was counted again. The filled husks were weighed on an analytical balance. The number of filled seeds was determined by counting the number of filled husks that remained after the separation step. The total seed yield was measured by weighing all filled husks harvested from a plant. Total seed number per plant was measured by counting the number of husks harvested from a plant. Thousand Kernel Weight (TKW) is extrapolated from the number of filled seeds counted and their total weight. The Harvest Index (HI) in the present invention is defined as the ratio between the total seed yield and the above ground area (mm²), multiplied by a factor 10⁶. The total number of flowers per panicle as defined in the present invention is the ratio between the total number of seeds and the number of mature primary panicles. The seed fill rate as defined in the present invention is the proportion (expressed as a %) of the number of filled seeds over the total number of seeds (or florets).

### Example 19

### Results of the Phenotypic Evaluation of the Transgenic Plants

The transgenic rice plants expressing the GRP nucleic acid sequence represented by SEQ ID NO: 104 under control of the HMGB promoter showed an increase of more than 5% for total weight of seeds, number of filled seeds, fill rate, and harvest index.

| | Overall average | Average % |
|---|---|---|
| | % increase in | increase in |
| | 5 events in the | 2 events in the |
| | T1 generation | T2 generation |
| Early vigour | 21% | 7% |
| Total seed yield per plant | 23% | 27% |
| Total number of filled seeds | 22% | 28% |
| Seed fill rate | 12% | 15% |
| Harvest index | 18% | 17% |
| Greenness index | 4% | 9% |

### Example 20

### Other Crop Transformation

### Corn Transformation

Transformation of maize (Zea mays) is performed with a modification of the method described by Ishida et al. (1996) Nature Biotech 14(6): 745-50. Transformation is genotype-dependent in corn and only specific genotypes are amenable to transformation and regeneration. The inbred line A188 (University of Minnesota) or hybrids with A188 as a parent are good sources of donor material for transformation, but other genotypes can be used successfully as well. Ears are harvested from corn plant approximately 11 days after pollination (DAP) when the length of the immature embryo is about 1 to 1.2 mm. Immature embryos are cocultivated with Agrobacterium tumefaciens containing the expression vector, and transgenic plants are recovered through organogenesis. Excised embryos are grown on callus induction medium, then maize regeneration medium, containing the selection agent (for example imidazolinone but various selection markers can be used). The Petri plates are incubated in the light at 25° C. for 2-3 weeks, or until shoots develop. The green shoots are transferred from each embryo to maize rooting medium and incubated at 25° C. for 2-3 weeks, until roots develop. The rooted shoots are transplanted to soil in the greenhouse. T1 seeds are produced from plants that exhibit tolerance to the selection agent and that contain a single copy of the T-DNA insert.

### Wheat Transformation

Transformation of wheat is performed with the method described by Ishida et al. (1996) Nature Biotech 14(6): 745-50. The cultivar Bobwhite (available from CIMMYT, Mexico) is commonly used in transformation. Immature embryos are co-cultivated with Agrobacterium tumefaciens containing the expression vector, and transgenic plants are recovered through organogenesis. After incubation with Agrobacterium, the embryos are grown in vitro on callus induction medium, then regeneration medium, containing the selection agent (for example imidazolinone but various selection markers can be used). The Petri plates are incubated in the light at 25° C. for 2-3 weeks, or until shoots develop. The green shoots are transferred from each embryo to rooting medium and incubated at 25° C. for 2-3 weeks, until roots develop. The rooted shoots are transplanted to soil in the greenhouse. T1 seeds are produced from plants that exhibit tolerance to the selection agent and that contain a single copy of the T-DNA insert.

### Soybean Transformation

Soybean is transformed according to a modification of the method described in the Texas A&M U.S. Pat. No. 5,164,310. Several commercial soybean varieties are amenable to transformation by this method. The cultivar Jack (available from the Illinois Seed foundation) is commonly used for transformation. Soybean seeds are sterilised for in vitro sowing. The hypocotyl, the radicle and one cotyledon are excised from seven-day old young seedlings. The epicotyl and the remaining cotyledon are further grown to develop axillary nodes. These axillary nodes are excised and incubated with Agrobacterium tumefaciens containing the expression vector. After the cocultivation treatment, the explants are washed and transferred to selection media. Regenerated shoots are excised and placed on a shoot elongation medium. Shoots no longer than 1 cm are placed on rooting medium until roots develop. The rooted shoots are transplanted to soil in the greenhouse. T1 seeds are produced from plants that exhibit tolerance to the selection agent and that contain a single copy of the T-DNA insert.

### Rapeseed/Canola Transformation

Cotyledonary petioles and hypocotyls of 5-6 day old young seedling are used as explants for tissue culture and transformed according to Babic et al. (1998, Plant Cell Rep 17: 183-188). The commercial cultivar Westar (Agriculture Canada) is the standard variety used for transformation, but other varieties can also be used. Canola seeds are surface-sterilized for in vitro sowing. The cotyledon petiole explants with the cotyledon attached are excised from the in vitro seedlings, and inoculated with Agrobacterium (containing the expression vector) by dipping the cut end of the petiole explant into the bacterial suspension. The explants are then cultured for 2 days on MSBAP-3 medium containing 3 mg/l BAP, 3% sucrose, 0.7% Phytagar at 23° C., 16 hr light. After two days of co-cultivation with Agrobacterium, the petiole explants are transferred to MSBAP-3 medium containing 3 mg/l BAP, cefotaxime, carbenicillin, or timentin (300 mg/l) for 7 days, and then cultured on MSBAP-3 medium with cefotaxime, carbenicillin, or timentin and selection agent until shoot regeneration. When the shoots are 5-10 mm in length, they are cut and transferred to shoot elongation medium (MSBAP-0.5, containing 0.5 mg/l BAP). Shoots of about 2 cm in length are transferred to the rooting medium (MS0) for root induction. The rooted shoots are transplanted to soil in the greenhouse. T1 seeds are produced from plants that exhibit tolerance to the selection agent and that contain a single copy of the T-DNA insert.

### Alfalfa Transformation

A regenerating clone of alfalfa (Medicago sativa) is transformed using the method of (McKersie et al., 1999 Plant Physiol 119: 839-847). Regeneration and transformation of alfalfa is genotype dependent and therefore a regenerating plant is required. Methods to obtain regenerating plants have been described. For example, these can be selected from the cultivar Rangelander (Agriculture Canada) or any other commercial alfalfa variety as described by Brown DCW and A Atanassov (1985. Plant Cell Tissue Organ Culture 4: 111-112). Alternatively, the RA3 variety (University of Wisconsin) has been selected for use in tissue culture (Walker et al., 1978 Am J Bot 65:654-659). Petiole explants are cocultivated with an overnight culture of Agrobacterium tumefaciens C58C1 pMP90 (McKersie et al., 1999 Plant Physiol 119: 839-847) or LBA4404 containing the expression vector. The explants are cocultivated for 3 d in the dark on SH induction medium containing 288 mg/L Pro, 53 mg/L thioproline, 4.35 g/L K2SO4, and 100 μm acetosyringinone. The explants are washed in half-strength Murashige-Skoog medium (Murashige and Skoog, 1962) and plated on the same SH induction medium without acetosyringinone but with a suitable selection agent and suitable antibiotic to inhibit Agrobacterium growth. After several weeks, somatic embryos are transferred to BOi2Y development medium containing no growth regulators, no antibiotics, and 50 g/L sucrose. Somatic embryos are subsequently germinated on half-strength Murashige-Skoog medium. Rooted seedlings were transplanted into pots and grown in a greenhouse. T1 seeds are produced from plants that exhibit tolerance to the selection agent and that contain a single copy of the T-DNA insert.

### Cotton Transformation

Cotton (Gossypium hirsutum L.) transformation is performed using Agrobacterium tumefaciens, on hypocotyls explants. The commercial cultivars such as Coker 130 or Coker 312 (SeedCo, Lubbock, Tex.) are standard varieties used for transformation, but other varieties can also be used. The seeds are surface sterilized and germinated in the dark. Hypocotyl explants are cut from the germinated seedlings to lengths of about 1-1.5 centimeter. The hypotocyl explant is submersed in the Agrobacterium tumefaciens inoculum containing the expression vector, for 5 minutes then co-cultivated for about 48 hours on MS+1.8 mg/l KNO3+2% glucose at 24° C., in the dark. The explants are transferred the same medium containing appropriate bacterial and plant selectable markers (renewed several times), until embryogenic calli is seen. The calli are separated and subcultured until somatic embryos appear. Plantlets derived from the somatic embryos are matured on rooting medium until roots develop. The rooted shoots are transplanted to potting soil in the greenhouse. T1 seeds are produced from plants that exhibit tolerance to the selection agent and that contain a single copy of the T-DNA insert.

### Example 21

### Abiotic Stress Screens

### Drought Screen

Rice plants from T1, T2 or further generations are grown in potting soil under normal conditions until they approached the heading stage. They are then transferred to a “dry” section where irrigation is withheld. Humidity probes are inserted in randomly chosen pots to monitor the soil water content (SWC). When SWC goes below certain thresholds, the plants are automatically re-watered continuously until a normal level is reached again. The plants are then re-transferred again to normal conditions. The rest of the cultivation (plant maturation, seed harvest) is the same as for plants not grown under abiotic stress conditions. Growth and yield parameters are recorded as detailed for growth under normal conditions.

### Salt Stress Screen

Rice plants from T1, T2 or further generations are grown on a substrate made of coco fibers and argex (3 to 1 ratio). A normal nutrient solution is used during the first two weeks after transplanting the plantlets in the greenhouse. After the first two weeks, 25 mM of salt (NaCl) is added to the nutrient solution, until the plants are harvested. Growth and yield parameters are recorded as detailed for growth under normal conditions.

### Nitrogen Use Efficiency Screen

Rice plants from T1, T2 or further generations are grown in potting soil under normal conditions except for the nutrient solution. The pots are watered from transplantation to maturation with a specific nutrient solution containing reduced N nitrogen (N) content, usually between 7 to 8 times less. The rest of the cultivation (plant maturation, seed harvest) is the same as for plants not grown under abiotic stress. Growth and yield parameters are recorded as detailed for growth under normal conditions.

### Example 22

### Cloning of the Nucleic Acid Sequence Used in the Methods of the Invention

### Cloning of SEQ ID NO: 131 (Encoding a GRP, Wherein Said GRP Polypeptide is an Isopentenyl Transferase (IPT) Polypeptide):

The nucleic acid sequence SEQ ID NO: 131 used in the methods of the invention was amplified by PCR using as template a custom-made Arabidopsis thaliana mixed tissues cDNA library (in pCMV Sport 6.0; Invitrogen, Paisley, UK). PCR was performed using Hifi Taq DNA polymerase in standard conditions, using 200 ng of template in a 50 μl PCR mix. The primers used were prm03095 (SEQ ID NO: 134; sense:

5′ ggggacaagtttgtacaaaaaagcaggcttcacaatgacagaactc
aacttccac 3′

and prm03096 (SEQ ID NO: 135; reverse, complementary):

5′ ggggaccactttgtacaagaaagctgggtaactaattttgcaccaa
atg 3′,

which include the AttB sites for Gateway recombination. The amplified PCR fragment was purified also using standard methods. The first step of the Gateway procedure, the BP reaction, was then performed, during which the PCR fragment recombines in vivo with the pDONR201 plasmid to produce, according to the Gateway terminology, an “entry clone”. Plasmid pDONR201 was purchased from Invitrogen, as part of the Gateway® technology.

The entry clone comprising SEQ ID NO: 131 was then used in an LR reaction with a destination vector used for Oryza sativa transformation. This vector contained as functional elements within the T-DNA borders: a plant selectable marker; a screenable marker expression cassette; and a Gateway cassette intended for LR in vivo recombination with the nucleic acid sequence of interest already cloned in the entry clone. A rice prolamin promoter (SEQ ID NO: 133) for seed-specific expression was located upstream of this Gateway cassette.

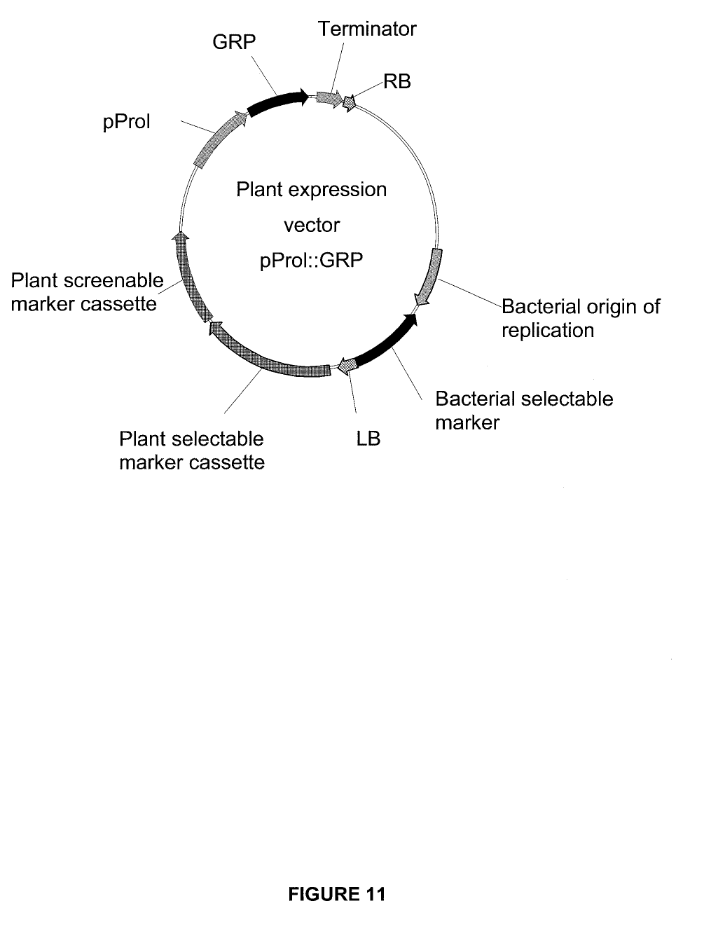

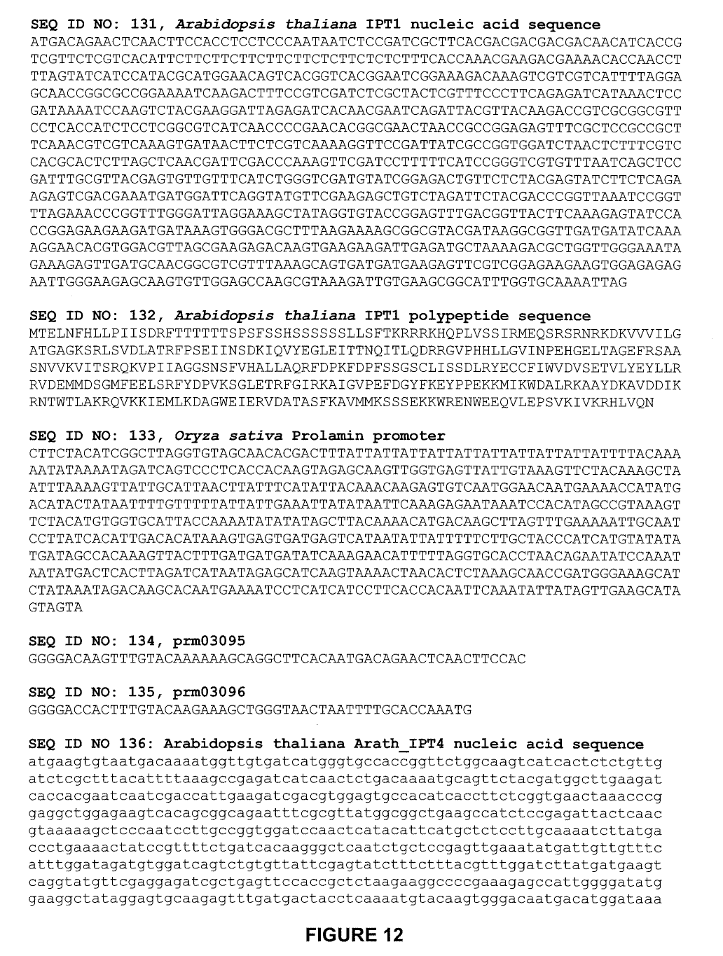

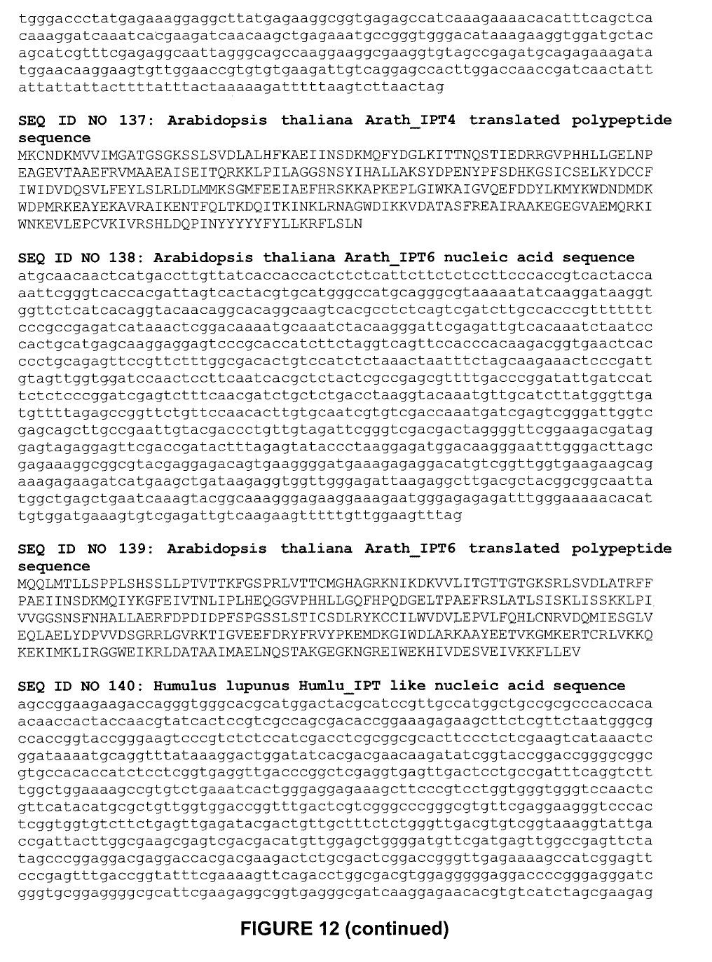

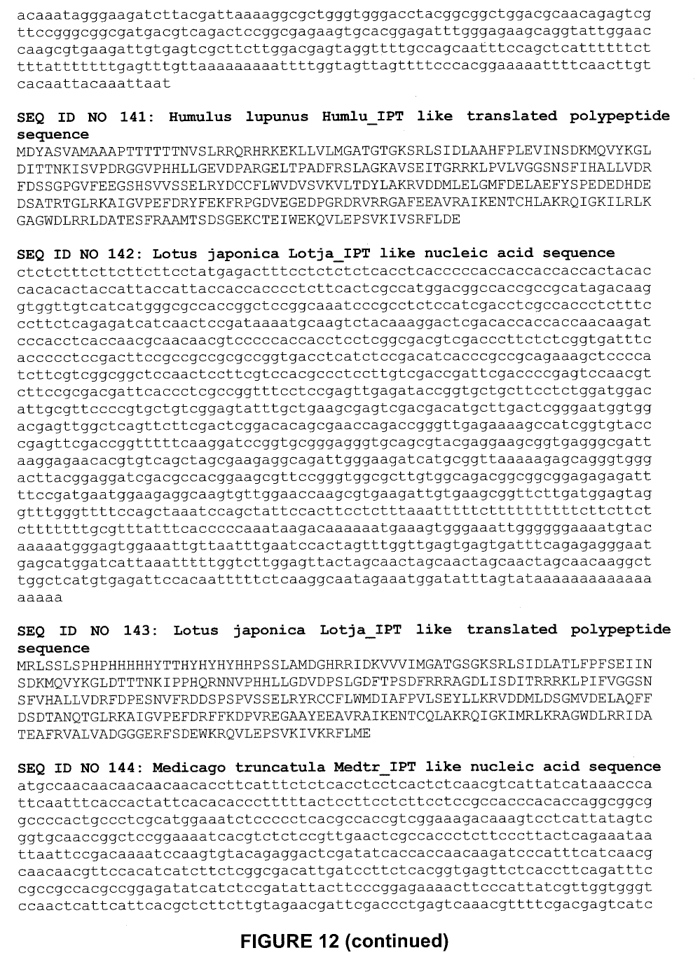

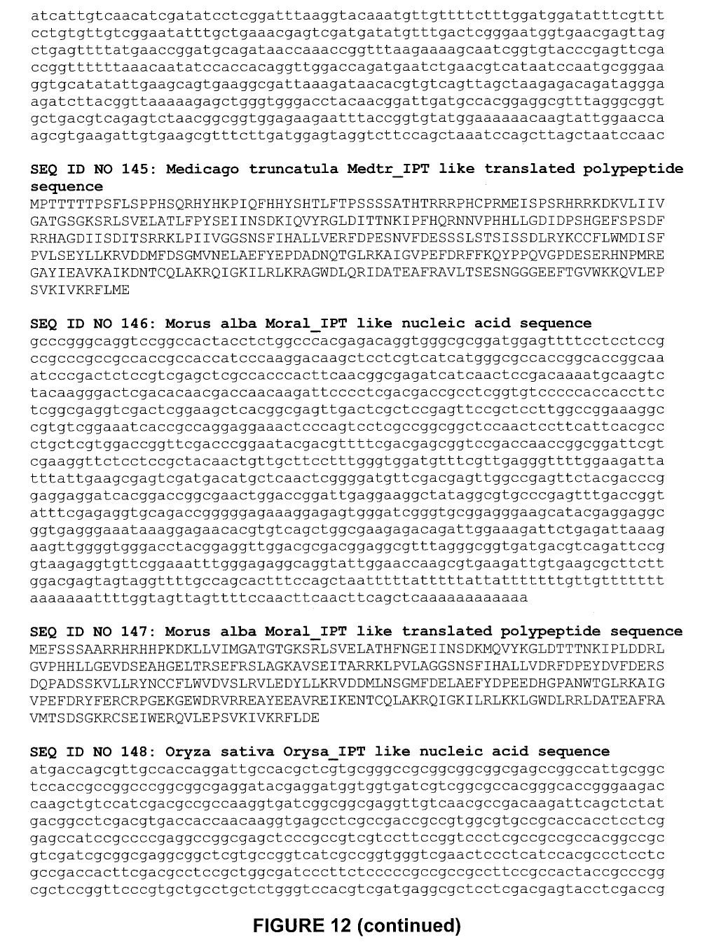

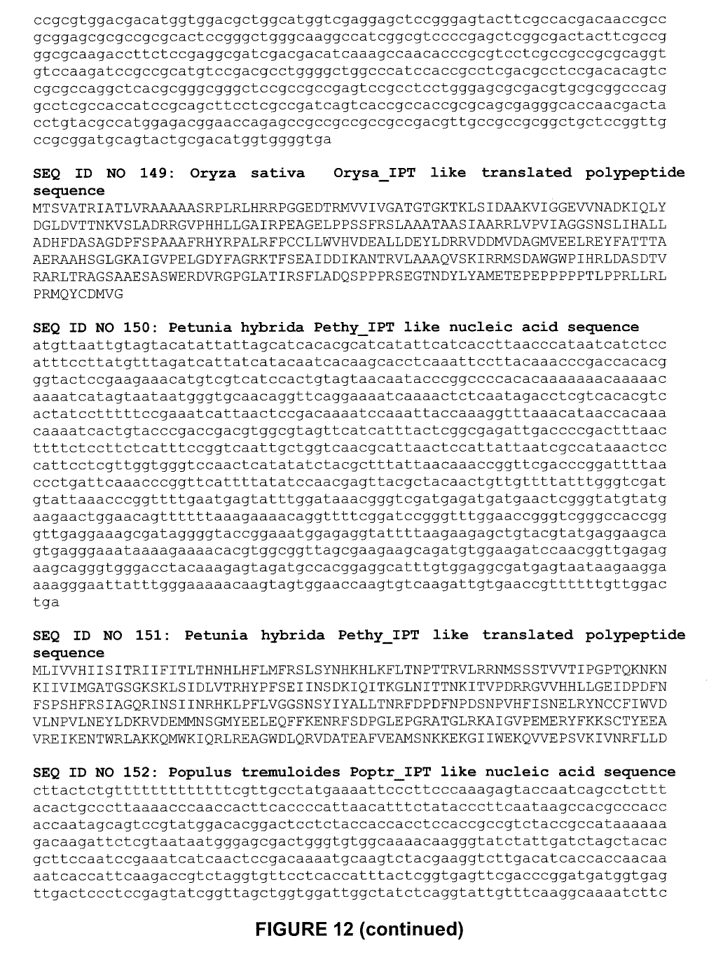

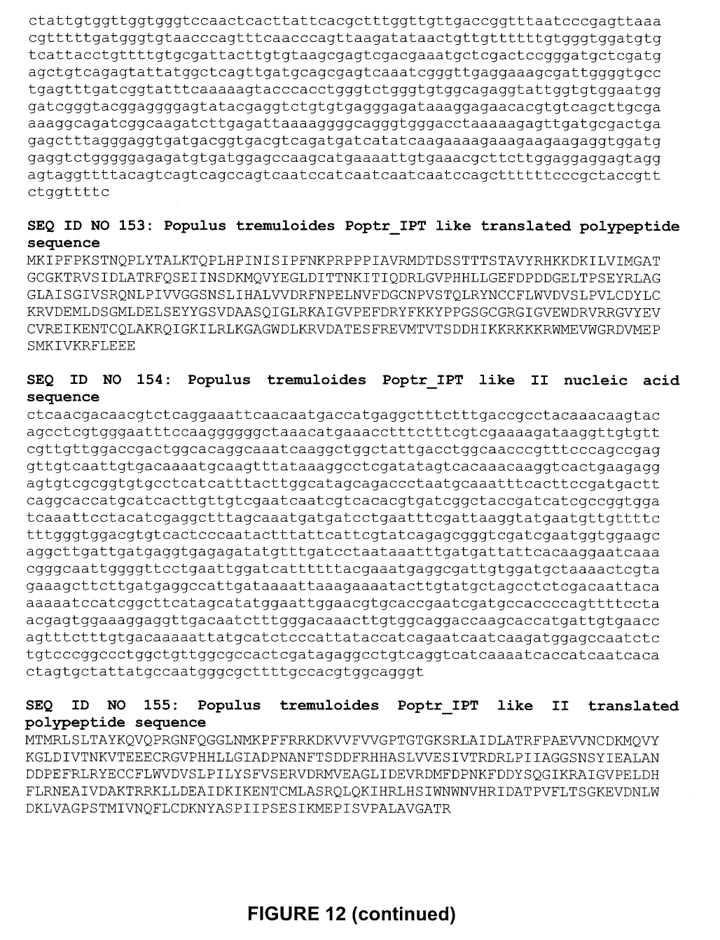

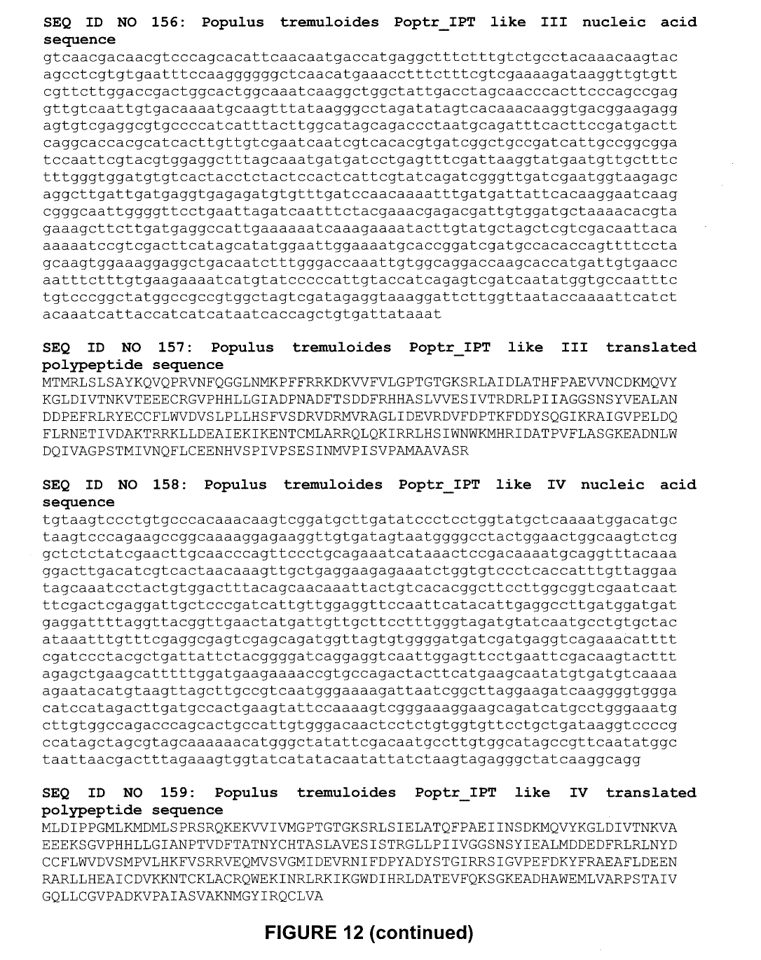

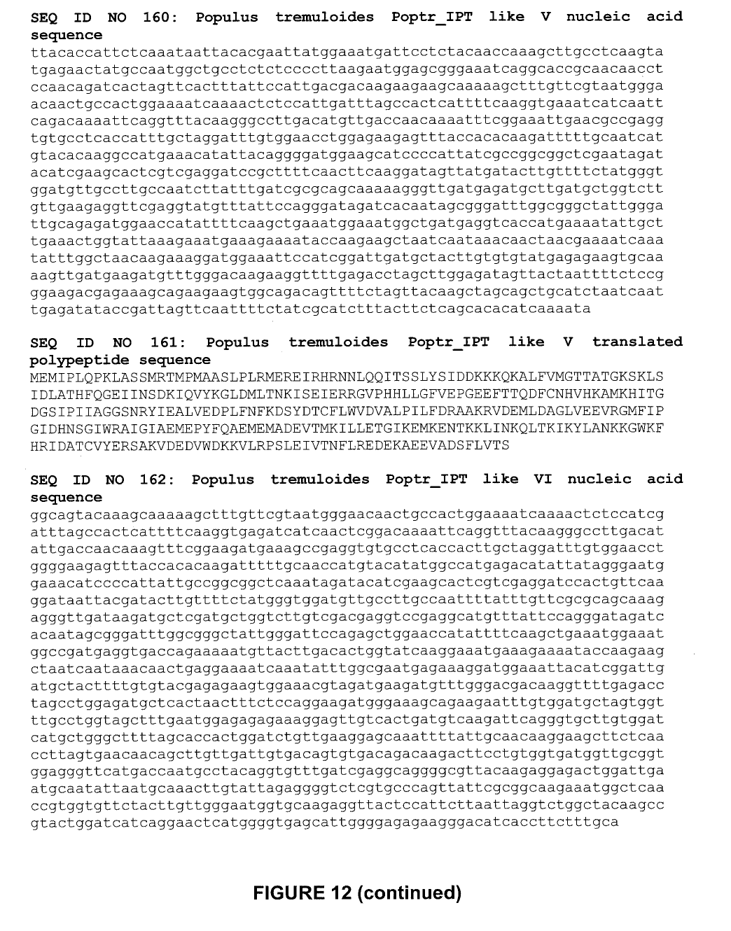

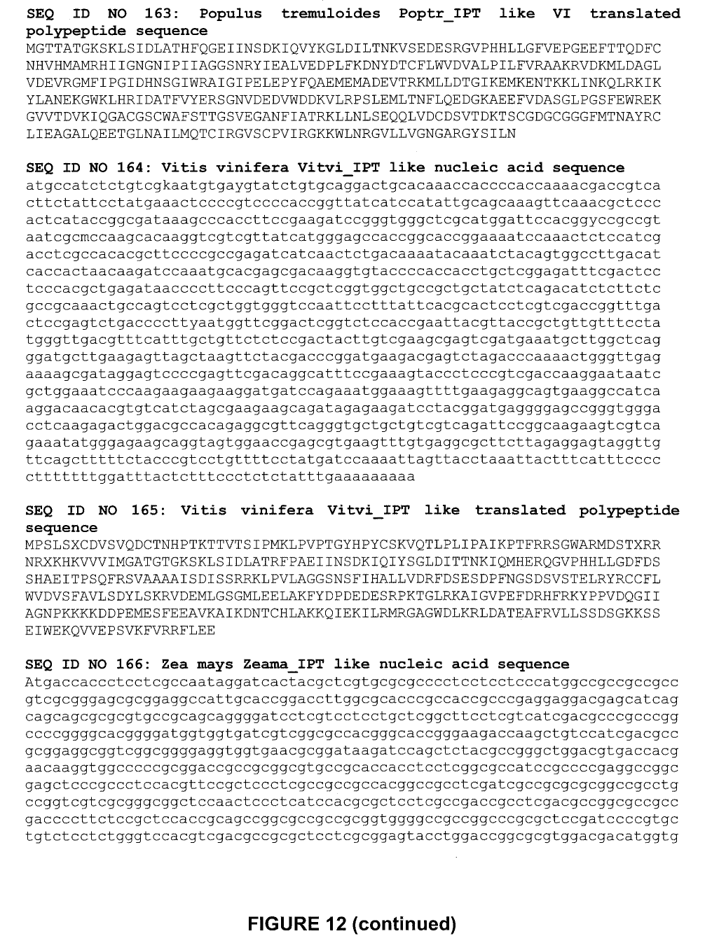

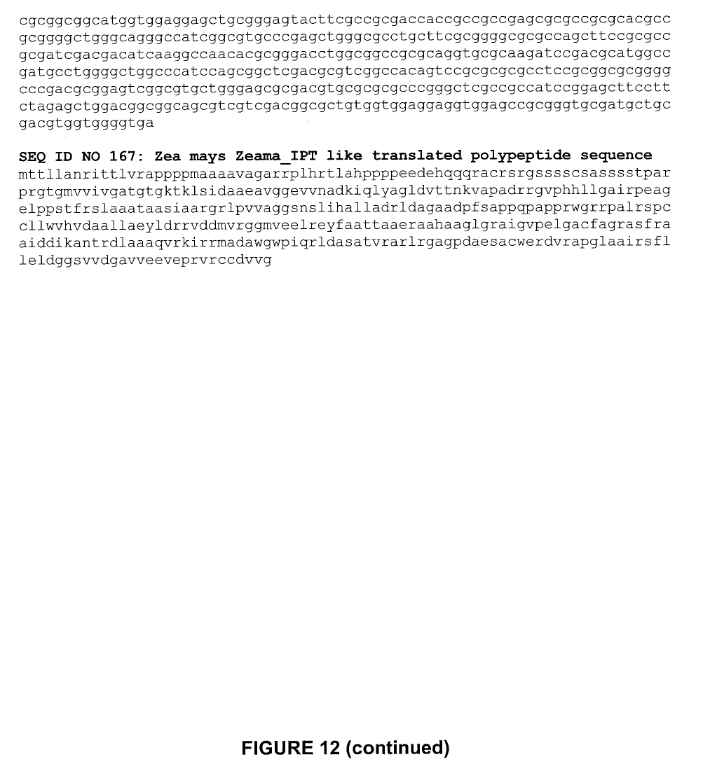

After the LR recombination step, the resulting expression vector pProl::GRP (FIG. 11) was transformed into Agrobacterium strain LBA4044 according to methods well known in the art.

### Example 23

### Plant Transformation

### Rice Transformation

The Agrobacterium containing the expression vector was used to transform Oryza sativa plants. Mature dry seeds of the rice japonica cultivar Nipponbare were dehusked. Sterilization was carried out by incubating for one minute in 70% ethanol, followed by 30 minutes in 0.2% HgCl₂, followed by a 6 times 15 minutes wash with sterile distilled water. The sterile seeds were then germinated on a medium containing 2,4-D (callus induction medium). After incubation in the dark for four weeks, embryogenic, scutellum-derived calli were excised and propagated on the same medium. After two weeks, the calli were multiplied or propagated by subculture on the same medium for another 2 weeks. Embryogenic callus pieces were sub-cultured on fresh medium 3 days before co-cultivatin (to boost cell division activity).

Agrobacterium strain LBA4404 containing the expression vector was used for co-cultivation. Agrobacterium was inoculated on AB medium with the appropriate antibiotics and cultured for 3 days at 28° C. The bacteria were then collected and suspended in liquid co-cultivation medium to a density (OD₆₀₀) of about 1. The suspension was then transferred to a Petri dish and the calli immersed in the suspension for 15 minutes. The callus tissues were then blotted dry on a filter paper and transferred to solidified, co-cultivation medium and incubated for 3 days in the dark at 25° C. Co-cultivated calli were grown on 2,4-D-containing medium for 4 weeks in the dark at 28° C. in the presence of a selection agent. During this period, rapidly growing resistant callus islands developed. After transfer of this material to a regeneration medium and incubation in the light, the embryogenic potential was released and shoots developed in the next four to five weeks. Shoots were excised from the calli and incubated for 2 to 3 weeks on an auxin-containing medium from which they were transferred to soil. Hardened shoots were grown under high humidity and short days in a greenhouse.

Approximately 35 independent T0 rice transformants were generated for one construct. The primary transformants were transferred from a tissue culture chamber to a greenhouse. After a quantitative PCR analysis to verify copy number of the T-DNA insert, only single copy transgenic plants that exhibit tolerance to the selection agent were kept for harvest of T1 seed. Seeds were then harvested three to five months after transplanting. The method yielded single locus transformants at a rate of over 50% (Aldemita and Hodges 1996, Chan et al. 1993, Hiei et al. 1994).

### Example 24

### Phenotypic Evaluation Procedure

### 24.1 Evaluation Setup

Approximately 35 independent T0 rice transformants were generated. The primary transformants were transferred from a tissue culture chamber to a greenhouse for growing and harvest of T1 seed. Six events, of which the T1 progeny segregated 3:1 for presence/absence of the transgene, were retained. For each of these events, approximately 10 T1 seedlings containing the transgene (hetero- and homo-zygotes) and approximately 10 T1 seedlings lacking the transgene (nullizygotes) were selected by monitoring visual marker expression. The transgenic plants and the corresponding nullizygotes were grown side-by-side at random positions. Greenhouse conditions were of shorts days (12 hours light), 28° C. in the light and 22° C. in the dark, and a relative humidity of 70%.

Four T1 events were further evaluated in the T2 generation following the same evaluation procedure as for the T1 generation but with more individuals per event.

From the stage of sowing until the stage of maturity the plants were passed several times through a digital imaging cabinet. At each time point digital images (2048×1536 pixels, 16 million colours) were taken of each plant from at least 6 different angles.

### 24.2 Statistical Analysis: F Test

A two factor ANOVA (analysis of variants) was used as a statistical model for the overall evaluation of plant phenotypic characteristics. An F test was carried out on all the parameters measured of all the plants of all the events transformed with the gene of the present invention. The F test was carried out to check for an effect of the gene over all the transformation events and to verify for an overall effect of the gene, also known as a global gene effect. The threshold for significance for a true global gene effect was set at a 5% probability level for the F test. A significant F test value points to a gene effect, meaning that it is not only the mere presence or position of the gene that is causing the differences in phenotype.

Because two experiments with overlapping events were carried out, a combined analysis is performed. This is useful to check consistency of the effects over the two experiments, and if this is the case, to accumulate evidence from both experiments in order to increase confidence in the conclusion. The method used is a mixed-model approach that takes into account the multilevel structure of the data (i.e. experiment-event-segregants). P values were obtained by comparing likelihood ratio test to chi square distributions.

### 24.3 Parameters Measured

### Biomass-Related Parameter Measurement

The plant aboveground area (or leafy biomass) was determined by counting the total number of pixels on the digital images from aboveground plant parts discriminated from the background. This value was averaged for the pictures taken on the same time point from the different angles and was converted to a physical surface value expressed in square mm by calibration. Experiments show that the aboveground plant area measured this way correlates with the biomass of plant parts above ground. The above ground area is the area measured at the time point at which the plant had reached its maximal leafy biomass.

### Seed-Related Parameter Measurements

The mature primary panicles were harvested, counted, bagged, barcode-labelled and then dried for three days in an oven at 37° C. The panicles were then threshed and all the seeds were collected and counted. The filled husks were separated from the empty ones using an air-blowing device. The empty husks were discarded and the remaining fraction was counted again. The filled husks were weighed on an analytical balance. The number of filled seeds was determined by counting the number of filled husks that remained after the separation step. The total seed yield was measured by weighing all filled husks harvested from a plant. Total seed number per plant was measured by counting the number of husks harvested from a plant. Thousand Kernel Weight (TKW) is extrapolated from the number of filled seeds counted and their total weight. The Harvest Index (HI) in the present invention is defined as the ratio between the total seed yield and the above ground area (mm²), multiplied by a factor 10⁶. The total number of flowers per panicle as defined in the present invention is the ratio between the total number of seeds and the number of mature primary panicles. The seed fill rate as defined in the present invention is the proportion (expressed as a %) of the number of filled seeds over the total number of seeds (or florets).

### Example 25

### Results of the Phenotypic Evaluation of the Transgenic Plants

The transgenic rice plants expressing the GRP nucleic acid sequence represented by SEQ ID NO: 131 under control of the prolamin promoter showed an increase of more than 5% for total seed yield per plant, number of filled seeds, total number of seeds, and harvest index.

| | Overall average % | Average % increase in |
|---|---|---|
| | increase in 4 events in | 4 events in the T2 |
| | the T1 generation | generation |
| Total seed yield per plant | 19% | 14% |
| Number of filled seeds | 20% | 16% |
| Total number of seeds | 16% | 8% |
| Harvest index | 9% | 15% |

### Example 26

### Other Crop Transformation

### Corn Transformation

Transformation of maize (Zea mays) is performed with a modification of the method described by Ishida et al. (1996) Nature Biotech 14(6): 745-50. Transformation is genotype-dependent in corn and only specific genotypes are amenable to transformation and regeneration. The inbred line A188 (University of Minnesota) or hybrids with A188 as a parent are good sources of donor material for transformation, but other genotypes can be used successfully as well. Ears are harvested from corn plant approximately 11 days after pollination (DAP) when the length of the immature embryo is about 1 to 1.2 mm. Immature embryos are cocultivated with Agrobacterium tumefaciens containing the expression vector, and transgenic plants are recovered through organogenesis. Excised embryos are grown on callus induction medium, then maize regeneration medium, containing the selection agent (for example imidazolinone but various selection markers can be used). The Petri plates are incubated in the light at 25° C. for 2-3 weeks, or until shoots develop. The green shoots are transferred from each embryo to maize rooting medium and incubated at 25° C. for 2-3 weeks, until roots develop. The rooted shoots are transplanted to soil in the greenhouse. T1 seeds are produced from plants that exhibit tolerance to the selection agent and that contain a single copy of the T-DNA insert.

### Wheat Transformation

Transformation of wheat is performed with the method described by Ishida et al. (1996) Nature Biotech 14(6): 745-50. The cultivar Bobwhite (available from CIMMYT, Mexico) is commonly used in transformation. Immature embryos are co-cultivated with Agrobacterium tumefaciens containing the expression vector, and transgenic plants are recovered through organogenesis. After incubation with Agrobacterium, the embryos are grown in vitro on callus induction medium, then regeneration medium, containing the selection agent (for example imidazolinone but various selection markers can be used). The Petri plates are incubated in the light at 25° C. for 2-3 weeks, or until shoots develop. The green shoots are transferred from each embryo to rooting medium and incubated at 25° C. for 2-3 weeks, until roots develop. The rooted shoots are transplanted to soil in the greenhouse. T1 seeds are produced from plants that exhibit tolerance to the selection agent and that contain a single copy of the T-DNA insert.

### Soybean Transformation

Soybean is transformed according to a modification of the method described in the Texas A&M U.S. Pat. No. 5,164,310. Several commercial soybean varieties are amenable to transformation by this method. The cultivar Jack (available from the Illinois Seed foundation) is commonly used for transformation. Soybean seeds are sterilised for in vitro sowing. The hypocotyl, the radicle and one cotyledon are excised from seven-day old young seedlings. The epicotyl and the remaining cotyledon are further grown to develop axillary nodes. These axillary nodes are excised and incubated with Agrobacterium tumefaciens containing the expression vector. After the cocultivation treatment, the explants are washed and transferred to selection media. Regenerated shoots are excised and placed on a shoot elongation medium. Shoots no longer than 1 cm are placed on rooting medium until roots develop. The rooted shoots are transplanted to soil in the greenhouse. T1 seeds are produced from plants that exhibit tolerance to the selection agent and that contain a single copy of the T-DNA insert.

Rapeseed/Canola Transformation

Cotyledonary petioles and hypocotyls of 5-6 day old young seedling are used as explants for tissue culture and transformed according to Babic et al. (1998, Plant Cell Rep 17: 183-188). The commercial cultivar Westar (Agriculture Canada) is the standard variety used for transformation, but other varieties can also be used. Canola seeds are surface-sterilized for in vitro sowing. The cotyledon petiole explants with the cotyledon attached are excised from the in vitro seedlings, and inoculated with Agrobacterium (containing the expression vector) by dipping the cut end of the petiole explant into the bacterial suspension. The explants are then cultured for 2 days on MSBAP-3 medium containing 3 mg/l BAP, 3% sucrose, 0.7% Phytagar at 23° C., 16 hr light. After two days of co-cultivation with Agrobacterium, the petiole explants are transferred to MSBAP-3 medium containing 3 mg/l BAP, cefotaxime, carbenicillin, or timentin (300 mg/l) for 7 days, and then cultured on MSBAP-3 medium with cefotaxime, carbenicillin, or timentin and selection agent until shoot regeneration. When the shoots are 5-10 mm in length, they are cut and transferred to shoot elongation medium (MSBAP-0.5, containing 0.5 mg/l BAP). Shoots of about 2 cm in length are transferred to the rooting medium (MS0) for root induction. The rooted shoots are transplanted to soil in the greenhouse. T1 seeds are produced from plants that exhibit tolerance to the selection agent and that contain a single copy of the T-DNA insert.

### Alfalfa Transformation

A regenerating clone of alfalfa (Medicago sativa) is transformed using the method of (McKersie et al., 1999 Plant Physiol 119: 839-847). Regeneration and transformation of alfalfa is genotype dependent and therefore a regenerating plant is required. Methods to obtain regenerating plants have been described. For example, these can be selected from the cultivar Rangelander (Agriculture Canada) or any other commercial alfalfa variety as described by

Brown DCW and A Atanassov (1985. Plant Cell Tissue Organ Culture 4: 111-112). Alternatively, the RA3 variety (University of Wisconsin) has been selected for use in tissue culture (Walker et al., 1978 Am J Bot 65:654-659). Petiole explants are cocultivated with an overnight culture of Agrobacterium tumefaciens C58C1 pMP90 (McKersie et al., 1999 Plant Physiol 119: 839-847) or LBA4404 containing the expression vector. The explants are cocultivated for 3 d in the dark on SH induction medium containing 288 mg/L Pro, 53 mg/L thioproline, 4.35 g/L K2SO4, and 100 μm acetosyringinone. The explants are washed in half-strength Murashige-Skoog medium (Murashige and Skoog, 1962) and plated on the same SH induction medium without acetosyringinone but with a suitable selection agent and suitable antibiotic to inhibit Agrobacterium growth. After several weeks, somatic embryos are transferred to BOi2Y development medium containing no growth regulators, no antibiotics, and 50 g/L sucrose. Somatic embryos are subsequently germinated on half-strength Murashige-Skoog medium. Rooted seedlings were transplanted into pots and grown in a greenhouse. T1 seeds are produced from plants that exhibit tolerance to the selection agent and that contain a single copy of the T-DNA insert.

### Cotton Transformation

Cotton (Gossypium hirsutum L.) transformation is performed using Agrobacterium tumefaciens, on hypocotyls explants. The commercial cultivars such as Coker 130 or Coker 312 (SeedCo, Lubbock, Tex.) are standard varieties used for transformation, but other varieties can also be used. The seeds are surface sterilized and germinated in the dark. Hypocotyl explants are cut from the germinated seedlings to lengths of about 1-1.5 centimeter. The hypotocyl explant is submersed in the Agrobacterium tumefaciens inoculum containing the expression vector, for 5 minutes then co-cultivated for about 48 hours on MS+1.8 mg/l KNO3+2% glucose at 24° C., in the dark. The explants are transferred the same medium containing appropriate bacterial and plant selectable markers (renewed several times), until embryogenic calli is seen. The calli are separated and subcultured until somatic embryos appear. Plantlets derived from the somatic embryos are matured on rooting medium until roots develop. The rooted shoots are transplanted to potting soil in the greenhouse. T1 seeds are produced from plants that exhibit tolerance to the selection agent and that contain a single copy of the T-DNA insert.

### Example 27

### Abiotic Stress Screens

### Drought Screen

Rice plants from T1, T2 or further generations are grown in potting soil under normal conditions until they approached the heading stage. They are then transferred to a “dry” section where irrigation is withheld. Humidity probes are inserted in randomly chosen pots to monitor the soil water content (SWC). When SWC goes below certain thresholds, the plants are automatically re-watered continuously until a normal level is reached again. The plants are then re-transferred again to normal conditions. The rest of the cultivation (plant maturation, seed harvest) is the same as for plants not grown under abiotic stress conditions. Growth and yield parameters are recorded as detailed for growth under normal conditions.

### Salt Stress Screen

Rice plants from T1, T2 or further generations are grown on a substrate made of coco fibers and argex (3 to 1 ratio). A normal nutrient solution is used during the first two weeks after transplanting the plantlets in the greenhouse. After the first two weeks, 25 mM of salt (NaCl) is added to the nutrient solution, until the plants are harvested. Growth and yield parameters are recorded as detailed for growth under normal conditions.

### Nitrogen Use Efficiency Screen

Rice plants from T1, T2 or further generations are grown in potting soil under normal conditions except for the nutrient solution. The pots are watered from transplantation to maturation with a specific nutrient solution containing reduced N nitrogen (N) content, usually between 7 to 8 times less. The rest of the cultivation (plant maturation, seed harvest) is the same as for plants not grown under abiotic stress. Growth and yield parameters are recorded as detailed for growth under normal conditions.

### Example 28

### Cloning of the Nucleic Acid Sequence Used in the Methods of the Invention, Coding for an STO (Salt Tolerance) Protein

The nucleic acid sequence used in the methods of the invention was amplified by PCR using as template a custom-made Oryza sativa seedlings cDNA library (in pCMV Sport 6.0; Invitrogen, Paisley, UK). PCR was performed using Hifi Taq DNA polymerase in standard conditions, using 200 ng of template in a 50 μl PCR mix. The primers used were prm1-STO (SEQ ID NO: 216; sense, start codon in bold):

5′-ggggacaagtttgtacaaaaaagcaggcttaaacaatgaaggtgcag
tgcga-3′

and prm2-STO (SEQ ID NO: 217; reverse, complementary):

5′-ggggacactttgtacaagaaagctgggttcaccagtacaagcagg
gag 3′,

which include the AttB sites for Gateway recombination. The amplified PCR fragment was purified also using standard methods. The first step of the Gateway procedure, the BP reaction, was then performed, during which the PCR fragment recombines in vivo with the pDONR201 plasmid to produce, according to the Gateway terminology, an “entry clone”, pSTO. Plasmid pDONR201 was purchased from Invitrogen, as part of the Gateway® technology.

The entry clone comprising SEQ ID NO: 168 was then used in an LR reaction with a destination vector used for Oryza sativa transformation. This vector contained as functional elements within the T-DNA borders: a plant selectable marker; a screenable marker expression cassette; and a Gateway cassette intended for LR in vivo recombination with the nucleic acid sequence of interest already cloned in the entry clone. A rice GOS2 promoter (SEQ ID NO: 218) for root specific expression was located upstream of this Gateway cassette.

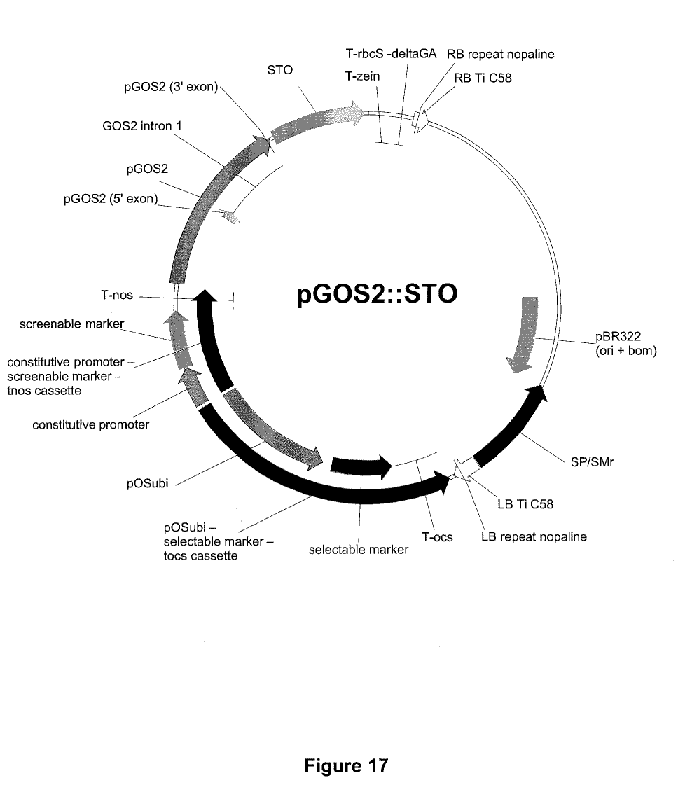

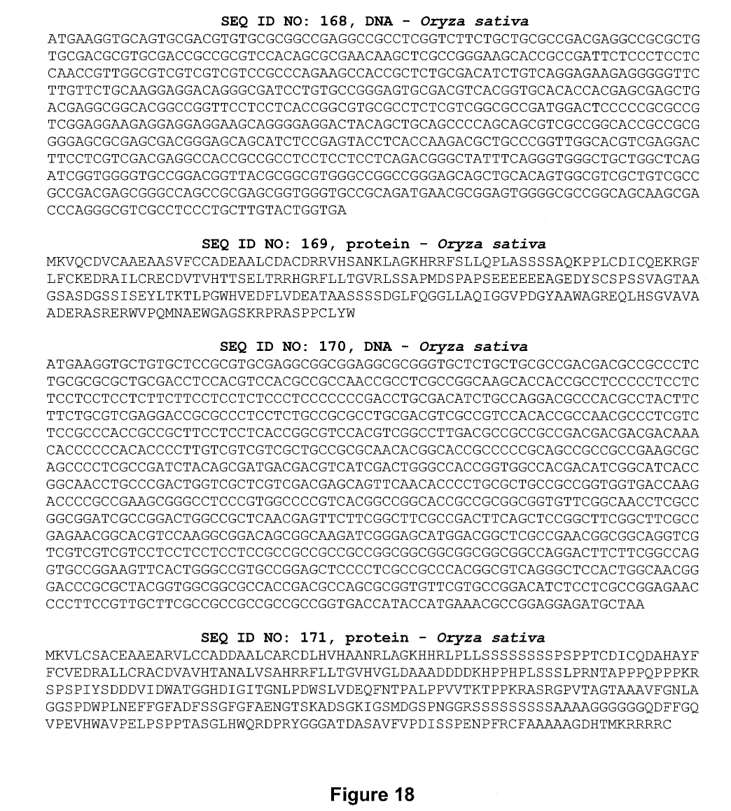

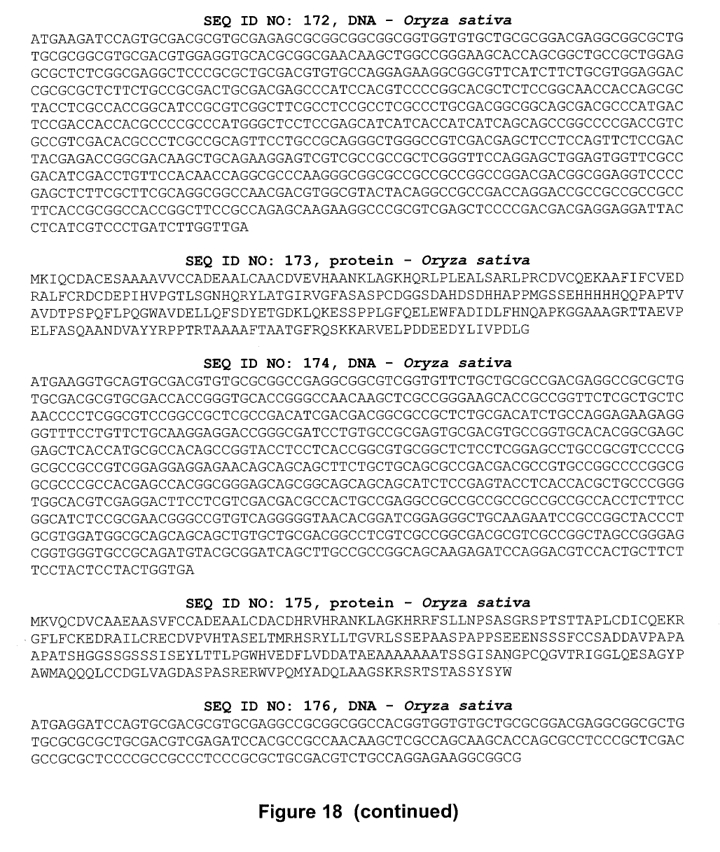

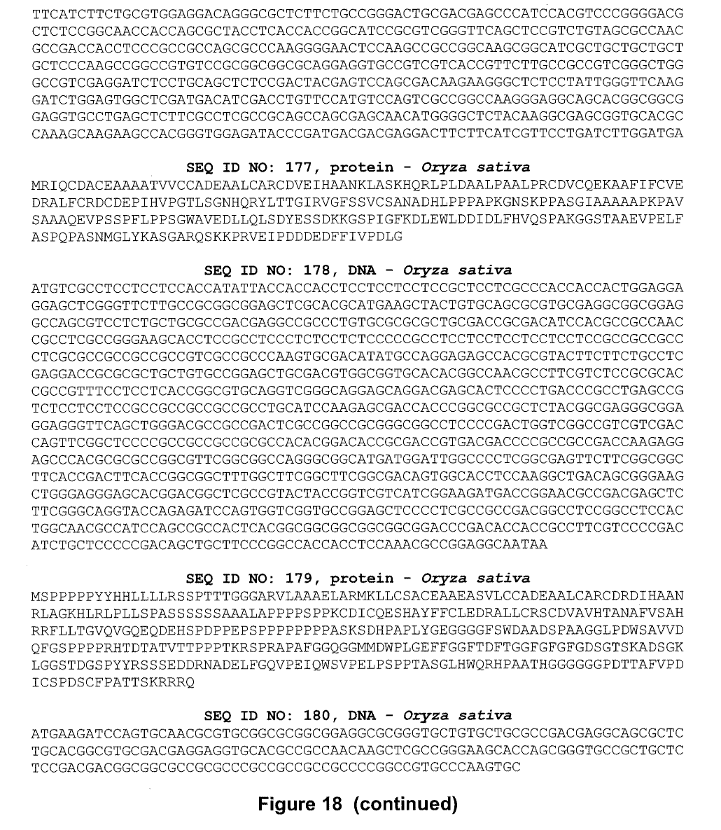

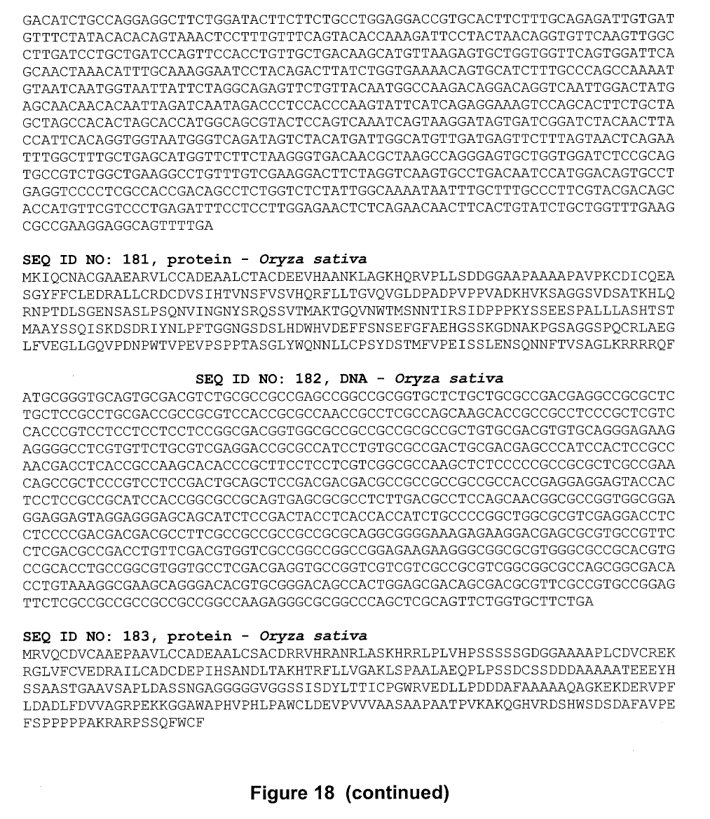

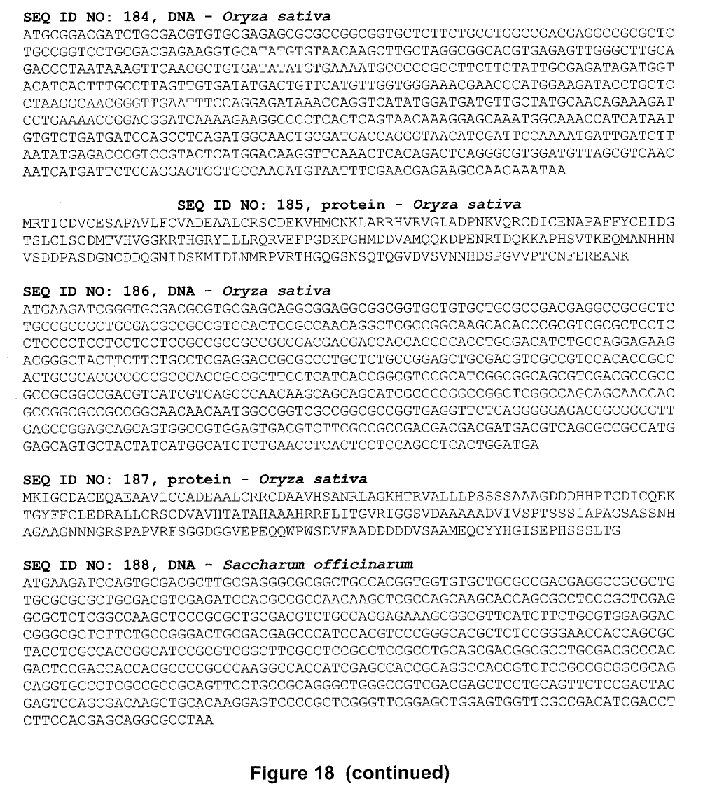

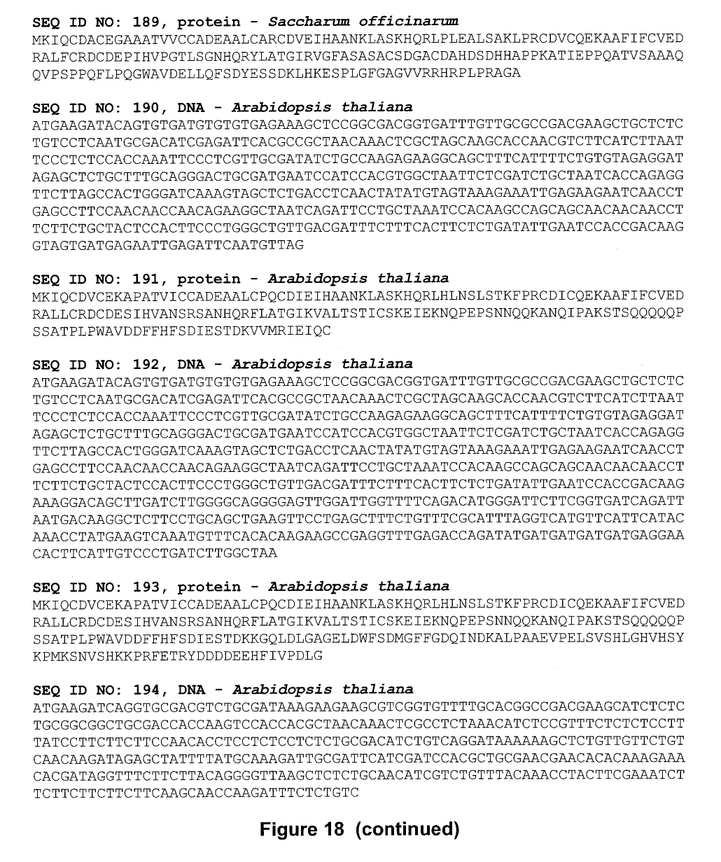

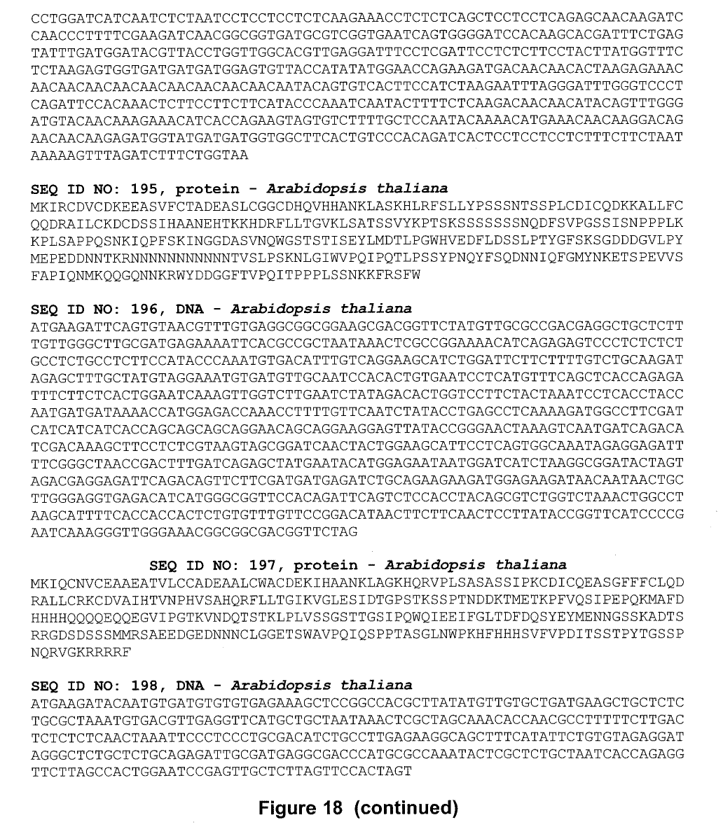

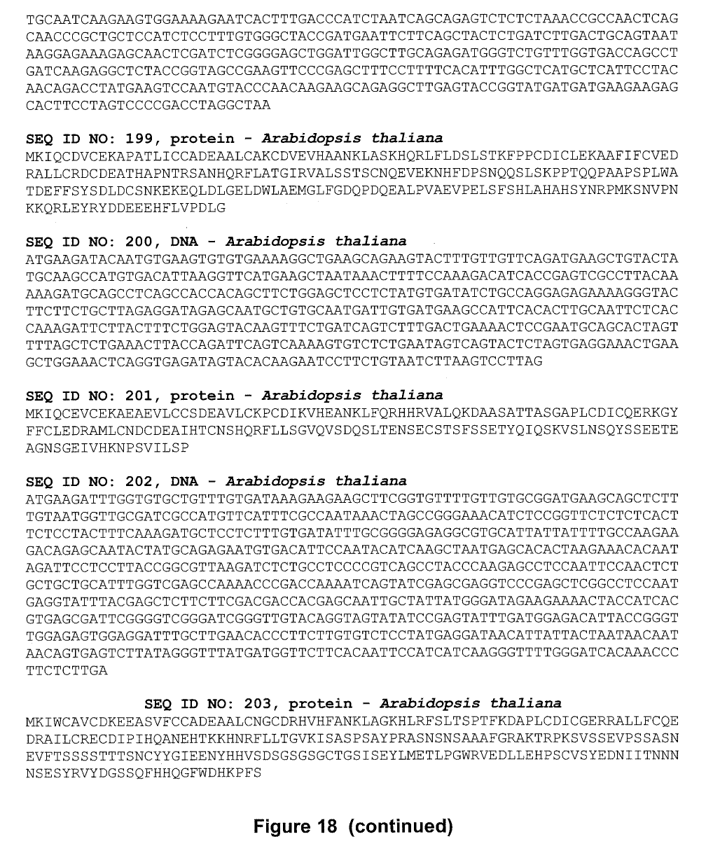

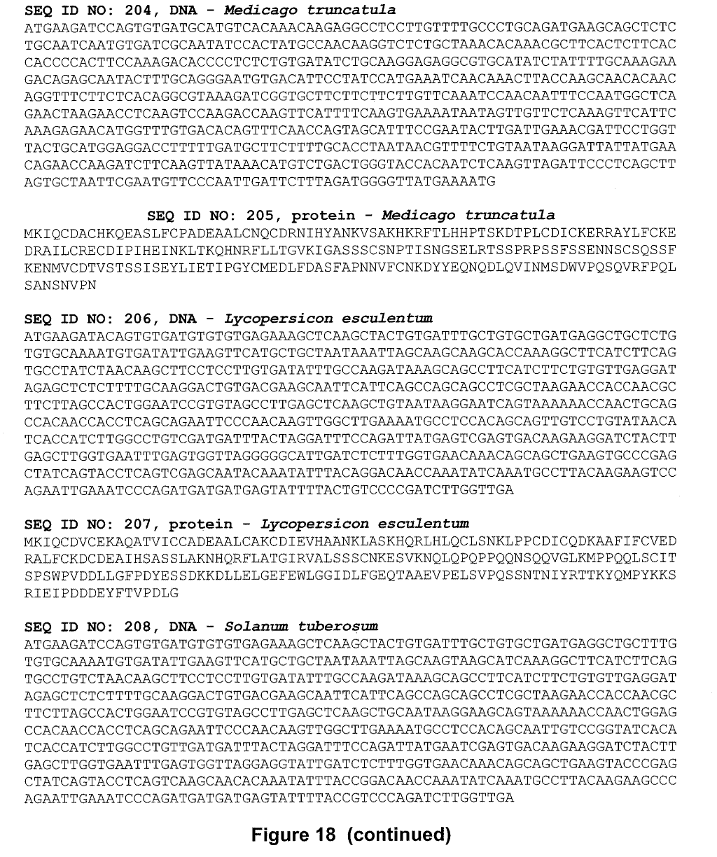

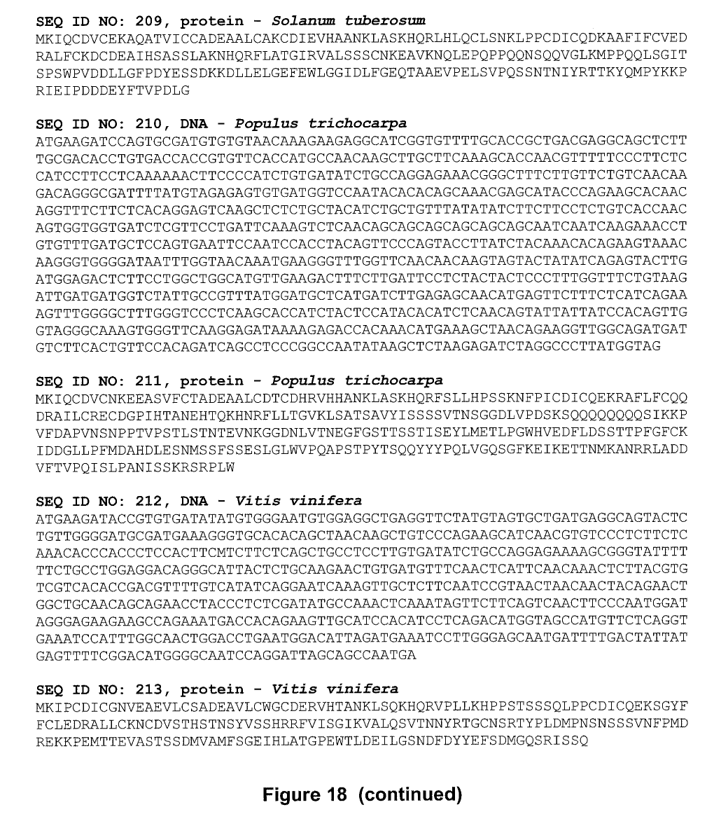

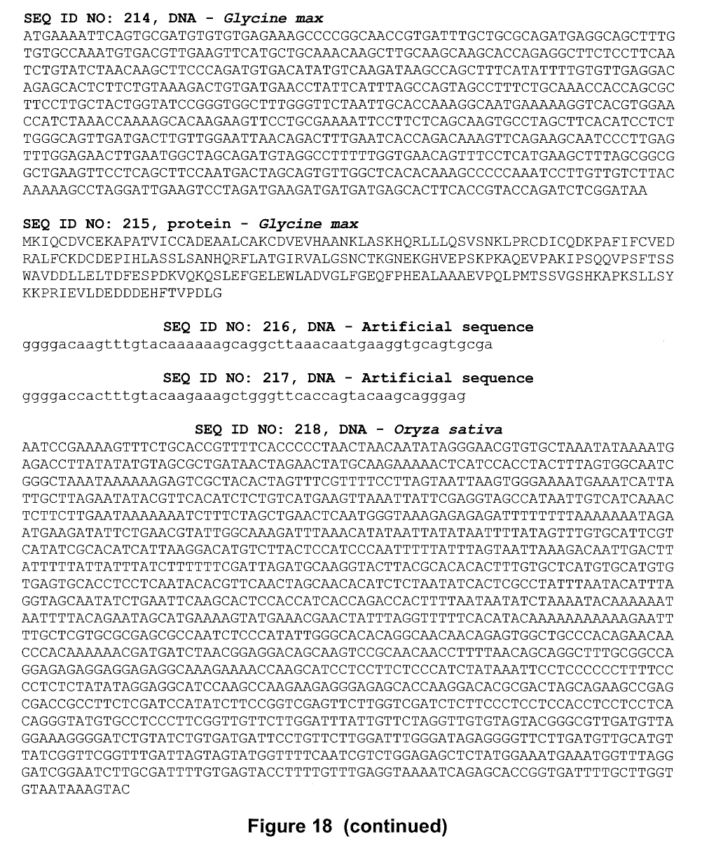

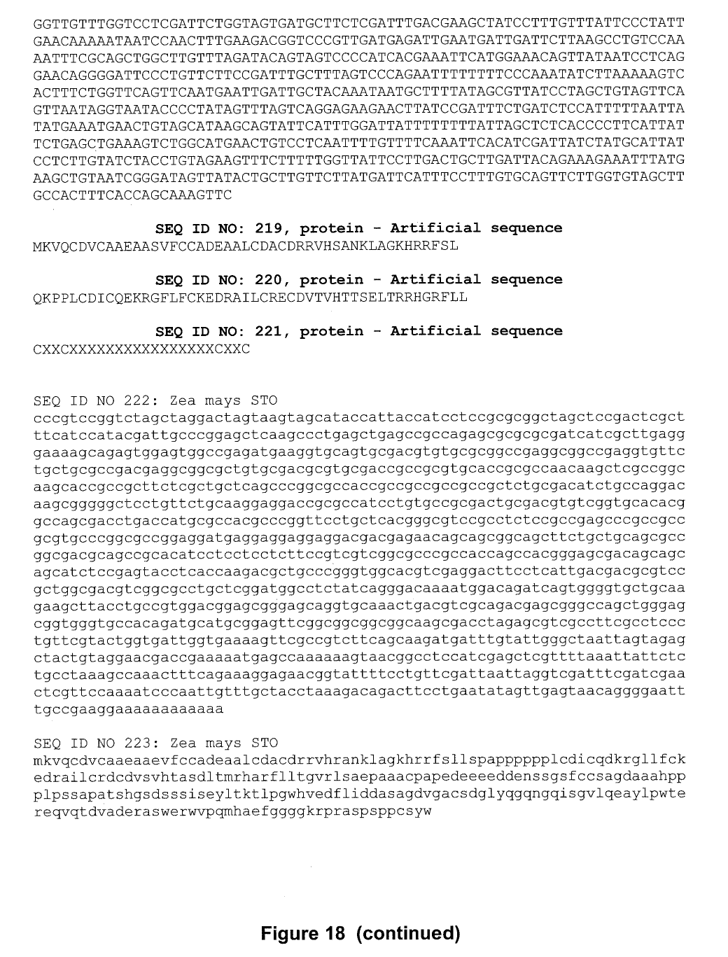

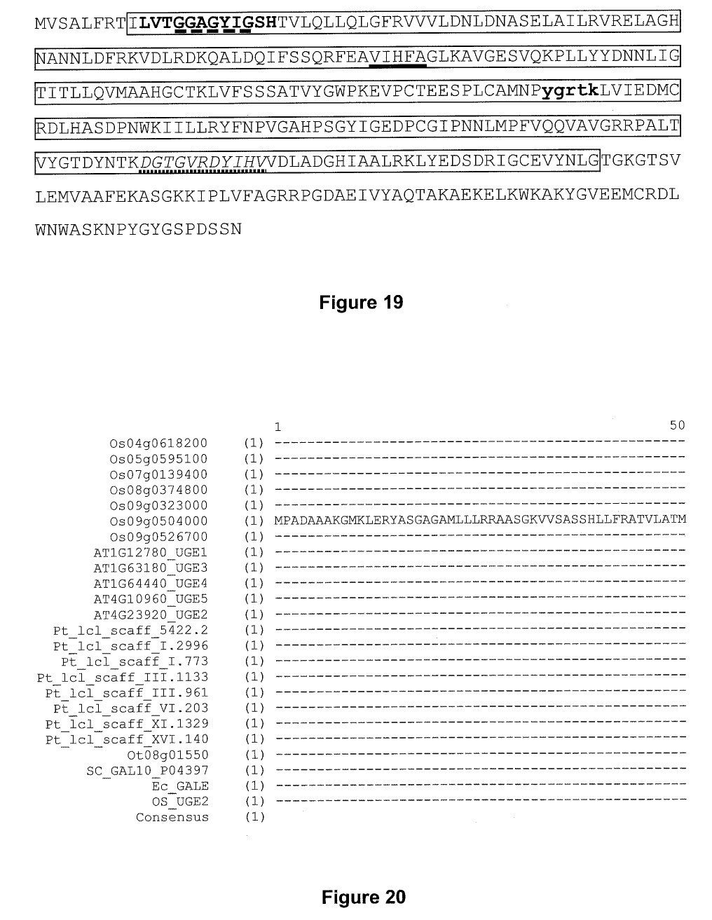

After the LR recombination step, the resulting expression vector pGOS2::STO (FIG. 17) was transformed into Agrobacterium strain LBA4044 according to methods well known in the art.

### Example 29

### Plant Transformation

### Rice Transformation

The Agrobacterium containing the expression vector was used to transform Oryza sativa plants. Mature dry seeds of the rice japonica cultivar Nipponbare were dehusked. Sterilization was carried out by incubating for one minute in 70% ethanol, followed by 30 minutes in 0.2% HgCl₂, followed by a 6 times 15 minutes wash with sterile distilled water. The sterile seeds were then germinated on a medium containing 2,4-D (callus induction medium). After incubation in the dark for four weeks, embryogenic, scutellum-derived calli were excised and propagated on the same medium. After two weeks, the calli were multiplied or propagated by subculture on the same medium for another 2 weeks. Embryogenic callus pieces were sub-cultured on fresh medium 3 days before co-cultivation (to boost cell division activity).

Agrobacterium strain LBA4404 containing the expression vector was used for co-cultivation. Agrobacterium was inoculated on AB medium with the appropriate antibiotics and cultured for 3 days at 28° C. The bacteria were then collected and suspended in liquid co-cultivation medium to a density (OD₆₀₀) of about 1. The suspension was then transferred to a Petri dish and the calli immersed in the suspension for 15 minutes. The callus tissues were then blotted dry on a filter paper and transferred to solidified, co-cultivation medium and incubated for 3 days in the dark at 25° C. Co-cultivated calli were grown on 2,4-D-containing medium for 4 weeks in the dark at 28° C. in the presence of a selection agent. During this period, rapidly growing resistant callus islands developed. After transfer of this material to a regeneration medium and incubation in the light, the embryogenic potential was released and shoots developed in the next four to five weeks. Shoots were excised from the calli and incubated for 2 to 3 weeks on an auxin-containing medium from which they were transferred to soil. Hardened shoots were grown under high humidity and short days in a greenhouse.

Approximately 35 independent T0 rice transformants were generated for one construct. The primary transformants were transferred from a tissue culture chamber to a greenhouse. After a quantitative PCR analysis to verify copy number of the T-DNA insert, only single copy transgenic plants that exhibit tolerance to the selection agent were kept for harvest of T1 seed. Seeds were then harvested three to five months after transplanting. The method yielded single locus transformants at a rate of over 50% (Aldemita and Hodges1996, Chan et al. 1993, Hiei et al. 1994).

### Corn Transformation

Transformation of maize (Zea mays) is performed with a modification of the method described by Ishida et al. (1996) Nature Biotech 14(6): 745-50. Transformation is genotype-dependent in corn and only specific genotypes are amenable to transformation and regeneration. The inbred line A188 (University of Minnesota) or hybrids with A188 as a parent are good sources of donor material for transformation, but other genotypes can be used successfully as well. Ears are harvested from corn plant approximately 11 days after pollination (DAP) when the length of the immature embryo is about 1 to 1.2 mm. Immature embryos are cocultivated with Agrobacterium tumefaciens containing the expression vector, and transgenic plants are recovered through organogenesis. Excised embryos are grown on callus induction medium, then maize regeneration medium, containing the selection agent (for example imidazolinone but various selection markers can be used). The Petri plates are incubated in the light at 25° C. for 2-3 weeks, or until shoots develop. The green shoots are transferred from each embryo to maize rooting medium and incubated at 25° C. for 2-3 weeks, until roots develop. The rooted shoots are transplanted to soil in the greenhouse. T1 seeds are produced from plants that exhibit tolerance to the selection agent and that contain a single copy of the T-DNA insert.

### Wheat Transformation

Transformation of wheat is performed with the method described by Ishida et al. (1996) Nature Biotech 14(6): 745-50. The cultivar Bobwhite (available from CIMMYT, Mexico) is commonly used in transformation. Immature embryos are co-cultivated with Agrobacterium tumefaciens containing the expression vector, and transgenic plants are recovered through organogenesis. After incubation with Agrobacterium, the embryos are grown in vitro on callus induction medium, then regeneration medium, containing the selection agent (for example imidazolinone but various selection markers can be used). The Petri plates are incubated in the light at 25° C. for 2-3 weeks, or until shoots develop. The green shoots are transferred from each embryo to rooting medium and incubated at 25° C. for 2-3 weeks, until roots develop. The rooted shoots are transplanted to soil in the greenhouse. T1 seeds are produced from plants that exhibit tolerance to the selection agent and that contain a single copy of the T-DNA insert.

### Soybean Transformation

Soybean is transformed according to a modification of the method described in the Texas A&M U.S. Pat. No. 5,164,310. Several commercial soybean varieties are amenable to transformation by this method. The cultivar Jack (available from the Illinois Seed foundation) is commonly used for transformation. Soybean seeds are sterilised for in vitro sowing. The hypocotyl, the radicle and one cotyledon are excised from seven-day old young seedlings. The epicotyl and the remaining cotyledon are further grown to develop axillary nodes. These axillary nodes are excised and incubated with Agrobacterium tumefaciens containing the expression vector. After the cocultivation treatment, the explants are washed and transferred to selection media. Regenerated shoots are excised and placed on a shoot elongation medium. Shoots no longer than 1 cm are placed on rooting medium until roots develop. The rooted shoots are transplanted to soil in the greenhouse. T1 seeds are produced from plants that exhibit tolerance to the selection agent and that contain a single copy of the T-DNA insert.

### Rapeseed/Canola Transformation

Cotyledonary petioles and hypocotyls of 5-6 day old young seedling are used as explants for tissue culture and transformed according to Babic et al. (1998, Plant Cell Rep 17: 183-188). The commercial cultivar Westar (Agriculture Canada) is the standard variety used for transformation, but other varieties can also be used. Canola seeds are surface-sterilized for in vitro sowing. The cotyledon petiole explants with the cotyledon attached are excised from the in vitro seedlings, and inoculated with Agrobacterium (containing the expression vector) by dipping the cut end of the petiole explant into the bacterial suspension. The explants are then cultured for 2 days on MSBAP-3 medium containing 3 mg/l BAP, 3% sucrose, 0.7% Phytagar at 23° C., 16 hr light. After two days of co-cultivation with Agrobacterium, the petiole explants are transferred to MSBAP-3 medium containing 3 mg/l BAP, cefotaxime, carbenicillin, or timentin (300 mg/l) for 7 days, and then cultured on MSBAP-3 medium with cefotaxime, carbenicillin, or timentin and selection agent until shoot regeneration. When the shoots are 5-10 mm in length, they are cut and transferred to shoot elongation medium (MSBAP-0.5, containing 0.5 mg/l BAP). Shoots of about 2 cm in length are transferred to the rooting medium (MS0) for root induction. The rooted shoots are transplanted to soil in the greenhouse. T1 seeds are produced from plants that exhibit tolerance to the selection agent and that contain a single copy of the T-DNA insert.

### Alfalfa Transformation

A regenerating clone of alfalfa (Medicago sativa) is transformed using the method of (McKersie et al., 1999 Plant Physiol 119: 839-847). Regeneration and transformation of alfalfa is genotype dependent and therefore a regenerating plant is required. Methods to obtain regenerating plants have been described. For example, these can be selected from the cultivar Rangelander (Agriculture Canada) or any other commercial alfalfa variety as described by Brown DCW and A Atanassov (1985. Plant Cell Tissue Organ Culture 4: 111-112). Alternatively, the RA3 variety (University of Wisconsin) has been selected for use in tissue culture (Walker et al., 1978 Am J Bot 65:654-659). Petiole explants are cocultivated with an overnight culture of Agrobacterium tumefaciens C58C1 pMP90 (McKersie et al., 1999 Plant Physiol 119: 839-847) or LBA4404 containing the expression vector. The explants are cocultivated for 3 d in the dark on SH induction medium containing 288 mg/L Pro, 53 mg/L thioproline, 4.35 g/L K2SO4, and 100 μm acetosyringinone. The explants are washed in half-strength Murashige-Skoog medium (Murashige and Skoog, 1962) and plated on the same SH induction medium without acetosyringinone but with a suitable selection agent and suitable antibiotic to inhibit Agrobacterium growth. After several weeks, somatic embryos are transferred to BOi2Y development medium containing no growth regulators, no antibiotics, and 50 g/L sucrose. Somatic embryos are subsequently germinated on half-strength Murashige-Skoog medium. Rooted seedlings were transplanted into pots and grown in a greenhouse. T1 seeds are produced from plants that exhibit tolerance to the selection agent and that contain a single copy of the T-DNA insert.

### Example 30

### Phenotypic Evaluation Procedure

### 30.1 Evaluation Setup

Approximately 35 independent T0 rice transformants were generated. The primary transformants were transferred from a tissue culture chamber to a greenhouse for growing and harvest of T1 seed. Six events, of which the T1 progeny segregated 3:1 for presence/absence of the transgene, were retained. For each of these events, approximately 10 T1 seedlings containing the transgene (hetero- and homo-zygotes) and approximately 10 T1 seedlings lacking the transgene (nullizygotes) were selected by monitoring visual marker expression. The transgenic plants and the corresponding nullizygotes were grown side-by-side at random positions. Greenhouse conditions were of shorts days (12 hours light), 28° C. in the light and 22° C. in the dark, and a relative humidity of 70%.

Four T1 events were further evaluated in the T2 generation following the same evaluation procedure as for the T1 generation but with more individuals per event. From the stage of sowing until the stage of maturity the plants were passed several times through a digital imaging cabinet. At each time Point digital images (2048×1536 pixels, 16 million colours) were taken of each plant from at least 6 different angles.

### Drought Screen

Plants from T2 seeds were grown in potting soil under normal conditions until they approached the heading stage. They were then transferred to a “dry” section where irrigation was withheld. Humidity probes were inserted in randomly chosen pots to monitor the soil water content (SWC). When SWC went below certain thresholds, the plants were automatically re-watered continuously until a normal level was reached again. The plants were then re-transferred again to normal conditions. The rest of the cultivation (plant maturation, seed harvest) was the same as for plants not grown under abiotic stress conditions. Growth and yield parameters were recorded as detailed for growth under normal conditions.

### Nitrogen Use Efficiency Screen

Rice plants from T2 seeds were grown in potting soil under normal conditions except for the nutrient solution. The pots were watered from transplantation to maturation with a specific nutrient solution containing reduced N nitrogen (N) content, usually between 7 to 8 times less. The rest of the cultivation (plant maturation, seed harvest) was the same as for plants not grown under abiotic stress. Growth and yield parameters are recorded as detailed for growth under normal conditions.

### 30.2 Statistical Analysis: F Test

A two factor ANOVA (analysis of variants) was used as a statistical model for the overall evaluation of plant phenotypic characteristics. An F test was carried out on all the parameters measured of all the plants of all the events transformed with the gene of the present invention. The F test was carried out to check for an effect of the gene over all the transformation events and to verify for an overall effect of the gene, also known as a global gene effect. The threshold for significance for a true global gene effect was set at a 5% probability level for the F test. A significant F test value Points to a gene effect, meaning that it is not only the mere presence or position of the gene that is causing the differences in phenotype.

Because two experiments with overlapping events were carried out, a combined analysis was performed. This is useful to check consistency of the effects over the two experiments, and if this is the case, to accumulate evidence from both experiments in order to increase confidence in the conclusion. The method used was a mixed-model approach that takes into account the multilevel structure of the data (i.e. experiment-event-segregants). P values were obtained by comparing likelihood ratio test to chi square distributions.

### 30.3 Parameters Measured

### Biomass-Related Parameter Measurement

From the stage of sowing until the stage of maturity the plants were passed several times through a digital imaging cabinet. At each time Point digital images (2048×1536 pixels, 16 million colours) were taken of each plant from at least 6 different angles.

The plant aboveground area (or leafy biomass) was determined by counting the total number of pixels on the digital images from aboveground plant parts discriminated from the background. This value was averaged for the pictures taken on the same time Point from the different angles and was converted to a physical surface value expressed in square mm by calibration. Experiments show that the aboveground plant area measured this way correlates with the biomass of plant parts above ground. The above ground area is the area measured at the time Point at which the plant had reached its maximal leafy biomass. The early vigour is the plant (seedling) aboveground area three weeks post-germination. Increase in root biomass is expressed as an increase in total root biomass (measured as maximum biomass of roots observed during the lifespan of a plant); or as an increase in the root/shoot index (measured as the ratio between root mass and shoot mass in the period of active growth of root and shoot).

Early vigour was determined by counting the total number of pixels from aboveground plant parts discriminated from the background. This value was averaged for the pictures taken on the same time Point from different angles and was converted to a physical surface value expressed in square mm by calibration. The results described below are for plants three weeks post-germination.

Flowering time was calculated as previously described (PCT/EP2007/001422). Briefly, the time elapsed between sowing and the moment when the first panicle appeared in the plant was calculated. Appearance of the first panicle refers the point in time when the panicle is visible and detectable in the photographic images of the plants processed as described in (PCT/EP2007/001422).

### Seed-Related Parameter Measurements

The mature primary panicles were harvested, counted, bagged, barcode-labelled and then dried for three days in an oven at 37° C. The panicles were then threshed and all the seeds were collected and counted. The filled husks were separated from the empty ones using an air-blowing device. The empty husks were discarded and the remaining fraction was counted again. The filled husks were weighed on an analytical balance. The number of filled seeds was determined by counting the number of filled husks that remained after the separation step.

The total seed yield was measured by weighing all filled husks harvested from a plant. Total seed number per plant was measured by counting the number of husks harvested from a plant. Thousand Kernel Weight (TKW) is extrapolated from the number of filled seeds counted and their total weight. The Harvest Index (HI) in the present invention is defined as the ratio between the total seed yield and the above ground area (mm²), multiplied by a factor 10⁶. The total number of flowers per panicle as defined in the present invention is the ratio between the total number of seeds and the number of mature primary panicles. The seed fill rate as defined in the present invention is the proportion (expressed as a %) of the number of filled seeds over the total number of seeds (or florets).

### Example 31

### Results of the Phenotypic Evaluation of the Transgenic Plants

The results of the evaluation of transgenic rice plants expressing an STO nucleic acid under non-stress conditions are presented below. An increase of at least 5% was observed for emergence vigour (early vigour), and between 3 and 6 days (6-9%), depending on the specific transgenic line, earlier flowering was observed in the transgenic plants when compared to relative control plant.

The results of the evaluation of transgenic rice plants expressing an STO nucleic acid under drought-stress conditions are presented hereunder. An increase was observed for total seed weight, number of filled seeds, fill rate, harvest index and thousand-kernel weight (Table D).

TABLE D
Result phenotypic evaluation of the transgenic STO plants.

| | Percentage difference |
|---|---|
| | for the transgenic |
| Parameter | versus the control plants |
| Emergence vigour | 15 |
| Flowering time (early/shorter) | 7 |

### Example 32

### Cloning of the Nucleic Acid Sequence Used in the Methods of the Invention Encoding a UGE (UDP-Glucose 4-Epimerase or UDP-Gal 4-Epimerase) Polypeptide

The nucleic acid sequence used in the methods of the invention was amplified by PCR using as template a custom-made Oryza sativa seedlings cDNA library (in pCMV Sport 6.0; Invitrogen, Paisley, UK). PCR was performed using Hifi Taq DNA polymerase in standard conditions, using 200 ng of template in a 50 μl PCR mix. The primers used were prm1 (SEQ ID NO: 272; sense, start codon in bold): 5′-ggggacaagtttgtacaaaaaagcaggcttaaacaatggtttc ggccttgttg-3′ and prm2 (SEQ ID NO: 273; reverse, complementary): 5′-ggggaccactttgtacaag aaagctgggtgctgctgctactggaggatt-3′, which include the AttB sites for Gateway recombination. The amplified PCR fragment was purified also using standard methods. The first step of the Gateway procedure, the BP reaction, was then performed, during which the PCR fragment recombines in vivo with the pDONR201 plasmid to produce, according to the Gateway terminology, an “entry clone”, pUGE. Plasmid pDONR201 was purchased from Invitrogen, as part of the Gateway® technology.

The entry clone comprising SEQ ID NO: 224 was then used in an LR reaction with a destination vector used for Oryza sativa transformation. This vector contained as functional elements within the T-DNA borders: a plant selectable marker; a screenable marker expression cassette; and a Gateway cassette intended for LR in vivo recombination with the nucleic acid sequence of interest already cloned in the entry clone. A rice GOS2 promoter (SEQ ID NO: 274) for root specific expression was located upstream of this Gateway cassette.

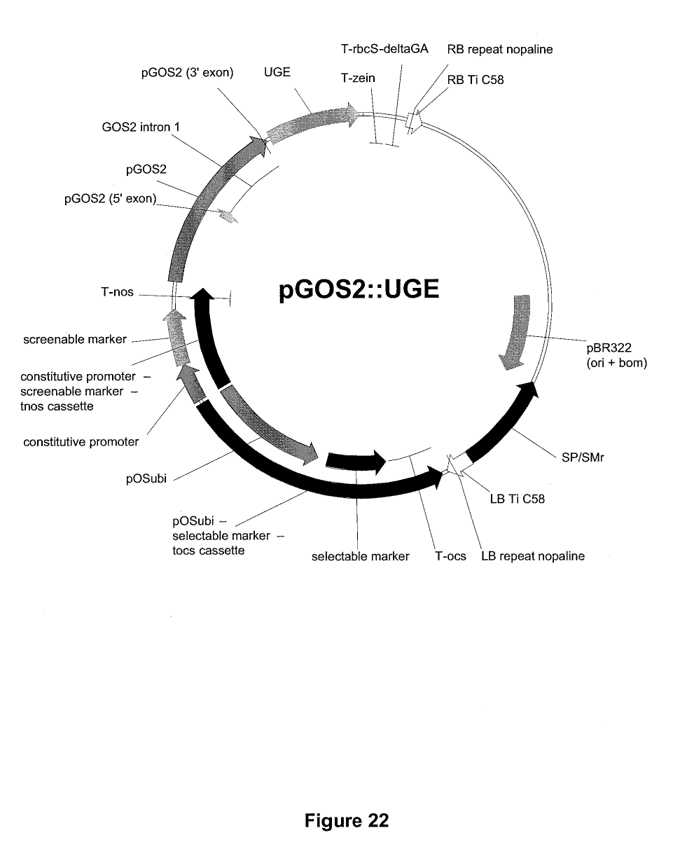

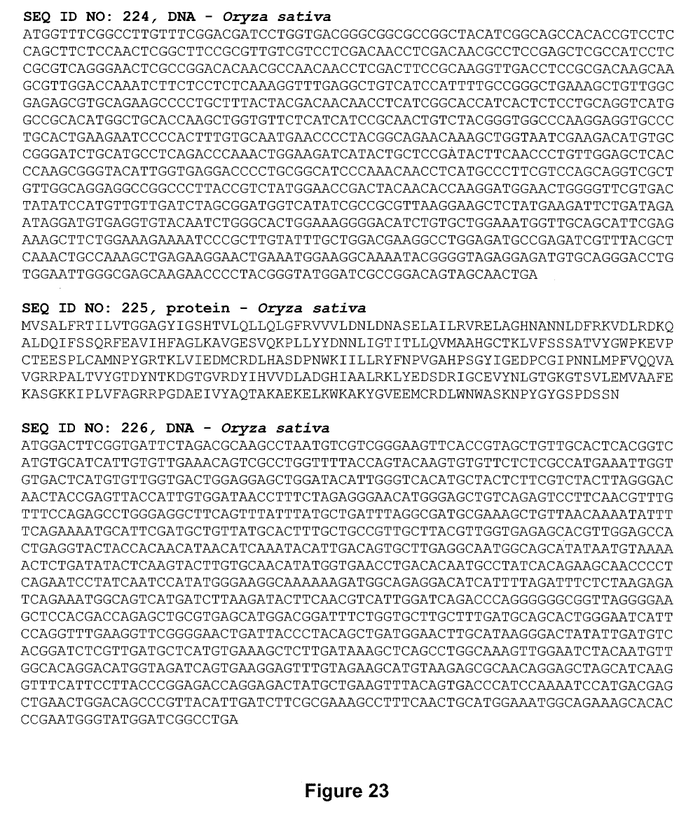

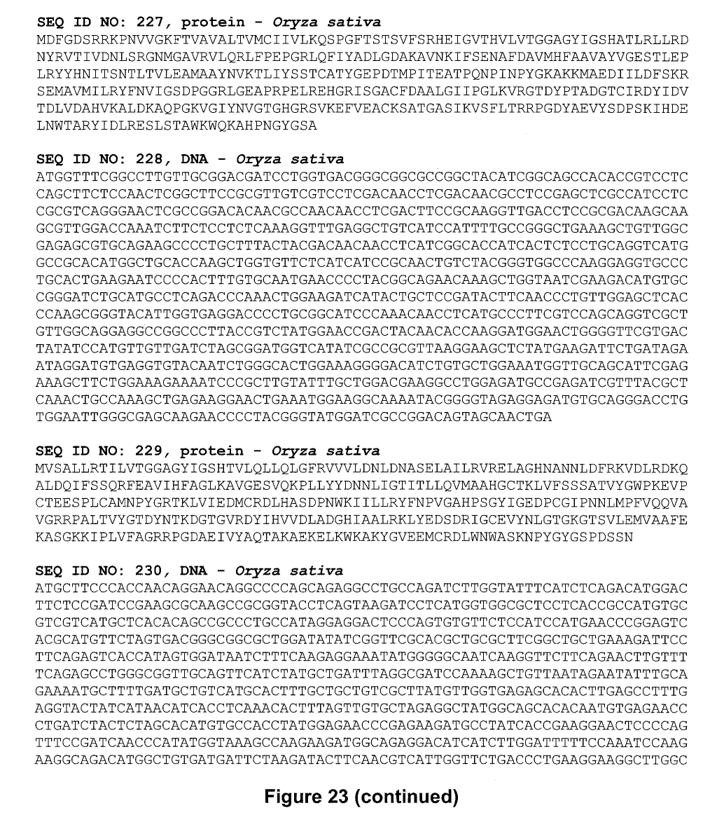

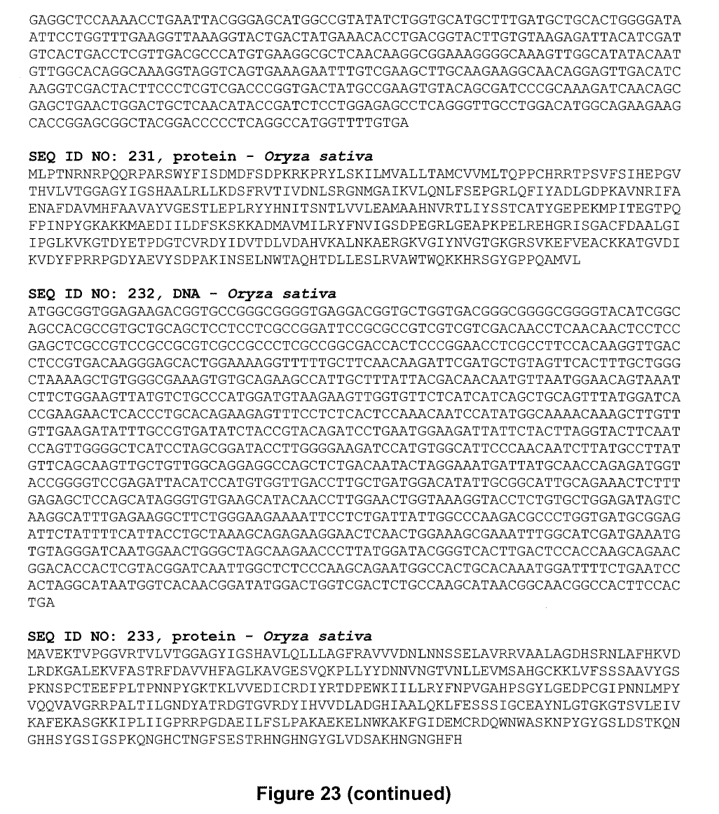

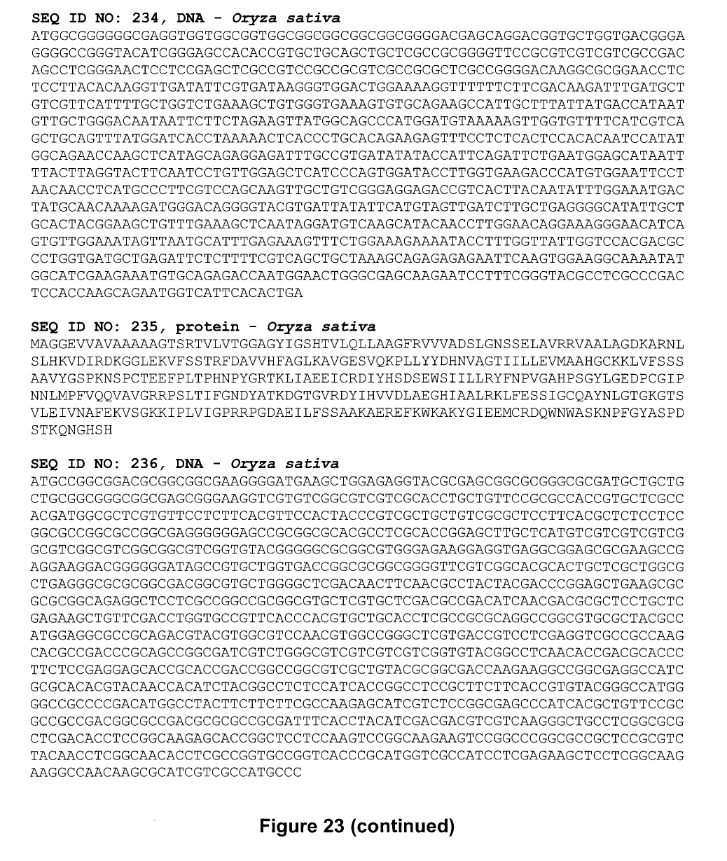

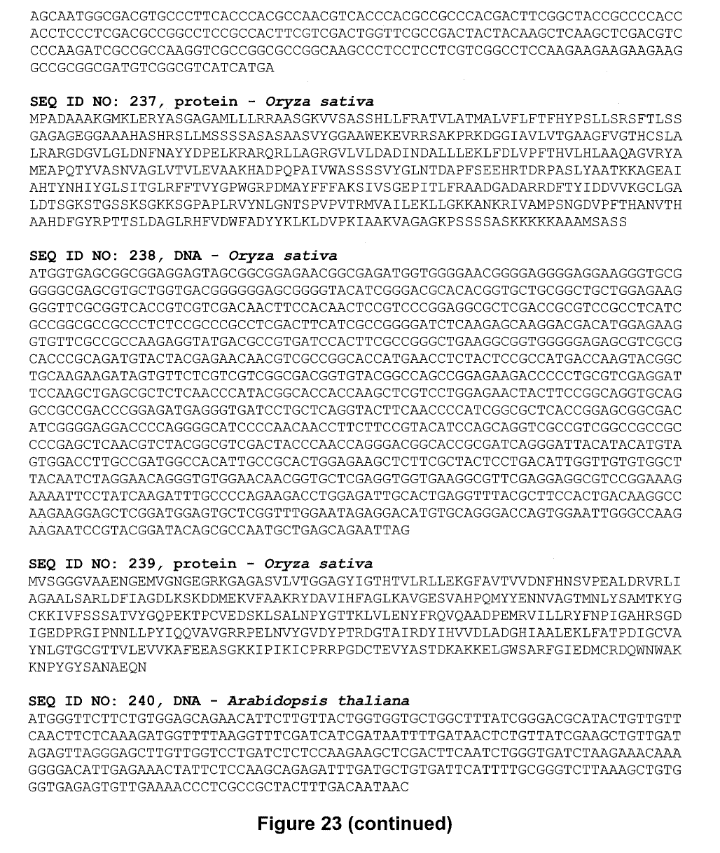

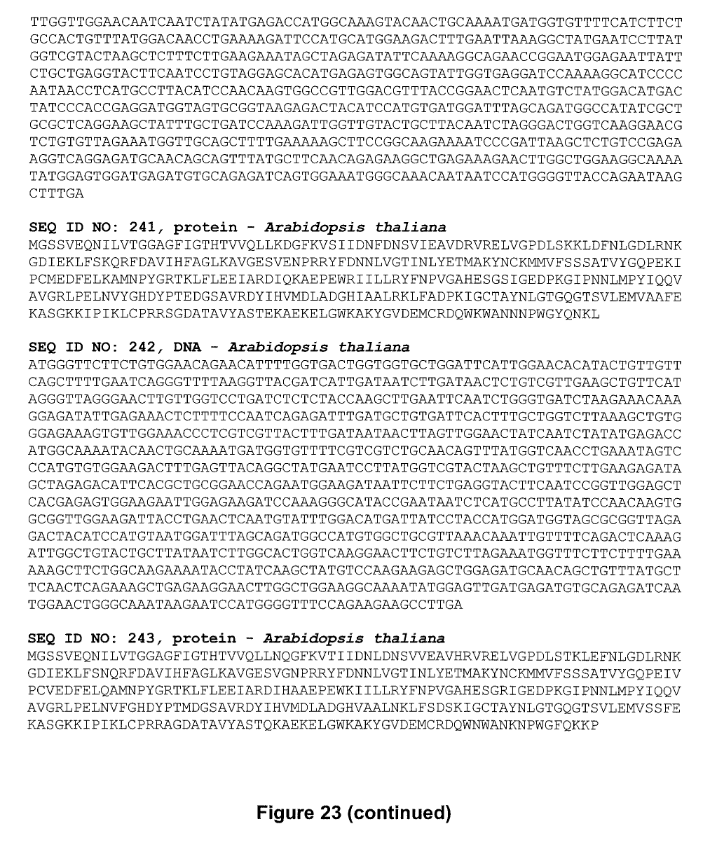

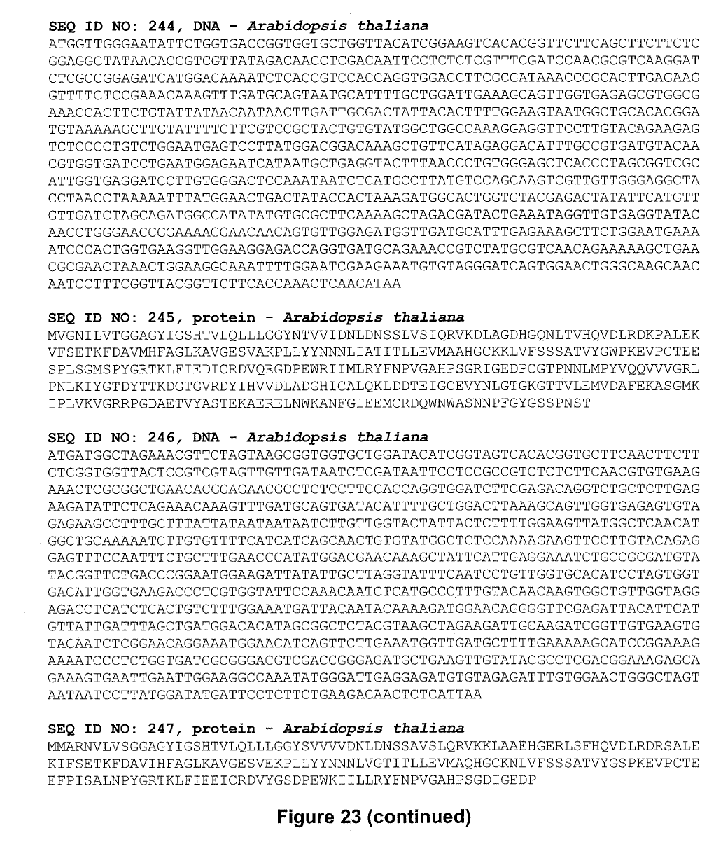

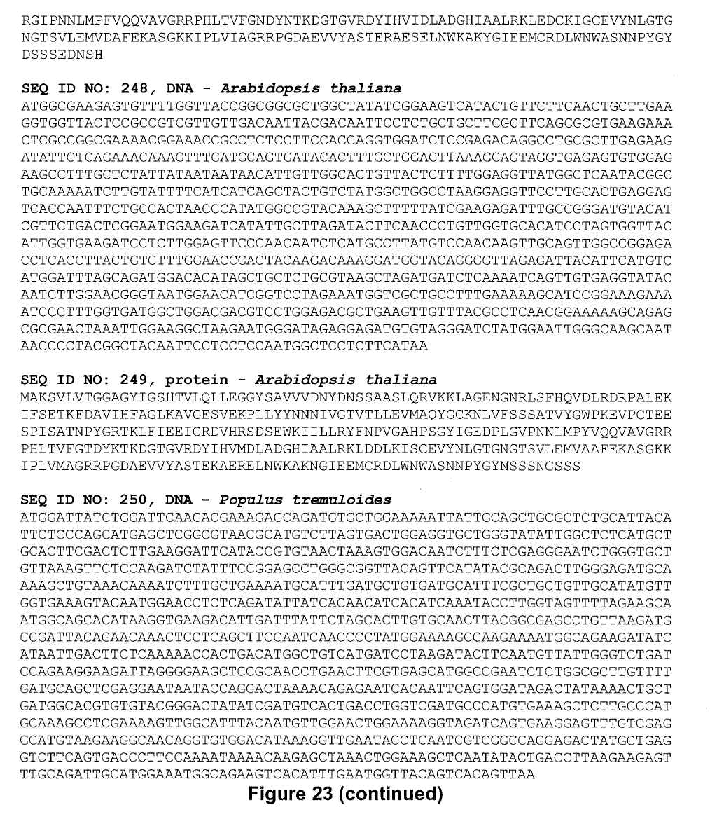

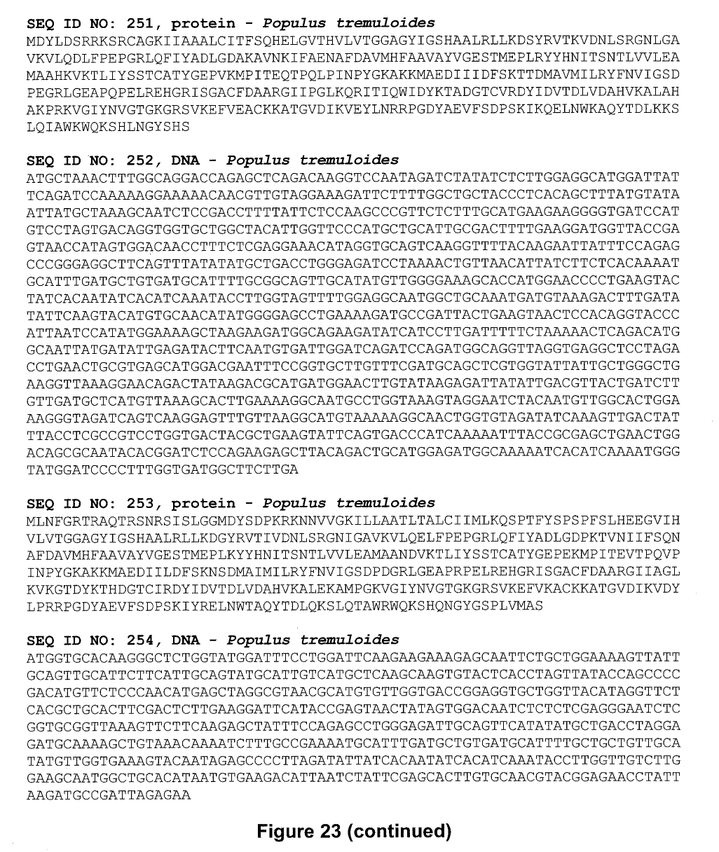

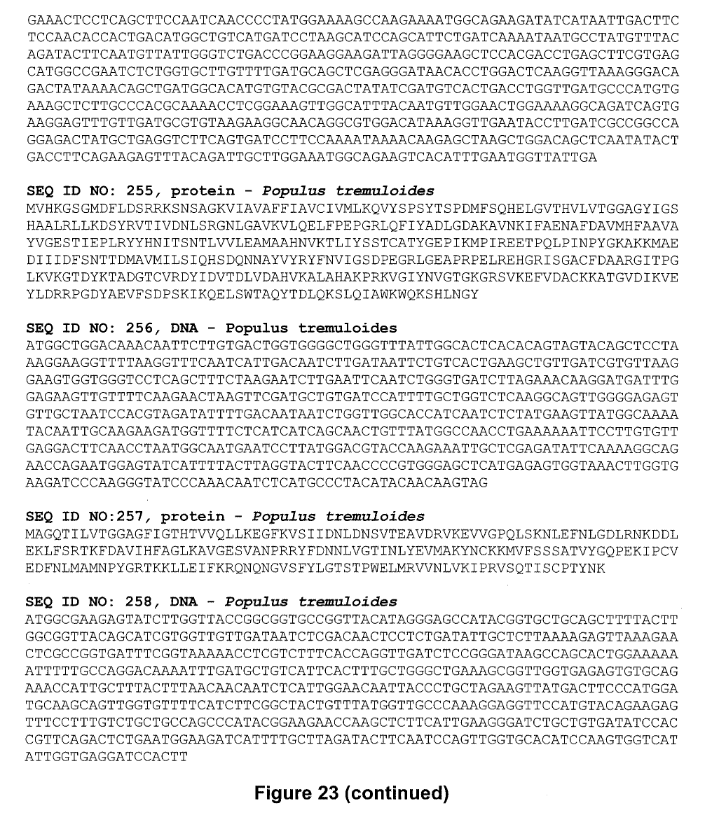

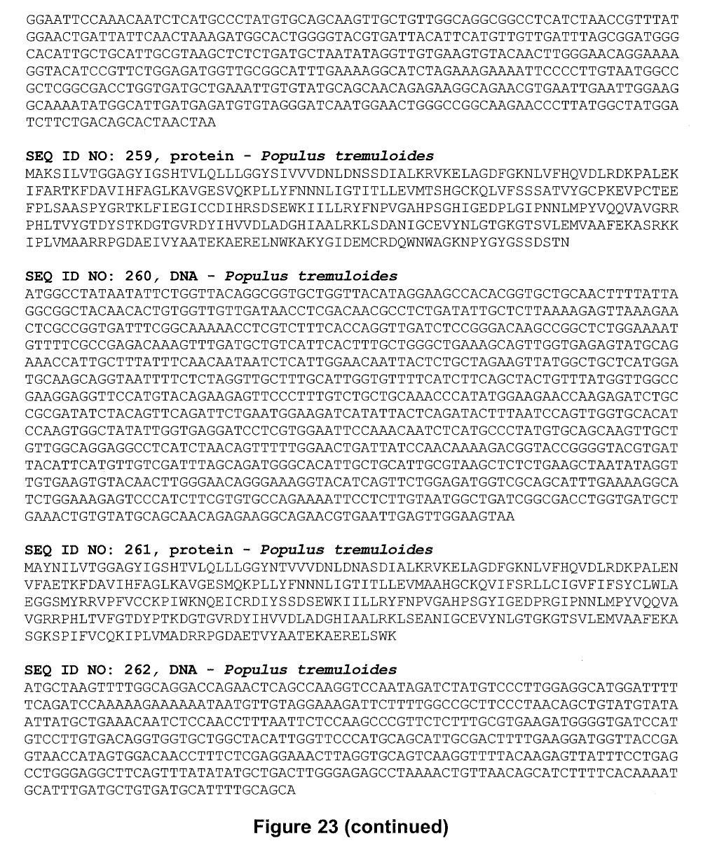

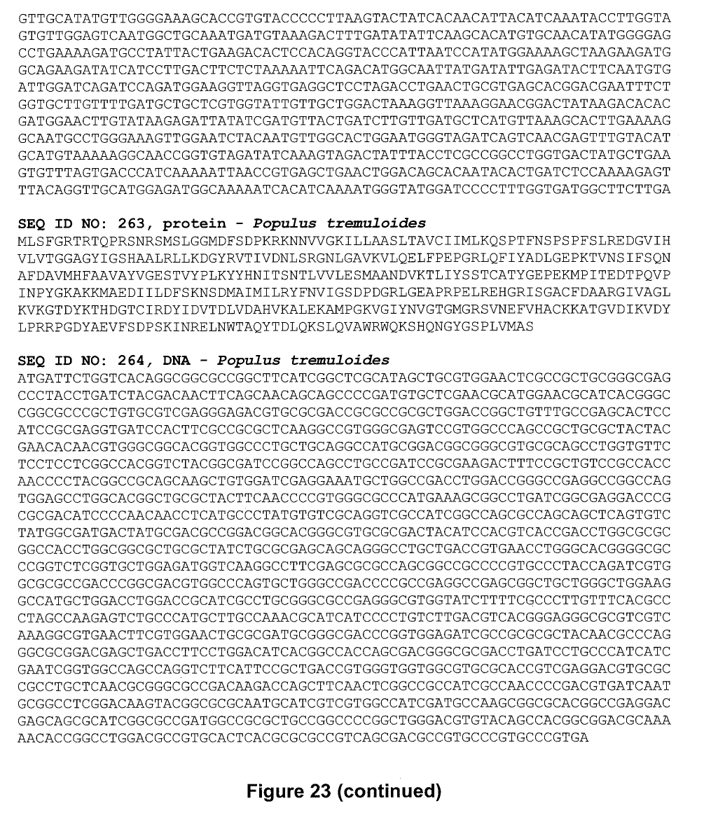

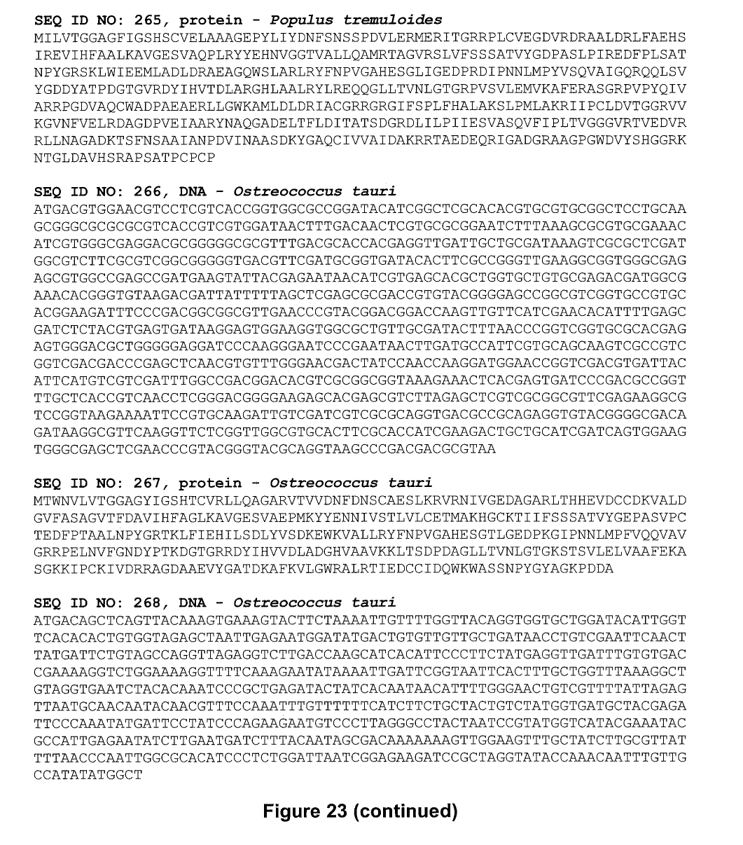

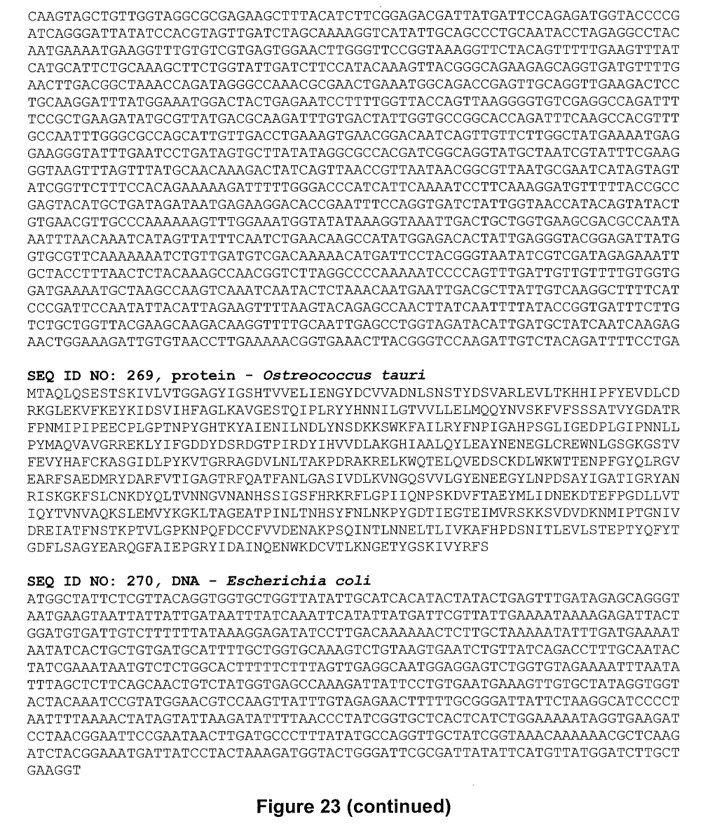

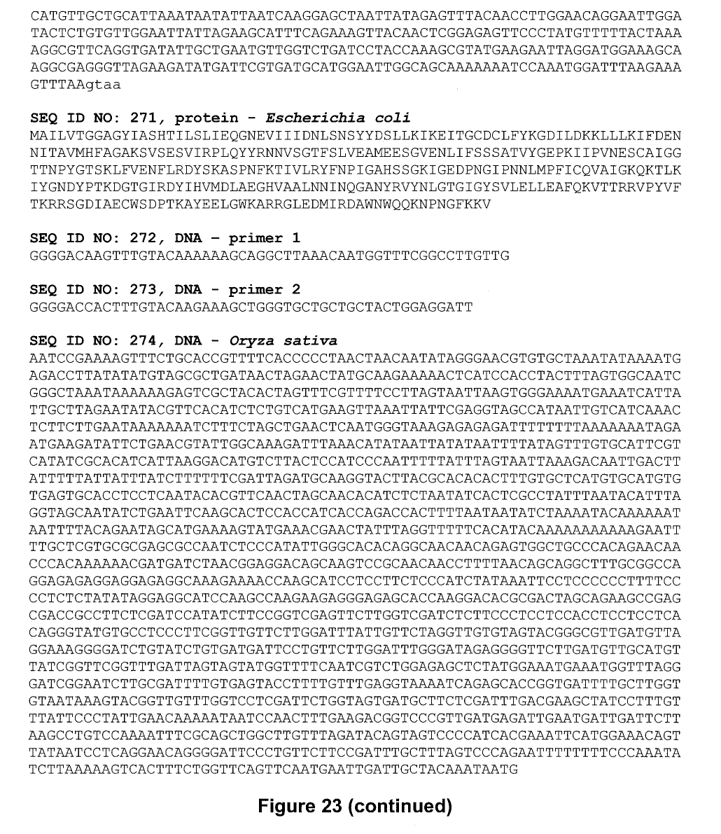

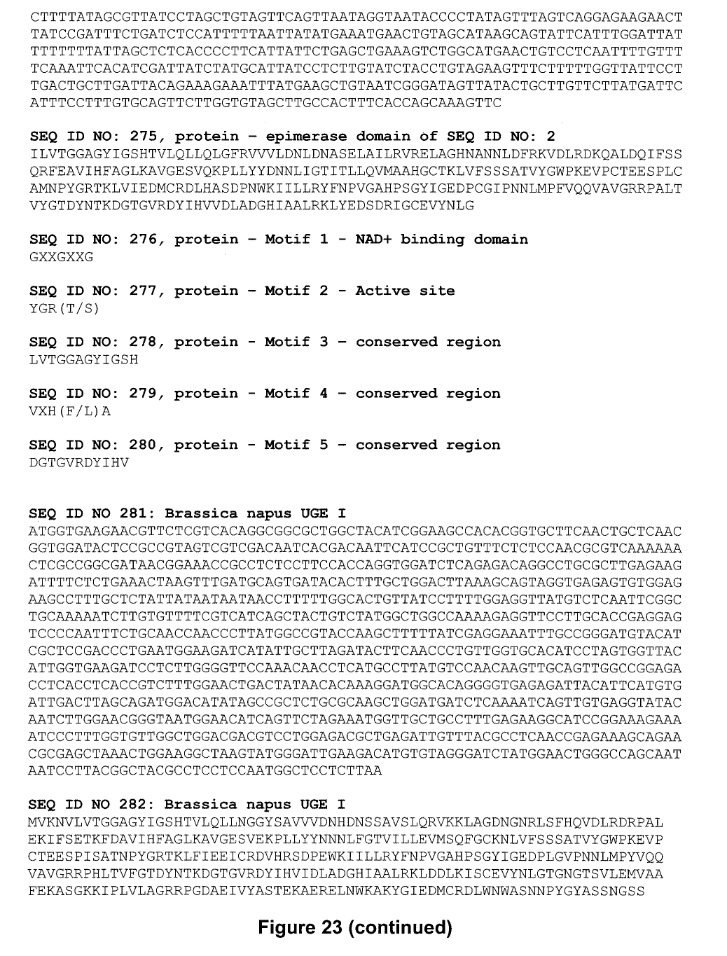

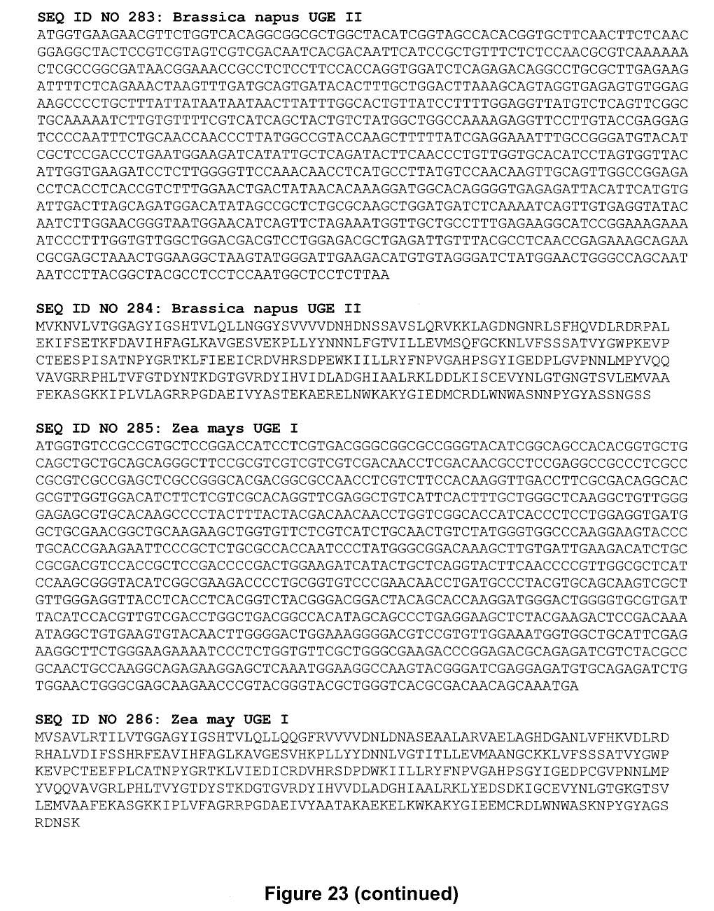

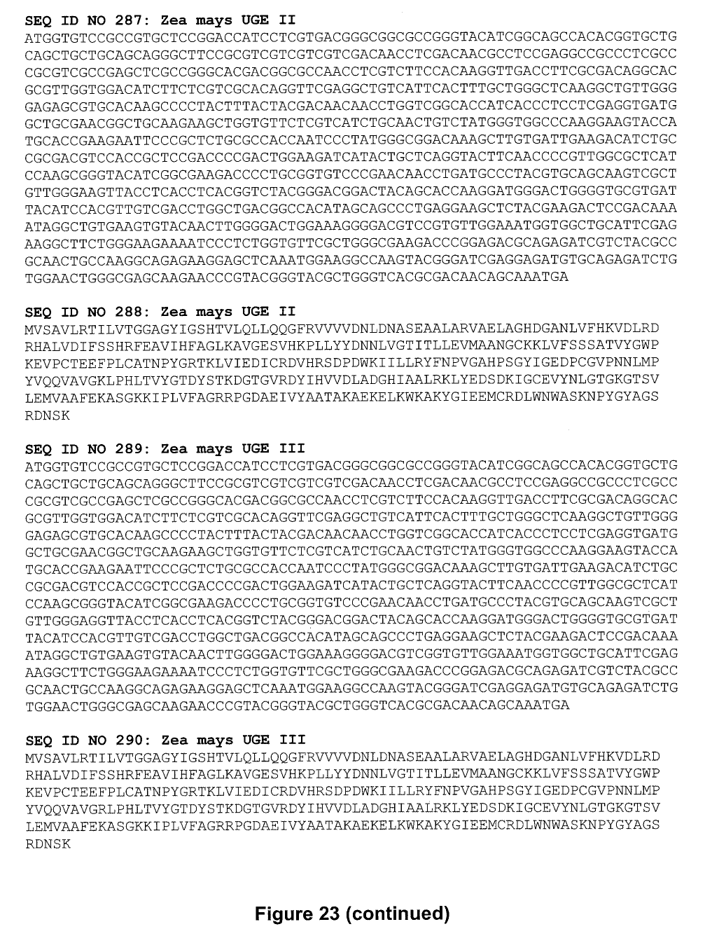

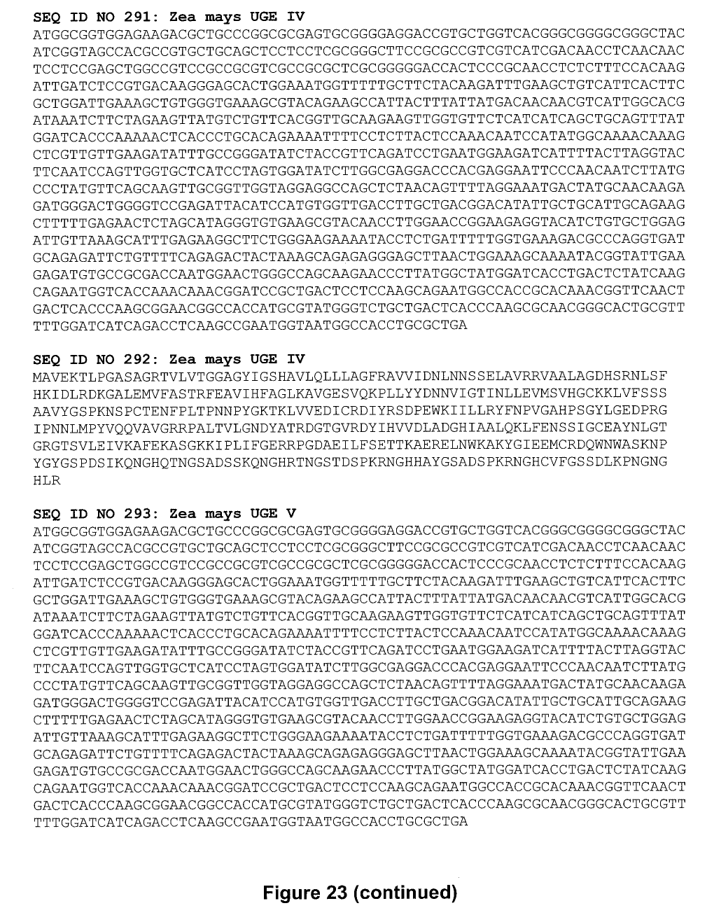

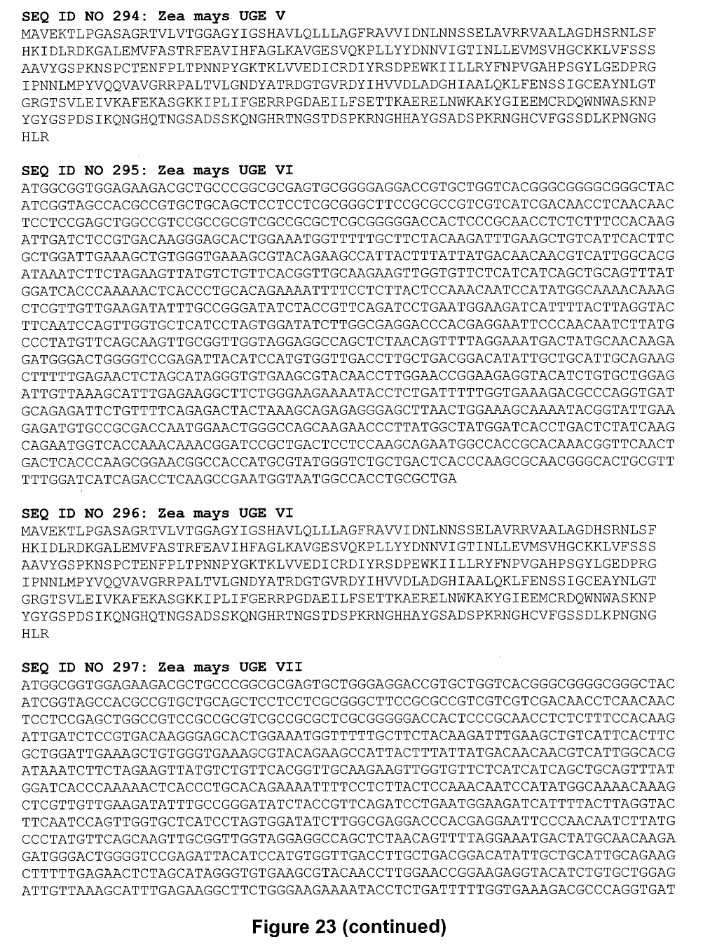

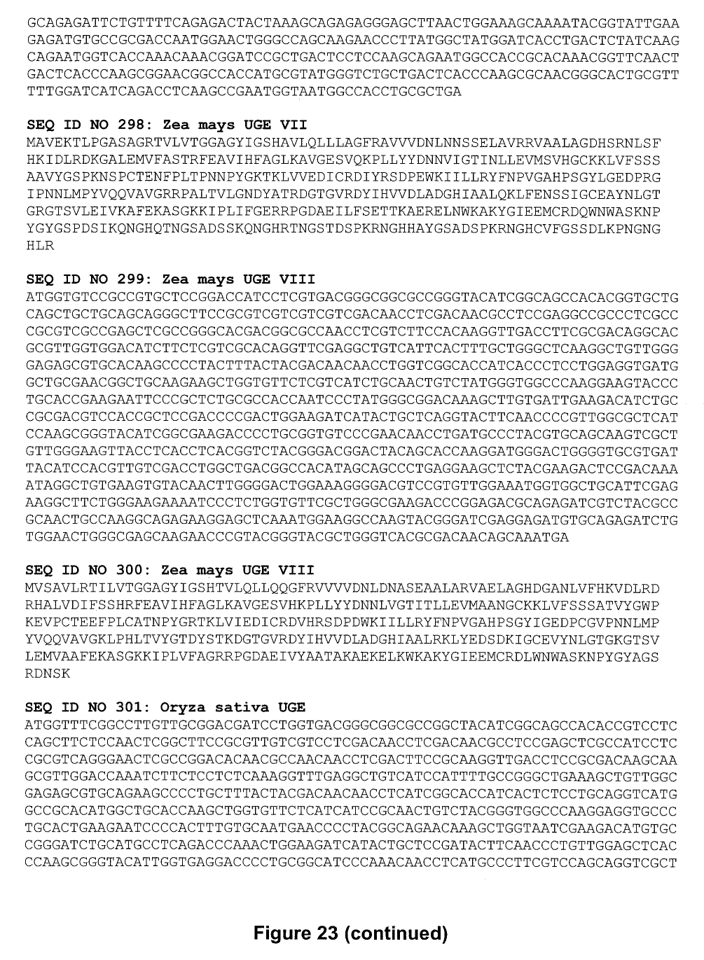

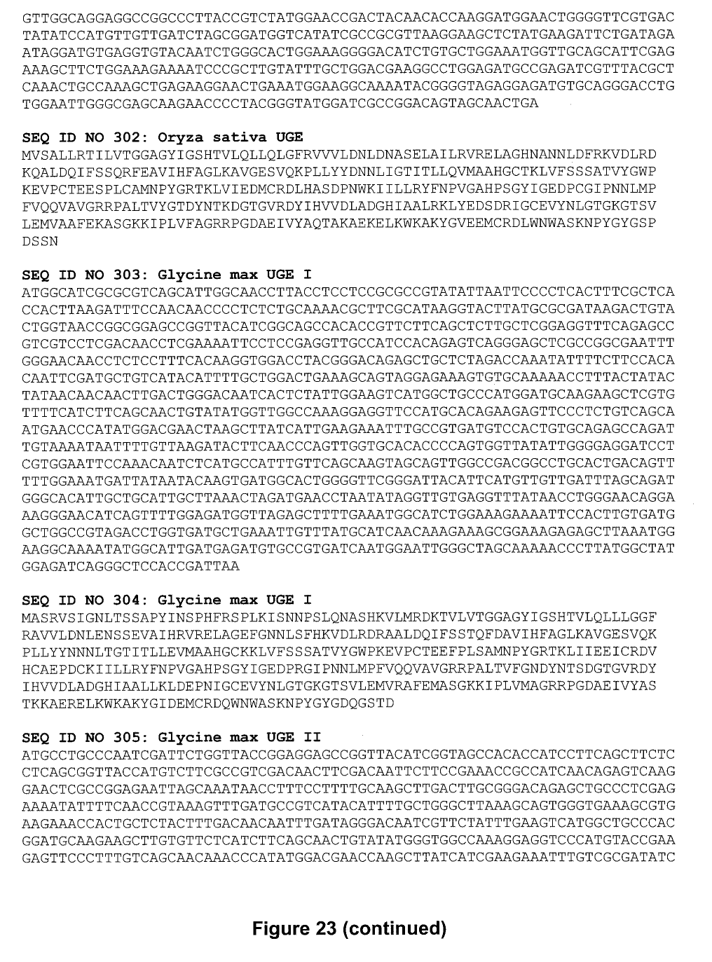

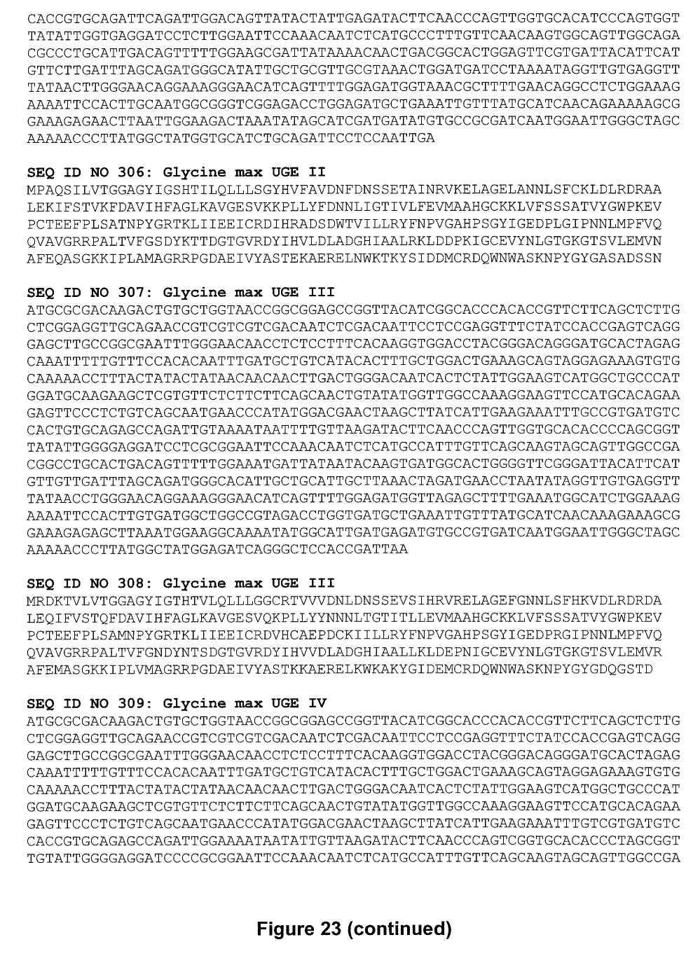

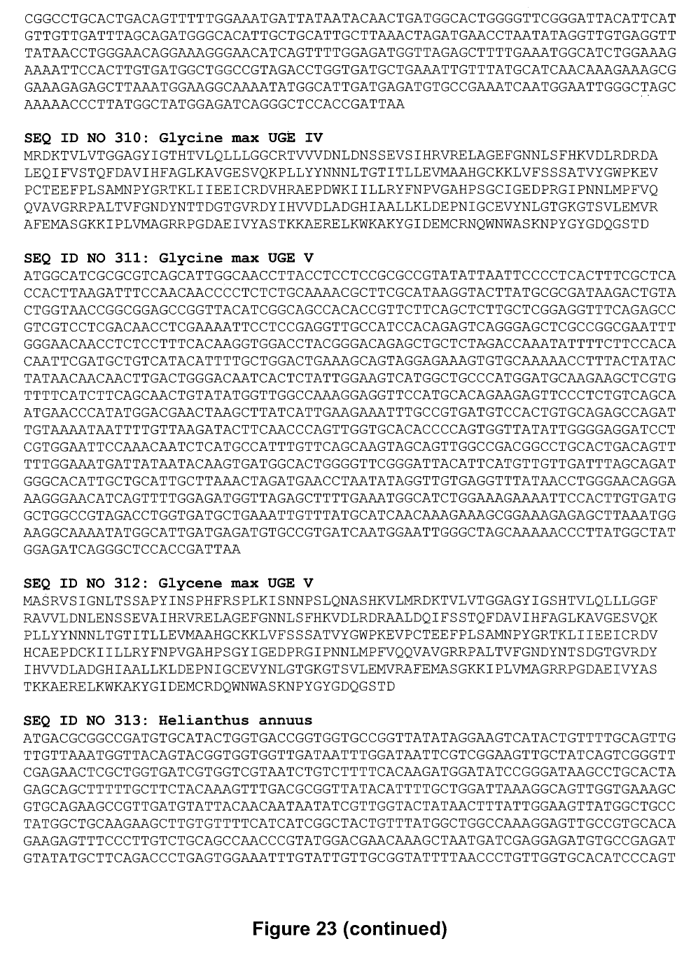

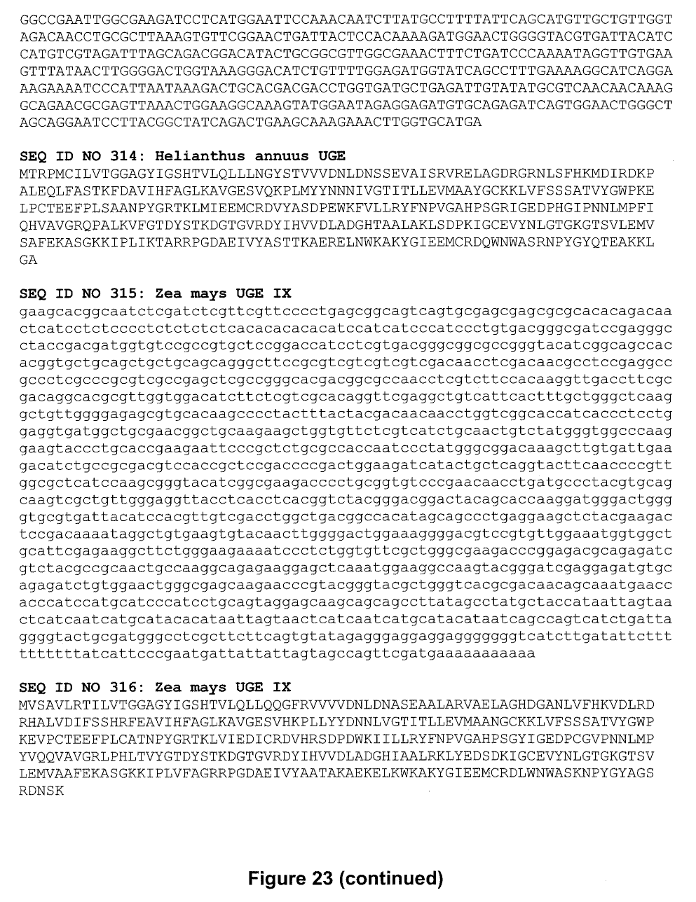

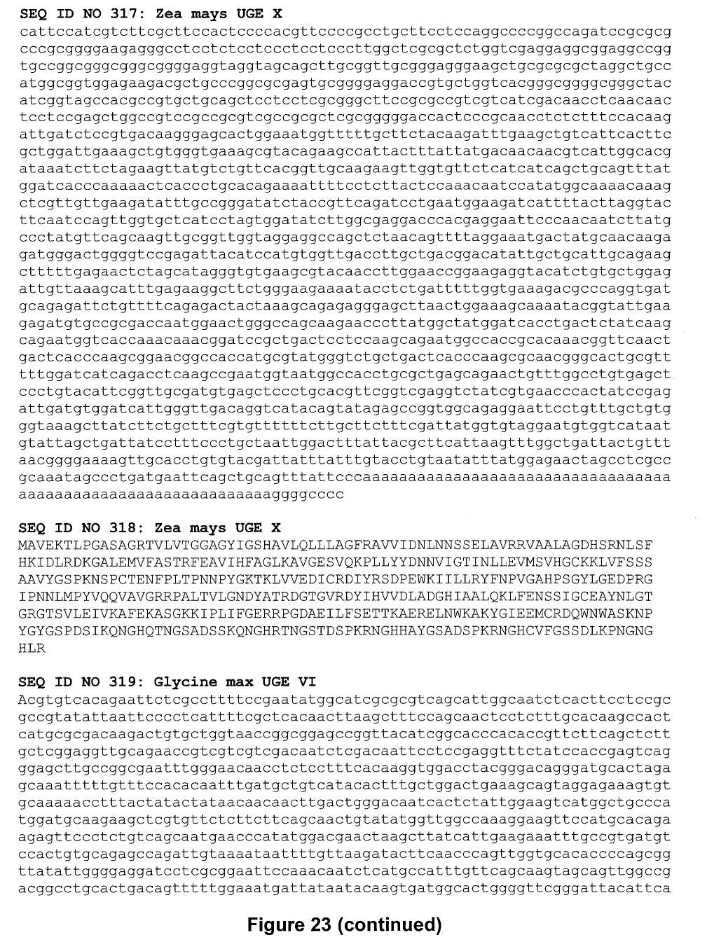

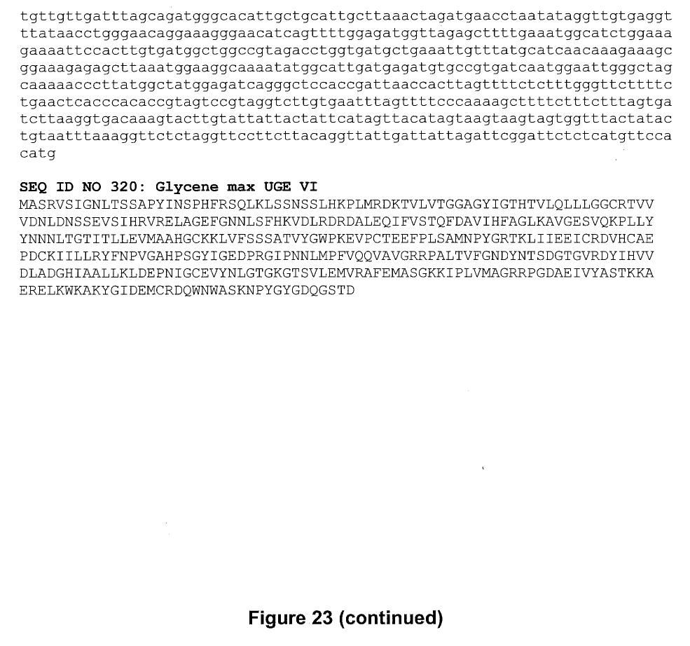

After the LR recombination step, the resulting expression vector pGOS2::UGE (FIG. 22) was transformed into Agrobacterium strain LBA4044 according to methods well known in the art.

### Example 33

### Plant Transformation

### Rice Transformation

The Agrobacterium containing the expression vector was used to transform Oryza sativa plants. Mature dry seeds of the rice japonica cultivar Nipponbare were dehusked. Sterilization was carried out by incubating for one minute in 70% ethanol, followed by 30 minutes in 0.2% HgCl₂, followed by a 6 times 15 minutes wash with sterile distilled water. The sterile seeds were then germinated on a medium containing 2,4-D (callus induction medium). After incubation in the dark for four weeks, embryogenic, scutellum-derived calli were excised and propagated on the same medium. After two weeks, the calli were multiplied or propagated by subculture on the same medium for another 2 weeks. Embryogenic callus pieces were sub-cultured on fresh medium 3 days before co-cultivation (to boost cell division activity).

Agrobacterium strain LBA4404 containing the expression vector was used for co-cultivation. Agrobacterium was inoculated on AB medium with the appropriate antibiotics and cultured for 3 days at 28° C. The bacteria were then collected and suspended in liquid co-cultivation medium to a density (OD₆₀₀) of about 1. The suspension was then transferred to a Petri dish and the calli immersed in the suspension for 15 minutes. The callus tissues were then blotted dry on a filter paper and transferred to solidified, co-cultivation medium and incubated for 3 days in the dark at 25° C. Co-cultivated calli were grown on 2,4-D-containing medium for 4 weeks in the dark at 28° C. in the presence of a selection agent. During this period, rapidly growing resistant callus islands developed. After transfer of this material to a regeneration medium and incubation in the light, the embryogenic potential was released and shoots developed in the next four to five weeks. Shoots were excised from the calli and incubated for 2 to 3 weeks on an auxin-containing medium from which they were transferred to soil. Hardened shoots were grown under high humidity and short days in a greenhouse.

Approximately 35 independent T0 rice transformants were generated for one construct. The primary transformants were transferred from a tissue culture chamber to a greenhouse. After a quantitative PCR analysis to verify copy number of the T-DNA insert, only single copy transgenic plants that exhibit tolerance to the selection agent were kept for harvest of T1 seed. Seeds were then harvested three to five months after transplanting. The method yielded single locus transformants at a rate of over 50% (Aldemita and Hodges1996, Chan et al. 1993, Hiei et al. 1994).

### Corn Transformation

Transformation of maize (Zea mays) is performed with a modification of the method described by Ishida et al. (1996) Nature Biotech 14(6): 745-50. Transformation is genotype-dependent in corn and only specific genotypes are amenable to transformation and regeneration. The inbred line A188 (University of Minnesota) or hybrids with A188 as a parent are good sources of donor material for transformation, but other genotypes can be used successfully as well. Ears are harvested from corn plant approximately 11 days after pollination (DAP) when the length of the immature embryo is about 1 to 1.2 mm. Immature embryos are cocultivated with Agrobacterium tumefaciens containing the expression vector, and transgenic plants are recovered through organogenesis. Excised embryos are grown on callus induction medium, then maize regeneration medium, containing the selection agent (for example imidazolinone but various selection markers can be used). The Petri plates are incubated in the light at 25° C. for 2-3 weeks, or until shoots develop. The green shoots are transferred from each embryo to maize rooting medium and incubated at 25° C. for 2-3 weeks, until roots develop. The rooted shoots are transplanted to soil in the greenhouse. T1 seeds are produced from plants that exhibit tolerance to the selection agent and that contain a single copy of the T-DNA insert.

### Wheat Transformation

Transformation of wheat is performed with the method described by Ishida et al. (1996) Nature Biotech 14(6): 745-50. The cultivar Bobwhite (available from CIMMYT, Mexico) is commonly used in transformation. Immature embryos are co-cultivated with Agrobacterium tumefaciens containing the expression vector, and transgenic plants are recovered through organogenesis. After incubation with Agrobacterium, the embryos are grown in vitro on callus induction medium, then regeneration medium, containing the selection agent (for example imidazolinone but various selection markers can be used). The Petri plates are incubated in the light at 25° C. for 2-3 weeks, or until shoots develop. The green shoots are transferred from each embryo to rooting medium and incubated at 25° C. for 2-3 weeks, until roots develop. The rooted shoots are transplanted to soil in the greenhouse. T1 seeds are produced from plants that exhibit tolerance to the selection agent and that contain a single copy of the T-DNA insert.

### Soybean Transformation

Soybean is transformed according to a modification of the method described in the Texas A&M U.S. Pat. No. 5,164,310. Several commercial soybean varieties are amenable to transformation by this method. The cultivar Jack (available from the Illinois Seed foundation) is commonly used for transformation. Soybean seeds are sterilised for in vitro sowing. The hypocotyl, the radicle and one cotyledon are excised from seven-day old young seedlings. The epicotyl and the remaining cotyledon are further grown to develop axillary nodes. These axillary nodes are excised and incubated with Agrobacterium tumefaciens containing the expression vector. After the cocultivation treatment, the explants are washed and transferred to selection media. Regenerated shoots are excised and placed on a shoot elongation medium. Shoots no longer than 1 cm are placed on rooting medium until roots develop. The rooted shoots are transplanted to soil in the greenhouse. T1 seeds are produced from plants that exhibit tolerance to the selection agent and that contain a single copy of the T-DNA insert.

### Rapeseed/Canola Transformation

Cotyledonary petioles and hypocotyls of 5-6 day old young seedling are used as explants for tissue culture and transformed according to Babic et al. (1998, Plant Cell Rep 17: 183-188). The commercial cultivar Westar (Agriculture Canada) is the standard variety used for transformation, but other varieties can also be used. Canola seeds are surface-sterilized for in vitro sowing. The cotyledon petiole explants with the cotyledon attached are excised from the in vitro seedlings, and inoculated with Agrobacterium (containing the expression vector) by dipping the cut end of the petiole explant into the bacterial suspension. The explants are then cultured for 2 days on MSBAP-3 medium containing 3 mg/l BAP, 3% sucrose, 0.7% Phytagar at 23° C., 16 hr light. After two days of co-cultivation with Agrobacterium, the petiole explants are transferred to MSBAP-3 medium containing 3 mg/l BAP, cefotaxime, carbenicillin, or timentin (300 mg/l) for 7 days, and then cultured on MSBAP-3 medium with cefotaxime, carbenicillin, or timentin and selection agent until shoot regeneration. When the shoots are 5-10 mm in length, they are cut and transferred to shoot elongation medium (MSBAP-0.5, containing 0.5 mg/l BAP). Shoots of about 2 cm in length are transferred to the rooting medium (MS0) for root induction. The rooted shoots are transplanted to soil in the greenhouse. T1 seeds are produced from plants that exhibit tolerance to the selection agent and that contain a single copy of the T-DNA insert.

### Alfalfa Transformation

A regenerating clone of alfalfa (Medicago sativa) is transformed using the method of (McKersie et al., 1999 Plant Physiol 119: 839-847). Regeneration and transformation of alfalfa is genotype dependent and therefore a regenerating plant is required. Methods to obtain regenerating plants have been described. For example, these can be selected from the cultivar Rangelander (Agriculture Canada) or any other commercial alfalfa variety as described by Brown DCW and A Atanassov (1985. Plant Cell Tissue Organ Culture 4: 111-112). Alternatively, the RA3 variety (University of Wisconsin) has been selected for use in tissue culture (Walker et al., 1978 Am J Bot 65:654-659). Petiole explants are cocultivated with an overnight culture of Agrobacterium tumefaciens C58C1 pMP90 (McKersie et al., 1999 Plant Physiol 119: 839-847) or LBA4404 containing the expression vector. The explants are cocultivated for 3 d in the dark on SH induction medium containing 288 mg/L Pro, 53 mg/L thioproline, 4.35 g/L K2SO4, and 100 μm acetosyringinone. The explants are washed in half-strength Murashige-Skoog medium (Murashige and Skoog, 1962) and plated on the same SH induction medium without acetosyringinone but with a suitable selection agent and suitable antibiotic to inhibit Agrobacterium growth. After several weeks, somatic embryos are transferred to BOi2Y development medium containing no growth regulators, no antibiotics, and 50 g/L sucrose. Somatic embryos are subsequently germinated on half-strength Murashige-Skoog medium. Rooted seedlings were transplanted into pots and grown in a greenhouse. T1 seeds are produced from plants that exhibit tolerance to the selection agent and that contain a single copy of the T-DNA insert.

### Example 34

### Phenotypic Evaluation Procedure

### 34.1 Evaluation Setup

Approximately 35 independent T0 rice transformants were generated. The primary transformants were transferred from a tissue culture chamber to a greenhouse for growing and harvest of T1 seed. Six events, of which the T1 progeny segregated 3:1 for presence/absence of the transgene, were retained. For each of these events, approximately 10 T1 seedlings containing the transgene (hetero- and homo-zygotes) and approximately 10 T1 seedlings lacking the transgene (nullizygotes) were selected by monitoring visual marker expression. The transgenic plants and the corresponding nullizygotes were grown side-by-side at random positions. Greenhouse conditions were of shorts days (12 hours light), 28° C. in the light and 22° C. in the dark, and a relative humidity of 70%.

Four T1 events were further evaluated in the T2 generation following the same evaluation procedure as for the T1 generation but with more individuals per event. From the stage of sowing until the stage of maturity the plants were passed several times through a digital imaging cabinet. At each time point digital images (2048×1536 pixels, 16 million colours) were taken of each plant from at least 6 different angles.

### Drought Screen

Plants from T2 seeds were grown in potting soil under normal conditions until they approached the heading stage. They were then transferred to a “dry” section where irrigation was withheld. Humidity probes were inserted in randomly chosen pots to monitor the soil water content (SWC). When SWC went below certain thresholds, the plants were automatically re-watered continuously until a normal level was reached again. The plants were then re-transferred again to normal conditions. The rest of the cultivation (plant maturation, seed harvest) was the same as for plants not grown under abiotic stress conditions. Growth and yield parameters are recorded as detailed for growth under normal conditions.

### Nitrogen Use Efficiency Screen

Rice plants from T2 seeds are grown in potting soil under normal conditions except for the nutrient solution. The pots were watered from transplantation to maturation with a specific nutrient solution containing reduced N nitrogen (N) content, usually between 7 to 8 times less. The rest of the cultivation (plant maturation, seed harvest) was the same as for plants not grown under abiotic stress. Growth and yield parameters are recorded as detailed for growth under normal conditions.

### 34.2 Statistical Analysis: F Test

A two factor ANOVA (analysis of variants) was used as a statistical model for the overall evaluation of plant phenotypic characteristics. An F test was carried out on all the parameters measured of all the plants of all the events transformed with the gene of the present invention. The F test was carried out to check for an effect of the gene over all the transformation events and to verify for an overall effect of the gene, also known as a global gene effect. The threshold for significance for a true global gene effect was set at a 5% probability level for the F test. A significant F test value points to a gene effect, meaning that it is not only the mere presence or position of the gene that is causing the differences in phenotype.

Because two experiments with overlapping events were carried out, a combined analysis was performed. This is useful to check consistency of the effects over the two experiments, and if this is the case, to accumulate evidence from both experiments in order to increase confidence in the conclusion. The method used was a mixed-model approach that takes into account the multilevel structure of the data (i.e. experiment-event-segregants). P values were obtained by comparing likelihood ratio test to chi square distributions.

### 34.3 Parameters Measured

Biomass-Related Parameter Measurement

From the stage of sowing until the stage of maturity the plants were passed several times through a digital imaging cabinet. At each time point digital images (2048×1536 pixels, 16 million colours) were taken of each plant from at least 6 different angles. The plant aboveground area (or leafy biomass) was determined by counting the total number of pixels on the digital images from aboveground plant parts discriminated from the background. This value was averaged for the pictures taken on the same time point from the different angles and was converted to a physical surface value expressed in square mm by calibration. Experiments show that the aboveground plant area measured this way correlates with the biomass of plant parts above ground. The above ground area is the area measured at the time point at which the plant had reached its maximal leafy biomass. The early vigour is the plant (seedling) aboveground area three weeks post-germination. Increase in root biomass is expressed as an increase in total root biomass (measured as maximum biomass of roots observed during the lifespan of a plant); or as an increase in the root/shoot index (measured as the ratio between root mass and shoot mass in the period of active growth of root and shoot).

Early vigour was determined by counting the total number of pixels from aboveground plant parts discriminated from the background. This value was averaged for the pictures taken on the same time point from different angles and was converted to a physical surface value expressed in square mm by calibration. The results described below are for plants three weeks post-germination.

### Seed-Related Parameter Measurements

The mature primary panicles were harvested, counted, bagged, barcode-labelled and then dried for three days in an oven at 37° C. The panicles were then threshed and all the seeds were collected and counted. The filled husks were separated from the empty ones using an air-blowing device. The empty husks were discarded and the remaining fraction was counted again. The filled husks were weighed on an analytical balance. The number of filled seeds was determined by counting the number of filled husks that remained after the separation step. The total seed yield was measured by weighing all filled husks harvested from a plant. Total seed number per plant was measured by counting the number of husks harvested from a plant. Thousand Kernel Weight (TKW) is extrapolated from the number of filled seeds counted and their total weight. The Harvest Index (HI) in the present invention is defined as the ratio between the total seed yield and the above ground area (mm²), multiplied by a factor 10⁶. The total number of flowers per panicle as defined in the present invention is the ratio between the total number of seeds and the number of mature primary panicles. The seed fill rate as defined in the present invention is the proportion (expressed as a %) of the number of filled seeds over the total number of seeds (or florets).

### Example 35

### Results of the Phenotypic Evaluation of the Transgenic Plants

The results of the evaluation of transgenic rice plants expressing a UGE nucleic acid under non-stress conditions and under drought-stress (water limiting) conditions are presented below. An increase of at least 5% was observed for total seed yield, number of filled seeds, fill rate, and harvest index. Table E shows the results of the phenotypic evaluation.

TABLE E
Result of the phenotypic evaluation of transgenic plants.

| | non-stress | drought-stress |
|---|---|---|
| | % Difference | % Difference |
| Yield trait | transgenic/control* | transgenic/control* |
| See yield (grams) | 15 | 45 |
| Nr of filled seed | 13 | 42 |
| Fill rate | 5 | 45 |
| Harvest index | 10 | 49 |

*Increase measured in the transgenic plants versus the corresponding control nullyzygous plants, expressed in percentage (%).

1. A method for enhancing yield-related traits in plants relative to control plants, comprising modulating expression in a plant of a nucleic acid encoding a yield increasing polypeptide selected from the group consisting of:
(i) a bHLH6-like (basic Helix-Loop-Helix 6-like) protein,
(ii) a GRP (Growth Regulating Protein), wherein said GRP is selected from the group consisting of:
(a) an RrmJ/FtsS ribosomal RNA methyltransferase polypeptide (RrmJ/FtsJ polypeptide),
(b) a basic-helix-loop-helix 4 (bHLH4) polypeptide, and
(c) an isopentenyl transferase (IPT) polypeptide
(iii) an STO (Salt Tolerance) (Salt Tolerance) protein, and
(iv) a UGE (UDP-Glucose-4-Epimerase or UDP-Gal-4-Epimerase), polypeptide.

2. The method according to claim 1, wherein said yield increasing polypeptide is selected from the group consisting of:
(i) a polypeptide with the activity of any of the polypeptides as depicted in table A1, A2, A3, A4, A5 and/or A6;
(ii) a polypeptide with a sequence according to any of the polypeptides as depicted in table A1, A2, A3, A4, A5 and/or A6;
(iii) a polypeptide encoded by any of the nucleic acids as depicted in Table A1, A2, A3, A4, A5 and/or A6 or of a nucleic acid capable of hybridising hybridizing with such a nucleic acid, and
(iv) a polypeptide comprising at least one of the motifs as depicted in motif 1 to 12 or as shown in the figures.

3. A method for enhancing yield-related traits in plants relative to control plants, comprising modulating expression in a plant of a nucleic acid encoding a bHLH6-like polypeptide, wherein said bHLH6-like polypeptide comprises a HLH domain.

4. The method according to claim 1, wherein said bHLH6-like polypeptide comprises one or more of the following motifs:
(i) Motif 1 (SEQ ID NO: 3),
(ii) Motif 2 (SEQ ID NO: 4),
(iii) Motif 3(SEQ ID NO: 5), and
(iv) Motif 7 (SEQ ID NO: 9) or a sequence that has at least 80% sequence identity to SEQ ID NO: 9.

5. The method according to claim 3, wherein said modulated expression is effected by introducing and expressing in a plant a nucleic acid encoding a bHLH6-like polypeptide.

6. The method according to claim 1, wherein said nucleic acid encoding a bHLH6-like polypeptide encodes any one of the proteins listed in Table A1 or is a portion of such a nucleic acid, or a nucleic acid capable of hybridizing with such a nucleic acid.

7. The method according to claim 1, wherein said nucleic acid sequence encodes an orthologue or paralogue of any of the proteins given in Table A1.

8. The method according to claim 1, wherein said enhanced yield-related traits comprise increased yield, increased emergence vigour, and/or increased seed yield relative to control plants.

9. The method according to claim 1, wherein said enhanced yield-related traits are obtained under non-stress conditions.

10. The method according to claim 5, wherein said nucleic acid is operably linked to a constitutive promoter, preferably to a GOS2 promoter, most preferably to a GOS2 promoter from rice.

11. The method according to claim 1, wherein said nucleic acid encoding a bHLH6-like polypeptide is of plant origin, preferably from a dicotyledonous plant, further preferably from the family Brassicaceae, more preferably from the genus Arabidopsis, most preferably from Arabidopsis thaliana.

12. A plant or part thereof, including seeds, obtained by the method according to claim 1, wherein said plant or part thereof comprises a recombinant nucleic acid encoding a bHLH6-like polypeptide.

13. A construct comprising:
(i) a nucleic acid encoding a bHLH6-like polypeptide as defined in claim 4;
(ii) one or more control sequences capable of driving expression of the nucleic acid sequence of (a); and optionally
(iii) a transcription termination sequence.

14. The construct according to claim 13, wherein one of said control sequences is a constitutive promoter, preferably a GOS2 promoter, most preferably a GOS2 promoter from rice.

15. A method for making plants having increased yield, increased emergence vigour, and/or increased seed yield relative to control plants, comprising utilizing the construct according to claim 13.

16. A plant, plant part or plant cell transformed with the construct according to claim 13.

17. A method for the production of a transgenic plant having increased yield, increased biomass, and/or increased seed yield relative to control plants, comprising:
(i) introducing and expressing in a plant a nucleic acid encoding a bHLH6-like polypeptide as defined in claim 4; and
(ii) cultivating the plant cell under conditions promoting plant growth and development.

18. A transgenic plant having increased yield, increased biomass, and/or increased seed yield, relative to control plants, resulting from modulated expression of a nucleic acid encoding a bHLH6-like polypeptide as defined in claim 4, or a transgenic plant cell derived from said transgenic plant.

19. The transgenic plant according to claim 18, or a transgenic plant cell derived thereof, wherein said plant is a crop plant or a monocot or a cereal, such as rice, maize, wheat, barley, millet, rye, triticale, sorghum emmer, spelt, secale, einkorn, teff, milo and oats.

20. Harvestable parts of the plant according to claim 19, wherein said harvestable parts are preferably shoot biomass and/or seeds.

21. Products derived from the plant according to claim 19 and/or from harvestable parts of said plant.

22. A method for increasing yield, increasing seed yield and/or shoot biomass in plants, relative to control plants, comprising utilizing a nucleic acid encoding a bHLH6-like polypeptide.
